(19) United States
(12) Patent Application Publication
(10) Pub. No.: US 2020/0303663 A1
(43) Pub. Date: Sep. 24, 2020

(54) CONDENSED-CYCLIC COMPOUND AND ORGANIC LIGHT-EMITTING DEVICE INCLUDING THE SAME

(71) Applicant: Samsung Electronics Co., Ltd., Suwon-si (KR)

(72) Inventors: Soonok JEON, Suwon-si (KR); Yeonsook CHUNG, Seoul (KR)

(21) Appl. No.: 16/583,431

(22) Filed: Sep. 26, 2019

(30) Foreign Application Priority Data
Mar. 19, 2019 (KR) 10-2019-0031464

(51) Int. Cl. H01L 51/00; C07D 405/14; C09K 11/06

(52) U.S. Cl. CPC H01L 51/0073; C07D 405/14; H01L 51/5012; H01L 51/0072; C09K 11/06

(57) ABSTRACT

A condensed-cyclic compound represented by Formula 1, wherein Y₁₁ is a group represented by Formulae 2-1 or 2-2:

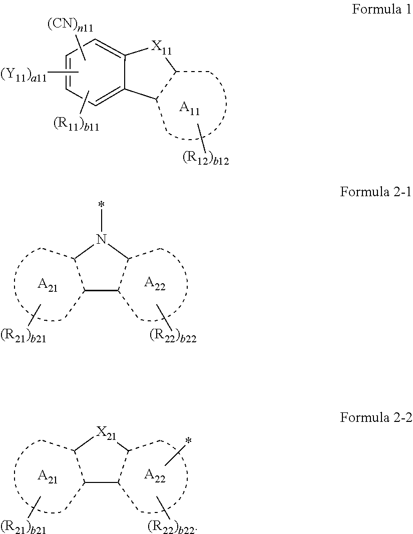

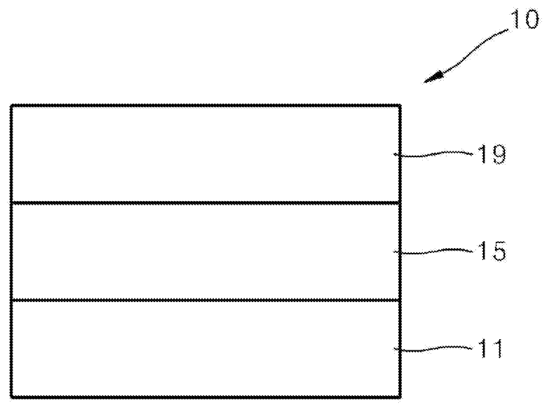

### CROSS-REFERENCE TO RELATED APPLICATION

This application claims priority to and the benefit of Korean Patent Application No. 10-2019-0031464, filed on Mar. 19, 2019, in the Korean Intellectual Property Office, and all the benefits accruing therefrom under 35 U.S.C. § 119, the content of which in its entirety is herein incorporated by reference.

### BACKGROUND

### 1. Field

One or more embodiments relate to a condensed-cyclic compound and an organic light-emitting device including the same.

### 2. Description of the Related Art

Organic light-emitting devices are self-emissive devices and, compared with devices of the related art, have a wide viewing angle, a high contrast ratio, and a short response time, and exhibit excellent characteristics in terms of luminance, driving voltage, and response speed.

In an example, an organic light-emitting device includes an anode, a cathode, and an organic layer between the anode and the cathode, wherein the organic layer includes an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. The holes and the electrons, which are carriers, recombine in the emission layer to produce excitons. These excitons transition from an excited state to a ground state, thereby generating light.

### SUMMARY

One or more embodiments include a condensed-cyclic compound having excellent delayed fluorescence emission characteristics and an organic light-emitting device that has high efficiency and/or a long lifespan due to the inclusion of the condensed-cyclic compound.

Additional aspects will be set forth in part in the description which follows and, in part, will be apparent from the description, or may be learned by practice of the presented embodiments.

According to one or more embodiments, a condensed-cyclic compound represented by Formula 1 is provided:

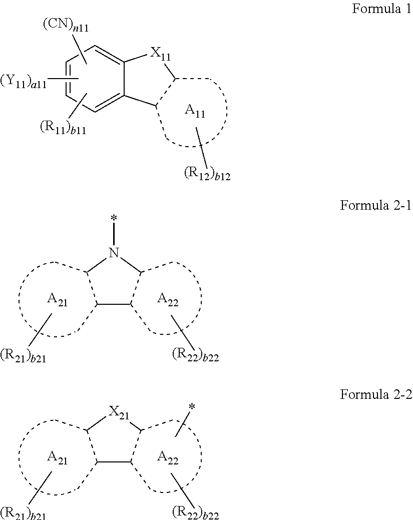

wherein, in Formulae 1, 2-1, and 2-2,
n11 may be 1 or 2;
Y₁₁ may be a group represented by Formulae 2-1 or 2-2;
a11 may be 2 or 3;
X₁₁ may be O, S, N(R₁₆), or C(R₁₆)(R₁₇);
A₁₁, A₂₁, and A₂₂ may each independently be a C₅-C₆₀ carbocyclic group or a C₁-C₆₀ heterocyclic group;
X₂₁ may be O, S, N(R₂₃), or C(R₂₃)(R₂₄);
R₁₁, R₁₂, and R₂₁ to R₂₄ may each independently be hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b11 may be 0 or 1;
b12, b21, and b22 may each independently be 1, 2, 3, 4, 5, 6, 7, 8, 9, or 10;
the sum of n11, a11, and b11 may be 4;
Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, a C₁-C₆₀ alkyl group which is substituted with deuterium, —F, a cyano group, a C₁-C₆₀ alkyl group, a C₆-C₆₀ aryl group which is substituted with deuterium, —F, a cyano group, a C₁-C₆₀ alkyl group, a C₆-C₆₀ aryl group, or a combination thereof, an unsubstituted C₆-C₆₀ aryl group, or a combination thereof; and
* indicates a binding site to a neighboring atom.

According to one or more embodiments, an organic light-emitting device includes a first electrode; a second electrode; and an organic layer including an emission layer between the first electrode and the second electrode, wherein the organic layer includes the condensed-cyclic compound.

### BRIEF DESCRIPTION OF THE DRAWING

These and/or other aspects will become apparent and more readily appreciated from the following description of the embodiments, taken in conjunction with

FIGURE which shows a schematic cross-sectional view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein.

Accordingly, the embodiments are merely described below, by referring to the FIGURES, to explain aspects of the present description.

It will be understood that when an element is referred to as being “on” another element, it can be directly on the other element or intervening elements may be present therebetween. In contrast, when an element is referred to as being “directly on” another element, there are no intervening elements present.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. Expressions such as “at least one of,” when preceding a list of elements, modify the entire list of elements and do not modify the individual elements of the list. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10%, or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

The condensed-cyclic compound may be represented by Formula 1.

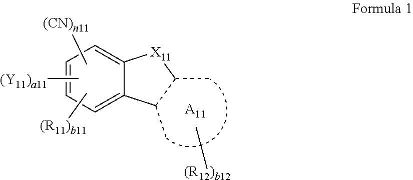

n11 in Formula 1 indicates the substitution number of cyano groups (CN), and n11 may be 1 or 2.

For example, n11 in Formula 1 may be 1, but embodiments are not limited thereto.

Y₁₁ in Formula 1 may be a group represented by one of Formulae 2-1 or 2-2:

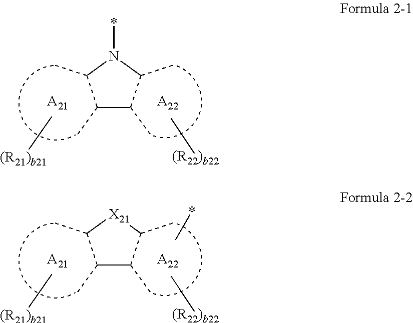

In Formulae 2-1 and 2-2, A₂₁, A₂₂, X₂₁, R₂₁, R₂₂, b21 and b22 may be understood by referring to the related description to be presented later, and * indicates a binding site to a neighboring atom.

a11 in Formula 1 indicates the substitution number of Y₁₁, and a11 may be 2 or 3. A plurality of Y₁₁(s) may be identical to or different from each other.

X₁₁ in Formula 1 may be O, S, N(R₁₆), or C(R₁₆)(R₁₇), and R₁₆ and R₁₇ may be understood by referring to the related description to be presented later.

For example, X₁₁ in Formula 1 may be O, S, or N(R₁₆), but embodiments are not limited thereto.

A₁₁, A₂₁ and A₂₂ in Formula 1, 2-1 and 2-2 may each independently be a C₅-C₆₀ carbocyclic group, or a C₁-C₆₀ heterocyclic group.

For example, A₁₁, A₂₁ and A₂₂ in Formulae 1, 2-1 and 2-2 may each independently be a benzene group, a naphthalene group, an anthracene group, a phenanthrene group, a phenalene group, a triphenylene group, a pyridine group, a pyrimidine group, a pyrazine group, a pyridazine group, a triazine group, a quinoline group, an isoquinoline group, a 2,6-naphthyridine group, a 1,8-naphthyridine group, a 1,5-naphthyridine group, a 1,6-naphthyridine group, a 1,7-naphthyridine group, a 2,7-naphthyridine group, a quinoxaline group, a phthalazine group, a quinazoline group, a phenanthroline group, a benzoquinoline group, a benzoisoquinoline group, a benzoquinoxaline group, a benzoquinazoline group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an indenopyridine group, an indolopyridine group, a benzofuropyridine group, a benzothienopyridine group, a benzosilolopyridine group, an indenopyrimidine group, an indolopyrimidine group, a benzofuropyrimidine group, a benzothienopyrimidine group or a benzosilolopyrimidine group, but embodiments of the present disclosure are not limited thereto.

In one or more embodiments, A₁₁ in Formulae 1, 2-1, and 2-2 may be a benzene group, or a naphthalene group;

A₂₁ and A₂₂ may each independently be a benzene group, a naphthalene group, a fluorene group, a carbazole group, a dibenzofuran group, or a dibenzothiophene group, but embodiments are not limited thereto.

In one or more embodiments, A₁₁ in Formulae 1, 2-1, and 2-2 may be a benzene group, or a naphthalene group;

A₂₁ may be a benzene group, or a naphthalene group;

A₂₂ may be a benzene group, a naphthalene group, a fluorene group, a carbazole group, a dibenzofuran group, or a dibenzothiophene group, but embodiments are not limited thereto.

In one or more embodiments, A₁₁ in Formulae 1, 2-1, and 2-2 may be a benzene group, or a naphthalene group;

A₂₁ may be a benzene group; and

A₂₂ may be a benzene group, a fluorene group, a carbazole group, a dibenzothiophene group, or a dibenzothiophene group, but embodiments are not limited thereto.

X₂₁ in Formulae 2-1 and 2-2 may be O, S, N(R₂₃), or C(R₂₃)(R₂₄), and R₂₃ and R₂₄ may be understood by referring to the related description to be presented later.

R₁₁, R₁₂, and R₂₁ to R₂₄ in Formulae 1, 2-1 and 2-2 may each independently be hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, a C₁-C₆₀ alkyl group which is substituted with deuterium, —F, a cyano group, a C₁-C₆₀ alkyl group, a C₆-C₆₀ aryl group which is substituted with deuterium, —F, a cyano group, a C₁-C₆₀ alkyl group, a C₆-C₆₀ aryl group, or a combination thereof, an unsubstituted C₆-C₆₀ aryl group, or a combination thereof.

For example, R₁₁, R₁₂ and R₂₁ to R₂₄ in Formulae 1, 2-1 and 2-2 may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cycloctyl group, a bicyclo[2.2.1]heptyl group, an adamantyl group, a norbornyl group, a norbornenyl group, a cyclopentenyl group, a cyclohexenyl group, a cycloheptenyl group, a phenyl group, a biphenyl group, a C₇-C₂₀ alkylphenyl group, a naphthyl group, a pyridinyl group, a pyrimidinyl group, or a combination thereof;
a cyclopentyl group, a cyclohexyl group, a cycloheptyl group, a cycloctyl group, a bicyclo[2.2.1]heptyl group, an adamantyl group, a norbornyl group, a norbornenyl group, a cyclopentenyl group, a cyclohexenyl group, a cycloheptenyl group, a phenyl group, a biphenyl group, a C₇-C₂₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, or an imidazopyrimidinyl group;
a cyclopentyl group, a cyclohexyl group, a cycloheptyl group, a cycloctyl group, a bicyclo[2.2.1]heptyl group, an adamantyl group, a norbornyl group, a norbornenyl group, a cyclopentenyl group, a cyclohexenyl group, a cycloheptenyl group, a phenyl group, a biphenyl group, a C₇-C₂₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, or an imidazopyrimidinyl group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cycloctyl group, a bicyclo[2.2.1]heptyl group, an adamantyl group, a norbornyl group, a norbornenyl group, a cyclopentenyl group, a cyclohexenyl group, a cycloheptenyl group, a phenyl group, a biphenyl group, a C₇-C₂₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, —Si(Q₁₁)(Q₁₂)(Q₁₃), —B(Q₁₁)(Q₁₂), —N(Q₁₁)(Q₁₂), or a combination thereof; or
—Si(Q₁)(Q₂)(Q₃), —B(Q₁)(Q₂), or —N(Q₁)(Q₂);
Q₁ to Q₃ and Q₁₁ to Q₁₃ may each independently be
a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, 3-pentyl group, 3-methyl-2-butyl group, a phenyl group, a biphenyl group, a C₇-C₂₀ alkylphenyl group, or a naphthyl group; or
a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, 3-pentyl group, 3-methyl-2-butyl group, a phenyl group, or a naphthyl group, each substituted with deuterium, a phenyl group, or a combination thereof, but embodiments of the present disclosure are not limited thereto.

In one embodiment, R₁₁, R₁₂ and R₂₁ to R₂₄ in Formulae 1, 2-1, and 2-2 may each independently be hydrogen, deuterium, —F, a cyano group, a nitro group, —CH₃, —CD₃, —CD₂H, —CDH₂, —CF₃, —CF₂H, —CFH₂, a group represented by one of Formulae 9-1 to 9-27, a group obtained by substituting hydrogen of the group represented by one of Formulae 9-1 to 9-27 with deuterium, a group represented by one of Formulae 10-1 to 10-226, —Si(Q₁)(Q₂)(Q₃), —B(Q₁)(Q₂), or —N(Q₁)(Q₂), but embodiments of the present disclosure are not limited thereto:

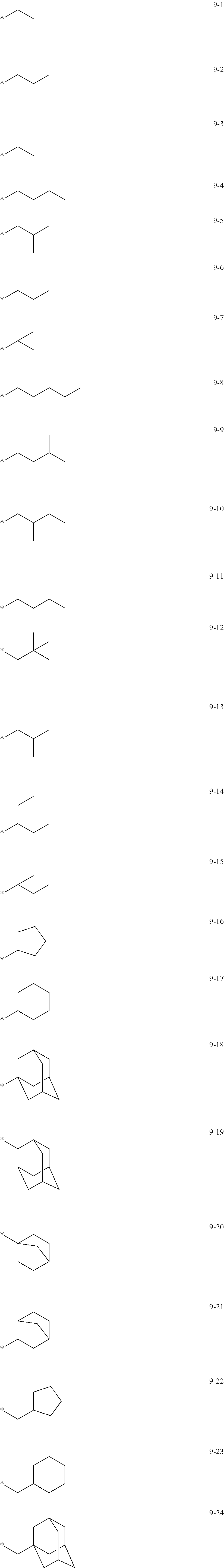

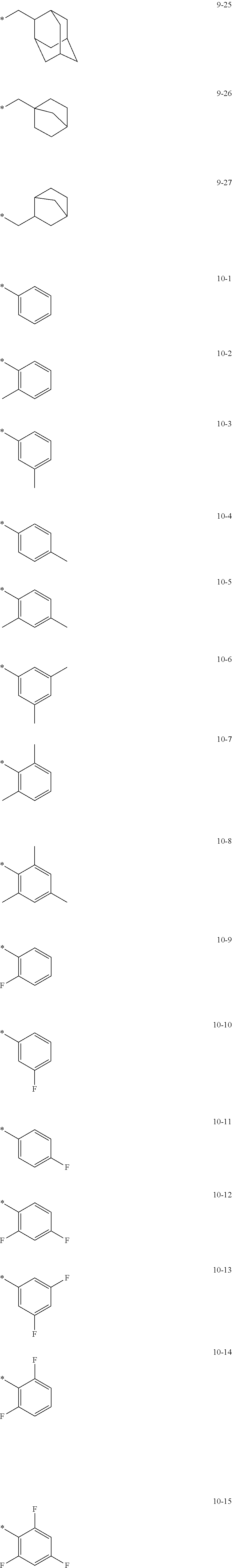

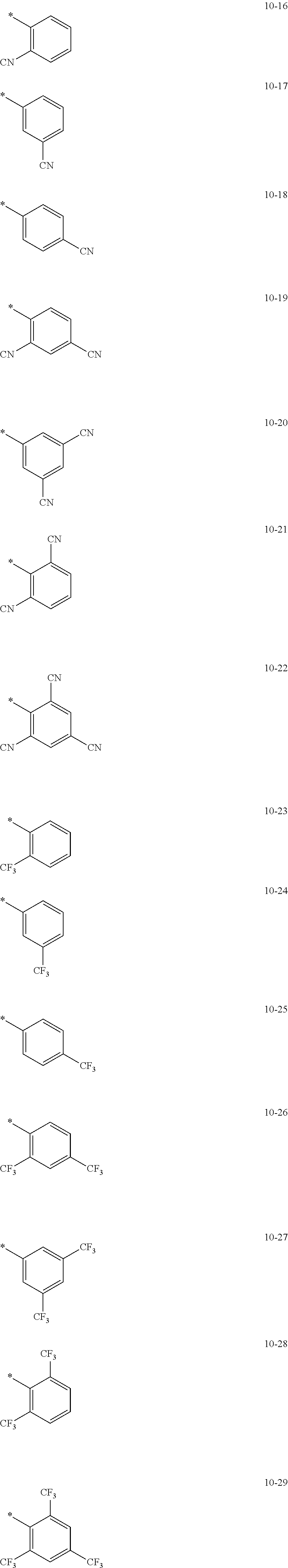

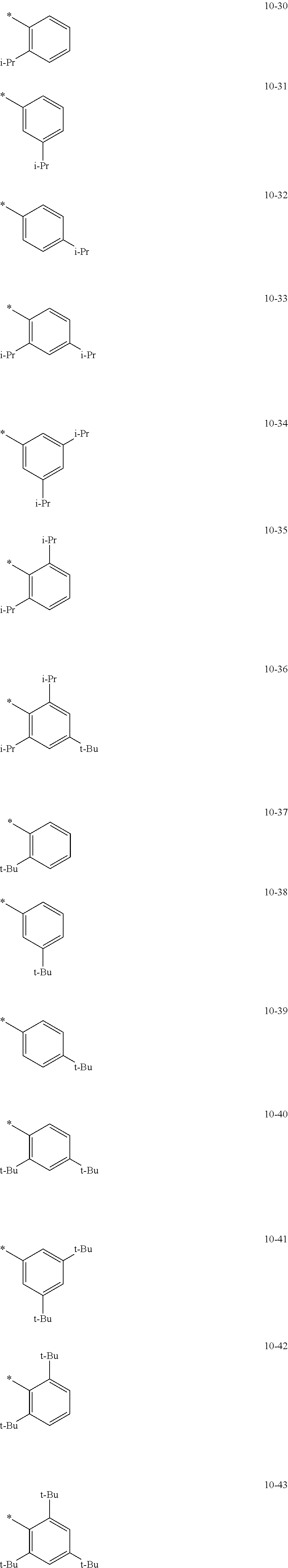

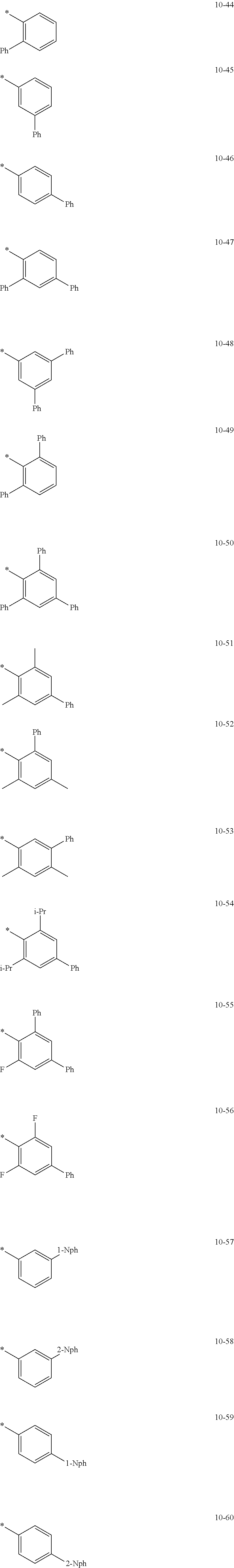

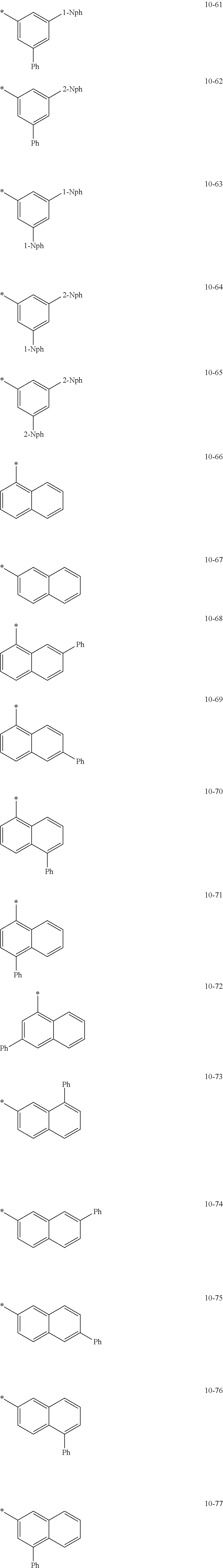

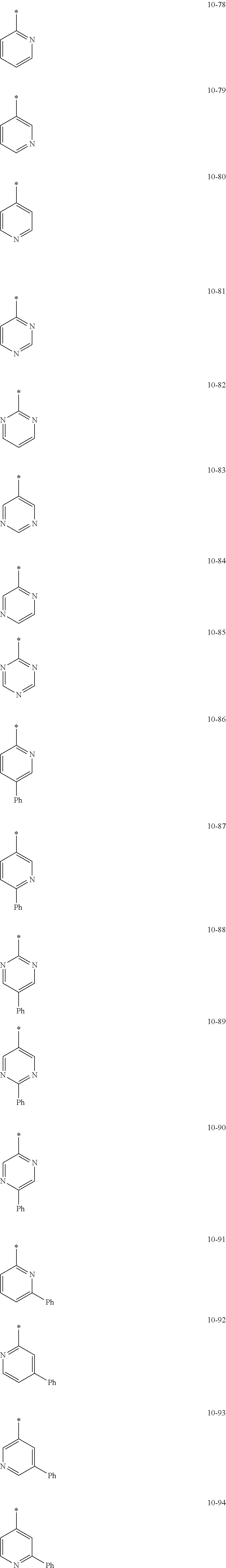

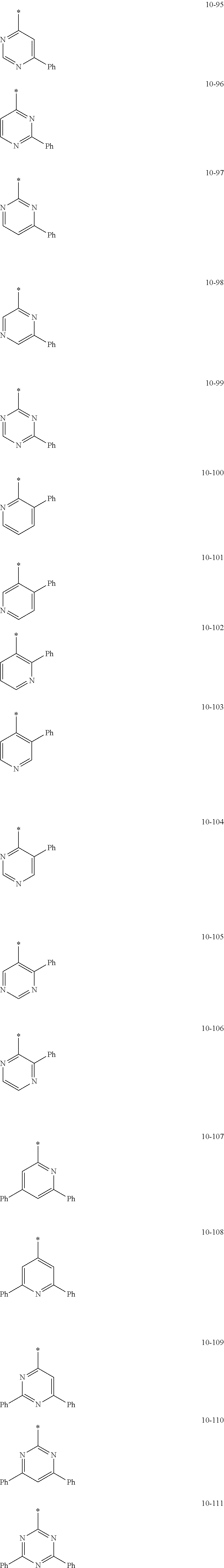

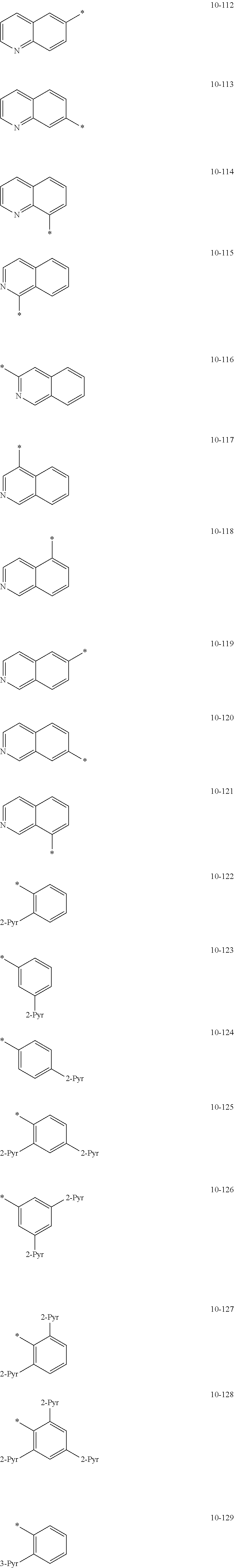

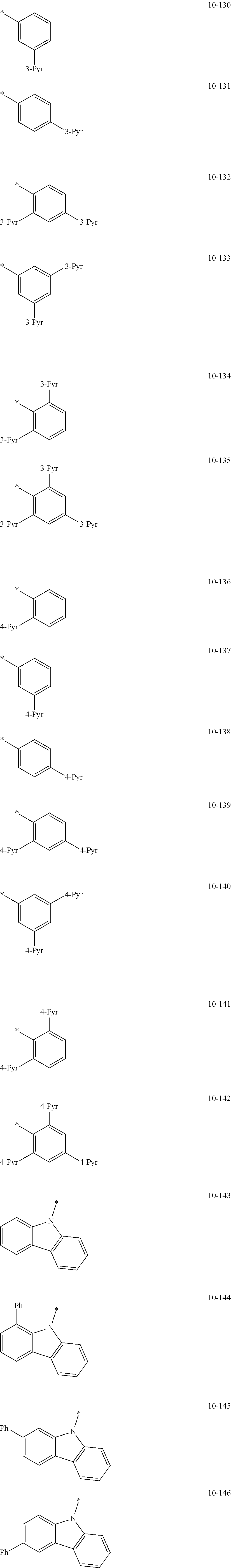

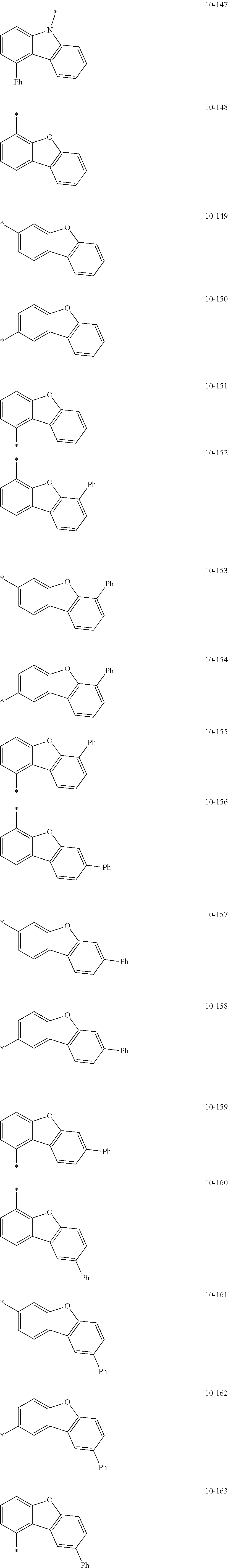

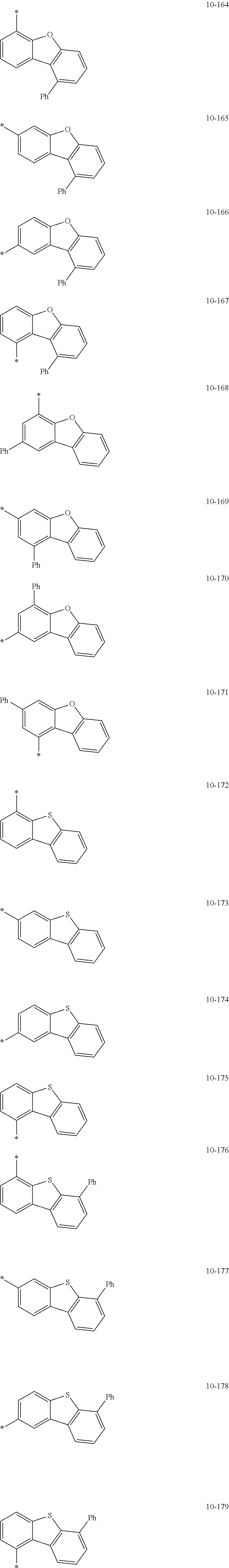

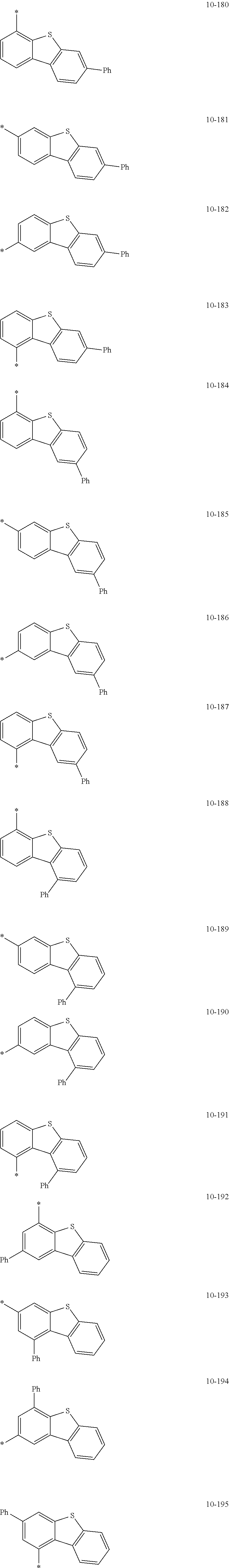

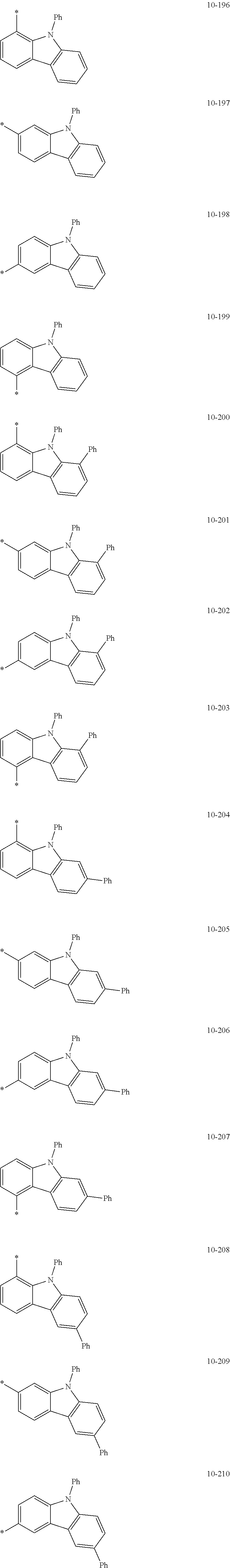

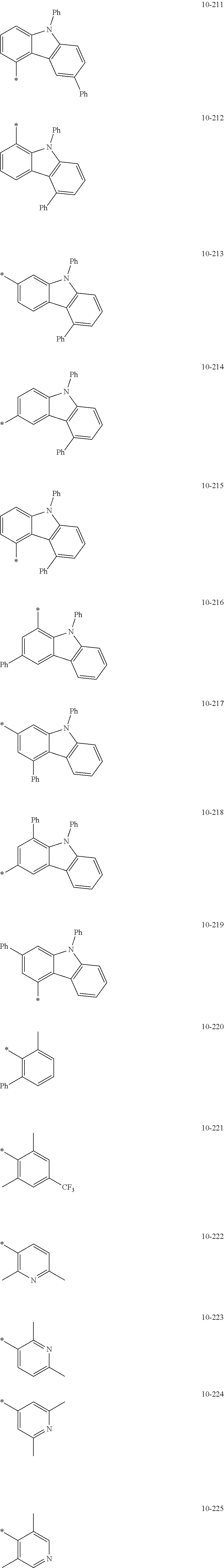

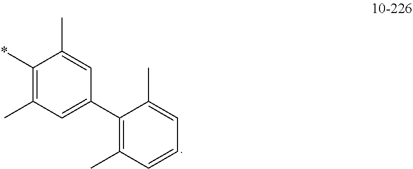 [SMILES: *c1c(C)cc(-c2c(C)cccc2C)cc1C]

In Formulae 9-1 to 9-27 and 10-1 to 10-226,
* indicates a binding site to a neighboring atom,
i-Pr is an isopropyl group, and t-Bu is a tert-butyl group,
Ph is a phenyl group;
1-Nph is a 1-naphthyl group, and 2-Nph is 2-naphthyl group,
2-Pyr is a 2-pyridyl group, 3-Pyr is a 3-pyridyl group, and 4-Pyr is a 4-pyridyl group,
Q₁ to Q₃ may each independently be
a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, 3-pentyl group, 3-methyl-2-butyl group, a phenyl group, a biphenyl group, a C₇-C₂₀ alkylphenyl group, or a naphthyl group; or
a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, 3-pentyl group, 3-methyl-2-butyl group, a phenyl group, or a naphthyl group, each substituted with deuterium, a phenyl group, or a combination thereof.

In one or more embodiments, R₁₂ in Formula 1 may be hydrogen or a group represented by one of Formulae 10-1 and 10-107 to 10-111, but embodiments are not limited thereto.

In one or more embodiments, R₂₁ and R₂₂ in Formulae 2-1 and 2-2 may each independently be hydrogen, a group represented by Formula 9-7 or a group represented by Formula 10-1, but embodiments are not limited thereto.

b11 in Formula 1 indicates the substitution number of R₁₁, and b11 may be 0 or 1.

The sum of n11, a11, and b11 in Formula 1 may be 4.

b12, b21, and b22 in Formulae 1, 2-1, and 2-2 may each independently be 1, 2, 3, 4, 5, 6, 7, 8, 9, or 10.

In one or more embodiments, Y₁₁ in Formula 1 may be represented by any one of Formulae 2-11 to 2-17, but embodiments are not limited thereto:

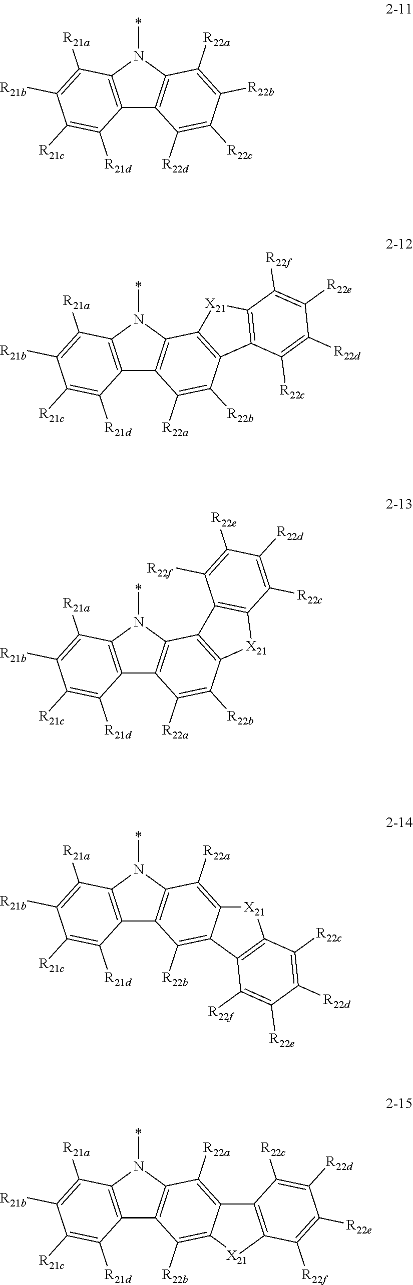

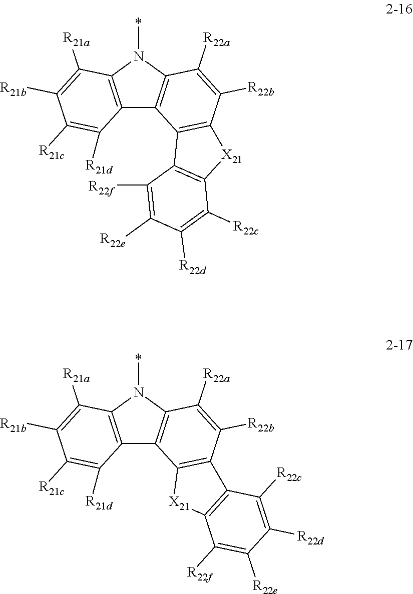

wherein, in Formulae 2-11 to 2-17,
X₂₁ may be O, S, N(R_{22g}), or C(R_{22g})(R₂₂ₕ);
R₂₁ₐ to R_{21d} may be understood by referring to the definition of R₂₁ of Formula 2-1;
R₂₂ₐ to R₂₂ₕ may be understood by referring to the definition of R₂₂ of Formula 2-1 and
* indicates a binding site to a neighboring atom.

In one embodiment, the condensed-cyclic compound represented by Formula 1 may be represented by one of Formulae 1-1 to 1-6, but embodiments are not limited thereto:

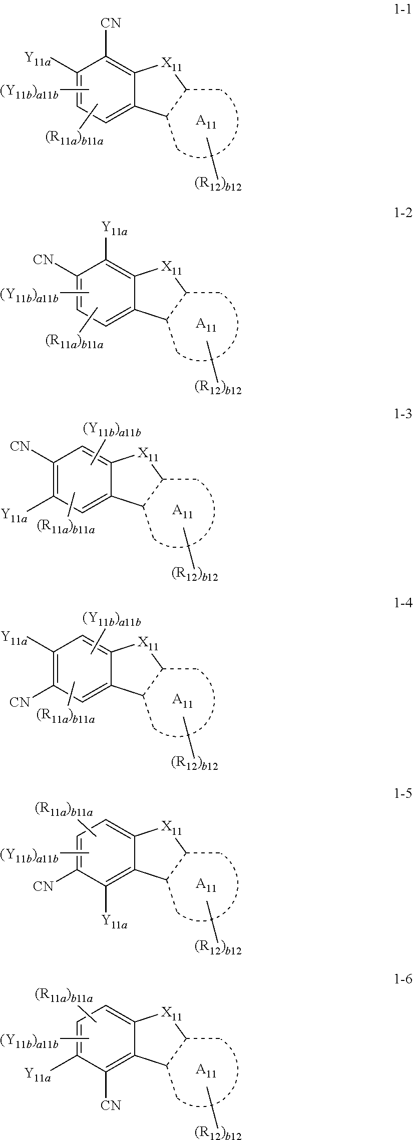

wherein, in Formulae 1-1 to 1-6,
Y₁₁ₐ and Y_{11b} may each be understood by referring to the definition of Y₁₁ of Formula 1,
R₁₁ₐ may be understood by referring to the definition of R₁₁ of Formula 1,
a11b may be 1 or 2;
X₁₁, A₁₁, R₁₂, and b12 may each be understood by referring to the definitions provided in connection with Formula 1,
A₂₁, A₂₂, X₂₁, R₂₁ to R₂₄, b21, and b22 may each be understood by referring to the definitions provided in connection with Formulae 2-1 and 2-2, and
the sum of a11b and b11a may be 3.

For example, A₁₁ in Formula 1-1 to 1-6 may be a benzene group, but embodiments are not limited thereto.

For example, Y₁₁ in Formulae 1-1 to 1-6 may be represented by any one of Formulae 2-11 to 2-17, but embodiments are not limited thereto.

In one or more embodiments, the condensed-cyclic compound represented by Formula 1 may be represented by any one of Formulae 1-11 to 1-23, but embodiments are not limited thereto:

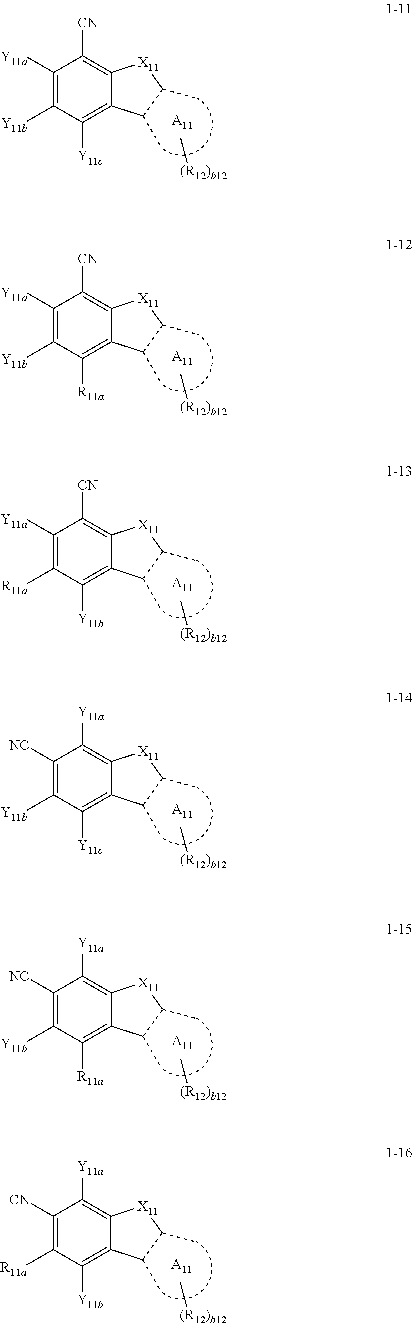

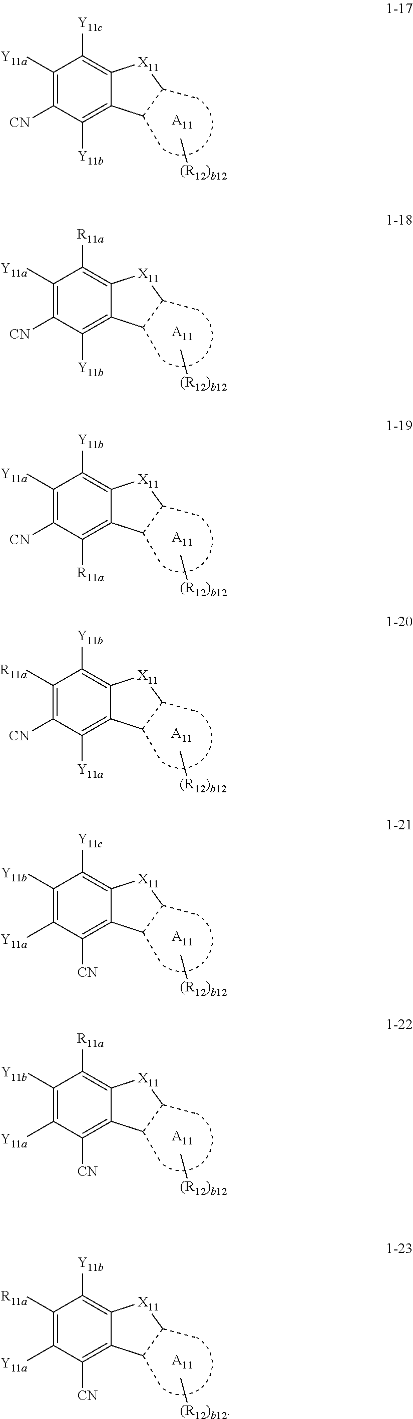

In Formulae 1-11 to 1-23,
Y₁₁ₐ, Y_{11b}, and Y₁₁ₑ may each be understood by referring to the definition of Y₁₁ of Formula 1,
R₁₁ₐ may be understood by referring to the definition of R₁₁ of Formula 1,
X₁₁, A₁₁, R₁₂, and b12 may each be understood by referring to the definitions provided in connection with Formula 1, and
A₂₁, A₂₂, X₂₁, R₂₁ to R₂₄, b21, and b22 may each be understood by referring to the definitions provided in connection with Formulae 2-1 and 2-2.

For example, A₁₁ in Formula 1-11 to 1-23 may be a benzene group, but embodiments are not limited thereto.

For example, Y₁₁ in Formula 1-11 to 1-23 may be represented by any one of Formulae 2-11 to 2-17, but embodiments are not limited thereto.

In one embodiment, the condensed-cyclic compound represented by Formula 1 may be Compounds 1 to 336, but embodiments are not limited thereto:

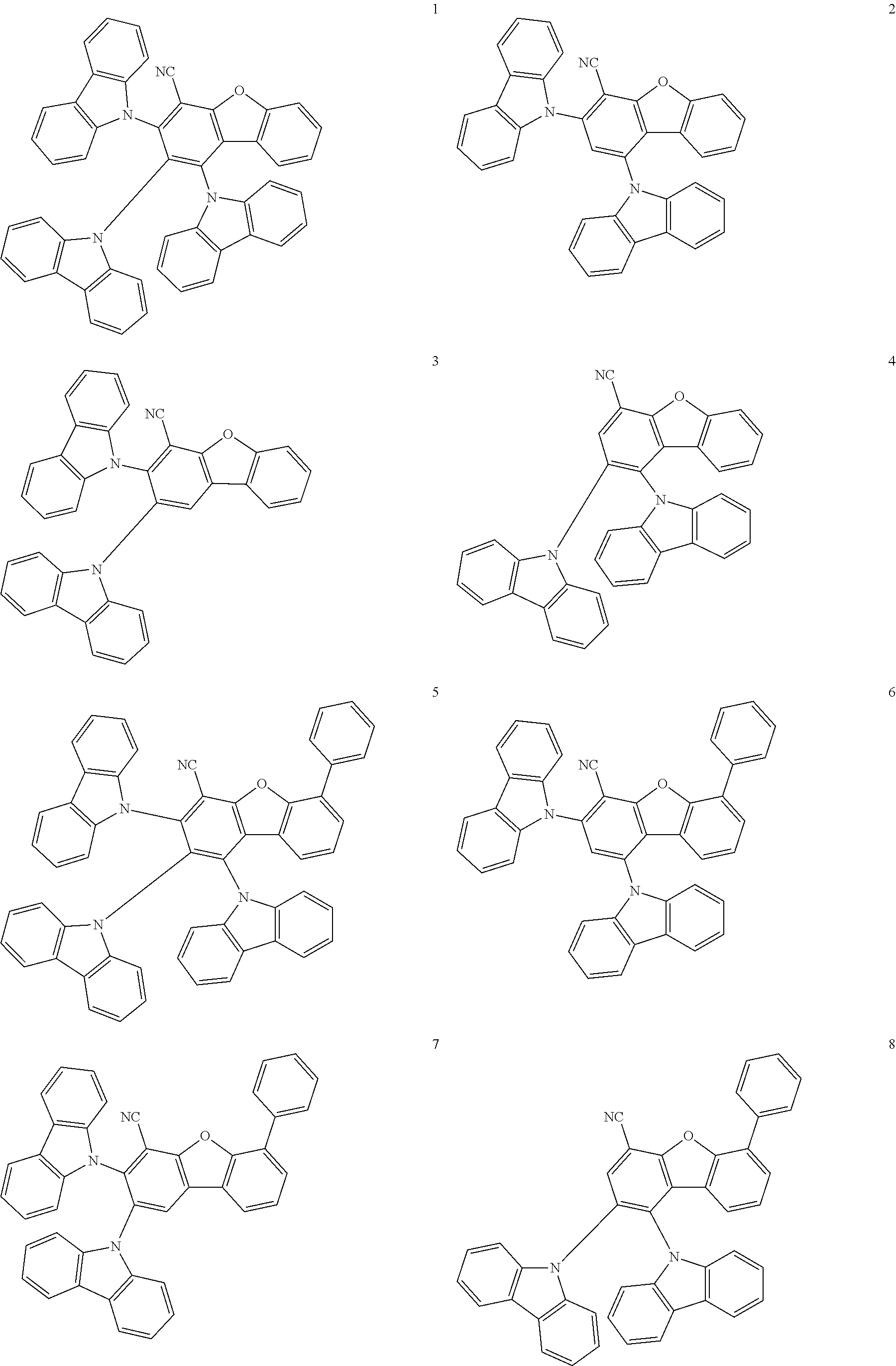

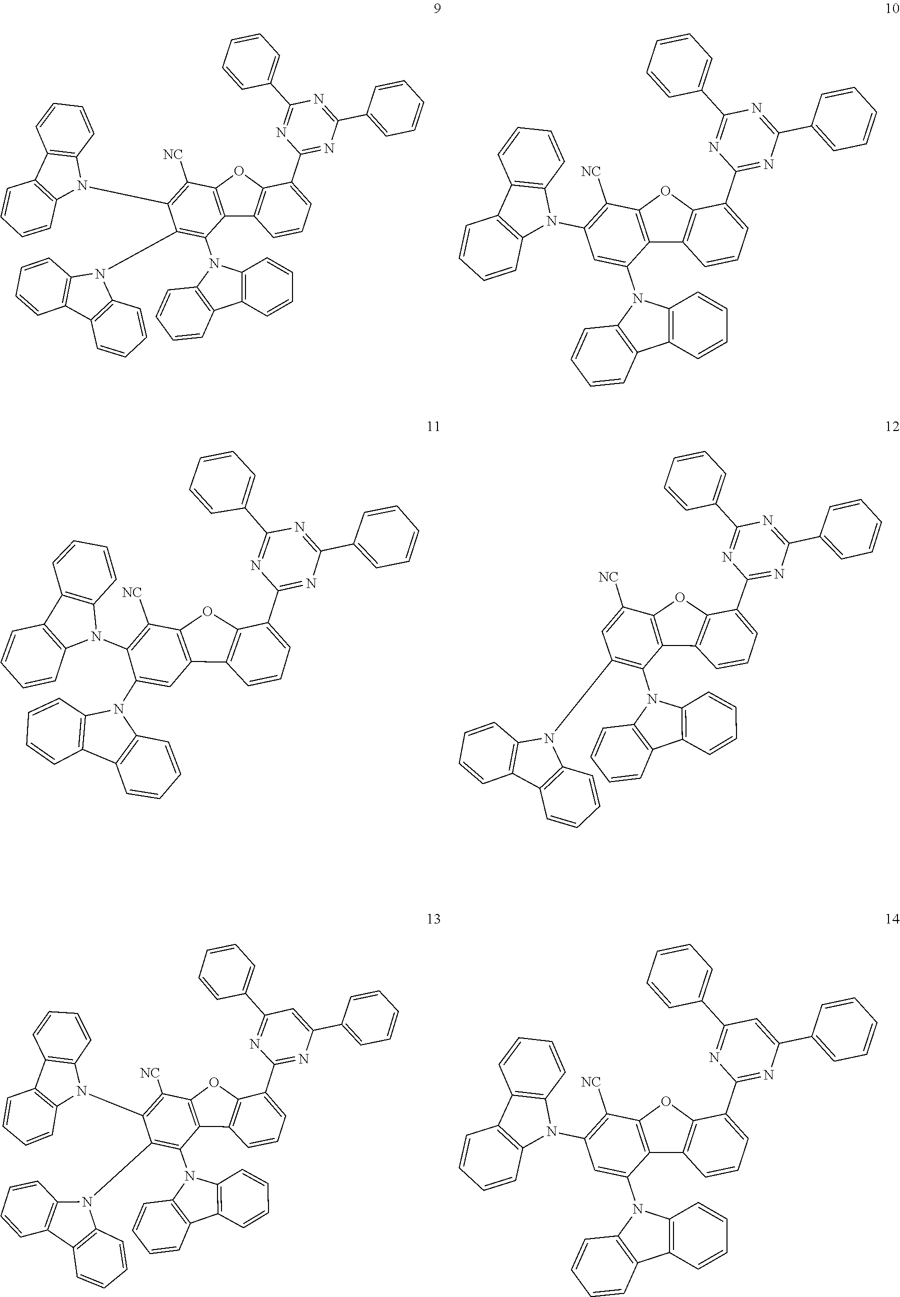

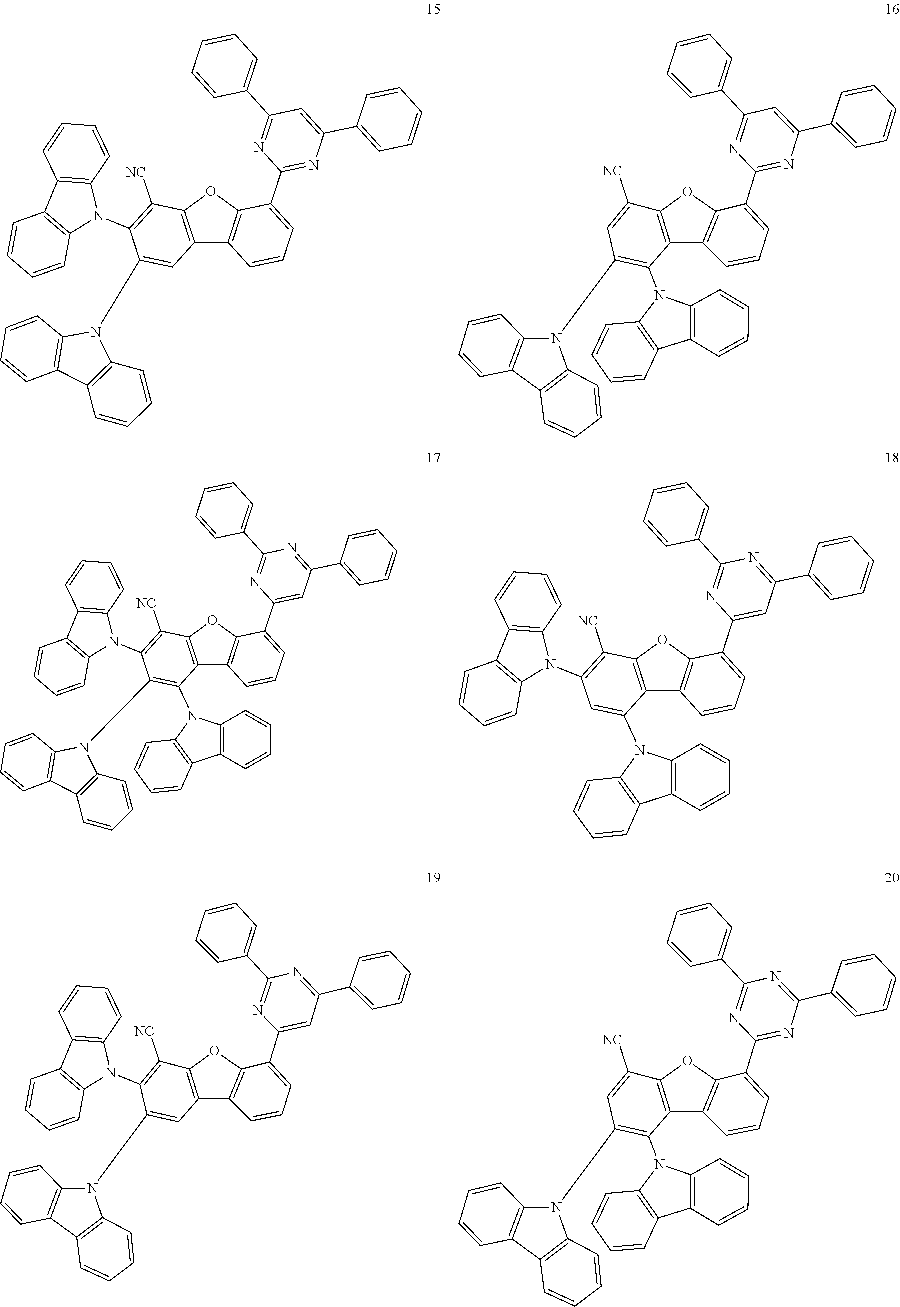

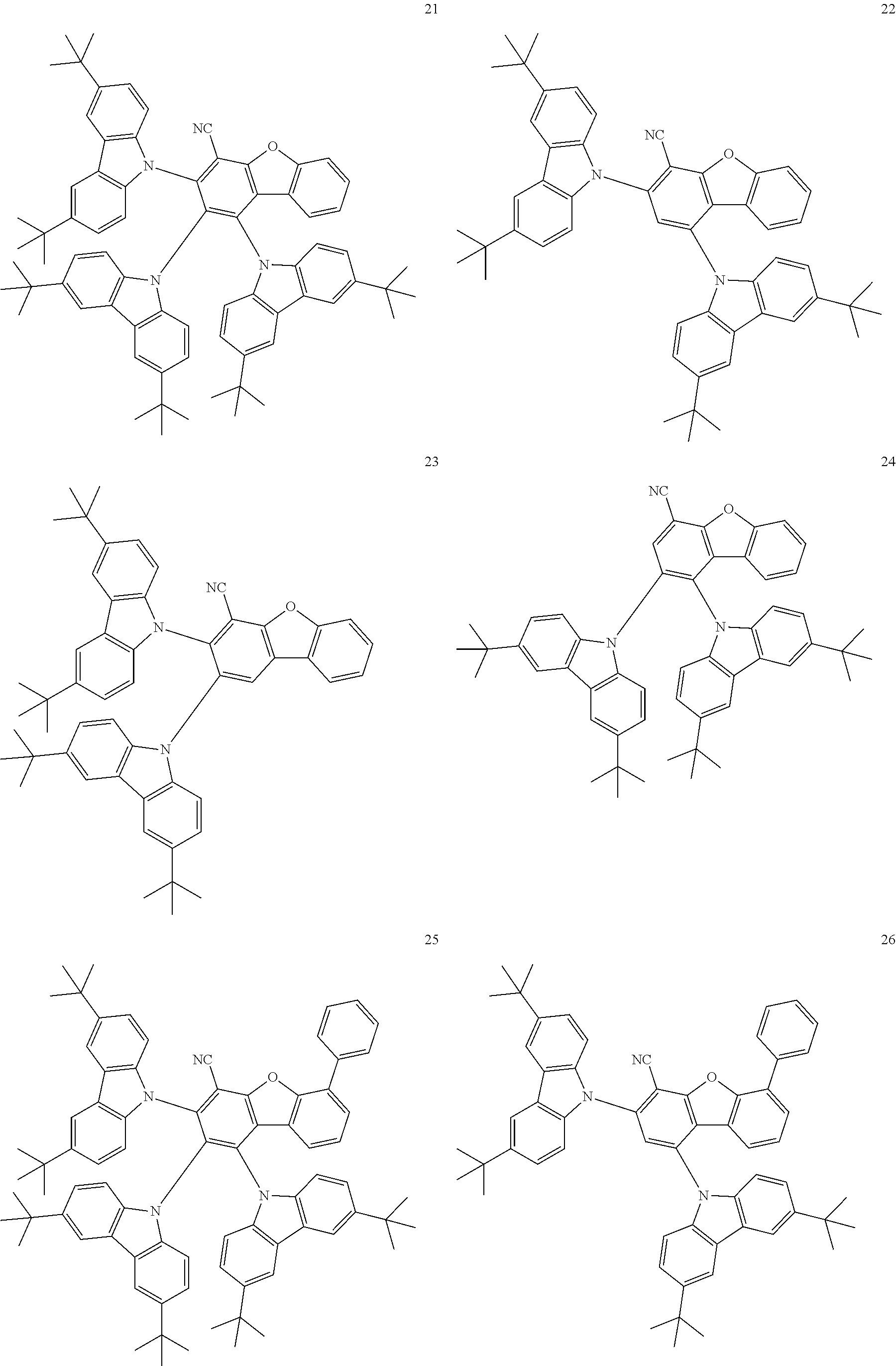

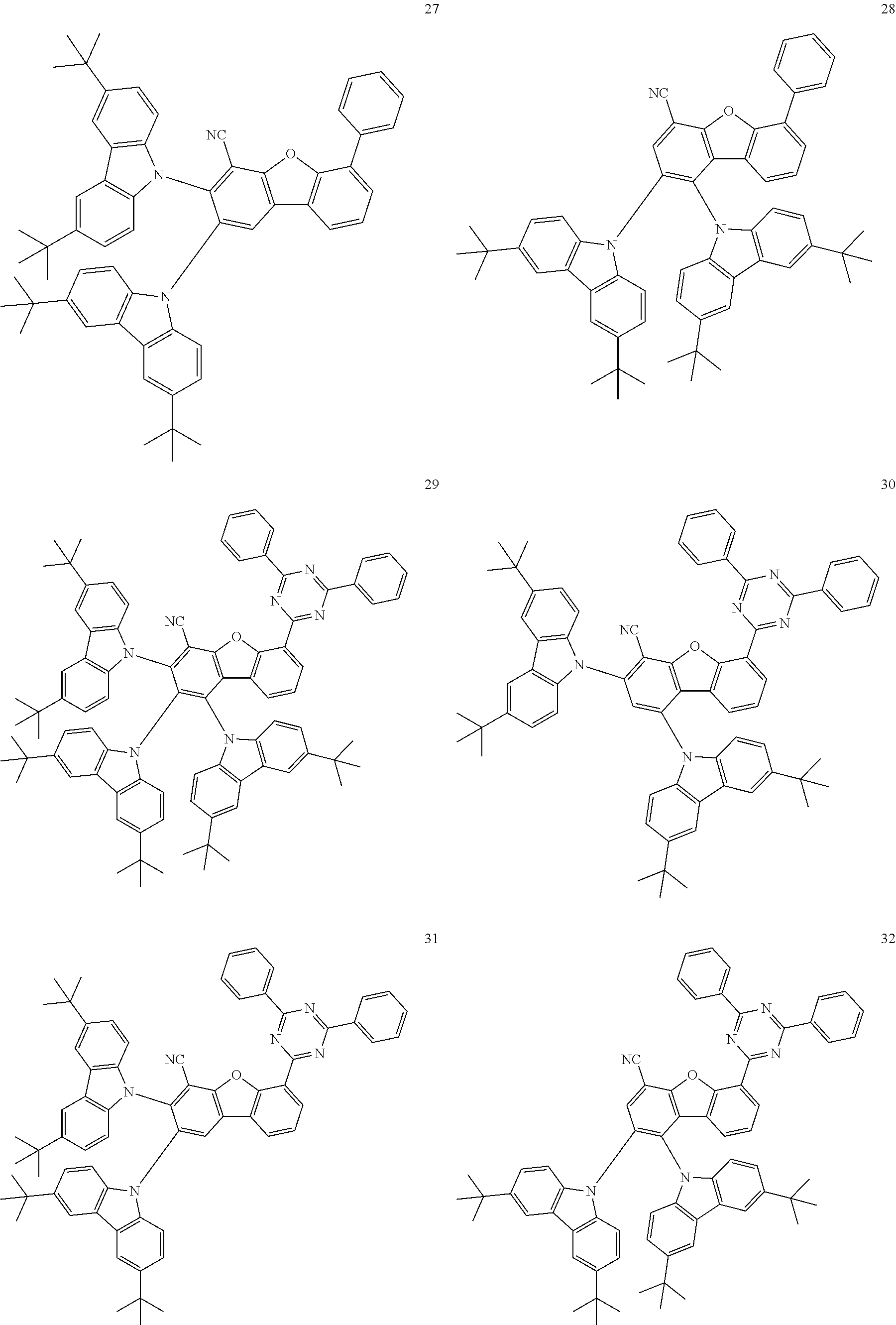

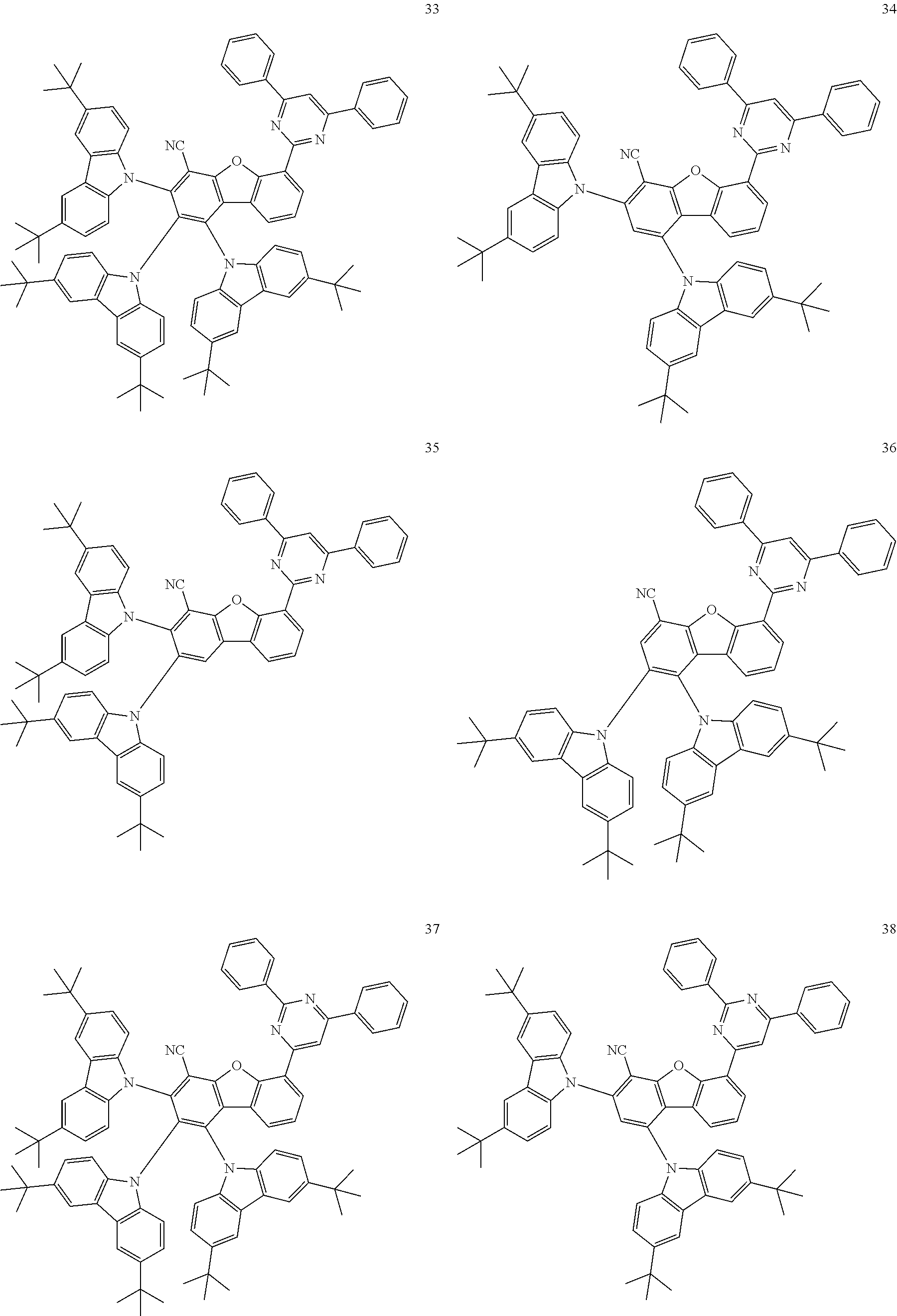

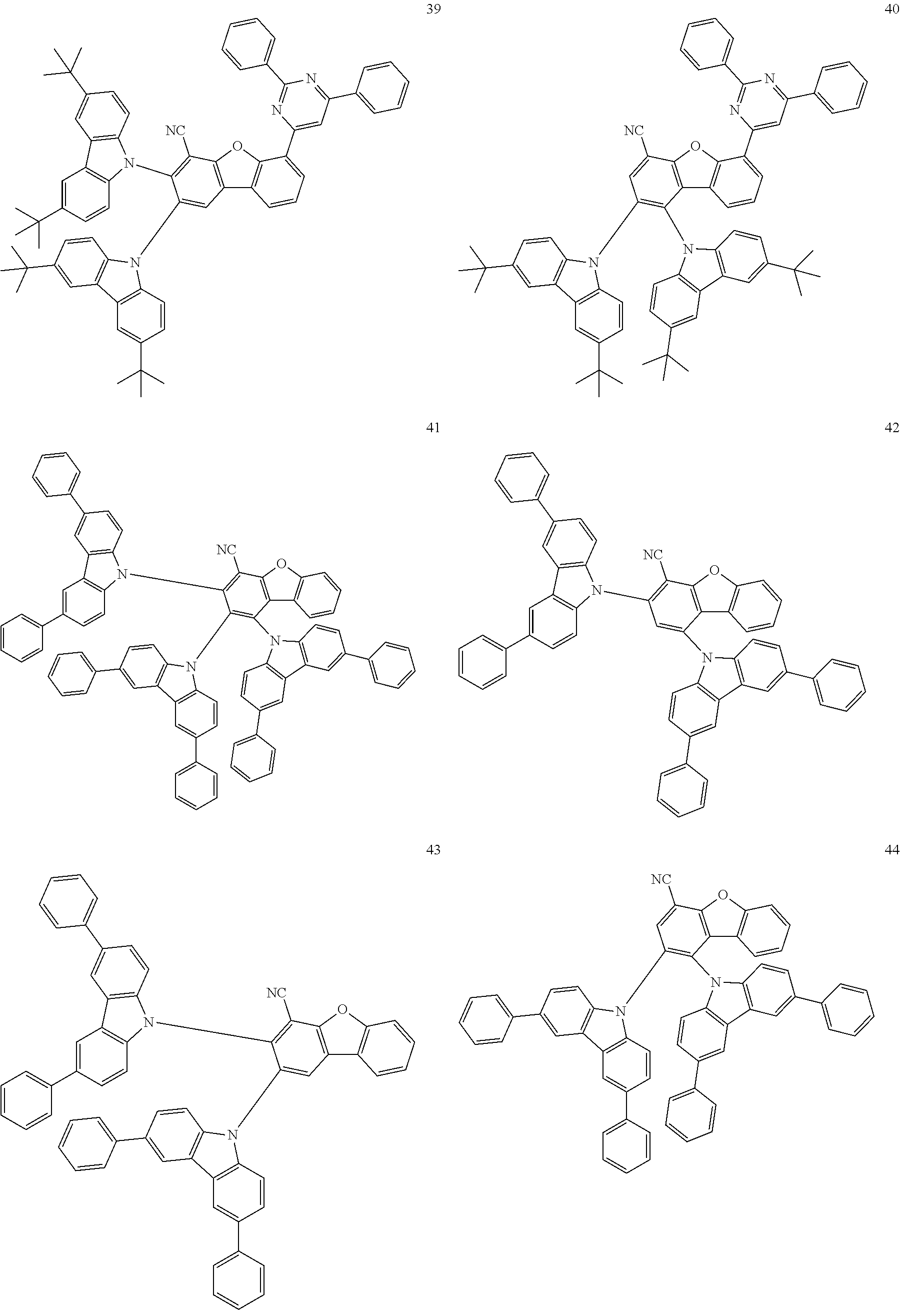

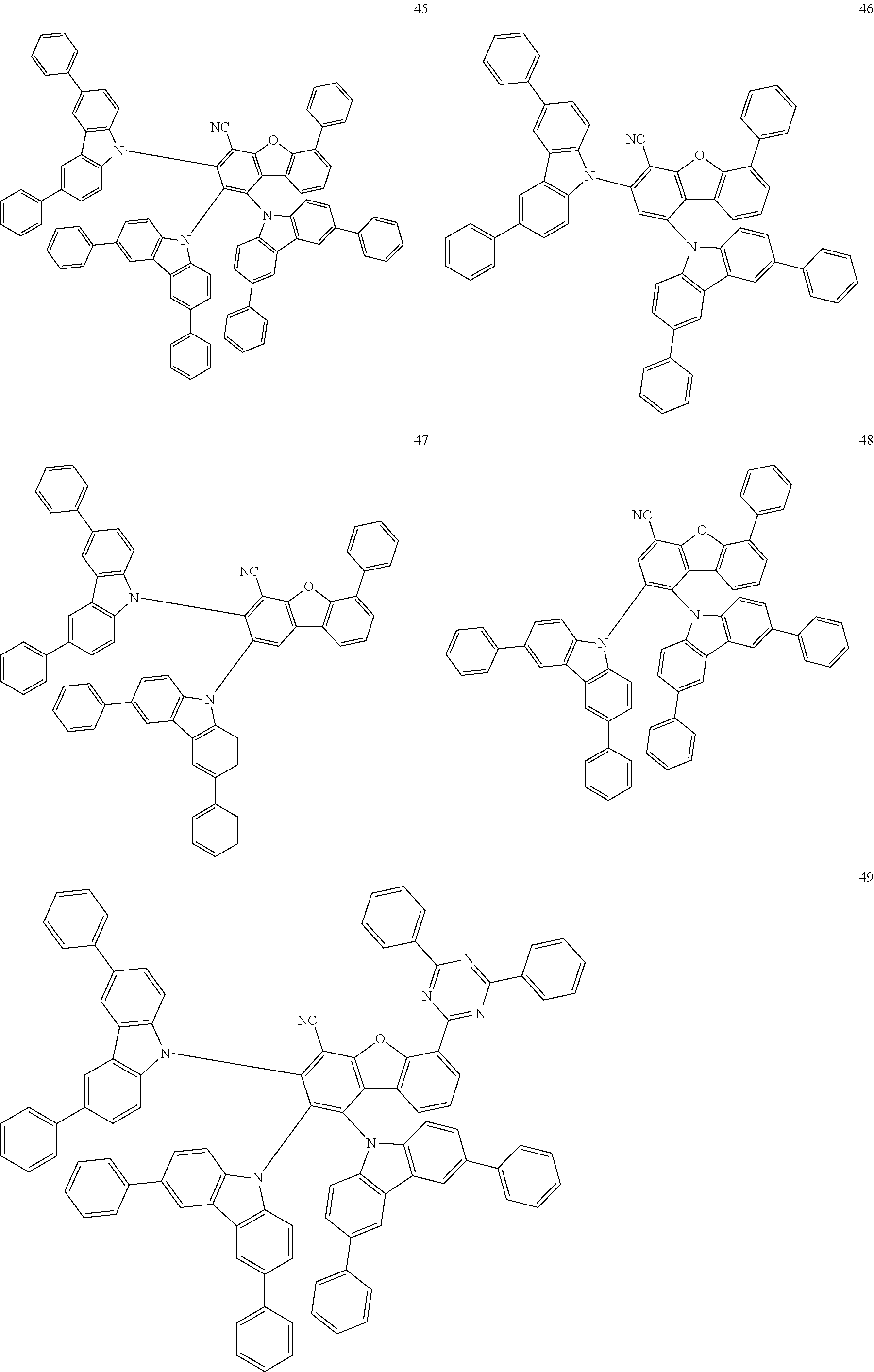

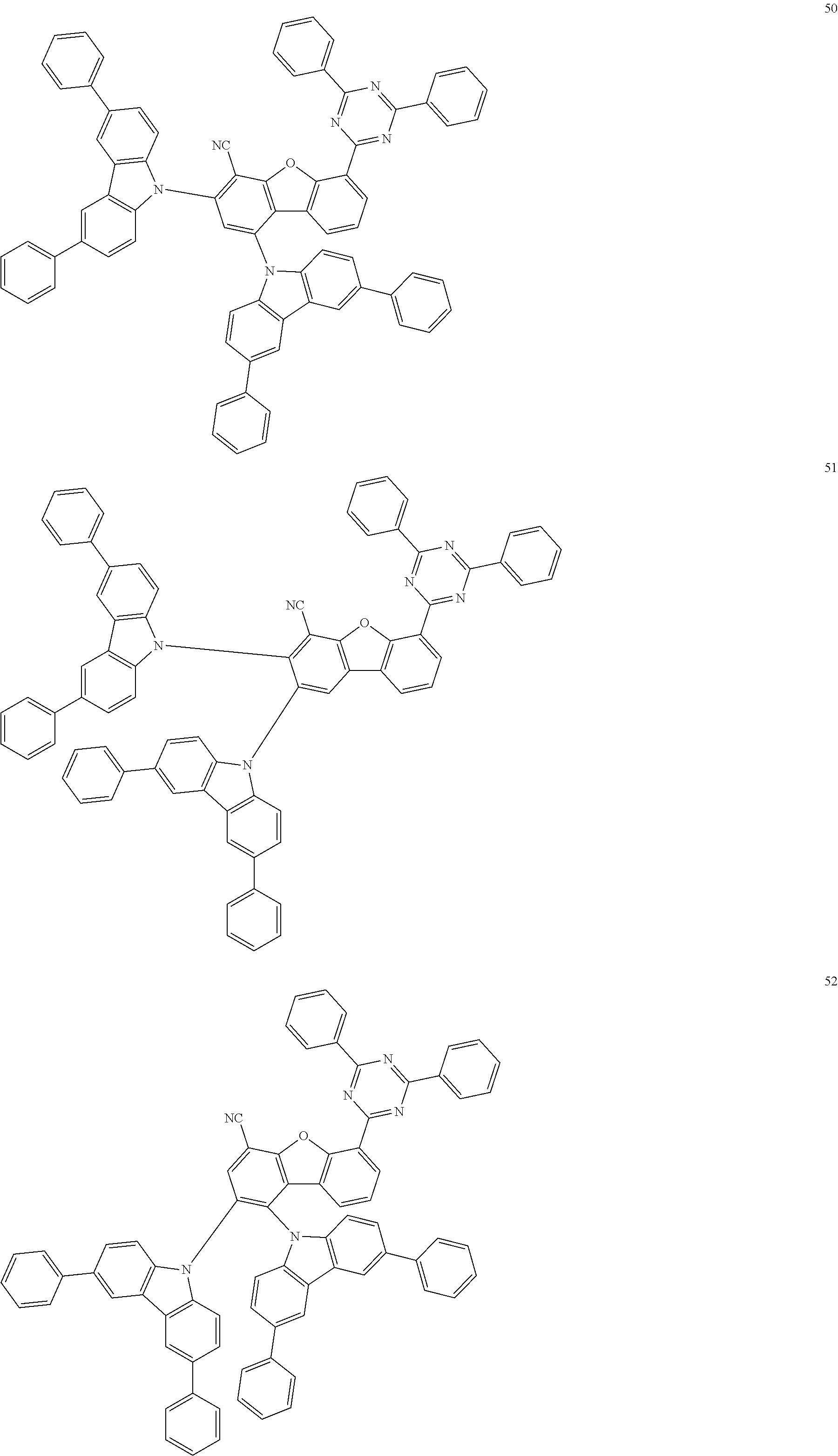 [SMILES: N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2c1oc1c(-c3nc(-c4ccccc4)nc(-c4ccccc4)n3)cccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2c1oc1c(-c3nc(-c4ccccc4)nc(-c4ccccc4)n3)cccc12 ; N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3nc(-c4ccccc4)nc(-c4ccccc4)n3)cccc12]

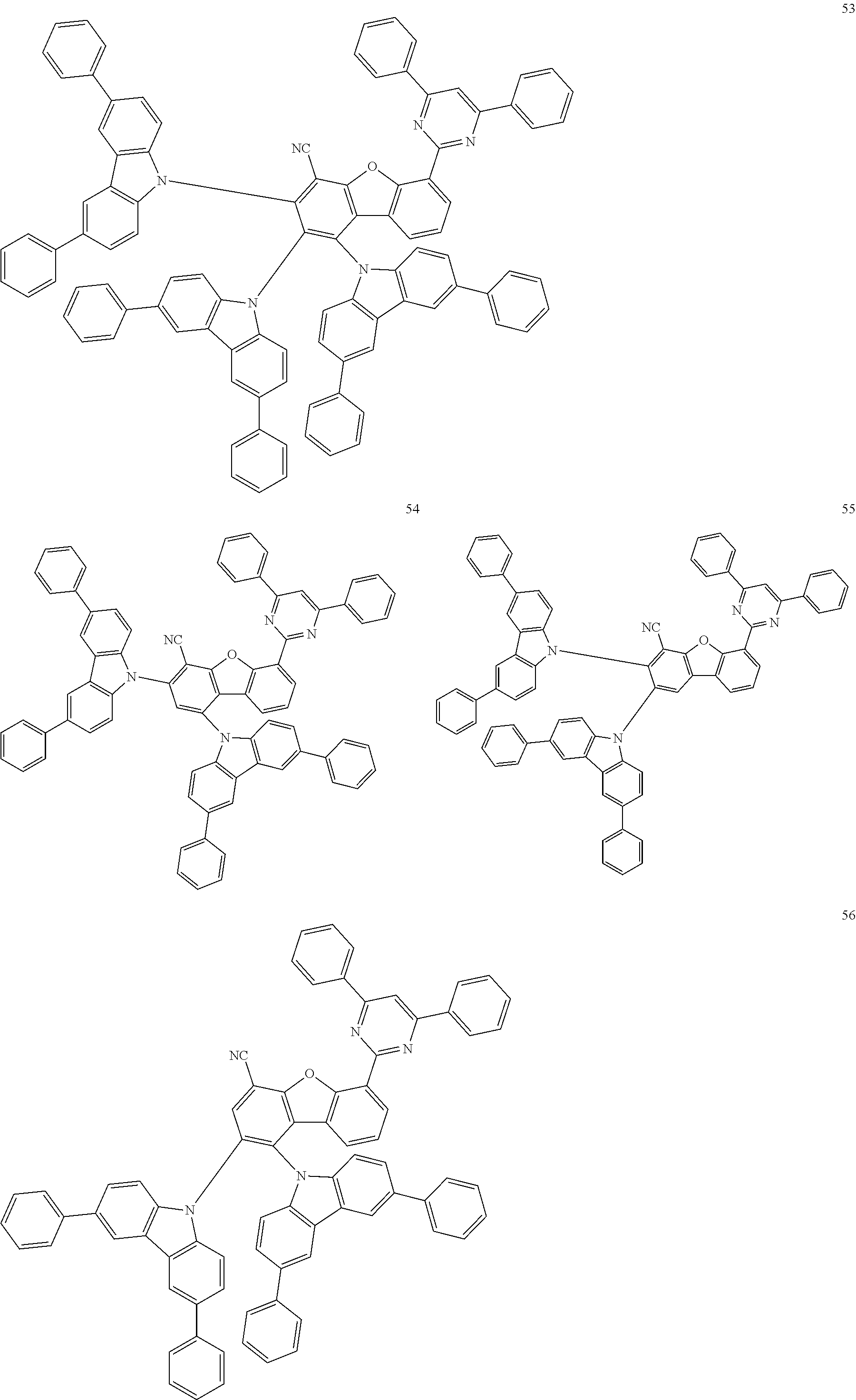

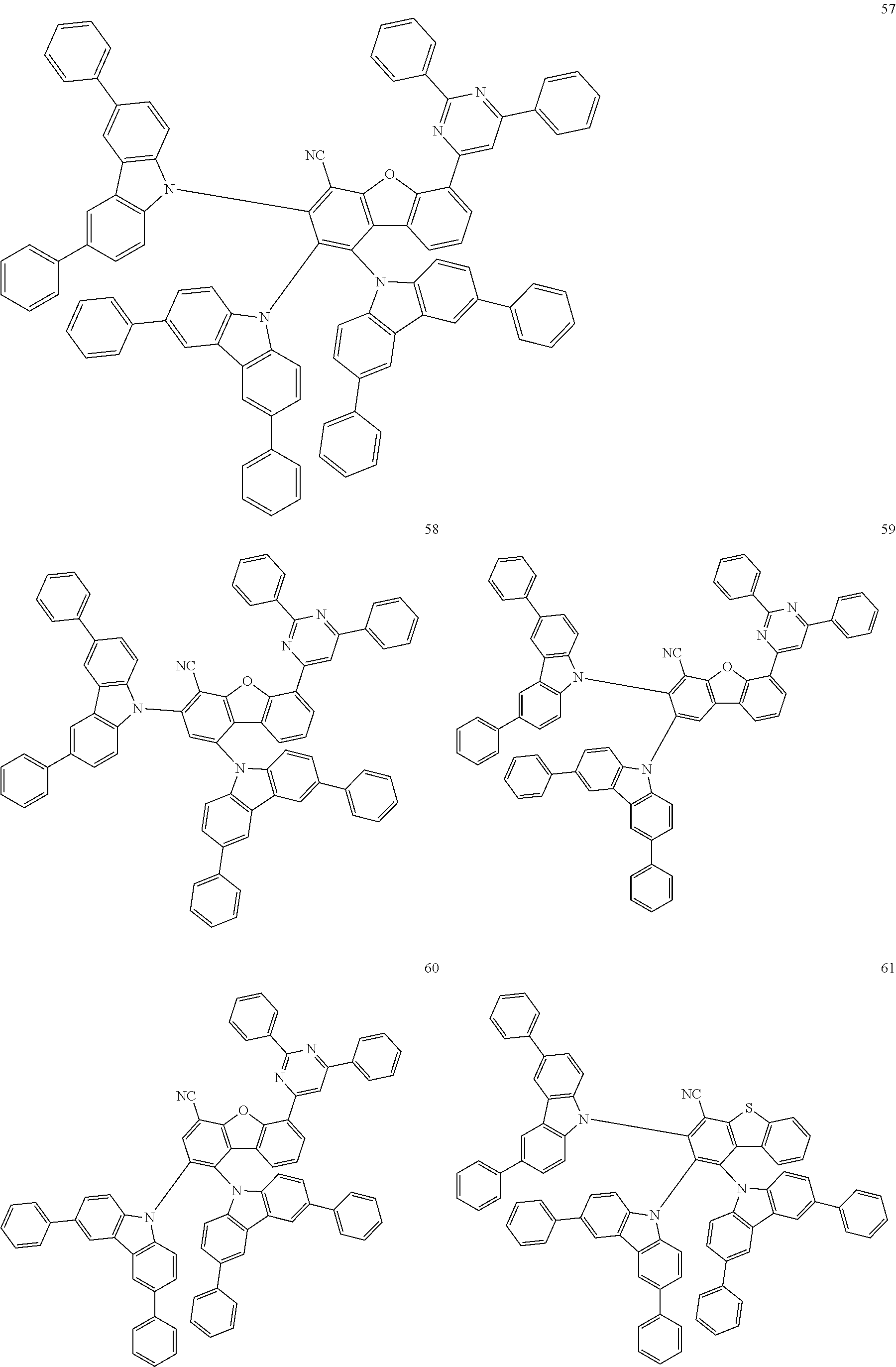

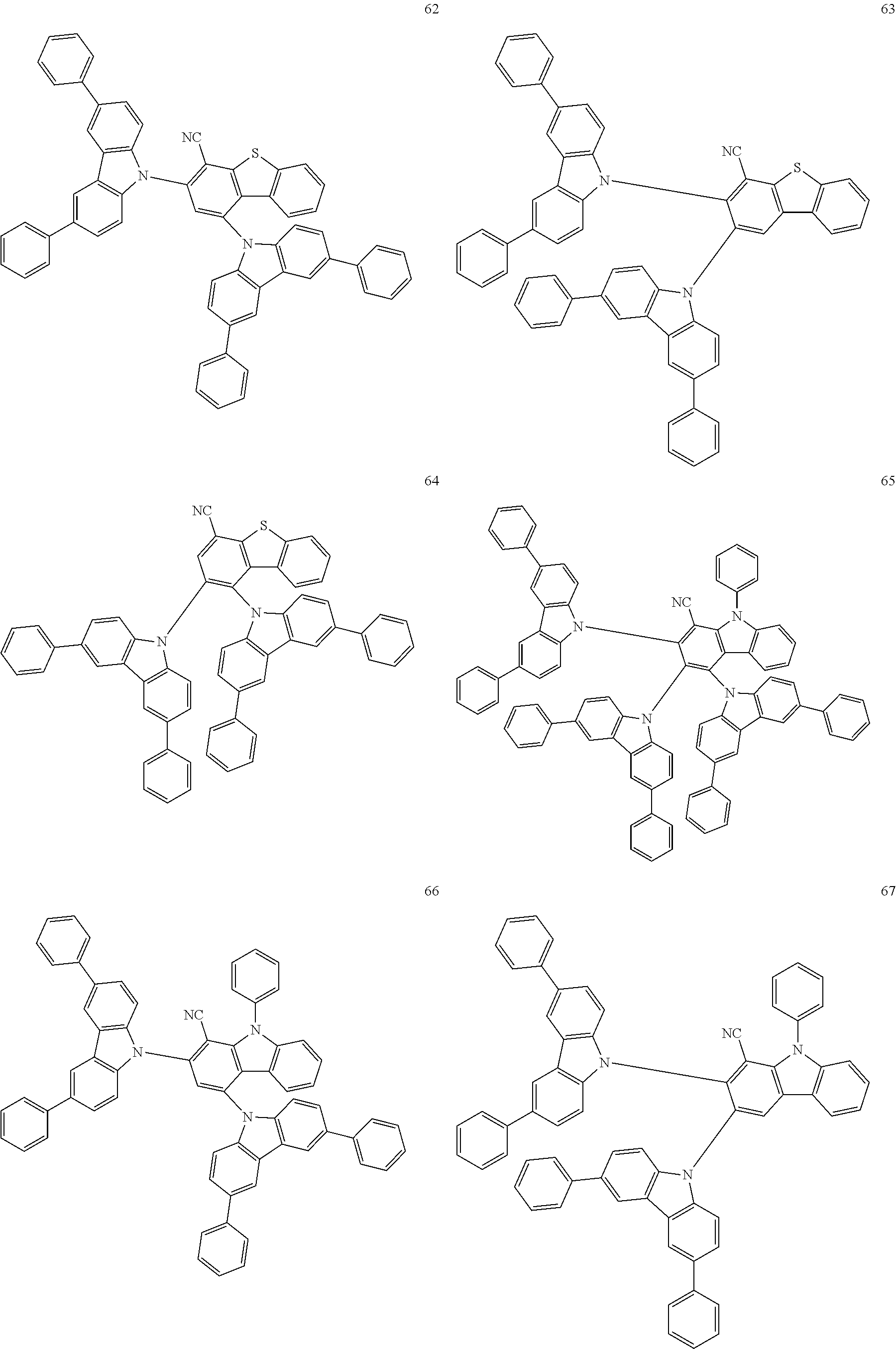

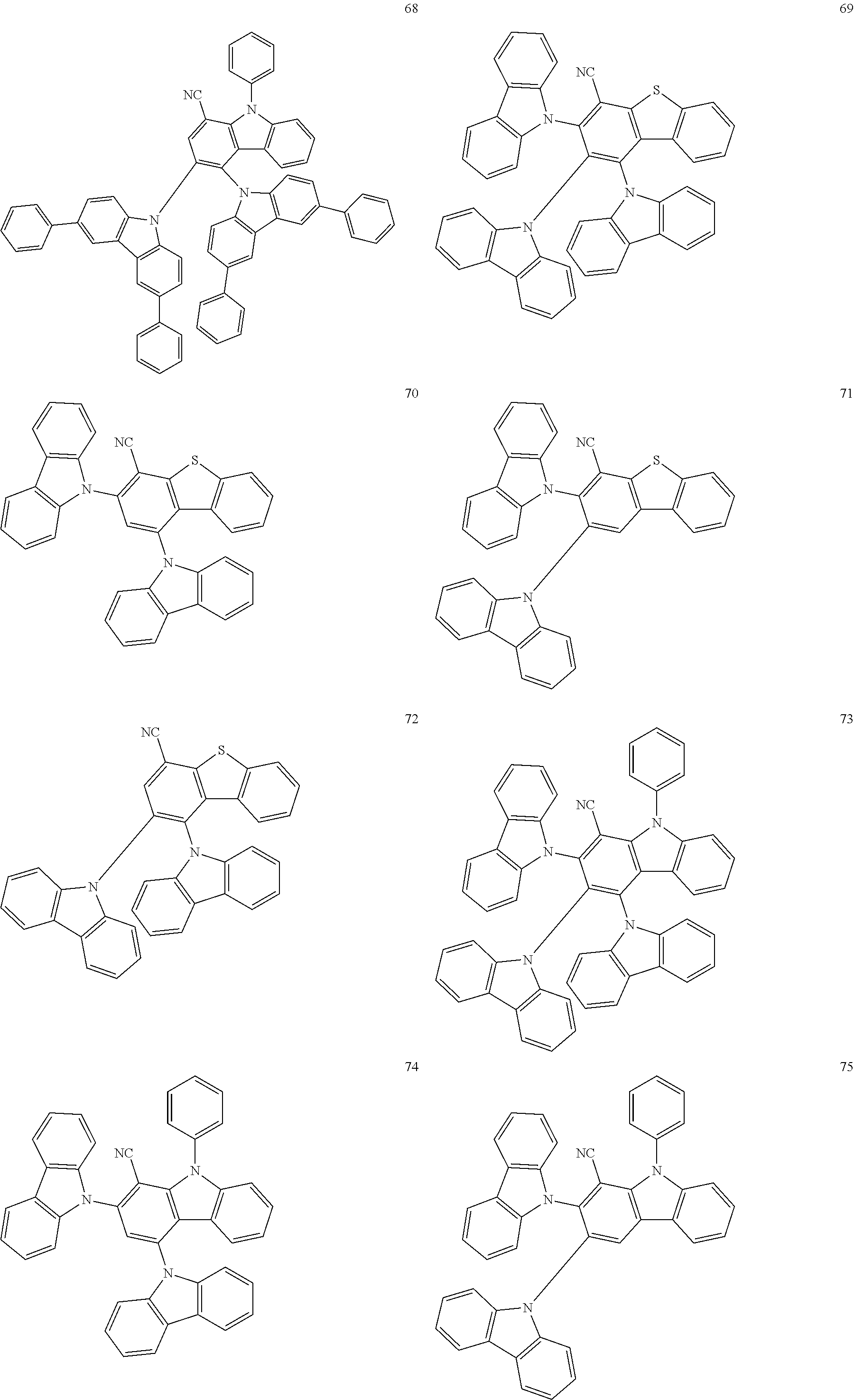

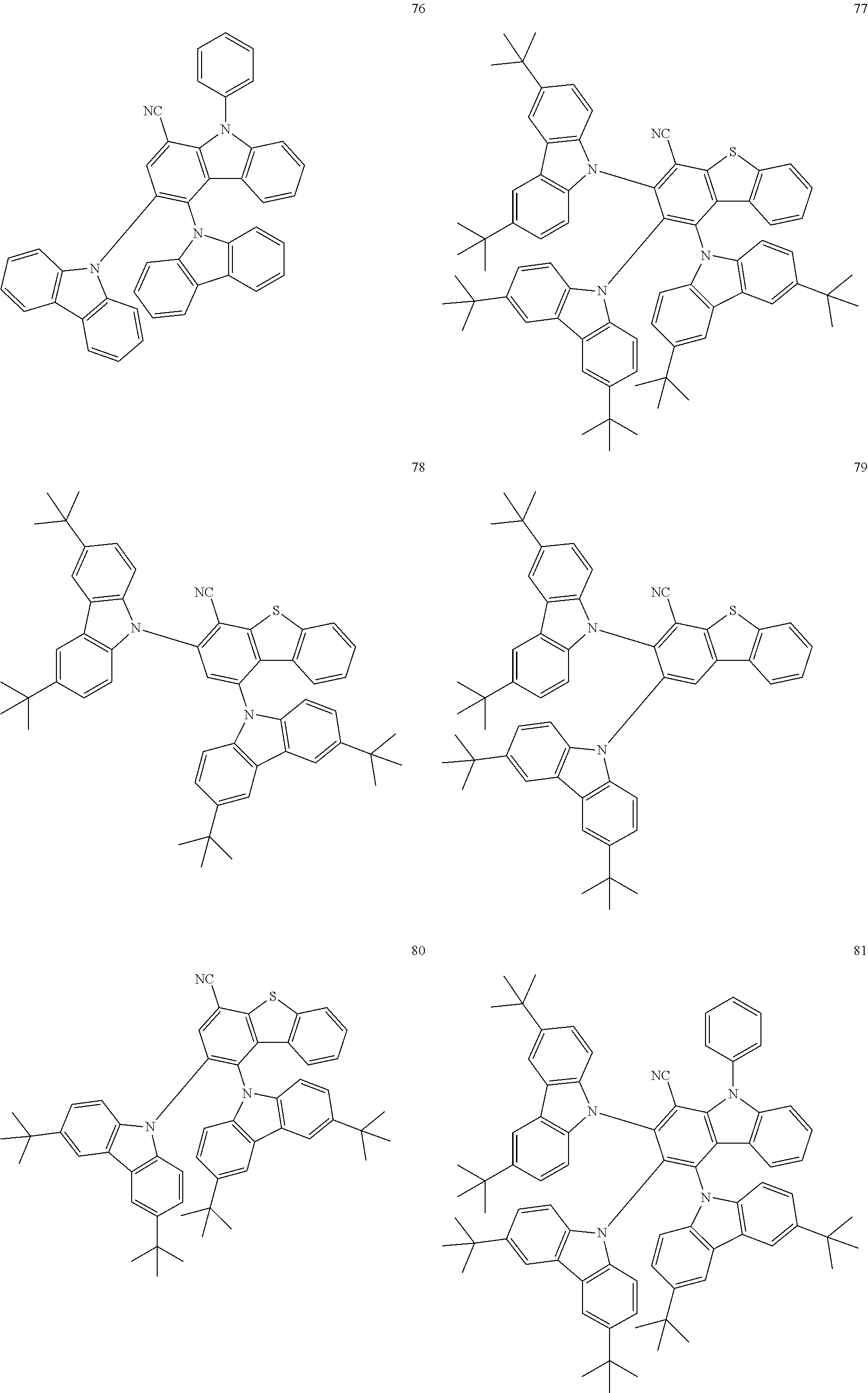

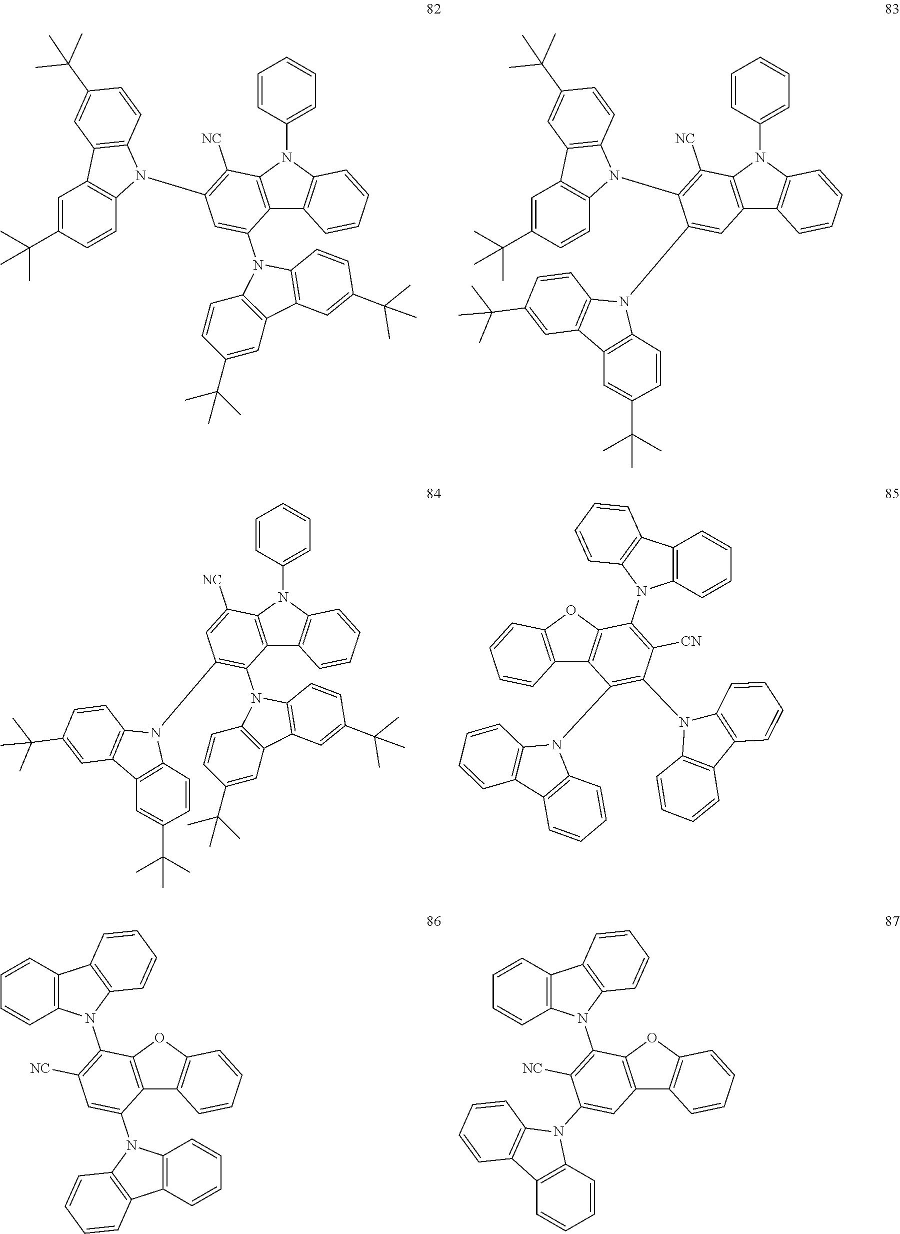

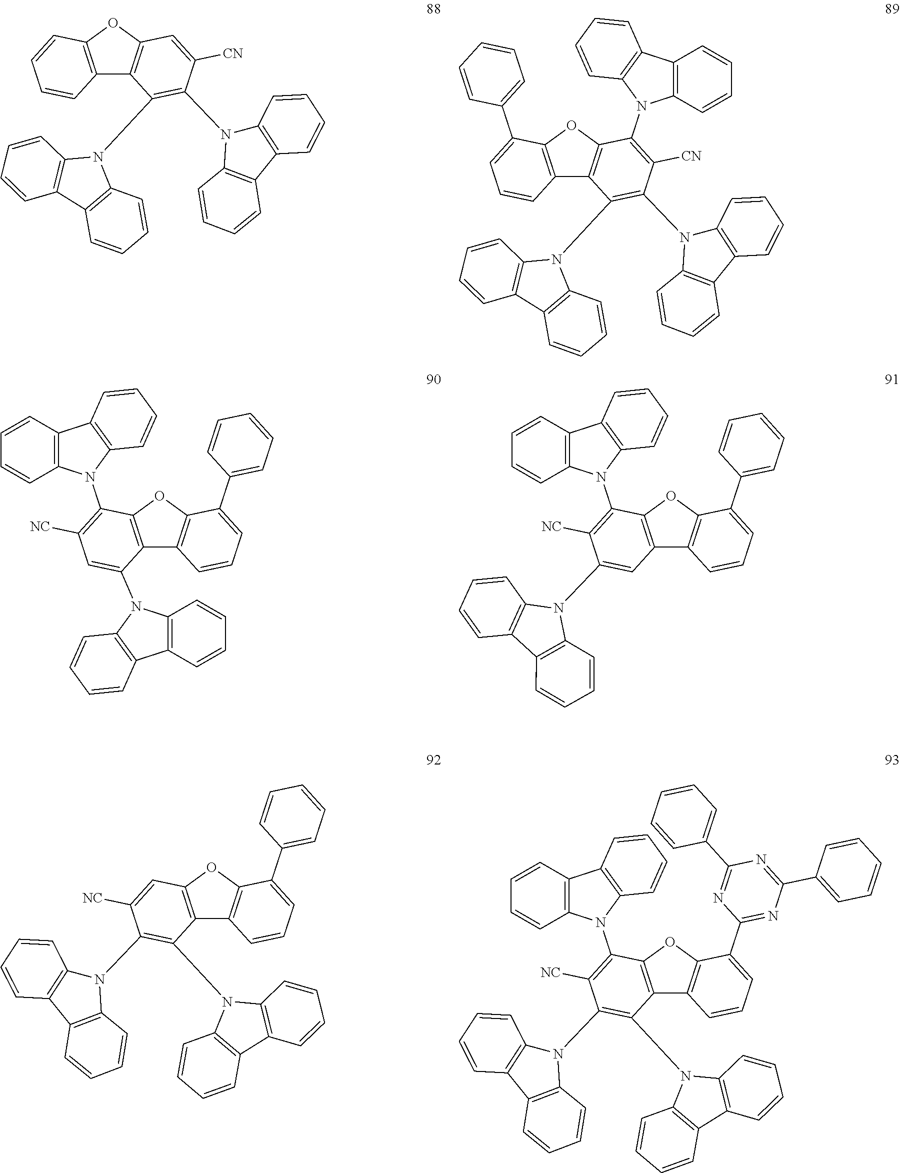

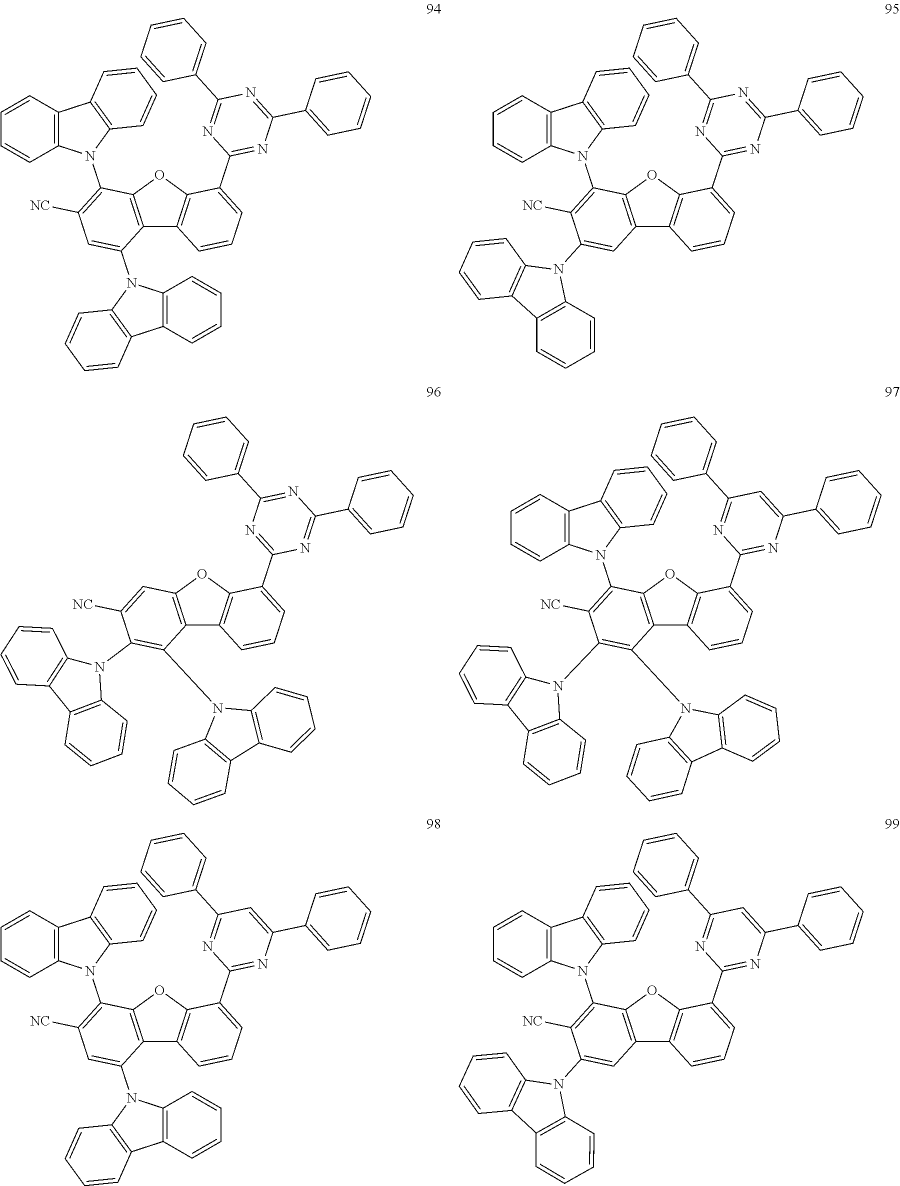

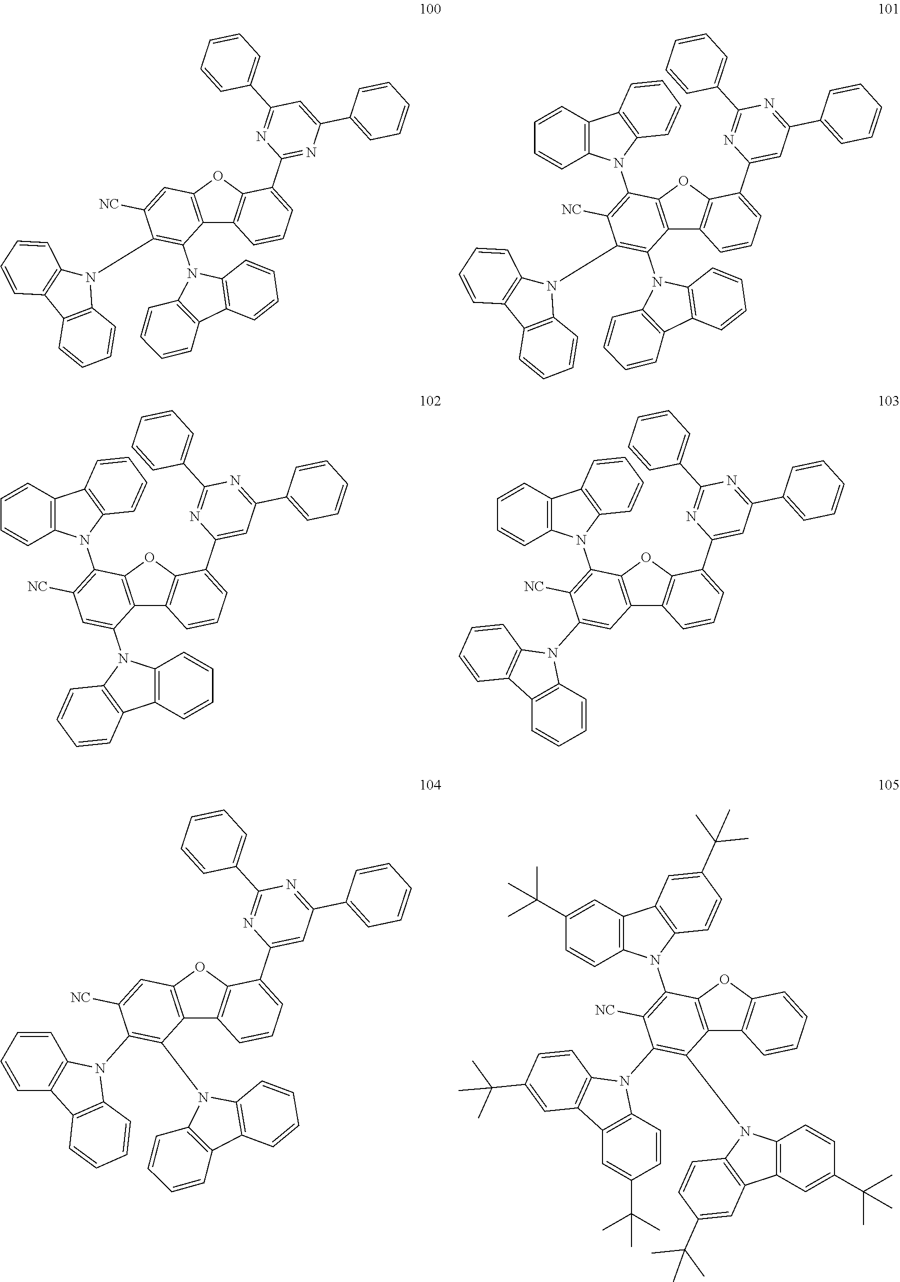

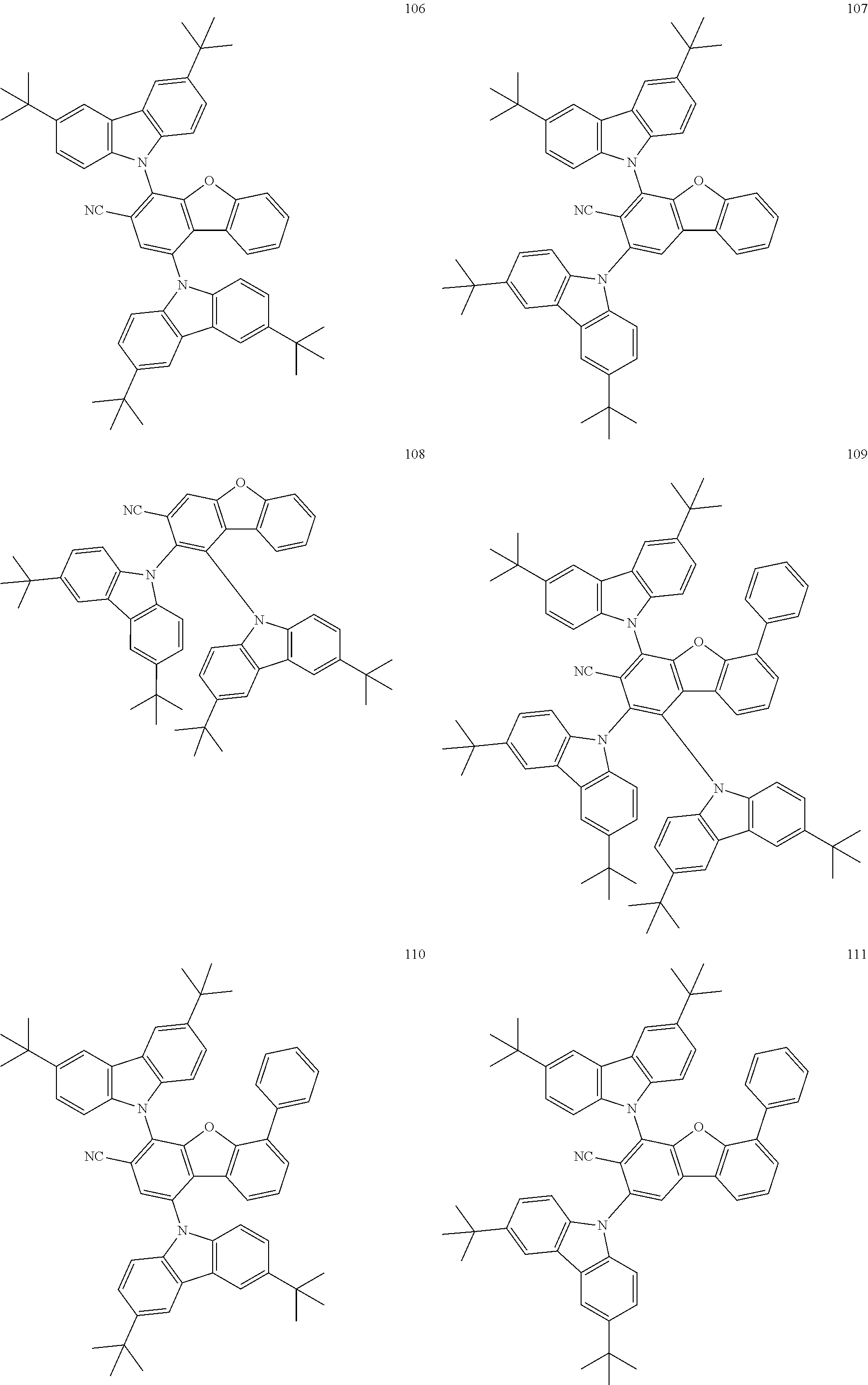

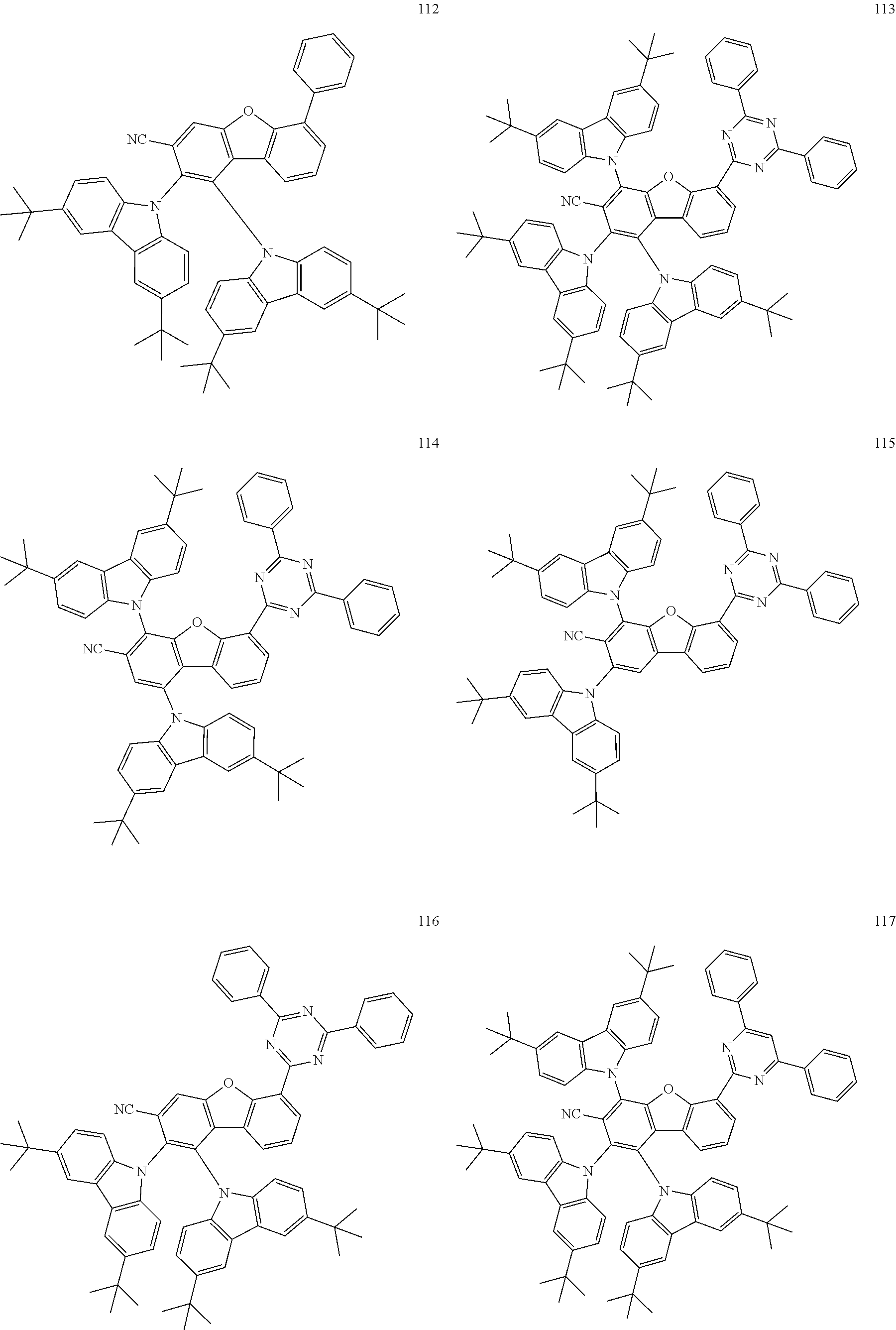

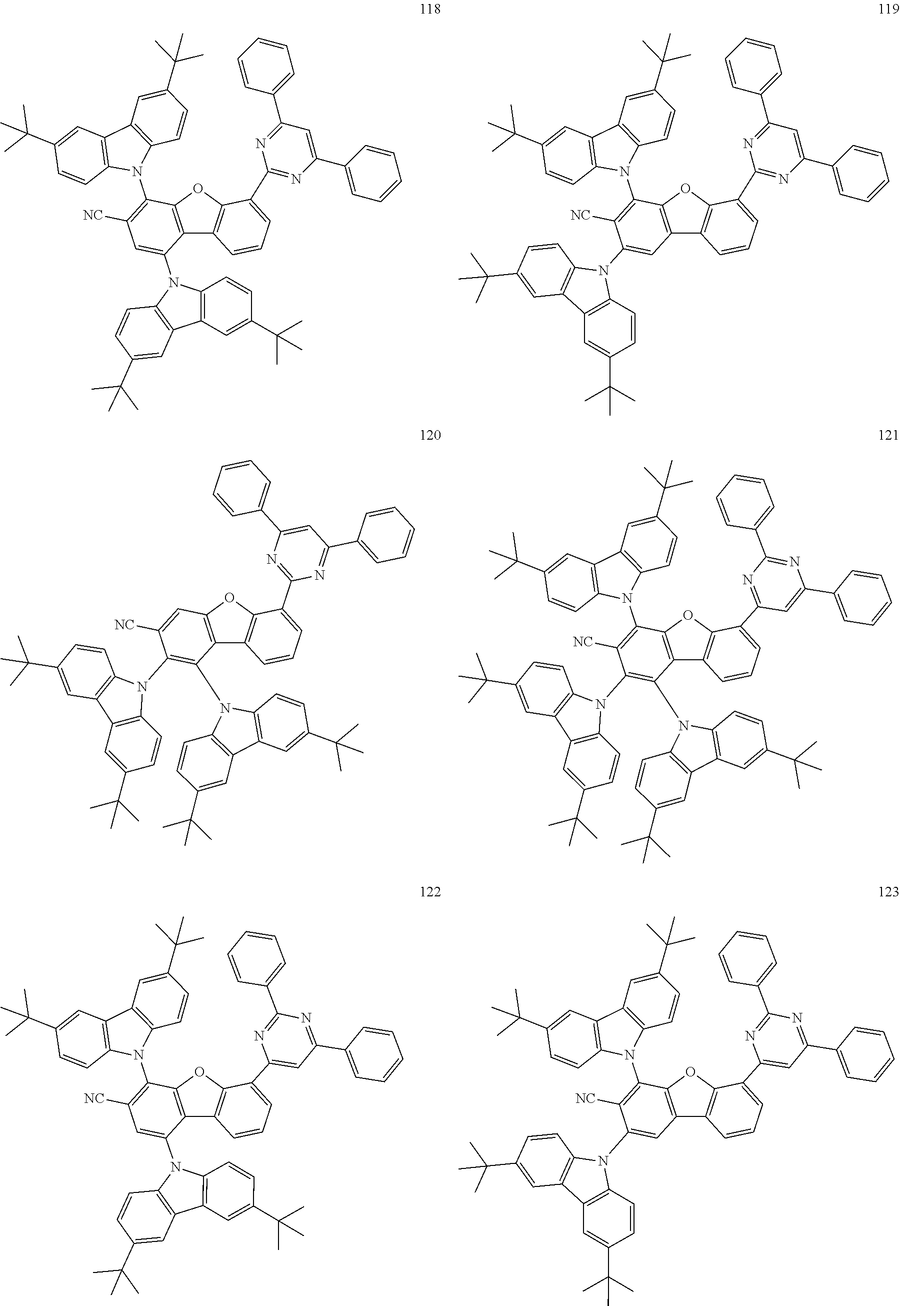

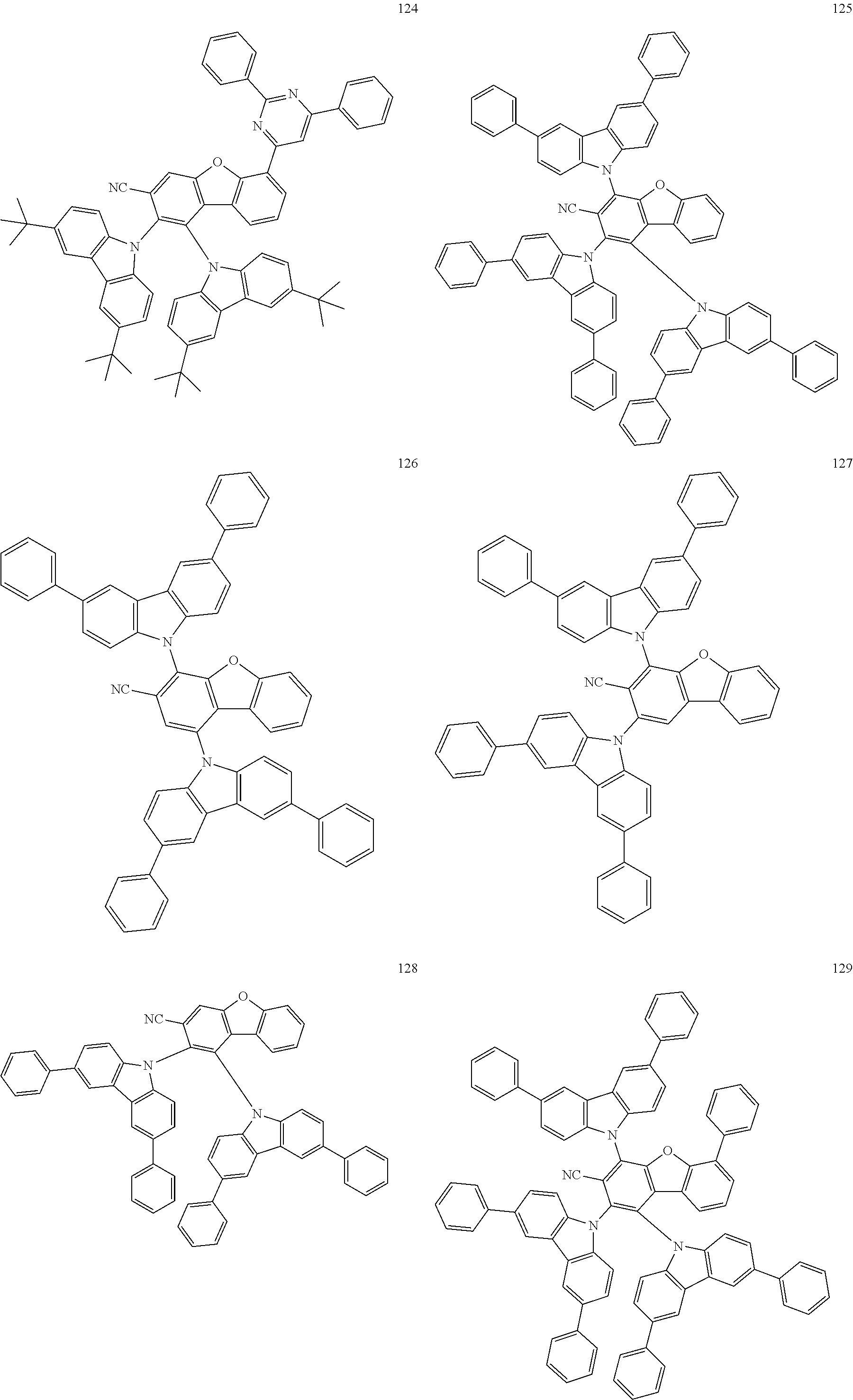

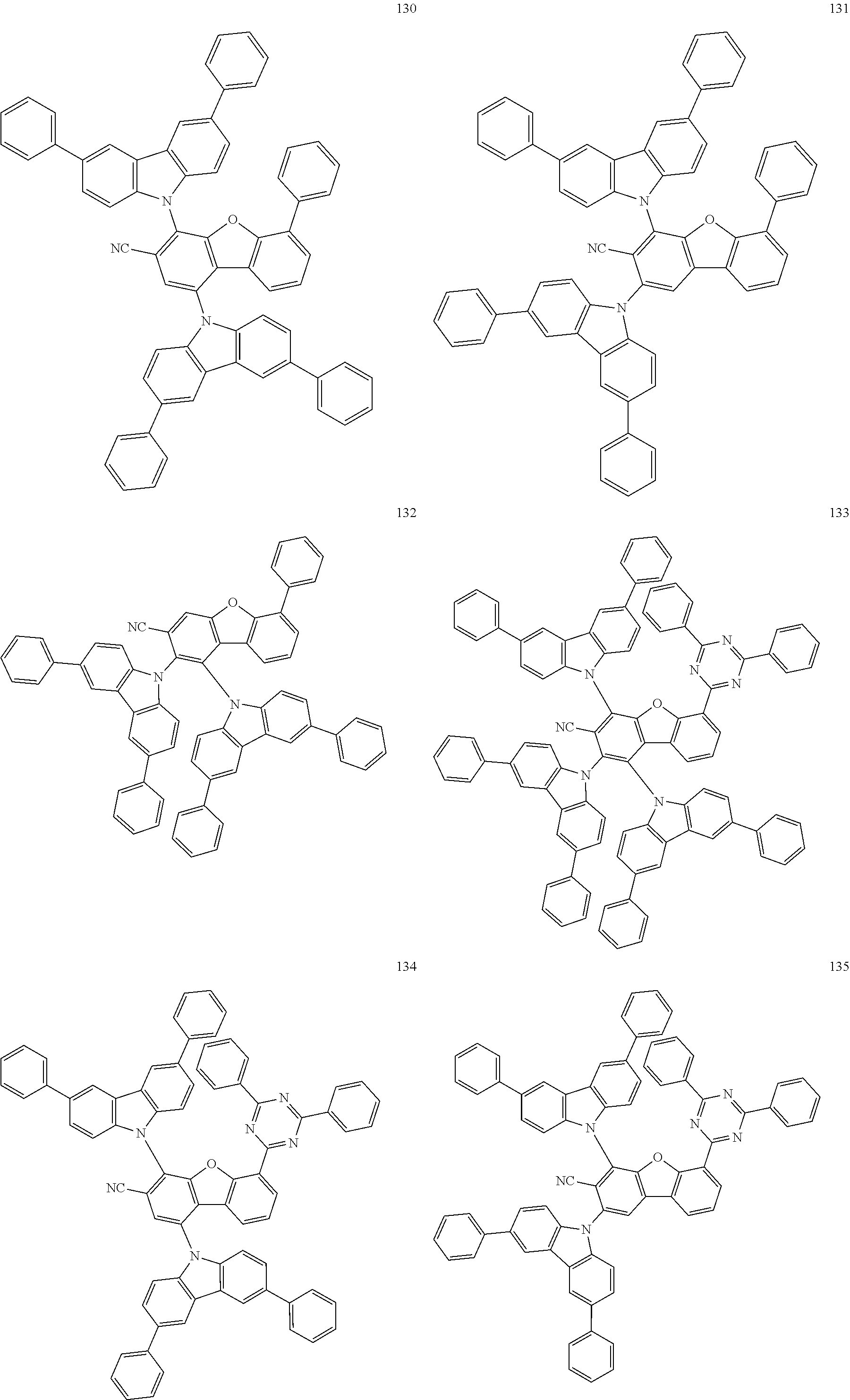

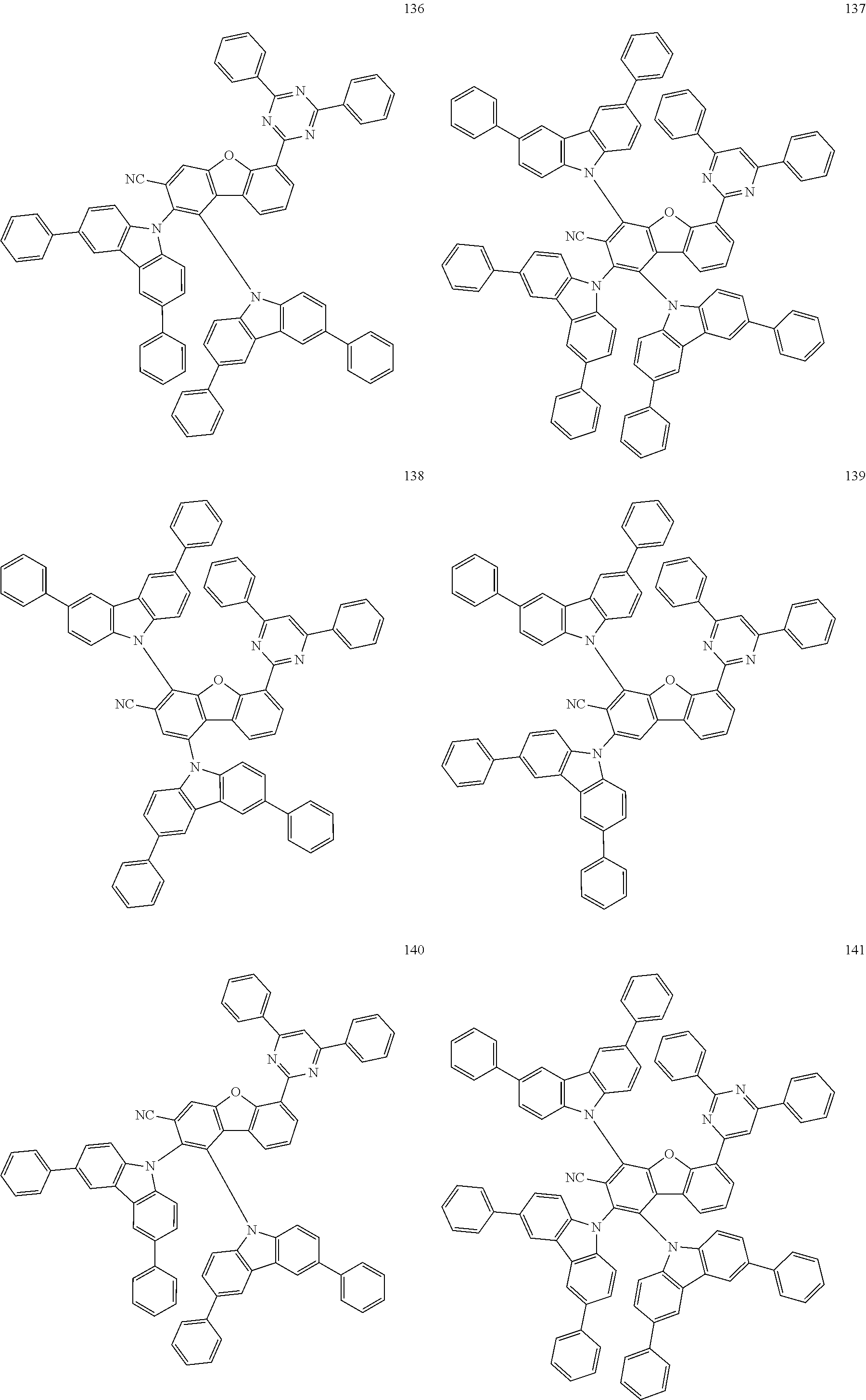

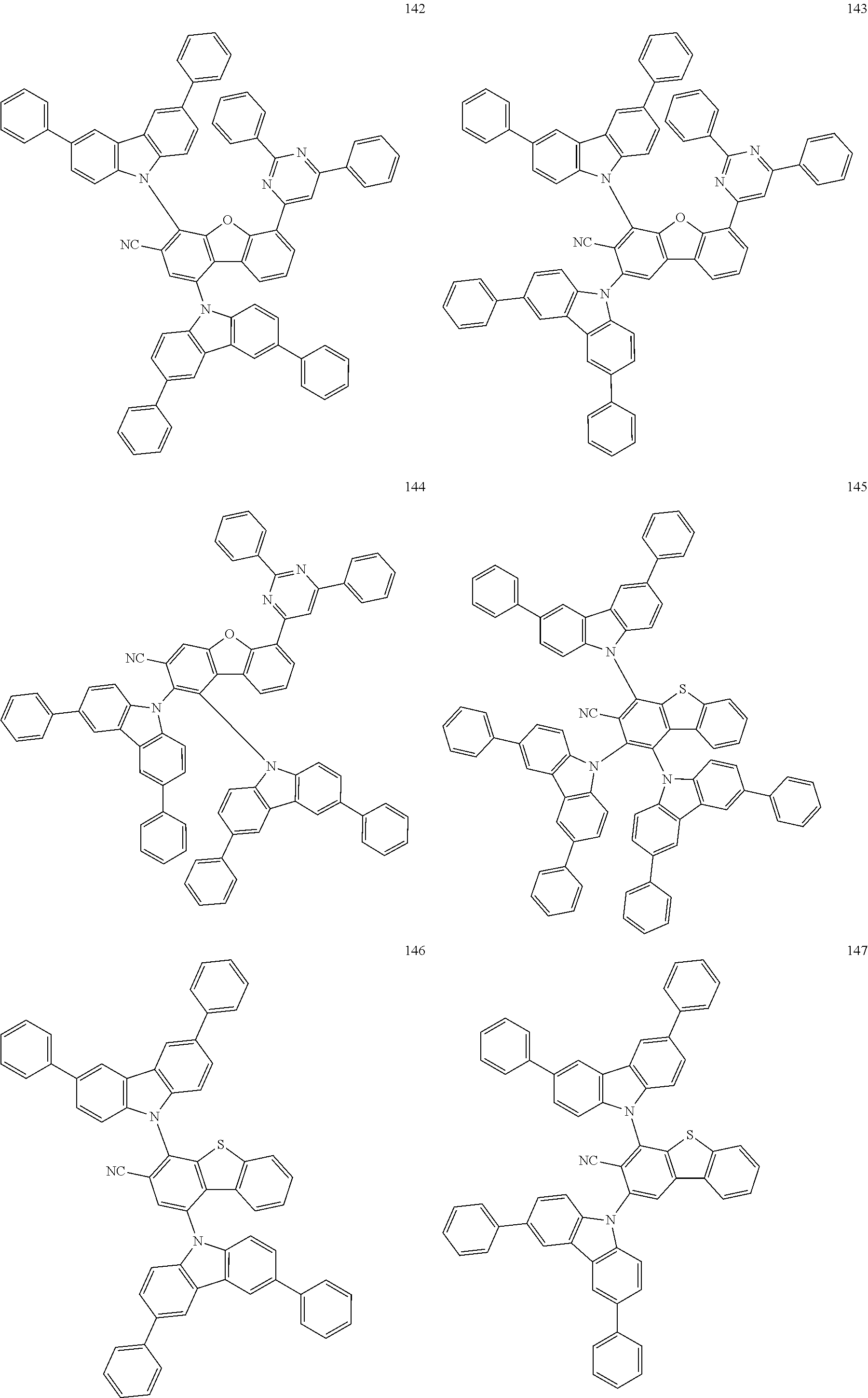

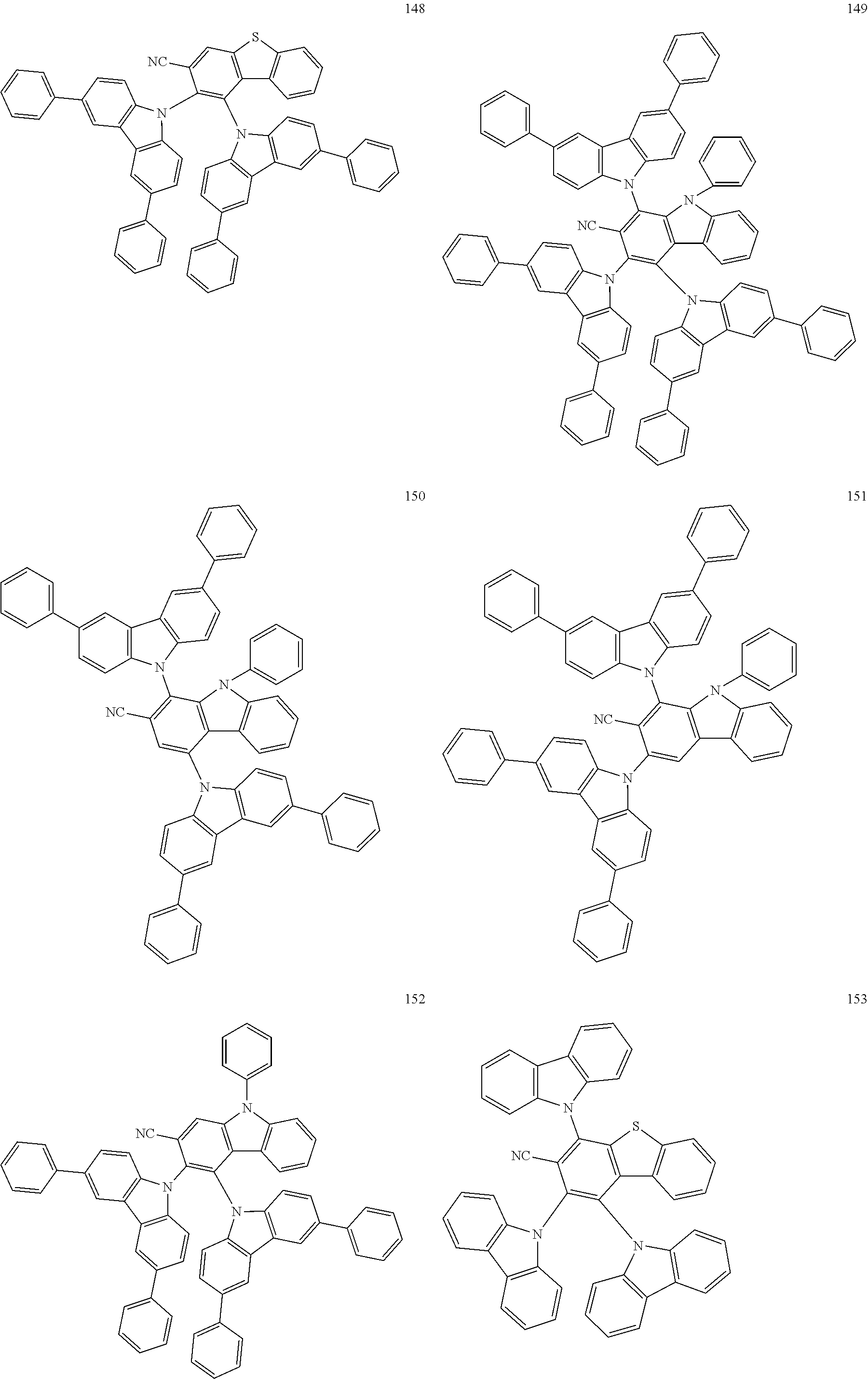

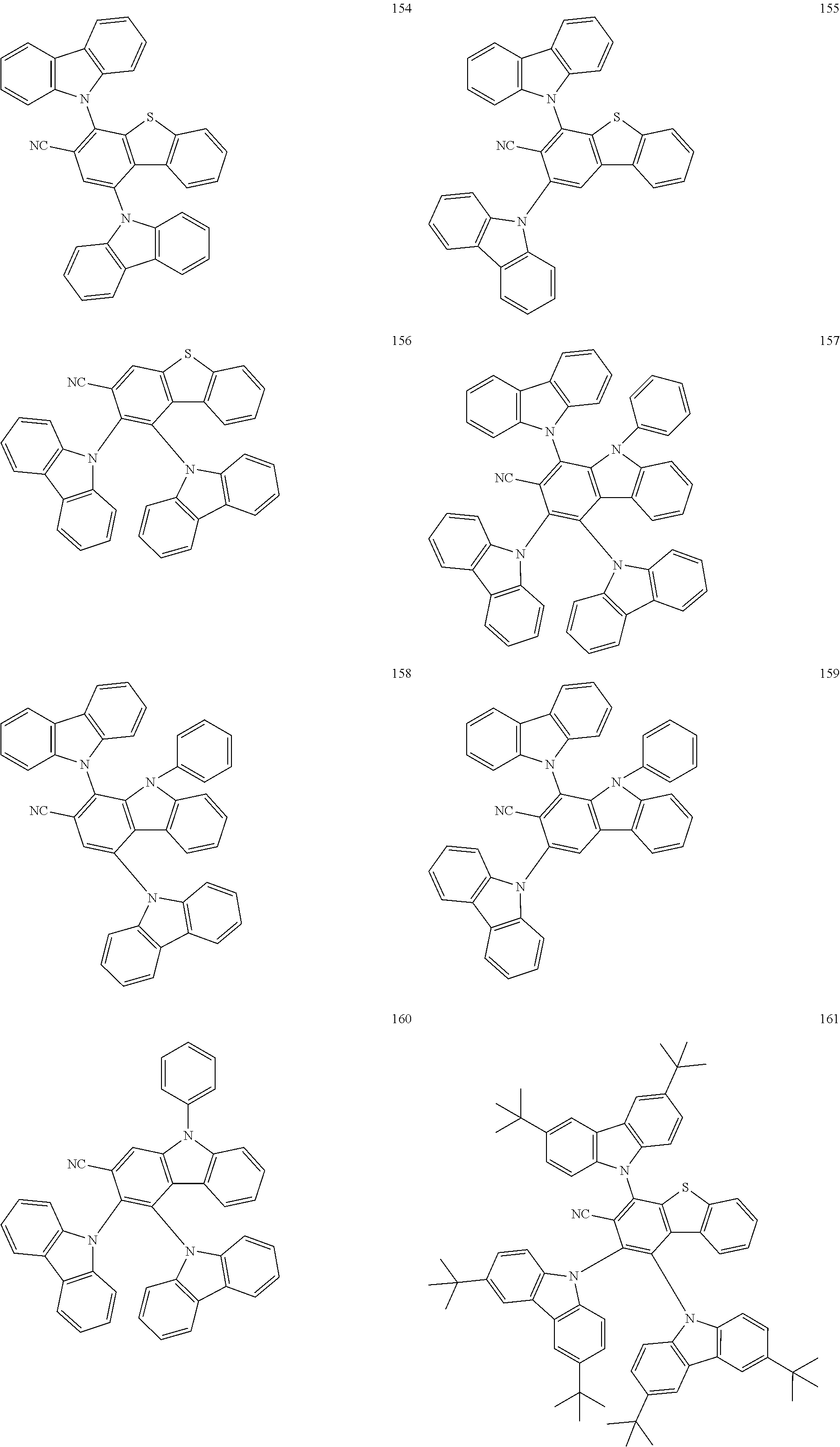

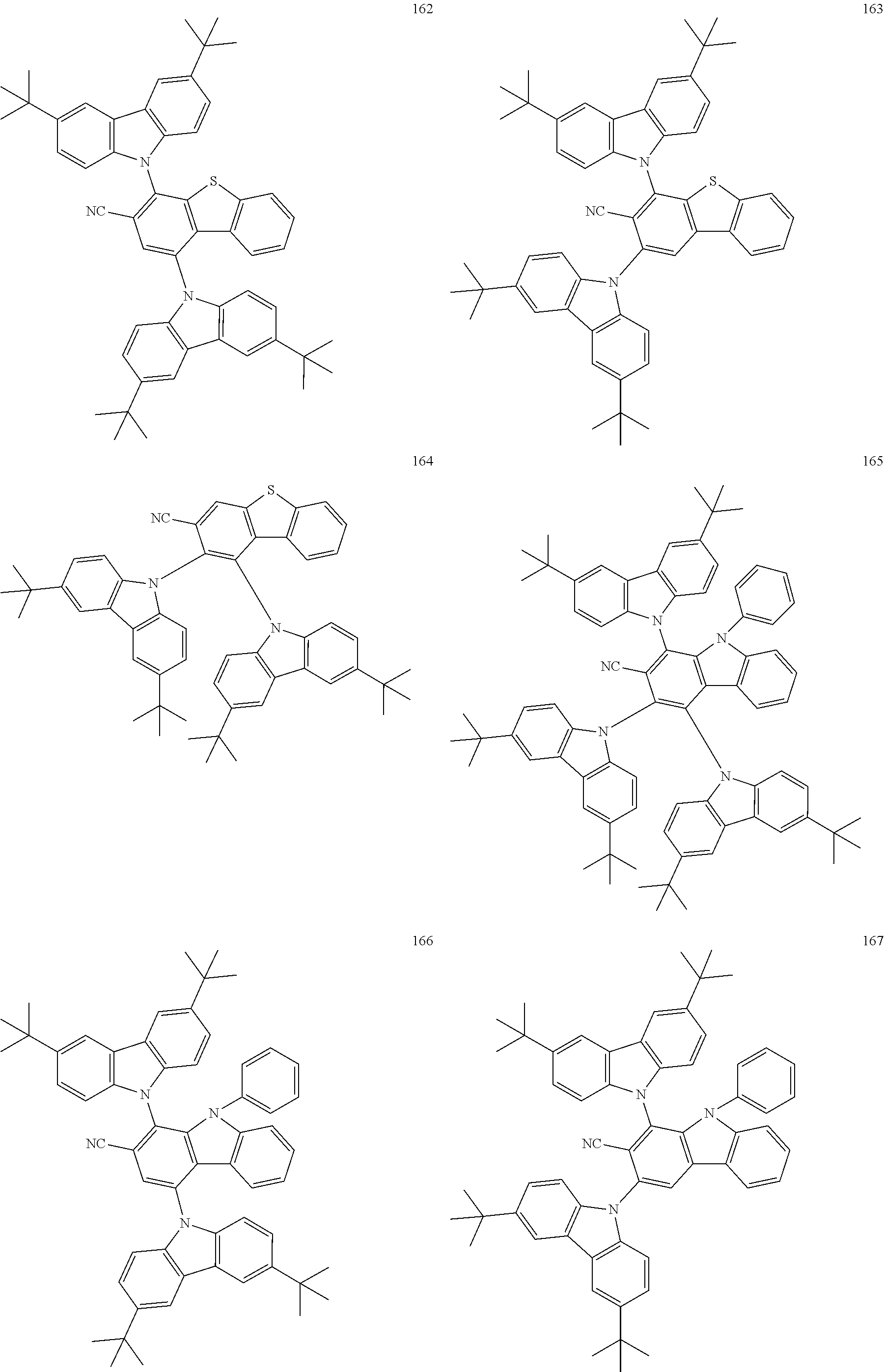

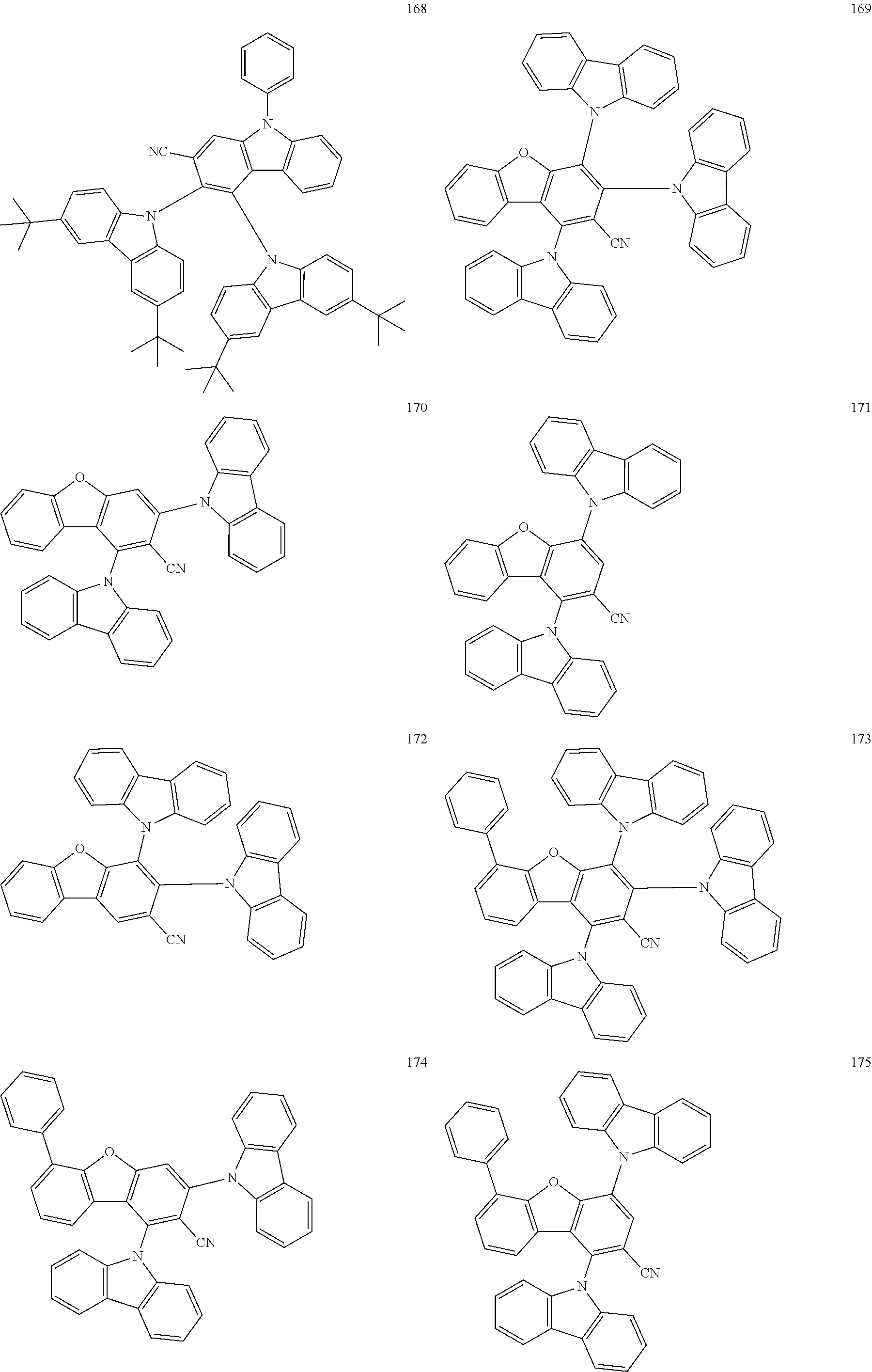

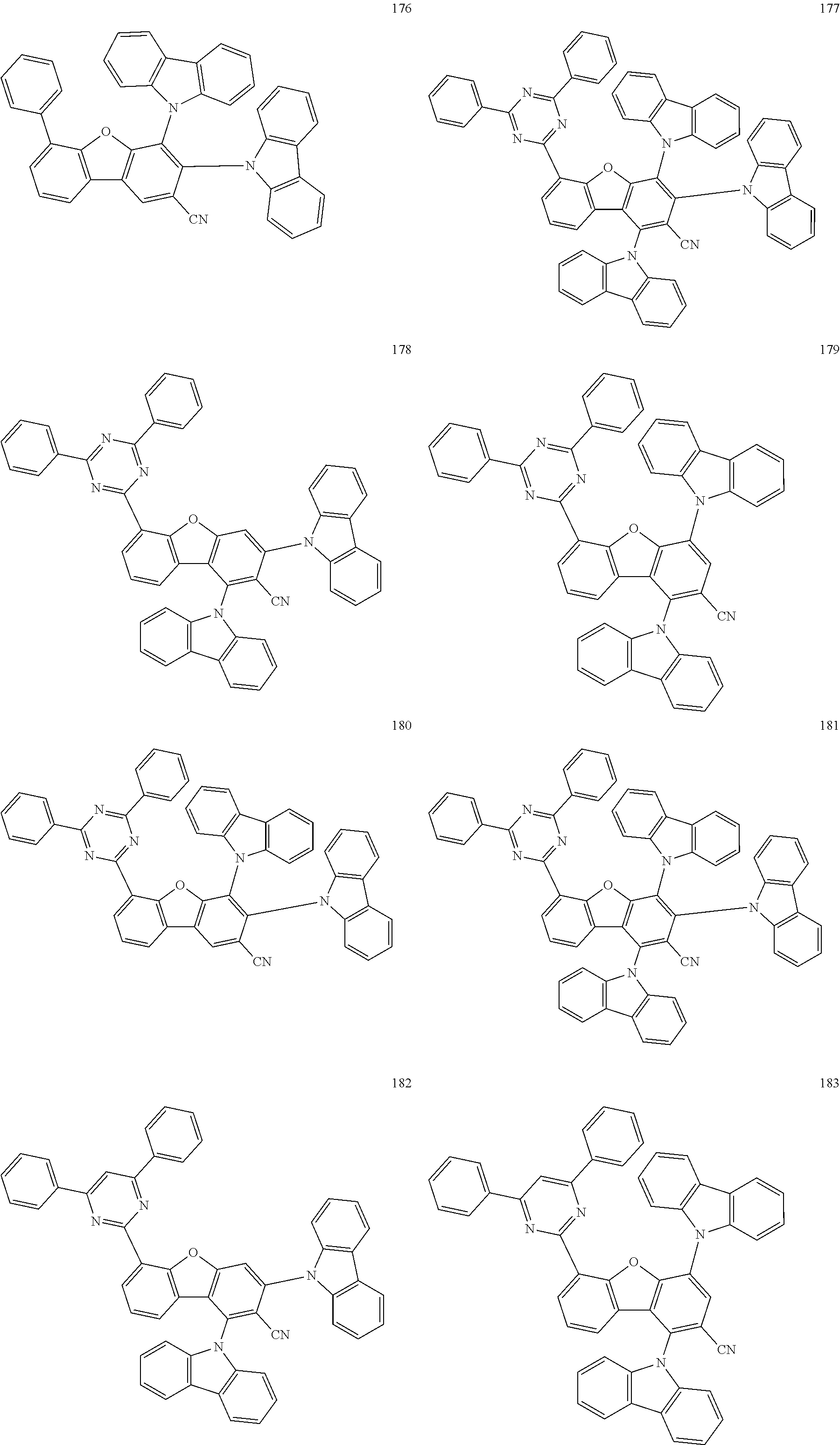

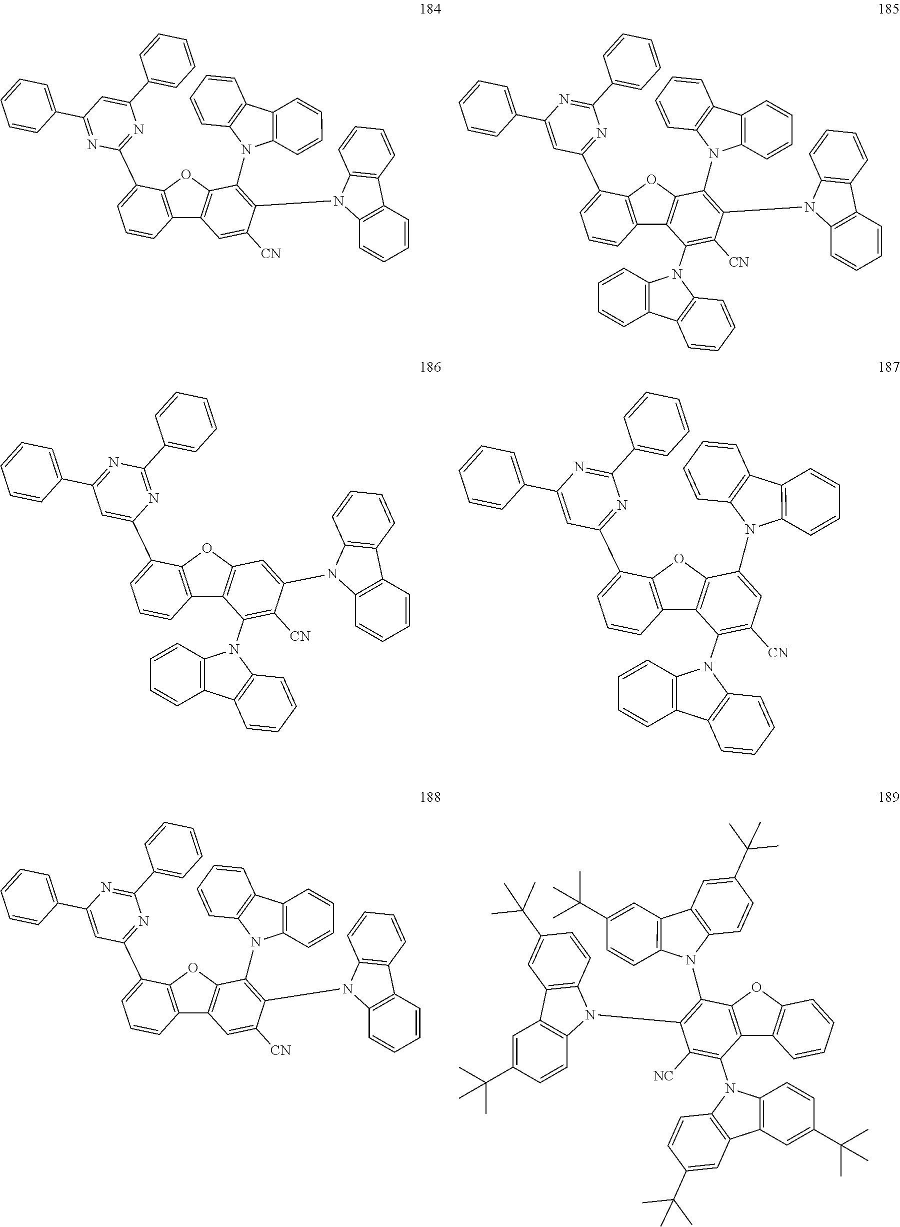

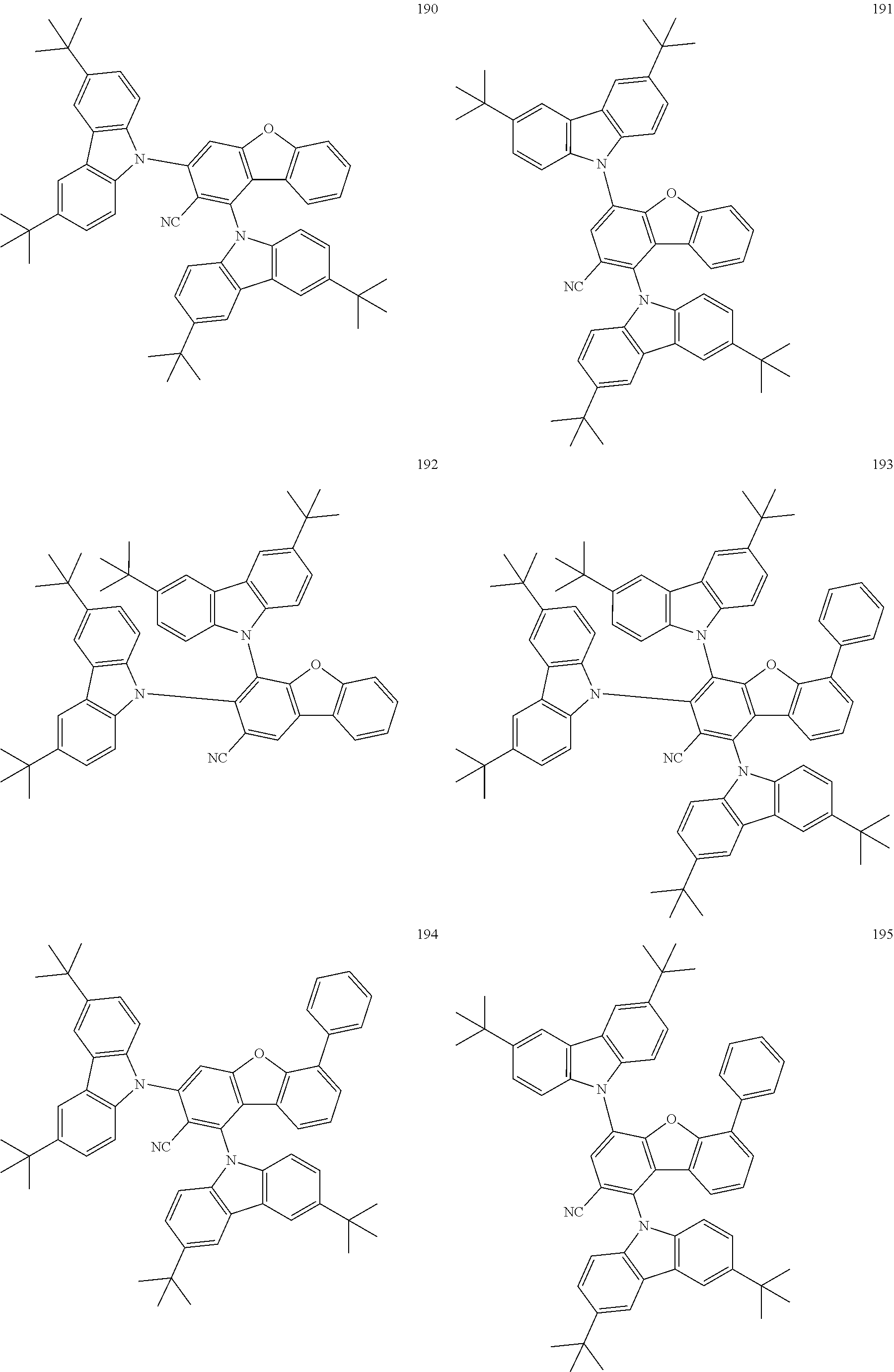

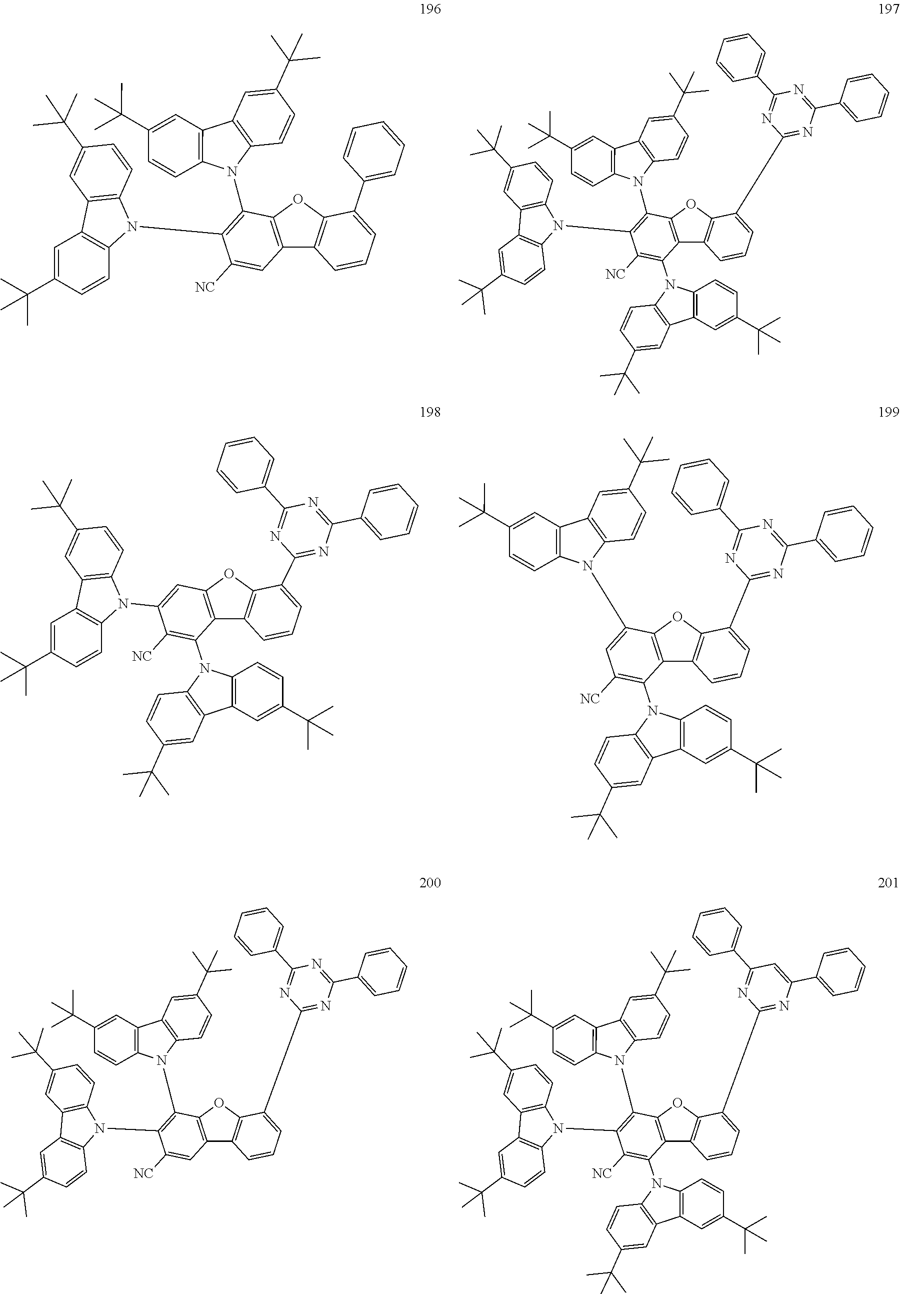

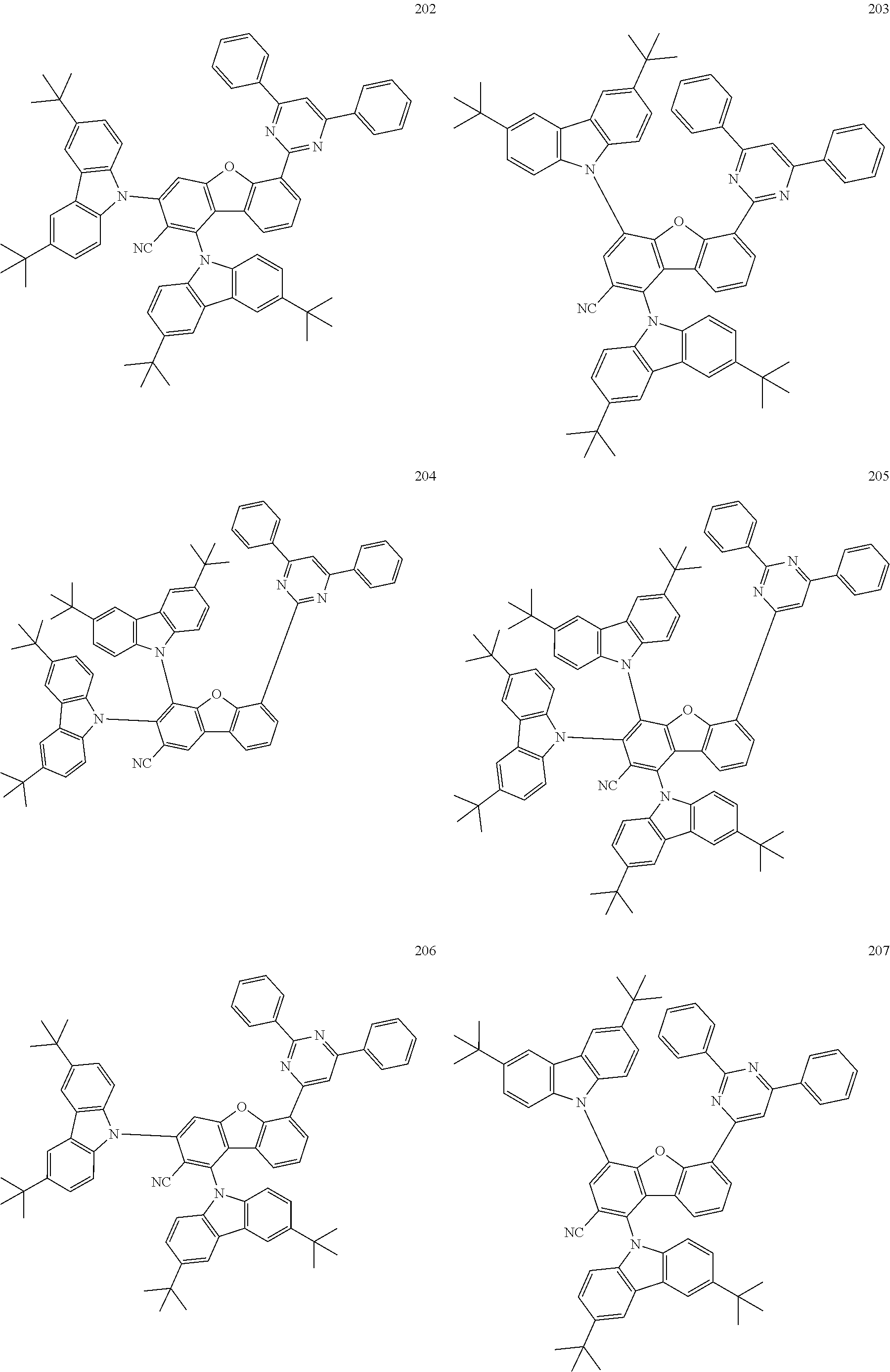

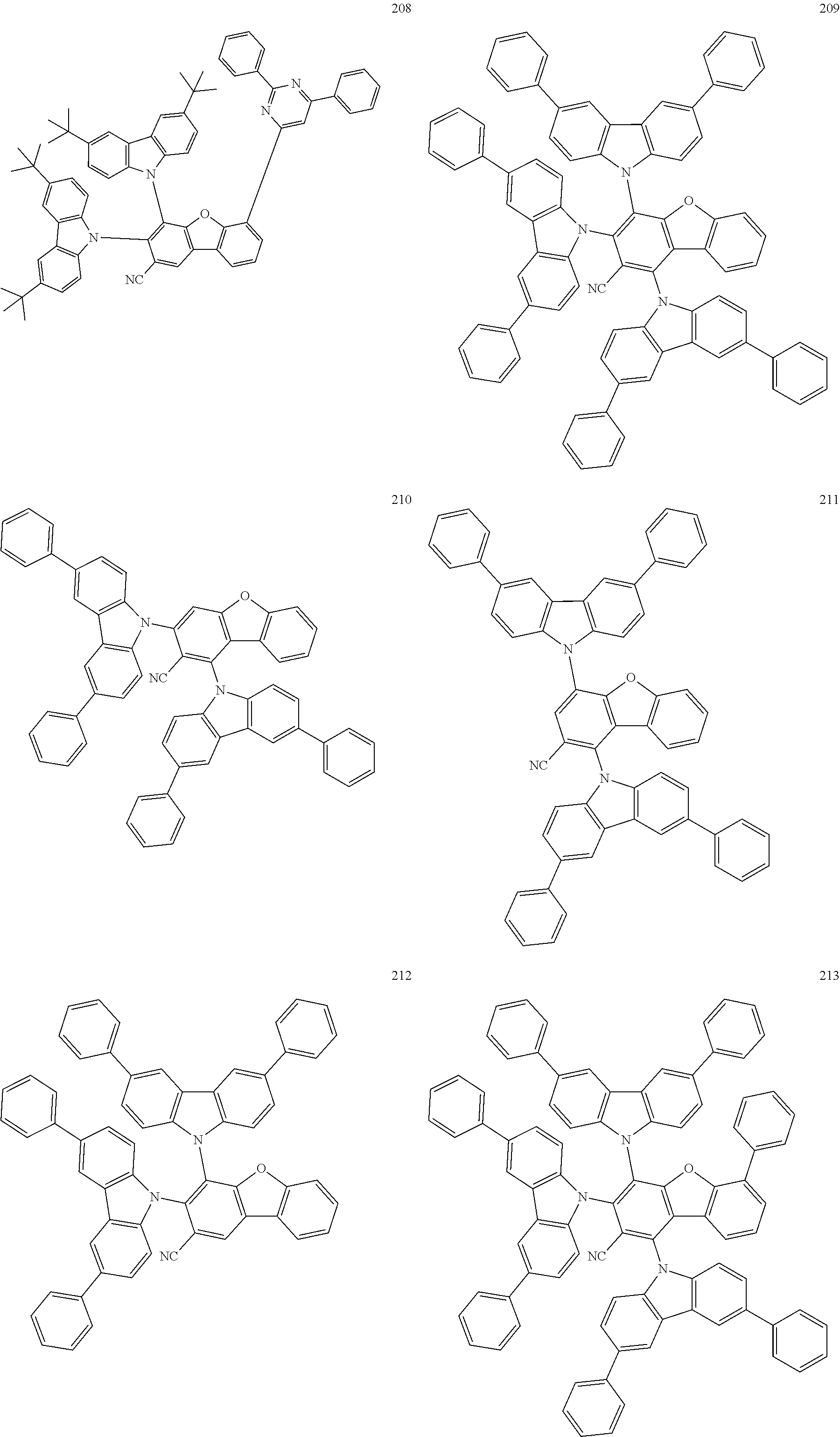

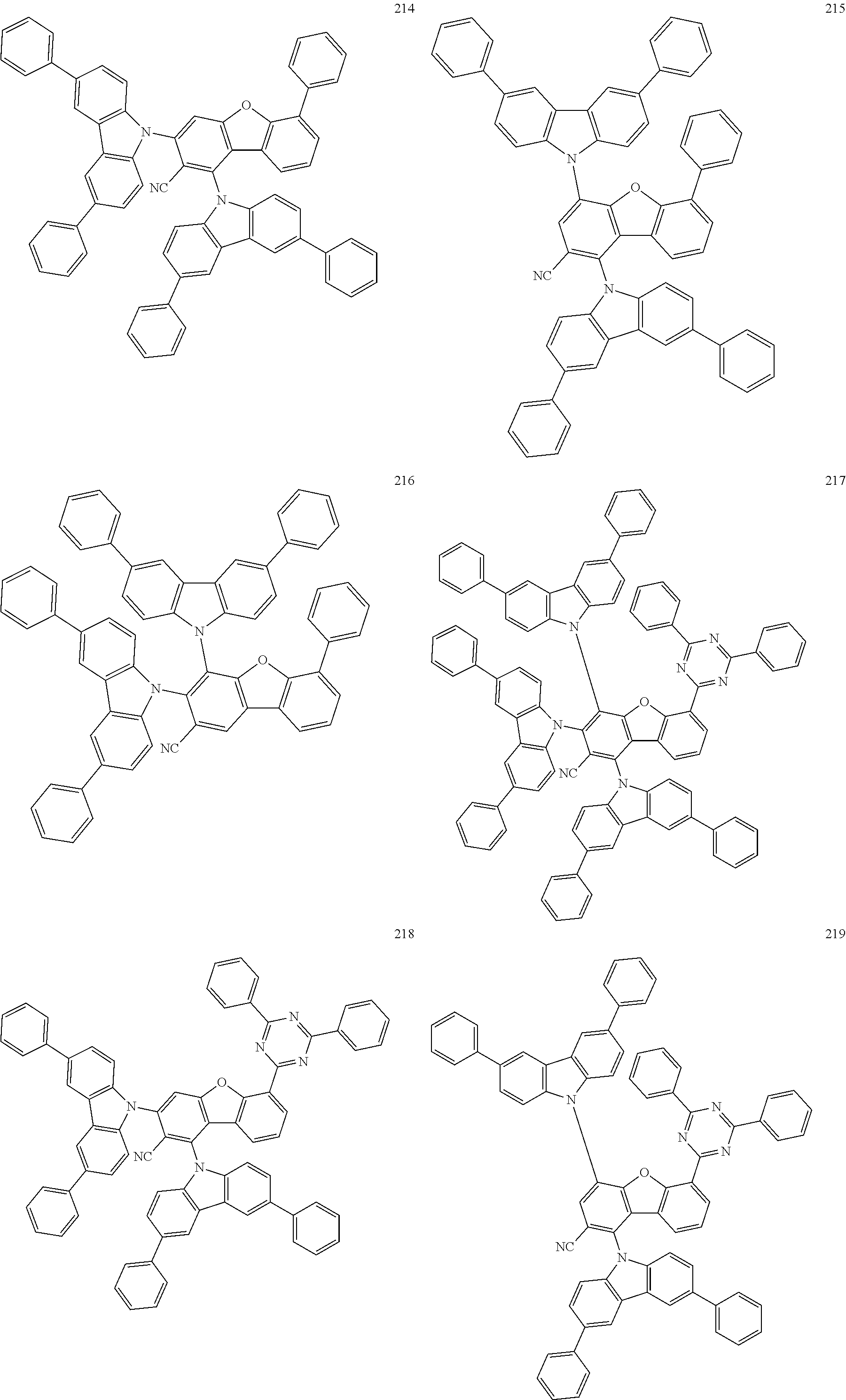

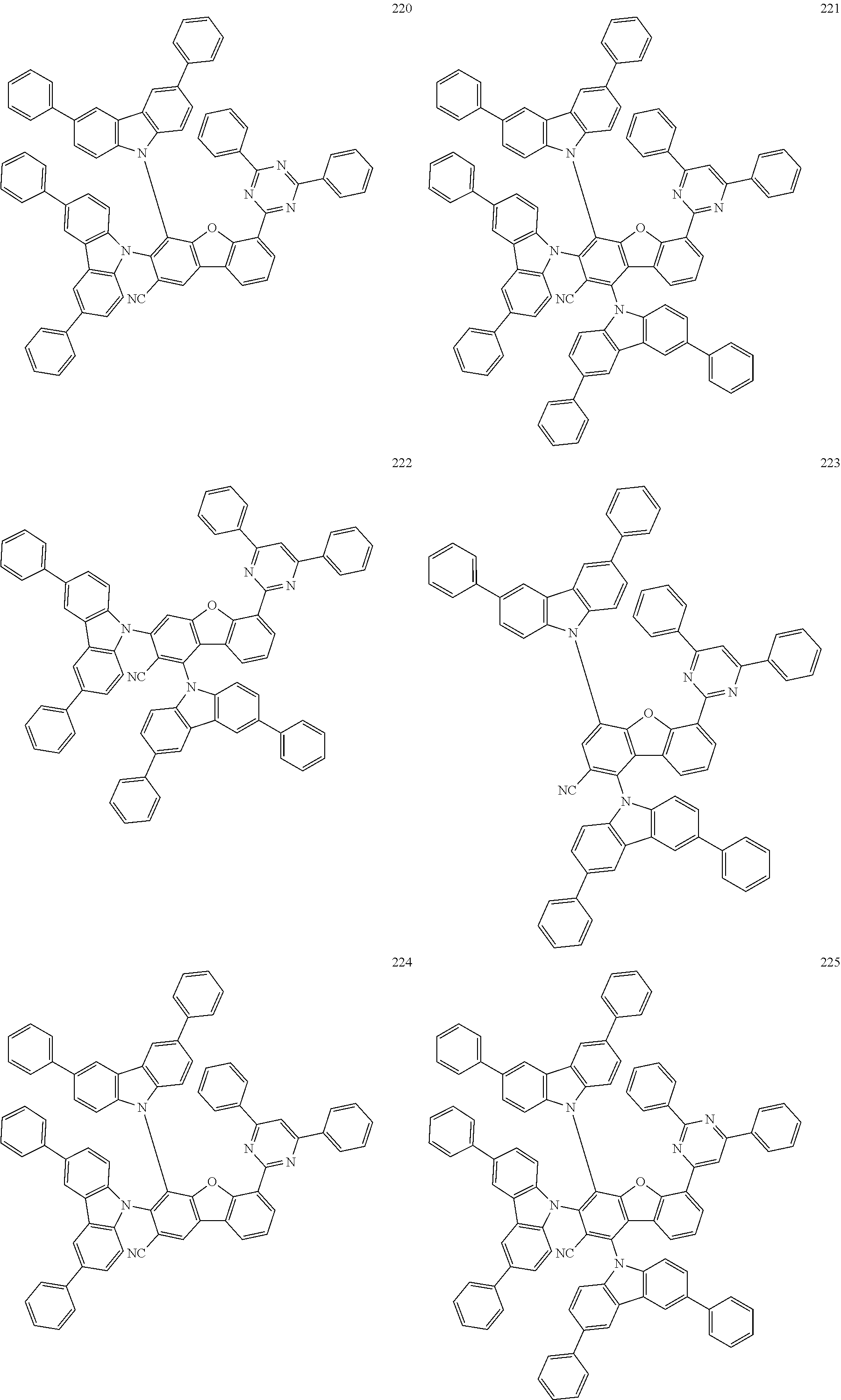

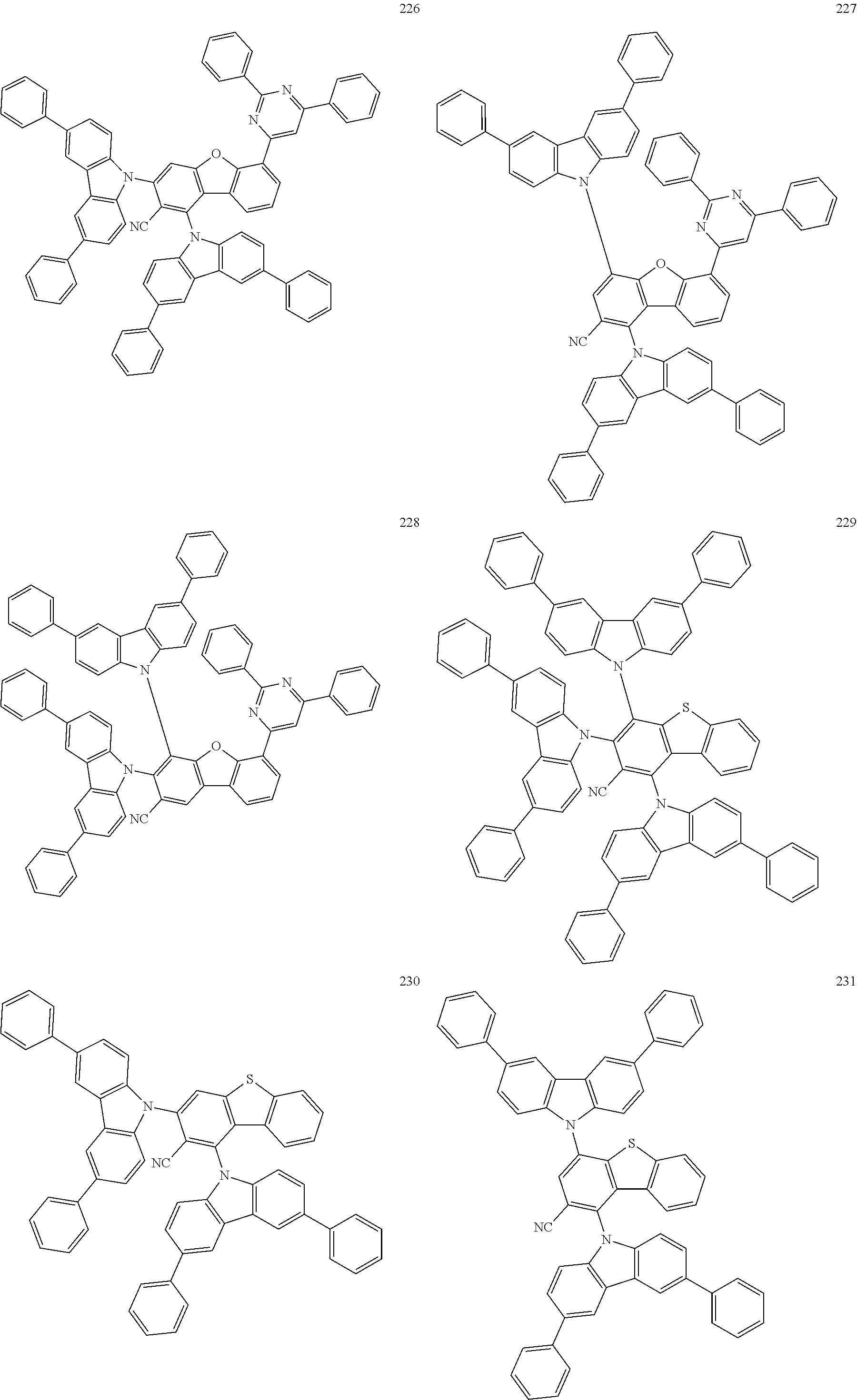

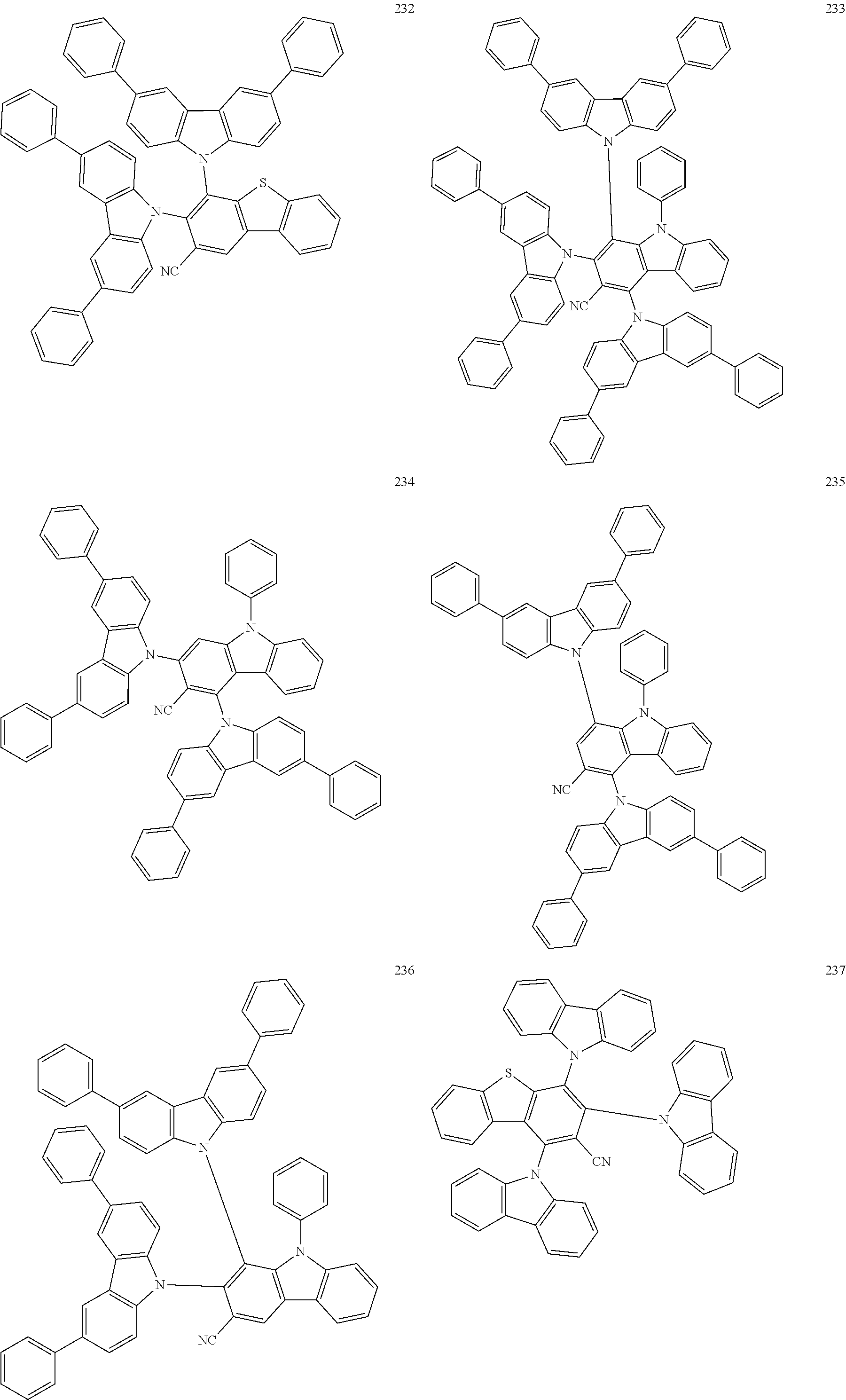

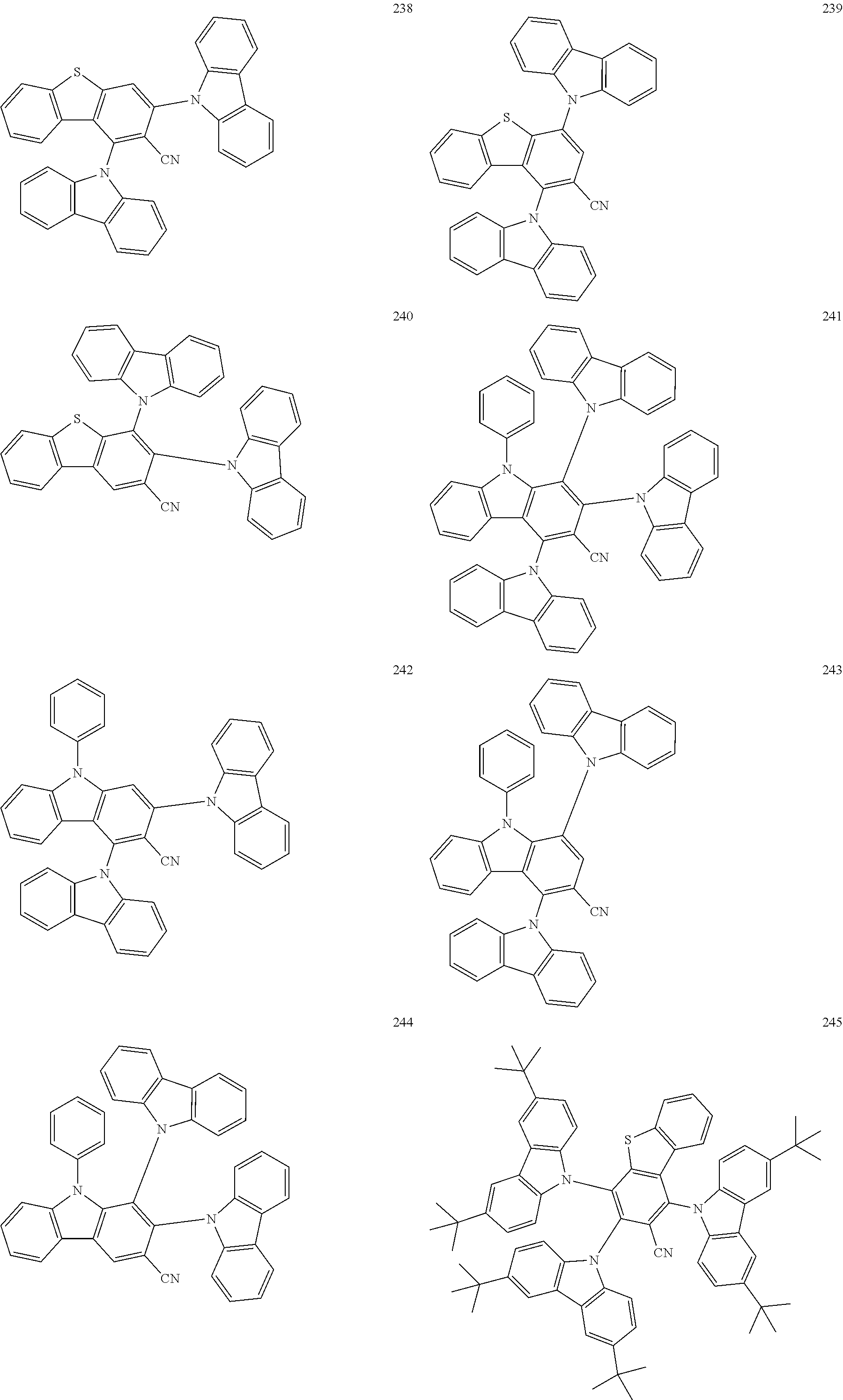

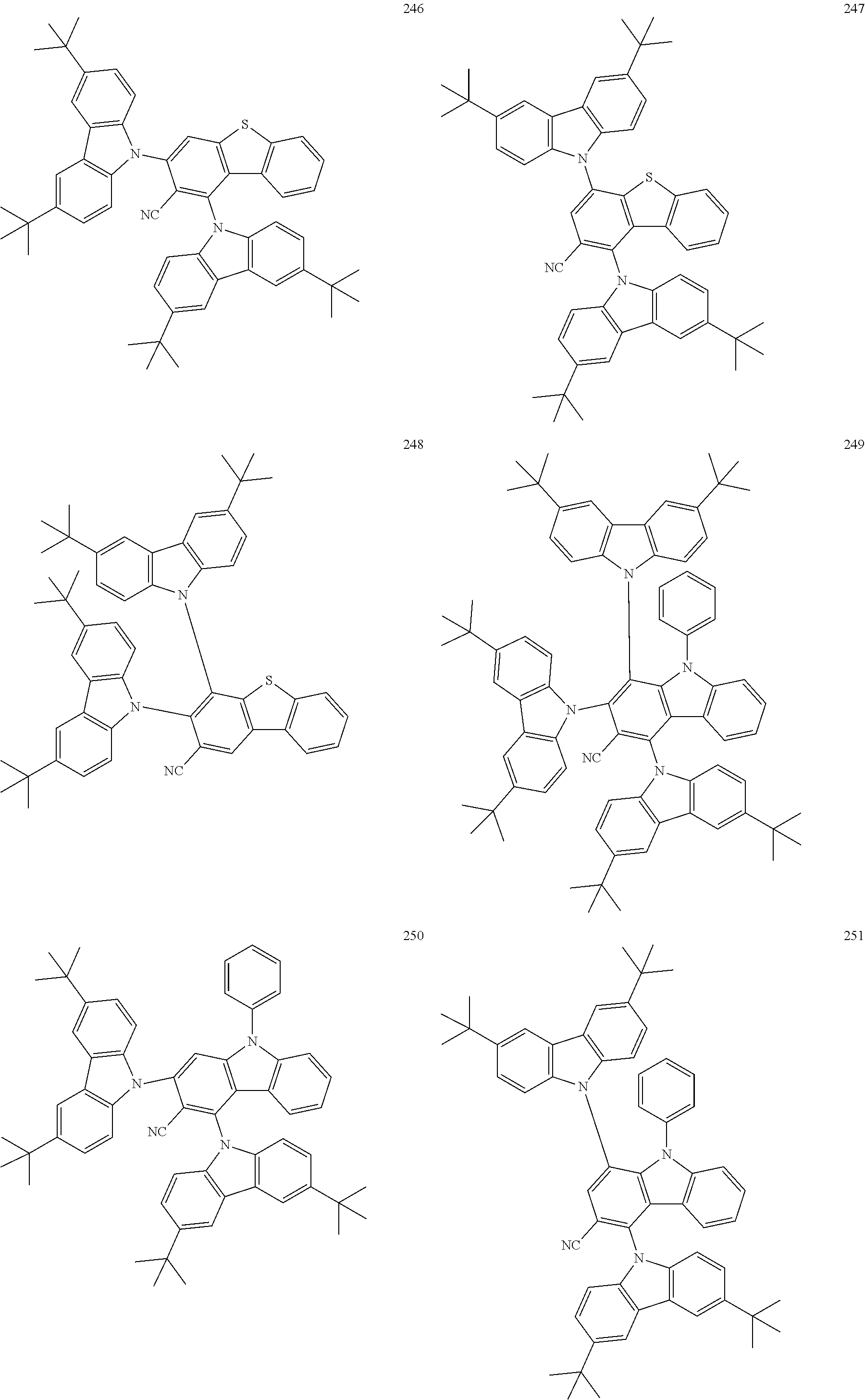

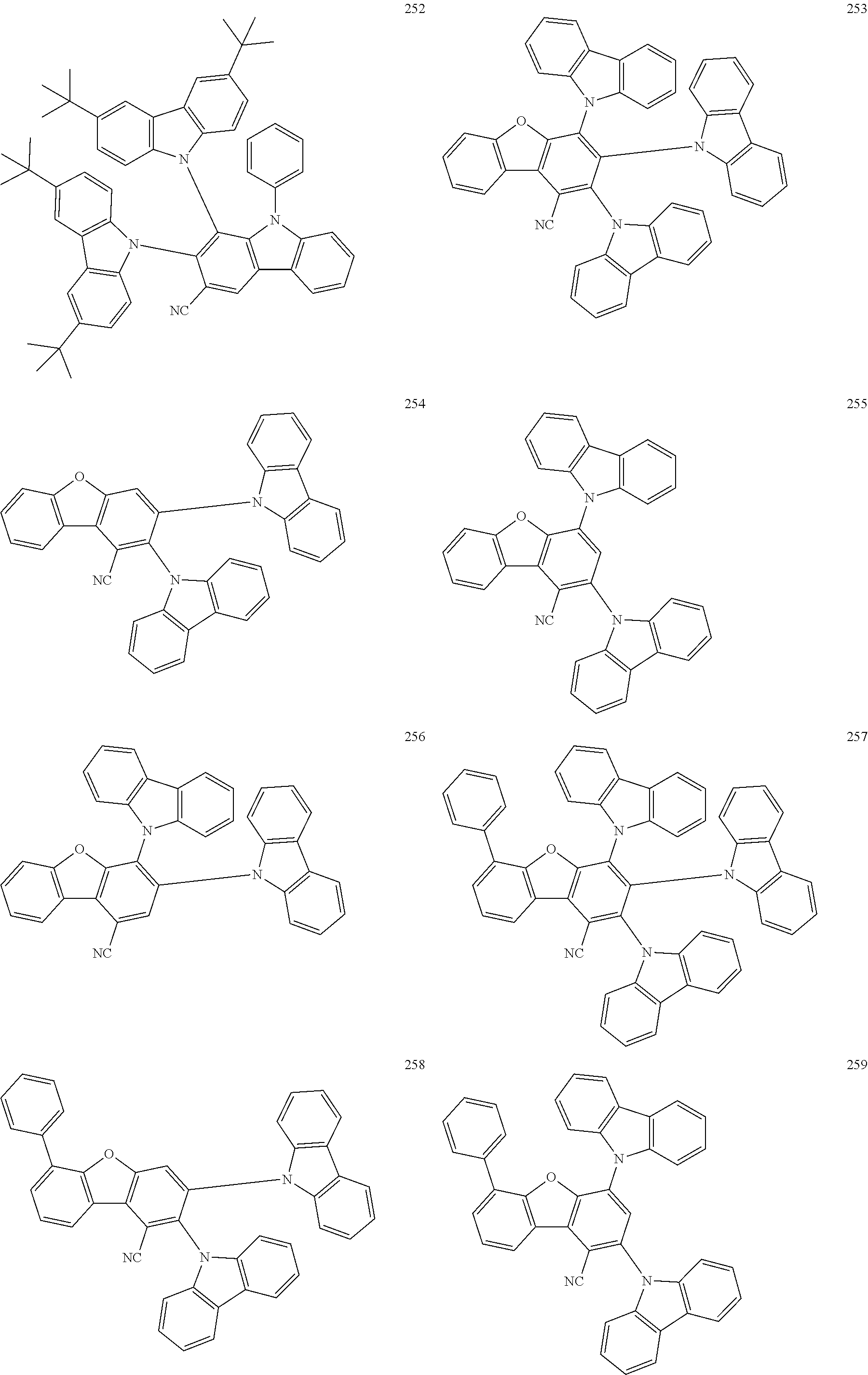

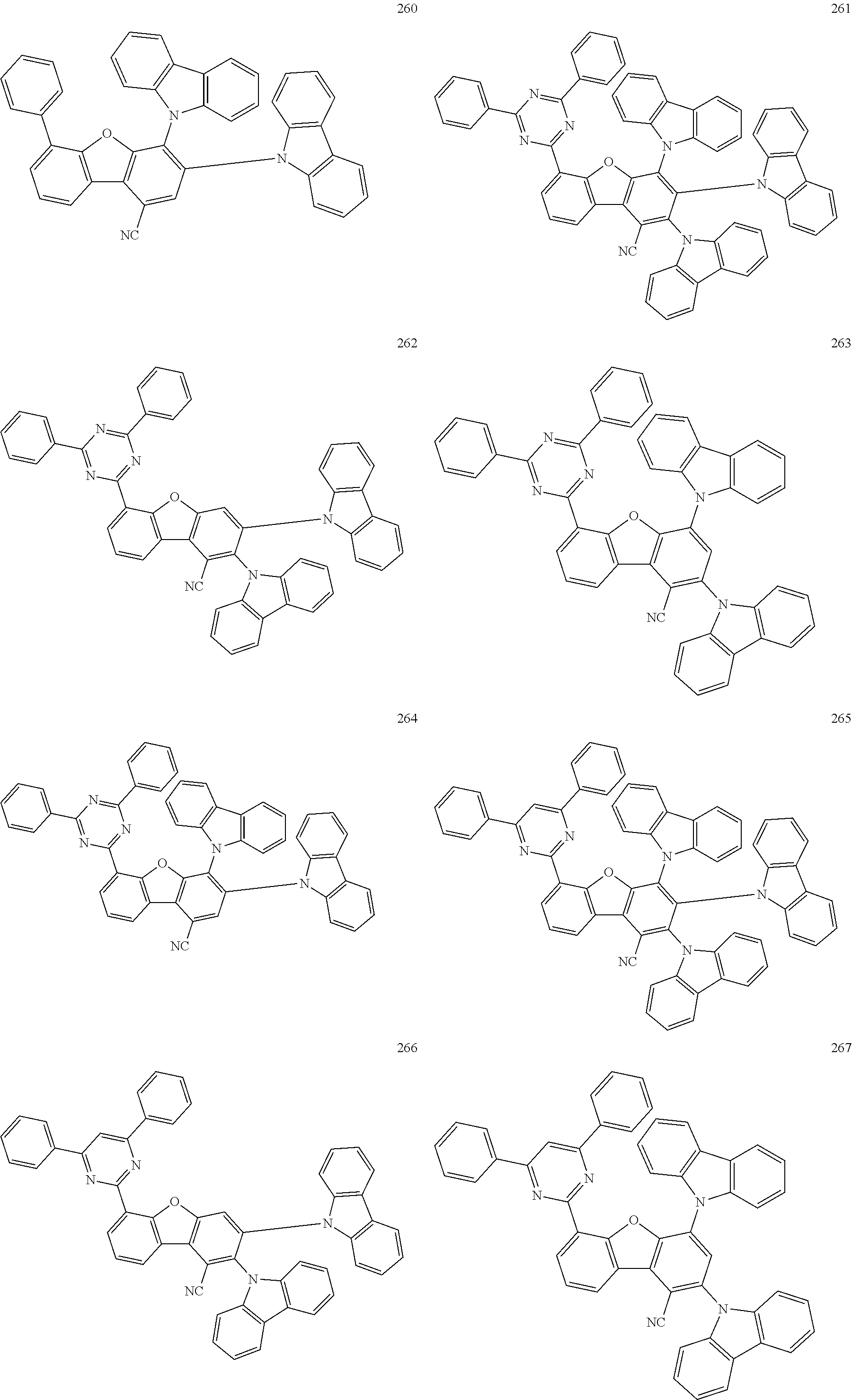

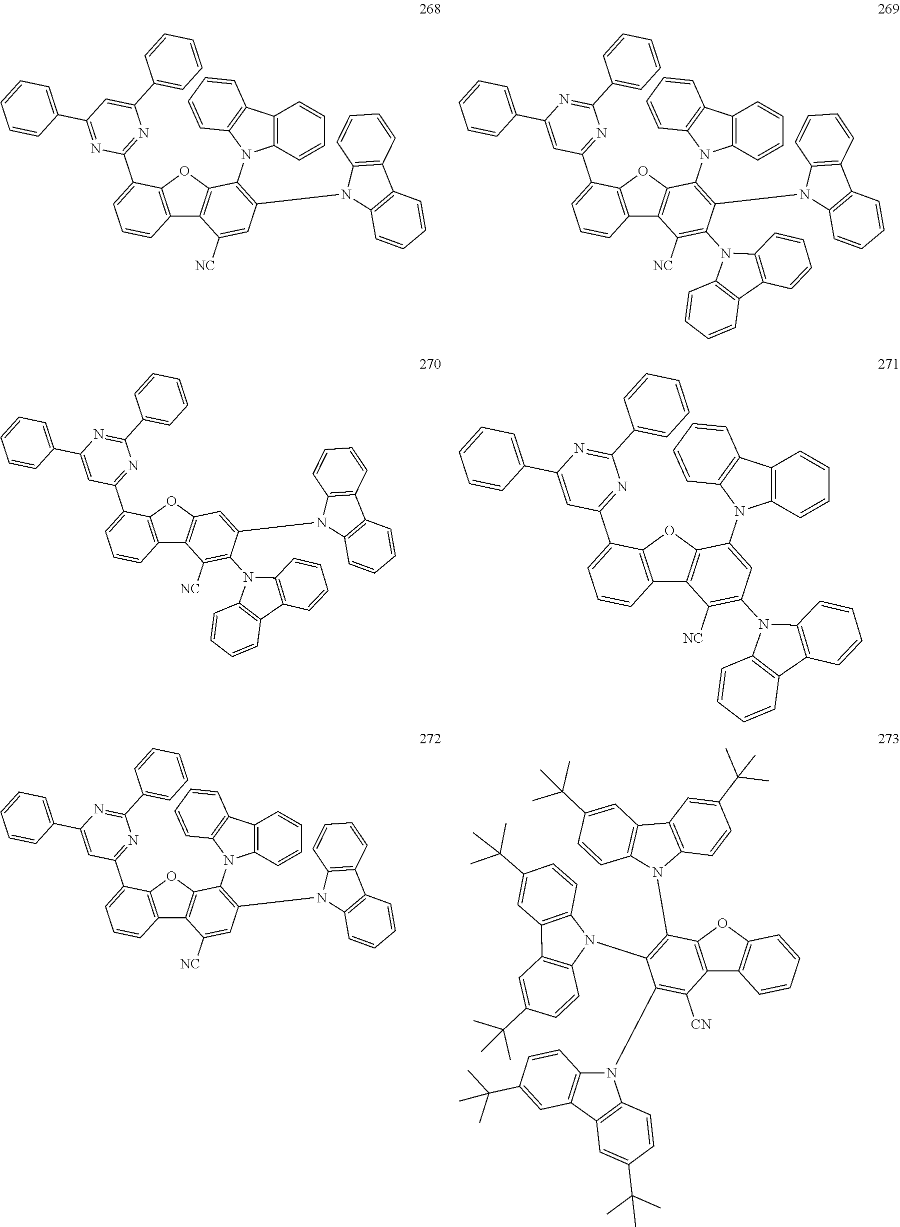

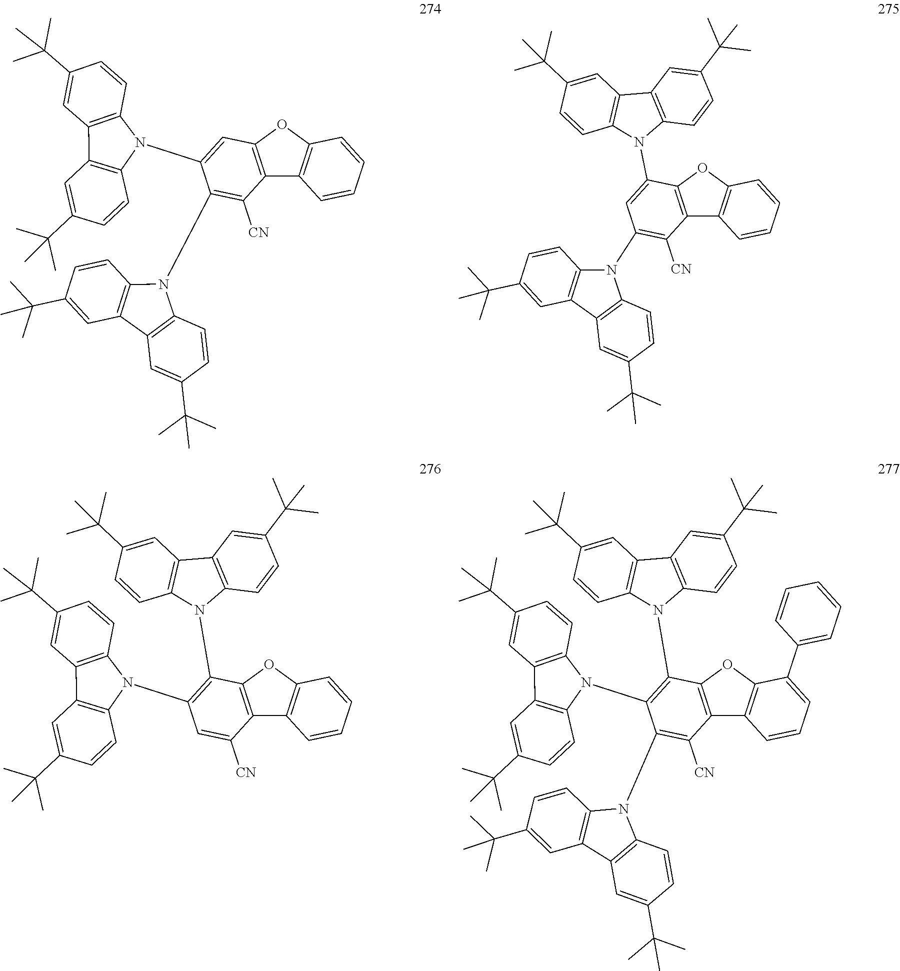 [SMILES: CC(C)(C)c1ccc2c(c1)c1cc(C(C)(C)C)ccc1n2-c1cc2oc3ccccc3c2c(C#N)c1-n1c2ccc(C(C)(C)C)cc2c2cc(C(C)(C)C)ccc21 ; CC(C)(C)c1ccc2c(c1)c1cc(C(C)(C)C)ccc1n2-c1cc(-n2c3ccc(C(C)(C)C)cc3c3cc(C(C)(C)C)ccc32)c2oc3ccccc3c2c1C#N ; CC(C)(C)c1ccc2c(c1)c1cc(C(C)(C)C)ccc1n2-c1cc(C#N)c2c(oc3ccccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1c(-n2c3ccc(C(C)(C)C)cc3c3cc(C(C)(C)C)ccc32)c(C#N)c2c(oc3c(-c4ccccc4)cccc32)c1-n1c2ccc(C(C)(C)C)cc2c2cc(C(C)(C)C)ccc21]

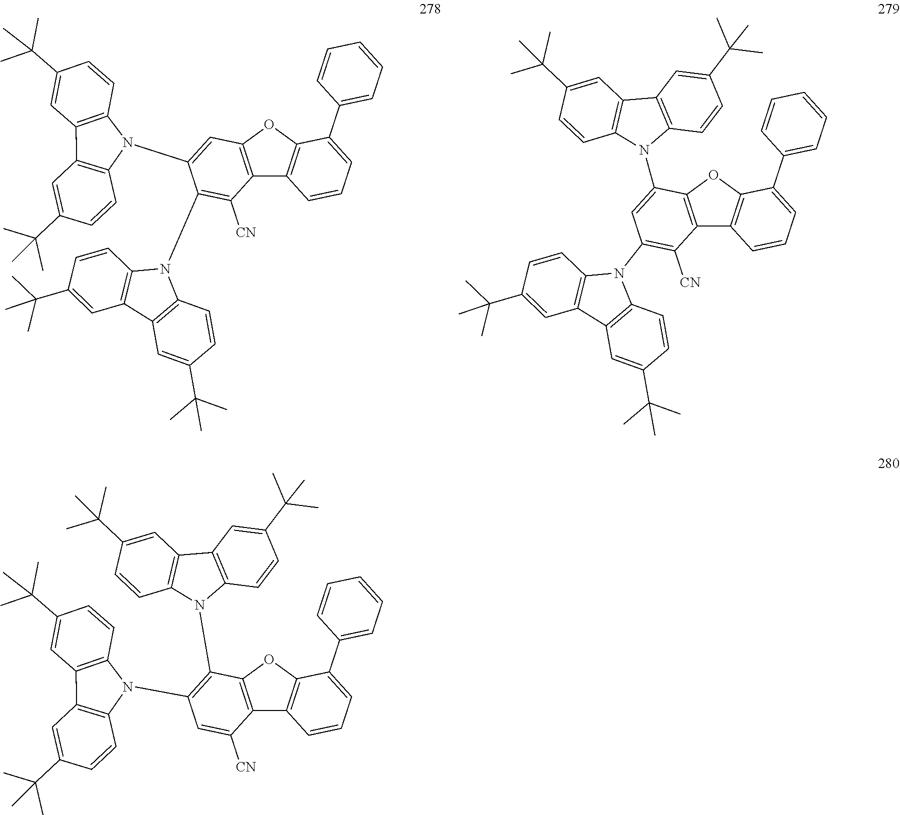 [SMILES: CC(C)(C)c1ccc2c(c1)c1cc(C(C)(C)C)ccc1n2-c1cc2oc3c(-c4ccccc4)cccc3c2c(C#N)c1-n1c2ccc(C(C)(C)C)cc2c2cc(C(C)(C)C)ccc21 ; CC(C)(C)c1ccc2c(c1)c1cc(C(C)(C)C)ccc1n2-c1cc(-n2c3ccc(C(C)(C)C)cc3c3cc(C(C)(C)C)ccc32)c2oc3c(-c4ccccc4)cccc3c2c1C#N ; CC(C)(C)c1ccc2c(c1)c1cc(C(C)(C)C)ccc1n2-c1cc(C#N)c2c(oc3c(-c4ccccc4)cccc32)c1-n1c2ccc(C(C)(C)C)cc2c2cc(C(C)(C)C)ccc21]

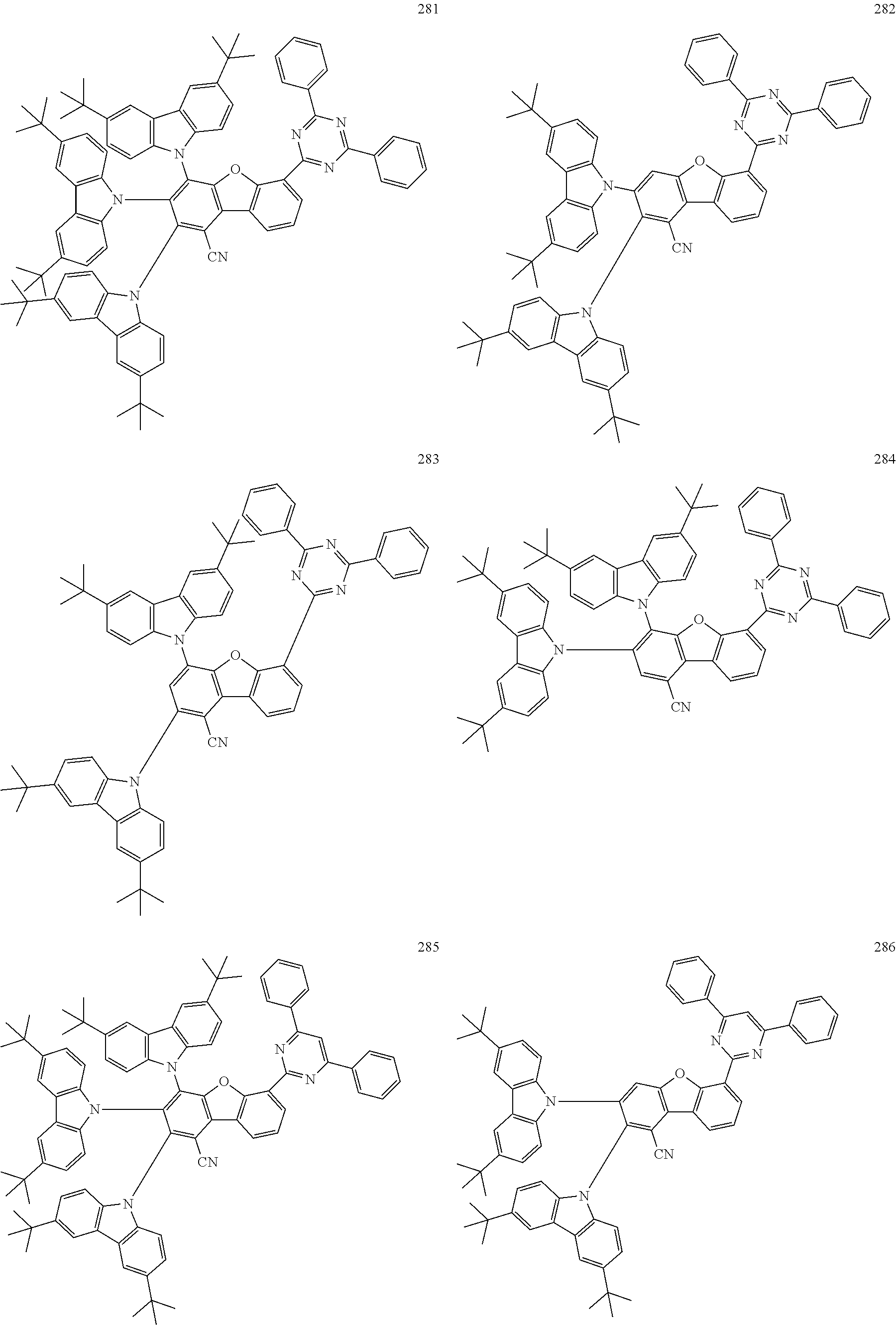

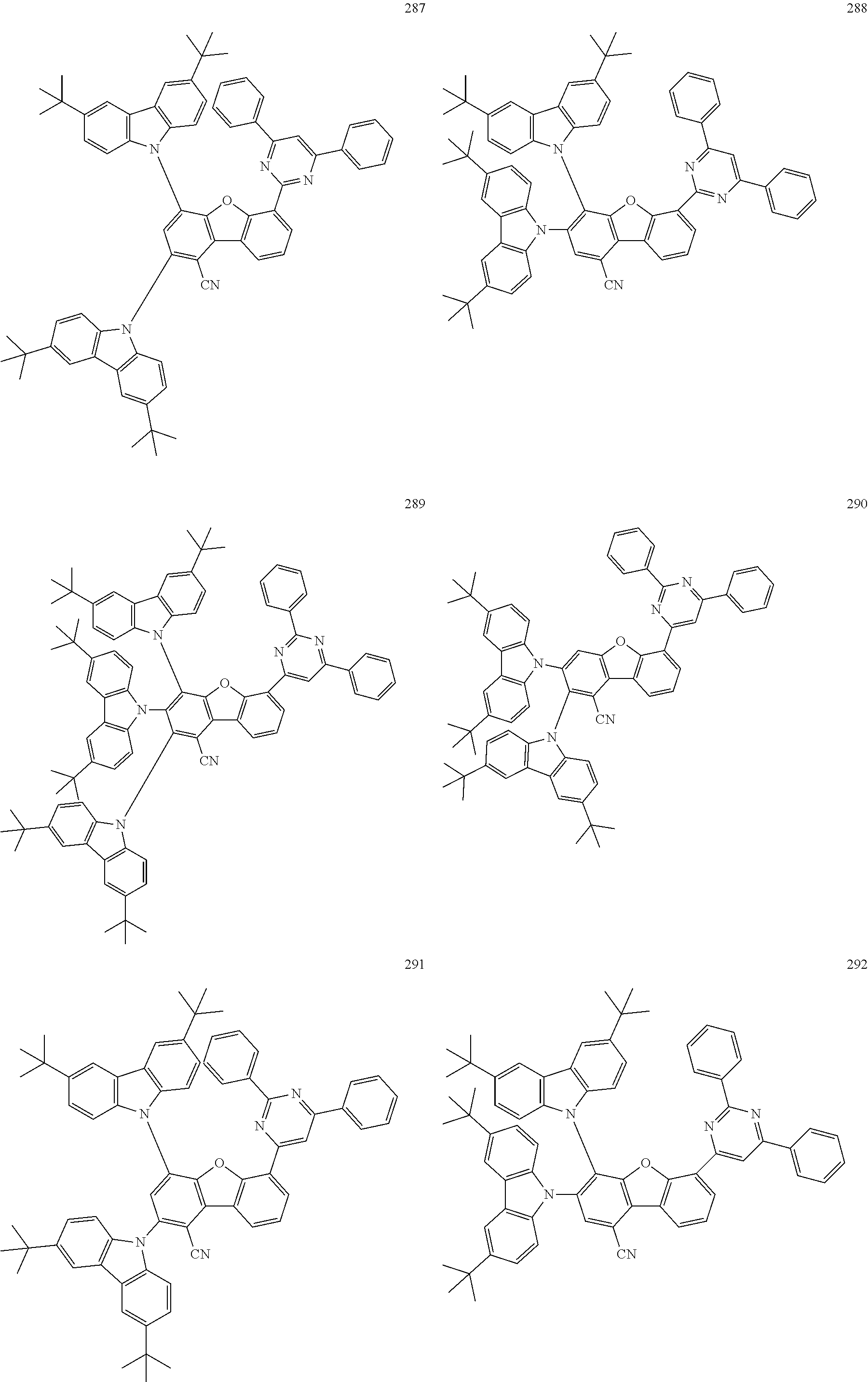

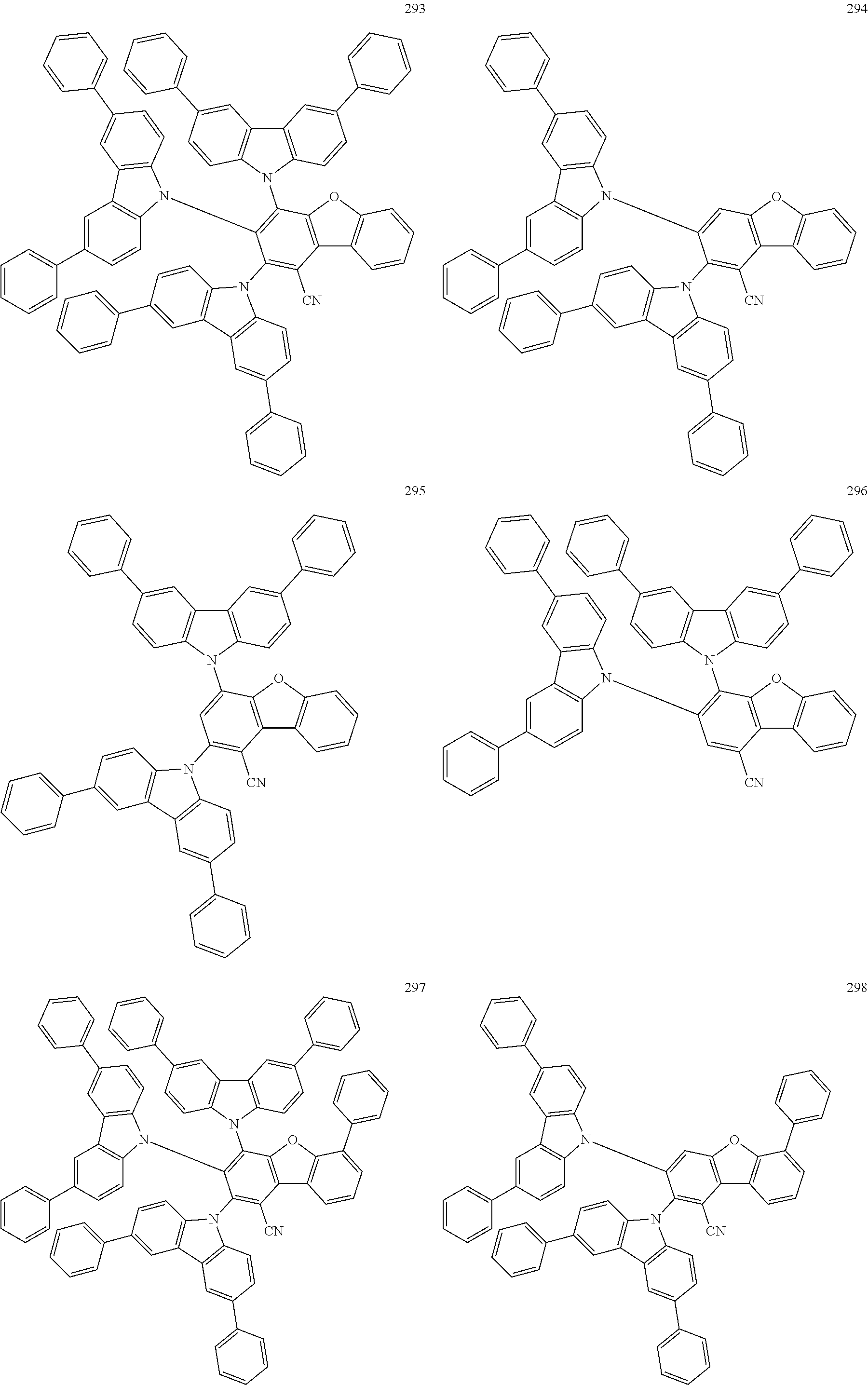

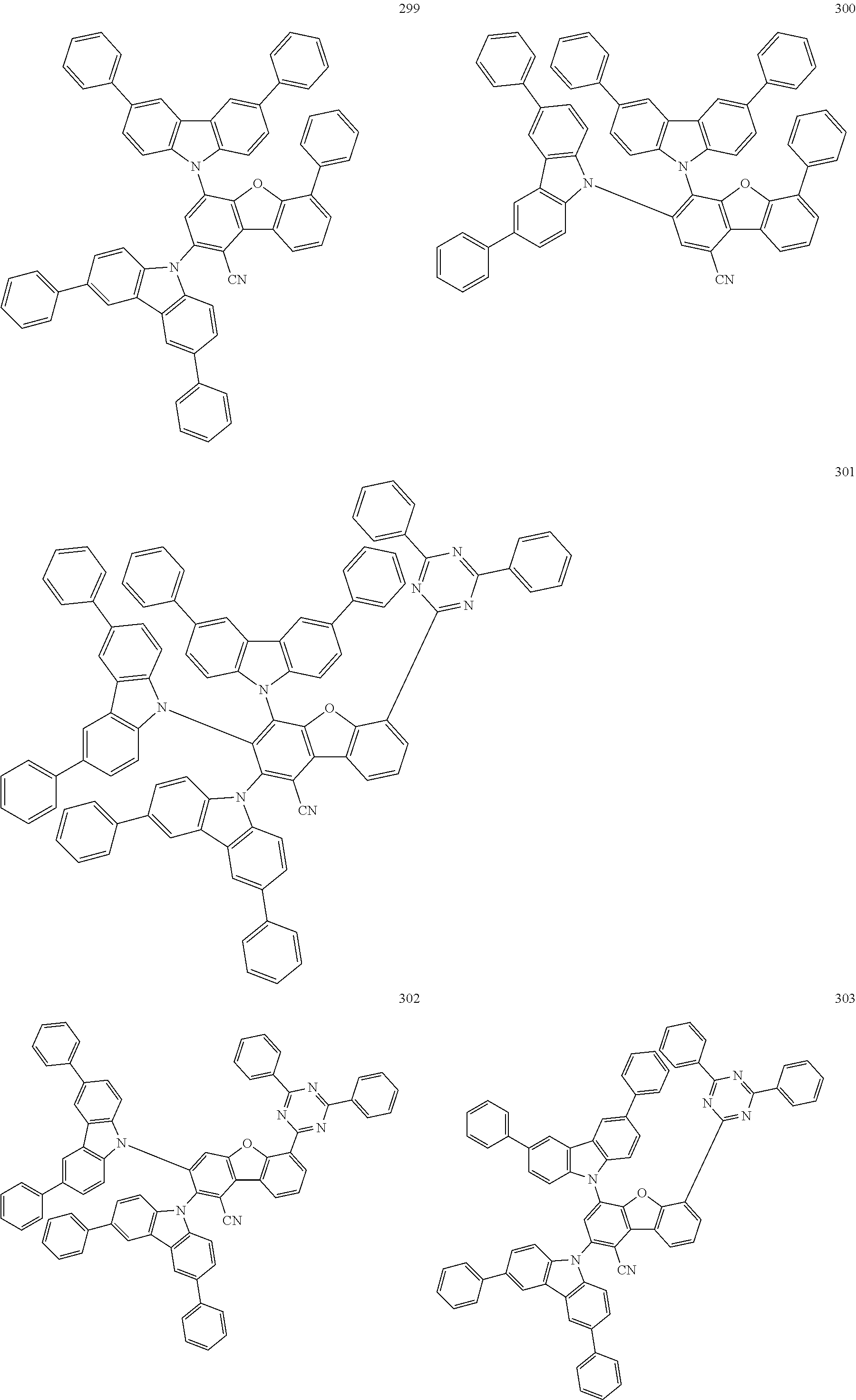

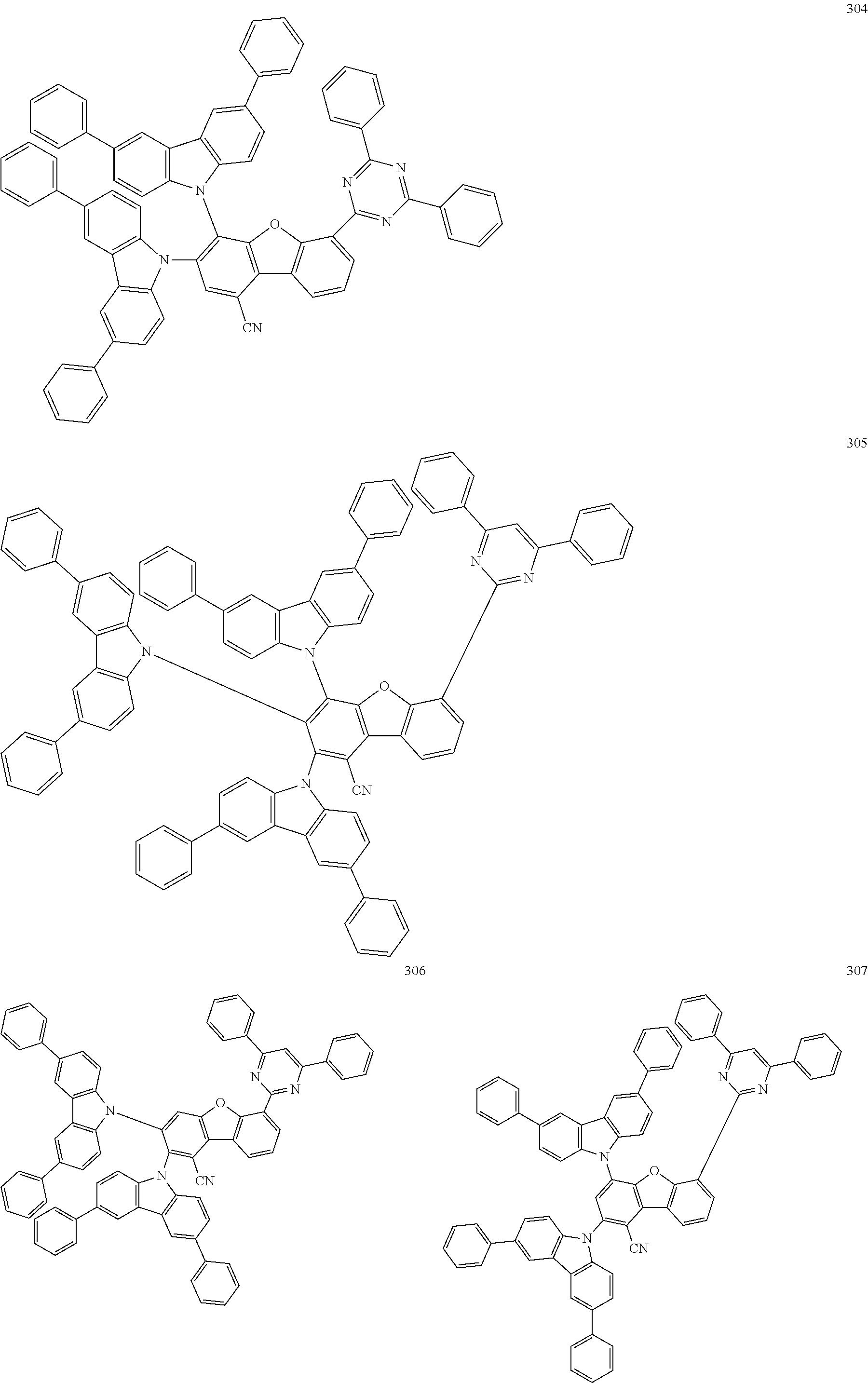

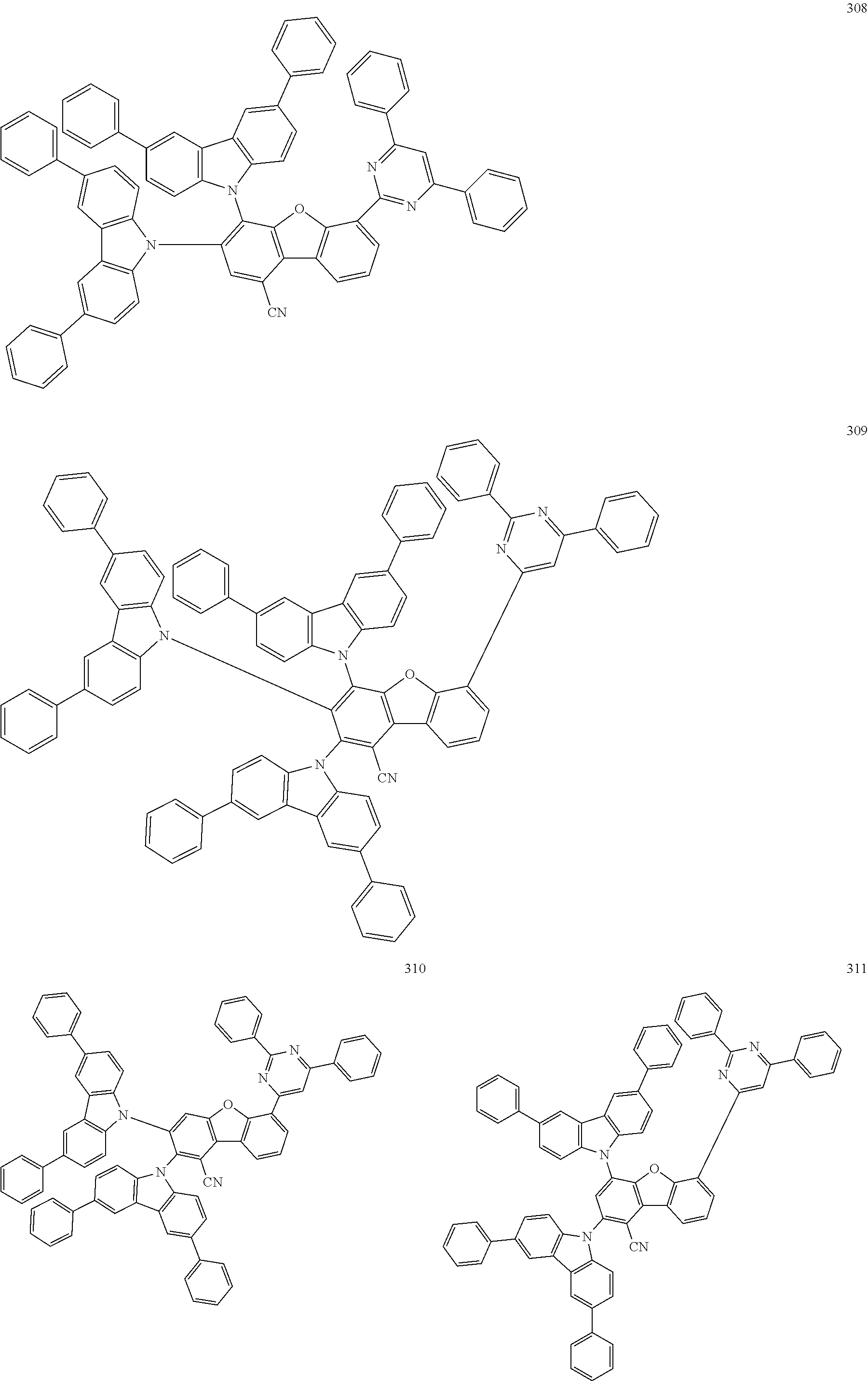

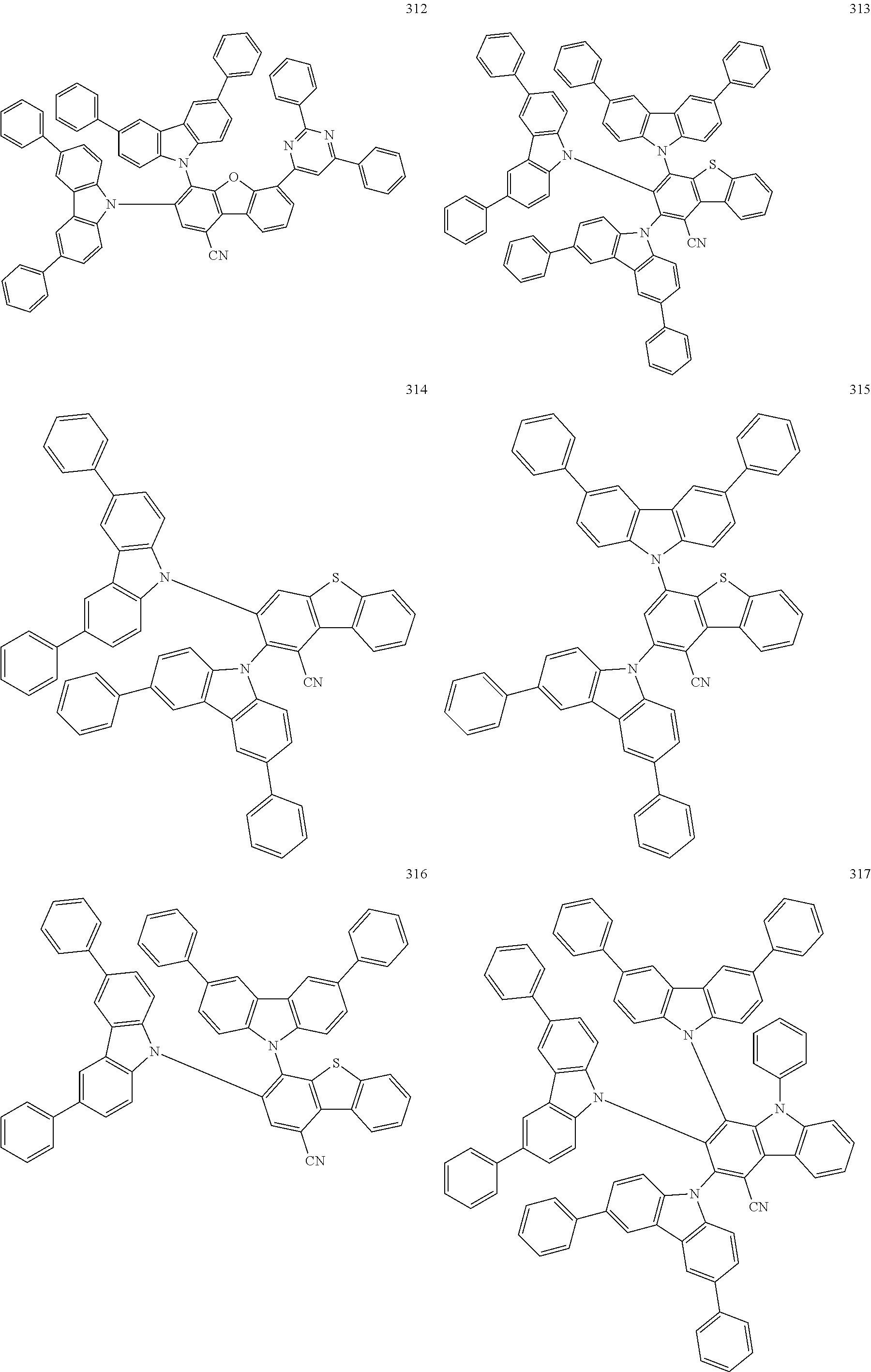

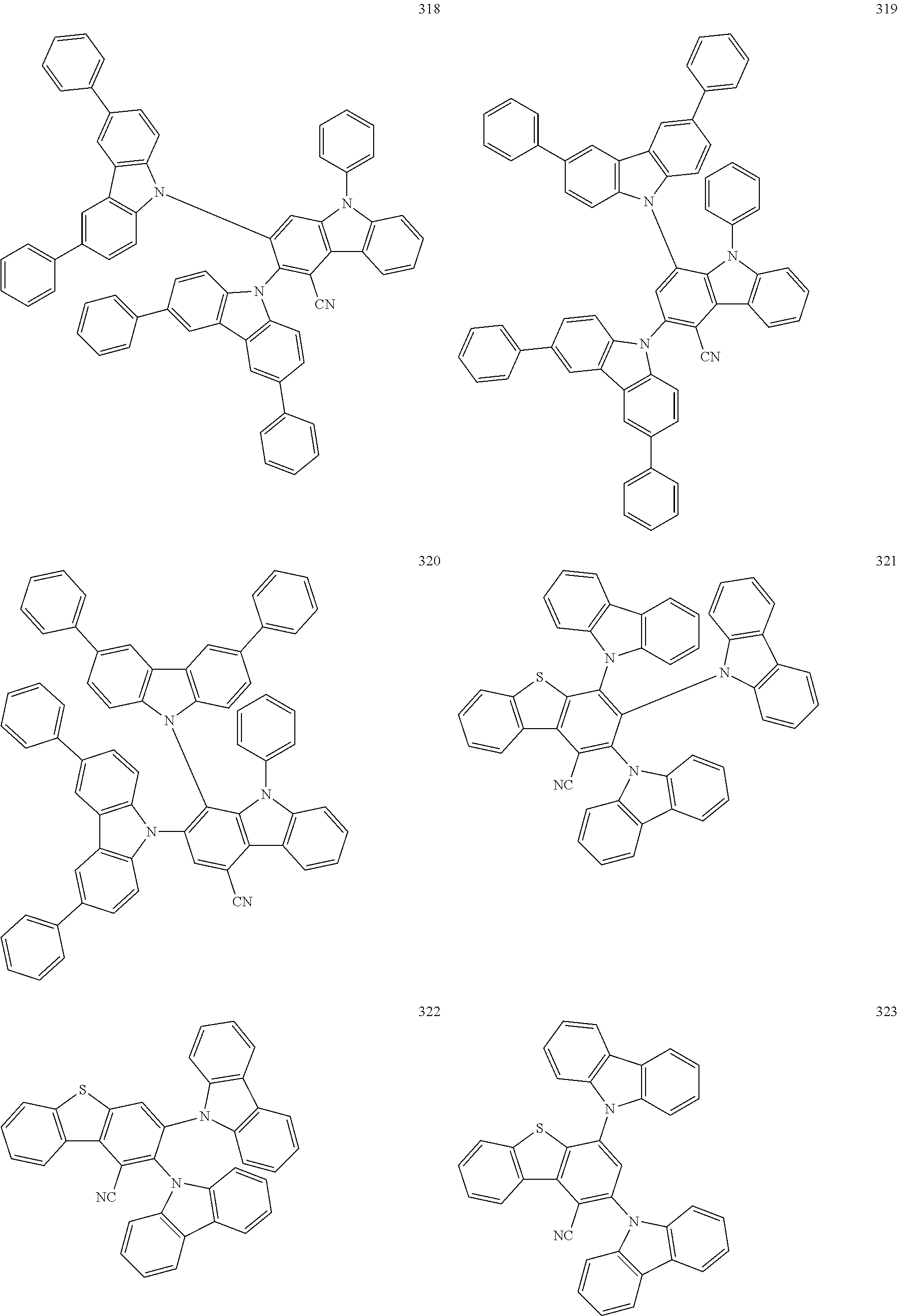

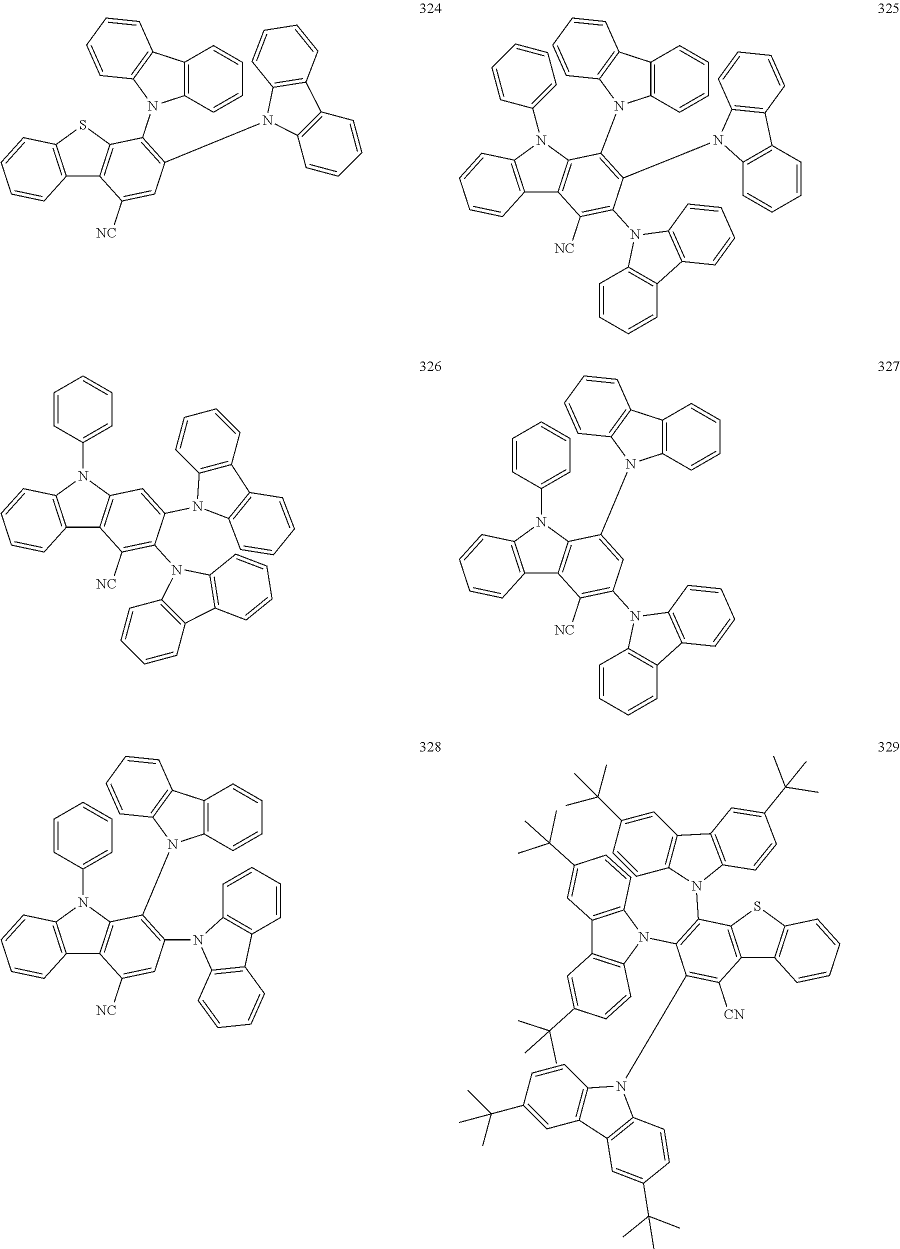

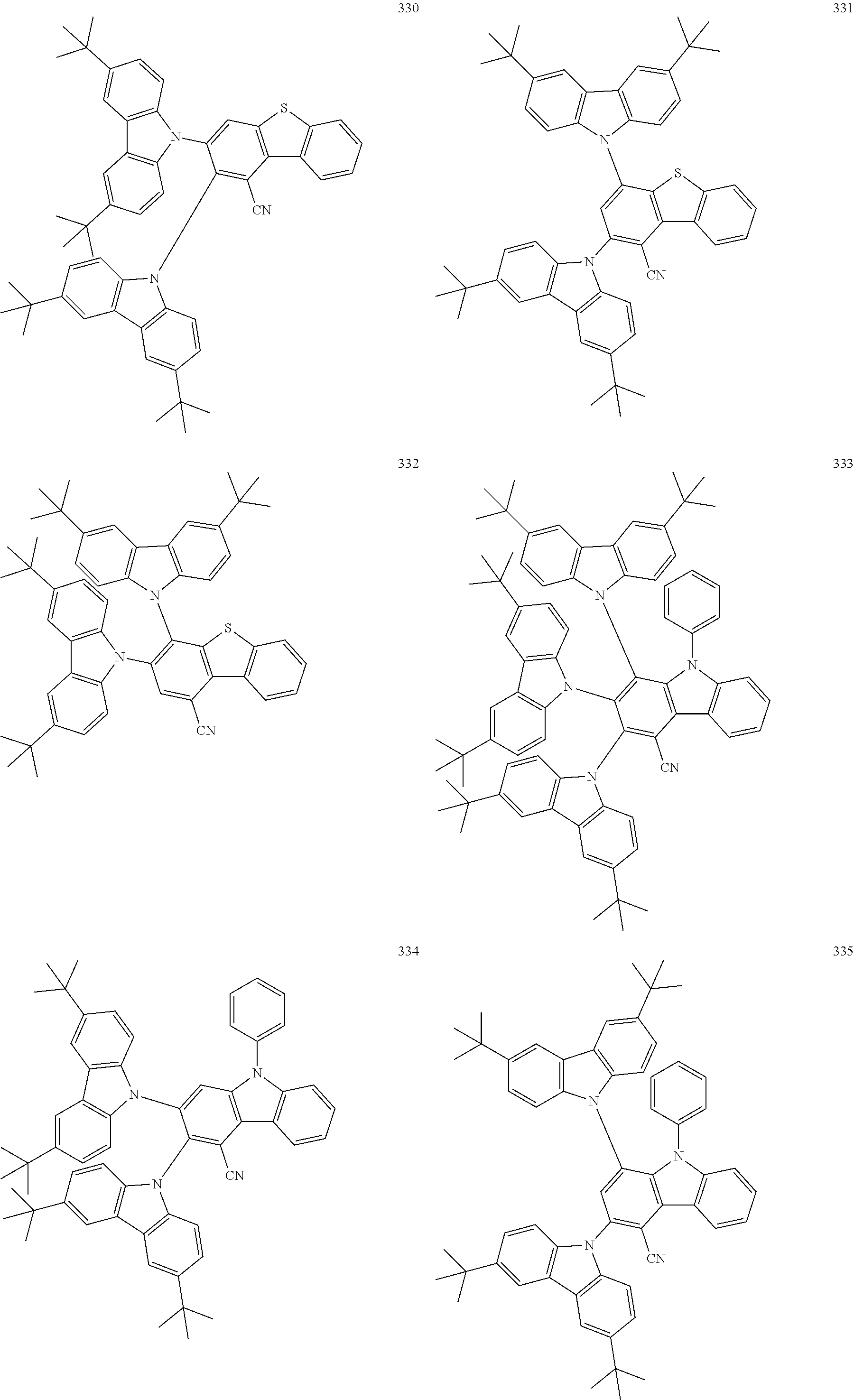

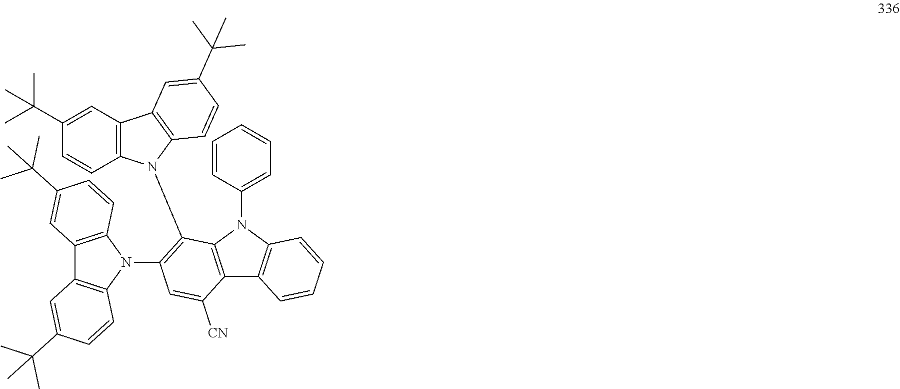 [SMILES: CC(C)(C)c1ccc2c(c1)c1cc(C(C)(C)C)ccc1n2-c1cc(C#N)c2c3ccccc3n(-c3ccccc3)c2c1-n1c2ccc(C(C)(C)C)cc2c2cc(C(C)(C)C)ccc21]

The condensed-cyclic compound represented by Formula 1 has ON, which acts as an electron-withdrawing group, and two or more groups represented by one of Formulae 2-1 and 2-2, which act as electron-donating groups in the same ring. Thus, since the electron-donating groups electronically shield the electron-withdrawing group, the stability of the condensed-cyclic compound may be improved. Accordingly, an organic light-emitting device with high efficiency and/or a long lifespan may be provided.

In detail, when two electron-donating groups are not located adjacent to an electron-withdrawing group, optical characteristics may be degraded. However, since the condensed-cyclic compound represented by Formula 1 has two electron-donating groups and an electron withdrawing group located adjacent thereto, CN is electrically shielded, which leads to an increase in delayed fluorescent characteristics.

The condensed-cyclic compound represented by Formula 1 uses a cyano group as an electron-withdrawing group. Accordingly, the condensed-cyclic compound may have increased thermal stability. Thus, an organic light-emitting device including the condensed-cyclic compound may have a longer lifespan.

In the case of the condensed-cyclic compound represented by Formula 1, an emission wavelength and an energy level thereof may be easily controlled by changing the number of the electron-donating groups and the electron withdrawing groups or the structure of the electron-donating group.

The condensed-cyclic compound represented by Formula 1 may have a narrow full width half-width. Accordingly, an organic light-emitting device including the condensed-cyclic compound may have high efficiency and high color purity.

Regarding the condensed-cyclic compound represented by Formula 1, a highest occupied molecular orbital (HOMO) and a lowest unoccupied molecular orbital (LUMO) may be spatially separated, and therefore, Δ E_{ST} (herein, Δ E_{ST} is the difference between a lowest excited singlet energy level (E_{S1}) and a lowest excited triplet energy level (E_{T1})) is reduced. Therefore, the condensed-cyclic compound represented by Formula 1 may experience reverse intersystem crossing even at low temperatures (for example, room temperature). Electronic devices, for example, organic light-emitting devices, including the condensed-cyclic compound may have high efficiency and/or a long lifespan.

The condensed-cyclic compound represented by Formula 1 may satisfy Equation 1:

0 eV<ΔE_{ST}≤0.5 eV Equation 1

In Equation 1,
Δ E_{ST} is the difference between the lowest excited singlet energy level (E_{S1}) of the condensed-cyclic compound represented by Formula 1 and the lowest excited triplet energy level (E_{T1}) of the condensed-cyclic compound represented by Formula 1. E_{T1} and E_{S1} are evaluated by using a DFT method of Gaussian program that is structurally optimized at the level of B3LYP/6-31G(d,p).

In detail, the condensed-cyclic compound represented by Formula 1 may satisfy the following Equation 1-1, but is not limited to:

0.01 eV<ΔE_{ST}≤0.3 eV Equation 1-1

The lowest excited singlet energy level of the condensed-cyclic compound represented by Formula 1 may be greater than 2.5 eV and less than 3.0 eV, but embodiments are not limited thereto.

For example, the HOMO, LUMO, T₁ energy level, S₁ energy level, photoluminescence (PL) maximum emission wavelength and oscillator intensity of some of the compounds were evaluated by using a DFT method of Gaussian program (optimized at the level of B3LYP, 6-31G(d,p)), and the obtained results are shown in Table 1.

TABLE 1

| | | | | | | The maximum | |
|---|---|---|---|---|---|---|---|
| | | | | | | emission | |
| Com- | | | | | S₁- | wavelength | Oscillator |
| pound | HOMO | LUMO | S₁ | T₁ | T₁ | (nm) of the | strength |
| No. | (eV) | (eV) | (eV) | (eV) | (eV) | PL spectrum | (f) |
| 5 | −5.5 | −2.2 | 2.711 | 2.593 | 0.118 | 456 | 0.0579 |
| 6 | −5.588 | −2.056 | 2.95 | 2.607 | 0.343 | 429 | 0.1221 |
| 7 | −5.576 | −2.057 | 2.94 | 2.723 | 0.217 | 430 | 0.0755 |
| 85 | −5.532 | −2.22 | 2.725 | 2.613 | 0.112 | 453 | 0.0053 |
| 87 | −5.565 | −2.146 | 2.822 | 2.692 | 0.131 | 436 | 0.0033 |
| 89 | −5.5 | −2.2 | 2.711 | 2.593 | 0.118 | 456 | 0.0467 |
| 91 | −5.533 | -2.137 | 2.801 | 2.688 | 0.114 | 435 | 0.0350 |
| 95 | −5.54 | −2.316 | 2.694 | 2.602 | 0.093 | 446 | 0.0327 |
| 127 | −5.373 | −2.225 | 2.619 | 2.526 | 0.093 | 466 | 0.0054 |
| 131 | −5.358 | −2.2 | 2.624 | 2.539 | 0.085 | 461 | 0.0523 |
| A | −5.569 | −2.128 | 2.941 | 2.747 | 0.194 | 416 | 0.00125 |
| B | −5.572 | −1.856 | 3.165 | 2.946 | 0.22 | 379 | 0.0285 |

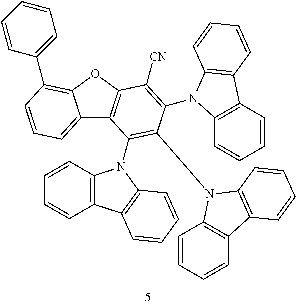 [SMILES: N#Cc1c(-n2c3ccccc3c3ccccc32)c(-n2c3ccccc3c3ccccc32)c(-n2c3ccccc3c3ccccc32)c2c1oc1c(-c3ccccc3)cccc12]

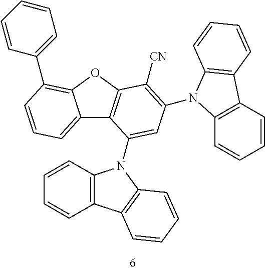 [SMILES: N#Cc1c(-n2c3ccccc3c3ccccc32)cc(-n2c3ccccc3c3ccccc32)c2c1oc1c(-c3ccccc3)cccc12]

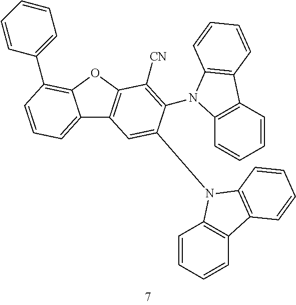 [SMILES: N#Cc1c(-n2c3ccccc3c3ccccc32)c(-n2c3ccccc3c3ccccc32)cc2c1oc1c(-c3ccccc3)cccc12]

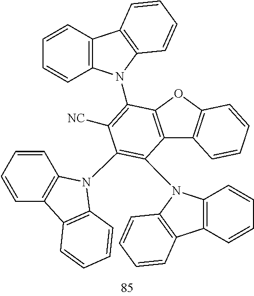 [SMILES: N#Cc1c(-n2c3ccccc3c3ccccc32)c(-n2c3ccccc3c3ccccc32)c2c(oc3ccccc32)c1-n1c2ccccc2c2ccccc21]

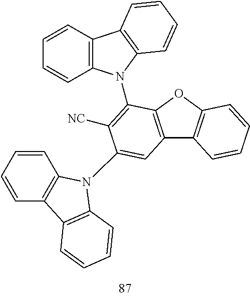 [SMILES: N#Cc1c(-n2c3ccccc3c3ccccc32)cc2c(oc3ccccc32)c1-n1c2ccccc2c2ccccc21]

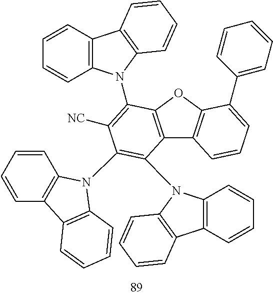 [SMILES: N#Cc1c(-n2c3ccccc3c3ccccc32)c(-n2c3ccccc3c3ccccc32)c2c(oc3c(-c4ccccc4)cccc32)c1-n1c2ccccc2c2ccccc21]

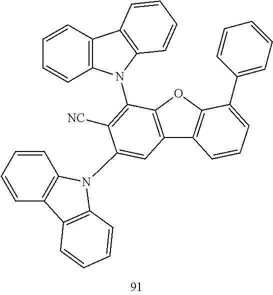 [SMILES: N#Cc1c(-n2c3ccccc3c3ccccc32)cc2c(oc3c(-c4ccccc4)cccc32)c1-n1c2ccccc2c2ccccc21]

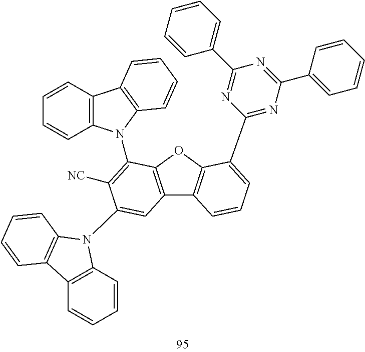 [SMILES: N#Cc1c(-n2c3ccccc3c3ccccc32)cc2c(oc3c(-c4nc(-c5ccccc5)nc(-c5ccccc5)n4)cccc32)c1-n1c2ccccc2c2ccccc21]

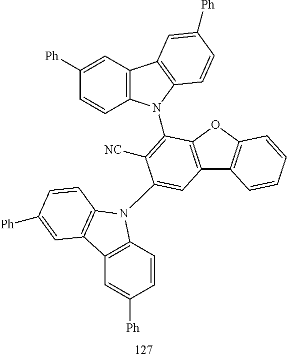 [SMILES: N#Cc1c(-n2c3ccc(-c4ccccc4)cc3c3cc(-c4ccccc4)ccc32)cc2c(oc3ccccc32)c1-n1c2ccc(-c3ccccc3)cc2c2cc(-c3ccccc3)ccc21]

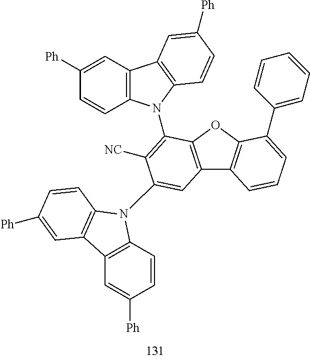 [SMILES: N#Cc1c(-n2c3ccc(-c4ccccc4)cc3c3cc(-c4ccccc4)ccc32)cc2c(oc3c(-c4ccccc4)cccc32)c1-n1c2ccc(-c3ccccc3)cc2c2cc(-c3ccccc3)ccc21]

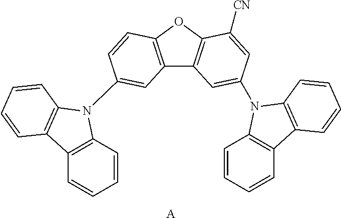

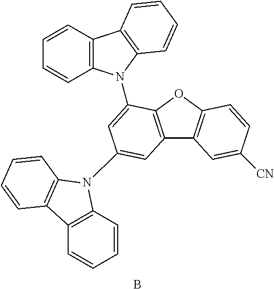

From Table 1, it can be seen that the compound represented by Formula 1 has a relatively small difference between a singlet energy level and a triplet energy level and large oscillator intensities. Therefore, it can be seen that electronic devices, for example, organic light-emitting devices, employing the compound represented by Formula 1 have high luminous efficiency.

Synthesis methods of the condensed-cyclic compound represented by Formula 1 may be understood by one of ordinary skill in the art by referring to Synthesis Examples provided below.

The condensed-cyclic compound represented by Formula 1 may be used as a material for electronic devices, for example, organic light-emitting devices. According to one or more embodiments, an organic light-emitting device includes a first electrode; a second electrode; and an organic layer including an emission layer between the first electrode and the second electrode, wherein the organic layer includes the condensed-cyclic compound represented by Formula 1.

When an organic layer including the condensed-cyclic compound represented by Formula 1 is included in an organic light-emitting device, the obtained organic light-emitting device may have low driving voltage, high efficiency, high luminance, high quantum luminous efficiency, and/or long lifespan.

The condensed-cyclic compound of Formula 1 may be used between a pair of electrodes of an organic light-emitting device. For example, the condensed-cyclic compound may be included in an emission layer, a hole transport region between a first electrode and an emission layer (for example, the hole transport region includes a hole injection layer, a hole transport layer, an electron blocking layer, or a combination thereof), an electron transport region between the emission layer and the second electrode (for example, the electron transport region includes a hole blocking layer, an electron transport layer, an electron injection layer, or a combination thereof), or a combination thereof.

Depending on the use of the condensed-cyclic compound represented by Formula 1, the emission layer of the organic light-emitting device may be embodied according to a first embodiment, a second embodiment, or a third embodiment.

### First Embodiment

The first embodiment is an embodiment in which the condensed-cyclic compound included in the emission layer is used as a fluorescent emitter, that is, the condensed-cyclic compound is a fluorescent emitter.

According to the first embodiment, the emission layer may include the condensed-cyclic compound alone; or
the emission layer may further include a host (hereinafter referred to as ‘host A’, wherein the host A is not the same as the condensed-cyclic compound).

Accordingly, according to the first embodiment, the ratio of an emission component emitted from the condensed-cyclic compound to the total emission components emitted from the emission layer may be 80% or more, for example, 90% or more. For example, the ratio of the emission component emitted from the condensed-cyclic compound to the total emission components emitted from the emission layer may be 95% or more. Herein, the condensed-cyclic compound emits fluorescence and/or delayed fluorescent light, and the emission component of the condensed-cyclic compound may be the sum of prompt emission component of the condensed-cyclic compound and delayed fluorescence component due to reverse intersystem crossing of the condensed-cyclic compound.

In the first embodiment, when the emission layer further includes host A in addition to the condensed-cyclic compound, the amount of the condensed-cyclic compound may be, based on 100 parts by weight of the emission layer, 50 parts by weight or less, for example, 30 parts by weight or less, and the amount of host A in the emission layer may be, based on 100 parts by weight of the emission layer, 50 parts by weight or more, for example, 70 parts by weight or more, but embodiments are not limited thereto.

In the first embodiment, when the emission layer further includes the host A in addition to the condensed-cyclic compound, the host A and the condensed-cyclic compound represented by Formula 1 may satisfy Equation 2:

E(H_{A})_{S1}>E_{S1} Equation 2
wherein, in Equation 2,
E(H_{A})_{S1} is the lowest excitation singlet energy level of the host A;

E_{S1} is the lowest excitation singlet energy level of the condensed-cyclic compound represented by Formula 1. E(H_{A})_{S1} and E_{S1} are evaluated by using a DFT method of Gaussian program that is structurally optimized at the level of B3LYP/6-31 G(d,p).

When the condensed-cyclic compound represented by Formula 1 satisfies Equation 1 and the condensed-cyclic compound represented by Formula 1 and the host A satisfy Equation 2, the condensed-cyclic compound represented by Formula 1 may emit fluorescent light and/or delayed fluorescent light. Therefore, the luminous efficiency of an organic light-emitting device including the condensed-cyclic compound represented by Formula 1 and the host A may be increased.

For example, the host A may be a host material to be described later, but embodiments are not limited thereto.

### Second Embodiment

The second embodiment is an embodiment in which the condensed-cyclic compound contained in the emission layer is used as a host.

According to the second embodiment, the emission layer includes a host and a dopant, and the host may include the condensed-cyclic compound represented by Formula 1. That is, the host may consist of the condensed-cyclic compound represented by Formula 1 alone, or may include other known hosts. The dopant may be, for example, a fluorescent dopant, a phosphorescent dopant, or a thermal activation delayed fluorescent dopant.

Therefore, according to the second embodiment, the ratio of the light of the dopant in the total emission components emitted from the emission layer may be 80% or more, for example, 90% or more (as another example, 95% or more).

In the second embodiment, the amount of dopant in the emission layer may be, based on 100 parts by weight of the emission layer, 50 parts by weight or less, for example, 30 parts by weight or less, and the amount of the host in the emission layer may be, based on 100 parts by weight of the emission layer, 50 parts by weight or more, for example, 70 parts by weight or more, but embodiments are not limited thereto.

For example, in the second embodiment, when the dopant is a fluorescent dopant (hereinafter ‘fluorescent dopant A’), the condensed-cyclic compound represented by Formula 1 and the fluorescent dopant A may satisfy Equation 3:

E_{S1}>E(F_{A})_{S1} Equation 3
wherein, in Equation 3,
E_{S1} is the lowest excitation singlet energy level of the condensed-cyclic compound represented by Formula 1; and
E(F_{A})_{S1} is the lowest excitation singlet energy level of the fluorescent dopant A.

E_{S1} and E(F_{A})_{S1} are evaluated by using a DFT method of Gaussian program that is structurally optimized at the level of B3LYP/6-31G(d,p).

When the condensed-cyclic compound represented by Formula 1 and the fluorescent dopant A satisfy Equation 3, Forster energy transfer from the condensed-cyclic compound represented by Formula 1 to the fluorescent dopant A may be promoted. Therefore, the luminous efficiency of an organic light-emitting device including the condensed-cyclic compound represented by Formula 1 and the fluorescent dopant A may be increased.

For example, the dopant may be a dopant material to be described later, but embodiments are not limited thereto.

When the host contains other known hosts, the other known hosts may be the host materials described below, but not necessarily limited.

### Third Embodiment

The third embodiment is an embodiment in which the condensed-cyclic compound contained in the emission layer is used as an auxiliary dopant.

According to the third embodiment, the emission layer includes a host, an auxiliary dopant, and a dopant, and the auxiliary dopant may include the condensed-cyclic compound. The dopant may be, for example, a fluorescent dopant, a phosphorescent dopant, or a thermal activation delayed fluorescent dopant.

Therefore, according to the third embodiment, the ratio of the light emission component of dopant in the total light emission components emitted from the emission layer may be 80% or more, for example, 90% or more (as another example, 95% or more).

In the third embodiment, the amount of dopant in the emission layer may be, based on 100 parts by weight of the emission layer, 50 parts by weight or less, for example, 30 parts by weight or less, the amount of the host in the emission layer may be, based on 100 parts by weight of the emission layer, 50 parts by weight or more, for example, 70 parts by weight or more, and the amount of the auxiliary dopant may be, based on 100 parts by weight of the emission layer, 30 parts by weight or less, for example, 20 parts by weight or less, but embodiments are not limited thereto.

For example, in the third embodiment, when the dopant is a fluorescent dopant (hereinafter, referred to as ‘fluorescent dopant B’), the host (hereinafter referred to as ‘host B), the condensed-cyclic compound represented by Formula 1 and the fluorescent dopant B may satisfy Equation 4:

E(H_{B})_{S1}>E_{S1}>E(F_{B})_{S1} Equation 4
wherein, in Equation 4,
E(H_{B})_{S1} is the lowest excitation singlet energy level of the host B;
E_{S1} is the lowest excitation singlet energy level of the condensed-cyclic compound represented by Formula 1; and
E(F_{B})_{S1} is the lowest excitation singlet energy level of the fluorescent dopant B.

E(H_{B})_{S1}, E_{S1′} and E(F_{B})_{S1} are evaluated by using a DFT method of Gaussian program that is structurally optimized at the level of B3LYP/6-31G(d,p).

When the host B, the condensed-cyclic compound represented by Formula 1 and the fluorescent dopant B satisfy Equation 4, Forster energy transfer from the condensed-cyclic compound represented by Formula 1 to the fluorescent dopant B may be promoted. Therefore, the luminous efficiency of an organic light-emitting device including host B, the condensed-cyclic compound represented by Formula 1 and the fluorescent dopant B may be increased.

The host B and the condensed-cyclic compound represented by Formula 1 may satisfy Equation 5:

E(H_{B})_{T1}−E_{T1}>0.05 eV Equation 5
wherein, in Equation 5,
E(H_{B})_{S1} is the lowest excitation triplet energy level of the host B; and
E_{T1} is the lowest excitation triplet energy level of the condensed-cyclic compound represented by Formula 1.

E(H_{B})_{T1} and E_{T1} are evaluated by using a DFT method of Gaussian program that is structurally optimized at the level of B3LYP/6-31G(d,p).

In the third embodiment, due to the satisfaction of Equation 5 (for example, E(H_{B})_{T1}−E_{T1} is 0.10 eV or more and 0.65 eV or less), the energy of the triplet exciton generated in the auxiliary dopant in the emission layer may not be transferred to the host B in the emission layer. Accordingly, the probability of the triplet exciton being lost in a path other than the light emission path is reduced. As a result, the organic light-emitting device may have high efficiency.

The condensed-cyclic compound represented by Formula 1 and the fluorescent dopant B may satisfy Equation 6:

E(F_{B})_{S1}−E_{S1}<0 eV Equation 6
wherein, in Equation 6,
E(F_{B})_{S1} is the lowest excitation singlet energy level of the fluorescent dopant, and
E_{S1} is the lowest excitation triplet energy level of the condensed-cyclic compound represented by Formula 1.

E(F_{B})_{S1} and E_{S1} are evaluated by using a DFT method of Gaussian program that is structurally optimized at the level of B3LYP/6-31G(d,p).

In the third embodiment, due to the satisfaction of Equation 6 (for example, E_{S1}(FD)−E_{S1(AD)} is −0.4 eV or more and −0.05 eV or less), the energy of singlet exciton generated in the auxiliary dopant in the emission layer may quickly move toward the fluorescent dopant B. Thus, substantially, only the fluorescent dopant B in the emission layer of the organic light-emitting device emits light, thereby realizing a fluorescence emission spectrum of excellent color purity based on the fluorescent dopant B. In addition, a fluorescence emission having a relatively short exciton lifespan may occur. Accordingly, the efficiency transfer phenomenon under high luminosity (that is, a roll-off phenomenon), which may occur due to the interaction among a plurality of excitons (exciton-exciton interaction) or exciton-charge (hole or electron) interaction (exciton-polaron interaction) may be suppressed, thereby leading to the embodiment of an organic light-emitting device having high efficiency. Furthermore, since the auxiliary dopant has a short exciton lifespan, the probability in which the auxiliary dopant experiences chemical or physical deterioration when in the exciton state may be reduced. Thus, an organic light-emitting device satisfying Equation 6 has increased durability.

In the third embodiment, the host may be a host material to be described later, but embodiments are not limited thereto.

In the third embodiment, the dopant may be a dopant material to be described later, but embodiments are not limited thereto.

For example, the host may have 2.9 eV or more of triplet energy level, for example, 2.9 eV or more and 4.5 eV or less than the triplet energy level. As a result, energy transfer from the host to a fluorescent dopant, a phosphorescent dopant and/or a delayed fluorescent dopant may be effectively performed, and the organic light-emitting device may have high efficiency.

For example, the host may include a fluorene-containing compound, a carbazole-containing compound, a dibenzothiophene-containing compound, a dibenzothiophene-containing compound, an indenocarbazole-containing compound, an indolocarbazole-containing compound, a benzofurocarbazole-containing compound, a benzothienocarbazole-containing compound, an acridine-containing compound, a dihydroacridine-containing compound, a triindolobenzene-containing compound, a pyridine-containing compound, a pyrimidine-containing compound, a triazine-containing compound, a silicon-containing compound, a cyano-containing compound, a phosphine oxide-containing compound, a sulfoxide-containing compound, or a combination thereof, but embodiments are not limited thereto.

In one embodiment, the host may include a compound including a carbazole ring and a cyano group.

For example, the host may include compounds represented by Formulae 11-1 to 11-3, but embodiments are not limited thereto:

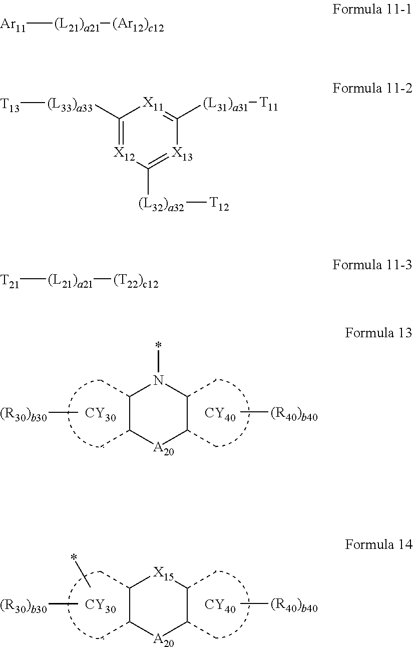

wherein, in Formulae 11-1 to 11-3, 13, and 14,
Ar₁₁ and Ar₁₂ may each independently be a group represented by one of Formulae 13 and 14,
X₁₅ may be N(R₂₀₀), O, or S,
X₁₁ may be N or C(T₁₄), X₁₂ may be N or C(T₁₅), and X₁₃ may be N or C(T₁₆), and X₁₁ to X₁₃ may be N,
T₂₁ and T₂₂ may each independently be *-(L₂₁)ₐ₂₁-Si(Q₄₁)(Q₄₂)(Q₄₃) or *-(L₂₁)ₐ₂₁-P(═O)(Q₅₁)(Q₅₂),
L₂₁ and L₃₁ to L₃₃ may each independently be
a single bond, O, S, Si(Q₆₁)(Q₆₂), a phenylene group, a pyridinylene group, a pyrimidinylene group, a pyrazinylene group, a pyridazinylene group, a triazinylene group, a naphthylene group, a fluorenylene group, a carbazolylene group, a dibenzofuranylene group, or a dibenzothiophenylene group; or
a phenylene group, a pyridinylene group, a pyrimidinylene group, a pyrazinylene group, a pyridazinylene group, a triazinylene group, a naphthylene group, a fluorenylene group, a carbazolylene group, a dibenzofuranylene group, or a dibenzothiophenylene group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, —CF₃, —CF₂H, —CFH₂, a phenyl group, a phenyl group substituted with a cyano group, a biphenyl group, a terphenyl group, a naphthyl group, a pyridinyl group, a pyrimidinyl group, a pyrazinyl group, a pyridazinyl group, a triazinyl group, a fluorenyl group, a carbazolyl group, a dibenzofuranyl group, a dibenzothiophenyl group, —Si(Q₇₁)(Q₇₂)(Q₇₃), or a combination thereof;
a21 and a31 to a33 may each independently be an integer from 0 to 5; when a21 is two or more, two or more L₂₁ (s) may be identical to or different from each other, when a31 is two or more, two or more L₃₁(s) may be identical to or different from each other, when a32 is two or more two or more L₃₂(s) may be identical to or different from each other, and when a33 is two or more two or more L₃₃(s) may be identical to or different from each other,
CY₃₀ and CY₄₀ may each independently be a benzene group, a naphthalene group, a fluorene group, a carbazole group, a benzocarbazole group, an indolocarbazole group, a dibenzofuran group, or a dibenzothiophene group,
A₂₀ may be:
a single bond, a C₁-C₄ alkylene group, or a C₂-C₄ alkenylene group; or
a C₁-C₄ alkylene group or a C₂-C₄ alkenylene group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, a phenyl group, a biphenyl group, a terphenyl group, a naphthyl group, a pyridinyl group, a pyrimidinyl group, a pyrazinyl group, a pyridazinyl group, a triazinyl group, a fluorenyl group, a carbazolyl group, a dibenzofuranyl group, a dibenzothiophenyl group, —Si(Q₈₁)(Q₈₂)(Q₈₃), or a combination thereof;
T₁₁ to T₁₆, R₂₀₀, R₃₀, and R₄₀ may each independently be hydrogen, deuterium, —F, —Cl, —Br, —I, a hydroxyl group, a cyano group (CN), a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, or —Si(Q₉₁)(Q₉₂)(Q₉₃),
b30 and b40 may each independently be an integer from 0 to 10,
c12 may be 0, 1, 2, or 3,
may be a binding site to a neighboring atom,
a substituent of the substituted C₁-C₆₀ alkyl group, substituted C₂-C₆₀ alkenyl group, substituted C₂-C₆₀ alkynyl group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group may be deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₀₁)(Q₁₀₂)(Q₁₀₃), or a combination thereof, and
Q₄₁ to Q₄₃, Q₅₁ to Q₅₂, Q₆₁ to Q₆₂, Q₇₁ to Q₇₃, Q₈₁ to Q₈₃, Q₉₁ to Q₉₃ and Q₁₀₁ to Q₁₀₃ may each independently be hydrogen, deuterium, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, or a monovalent non-aromatic condensed heteropolycyclic group.

For example, the host may include Compounds H-1 to H-27, but embodiments are not limited thereto:

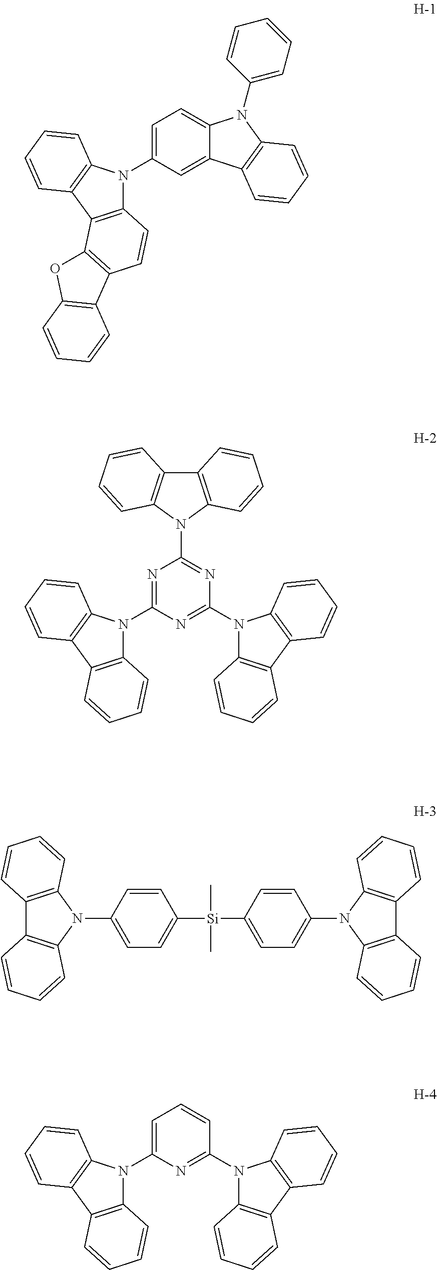 [SMILES: c1ccc(-n2c3ccccc3c3cc(-n4c5ccccc5c5c6oc7ccccc7c6ccc54)ccc32)cc1 ; c1ccc2c(c1)c1ccccc1n2-c1nc(-n2c3ccccc3c3ccccc32)nc(-n2c3ccccc3c3ccccc32)n1 ; C[Si](C)(c1ccc(-n2c3ccccc3c3ccccc32)cc1)c1ccc(-n2c3ccccc3c3ccccc32)cc1 ; c1cc(-n2c3ccccc3c3ccccc32)nc(-n2c3ccccc3c3ccccc32)c1]

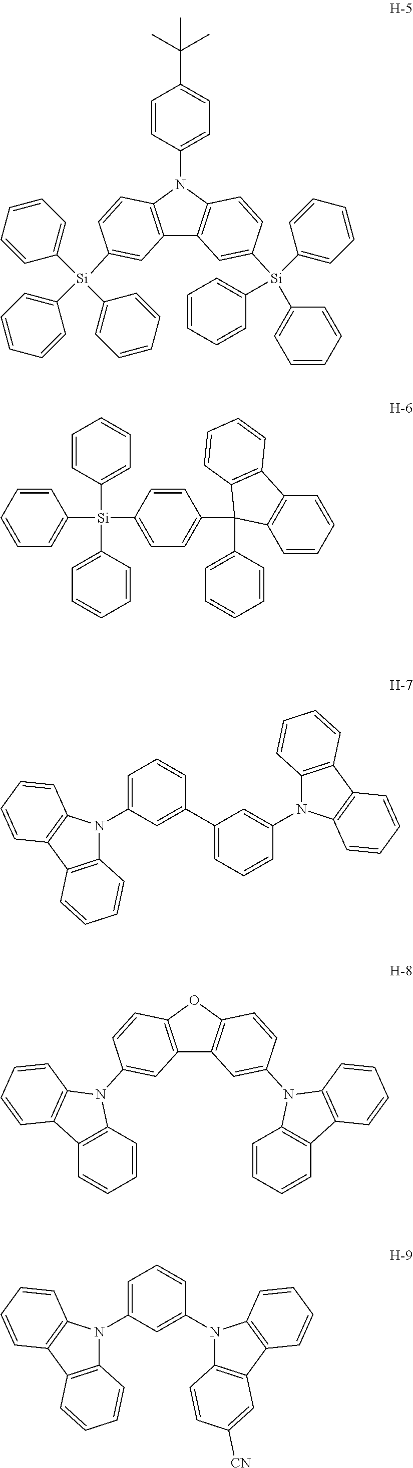 [SMILES: CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1 ; c1ccc(C2(c3ccc([Si](c4ccccc4)(c4ccccc4)c4ccccc4)cc3)c3ccccc3-c3ccccc32)cc1 ; c1cc(-c2cccc(-n3c4ccccc4c4ccccc43)c2)cc(-n2c3ccccc3c3ccccc32)c1 ; c1ccc2c(c1)c1ccccc1n2-c1ccc2oc3ccc(-n4c5ccccc5c5ccccc54)cc3c2c1 ; N#Cc1ccc2c(c1)c1ccccc1n2-c1cccc(-n2c3ccccc3c3ccccc32)c1]

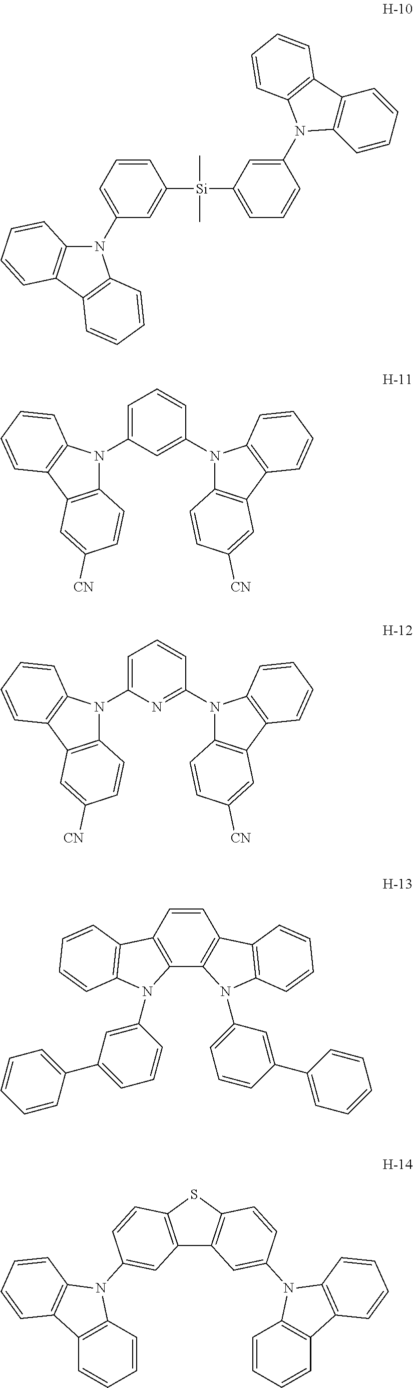

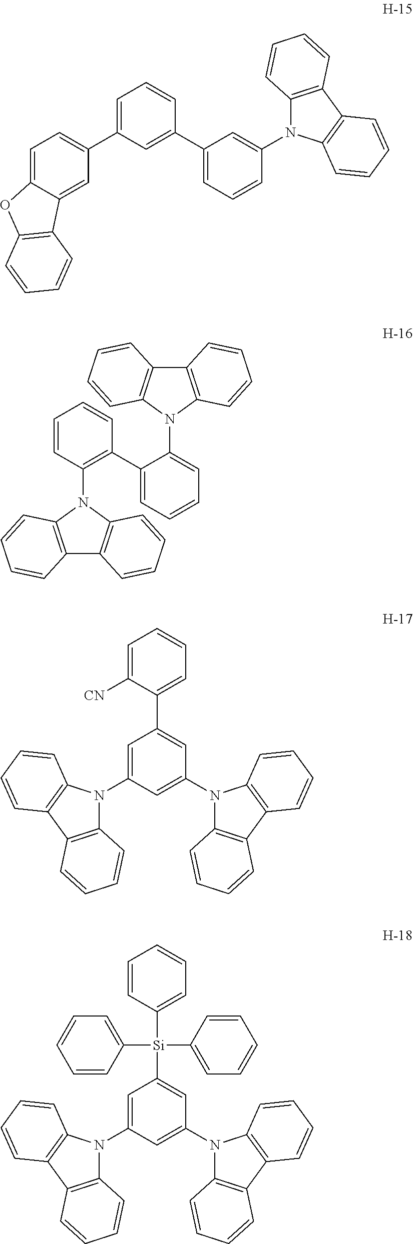

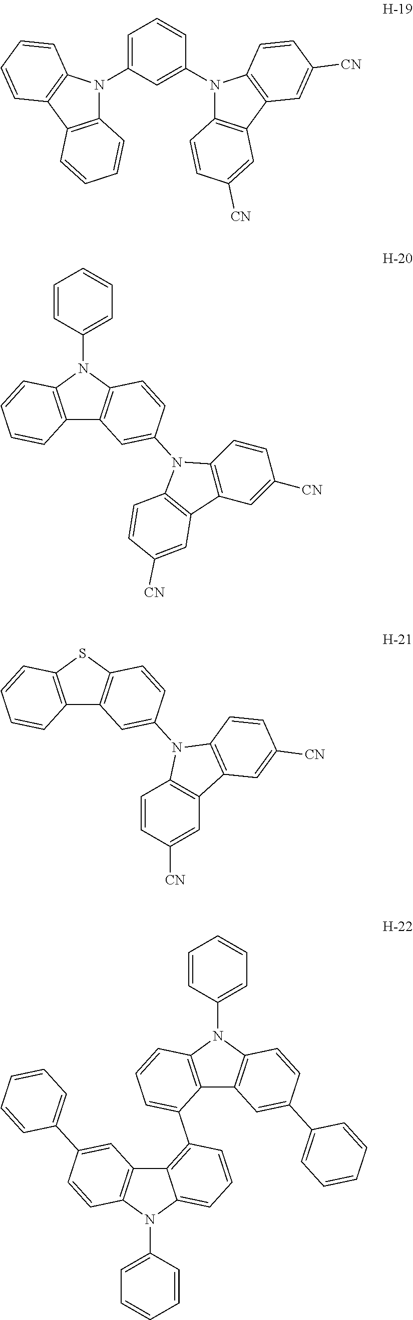 [SMILES: N#Cc1ccc2c(c1)c1cc(C#N)ccc1n2-c1cccc(-n2c3ccccc3c3ccccc32)c1 ; N#Cc1ccc2c(c1)c1cc(C#N)ccc1n2-c1ccc2c(c1)c1ccccc1n2-c1ccccc1 ; N#Cc1ccc2c(c1)c1cc(C#N)ccc1n2-c1ccc2sc3ccccc3c2c1 ; c1ccc(-c2ccc3c(c2)c2c(-c4cccc5c4c4cc(-c6ccccc6)ccc4n5-c4ccccc4)cccc2n3-c2ccccc2)cc1]

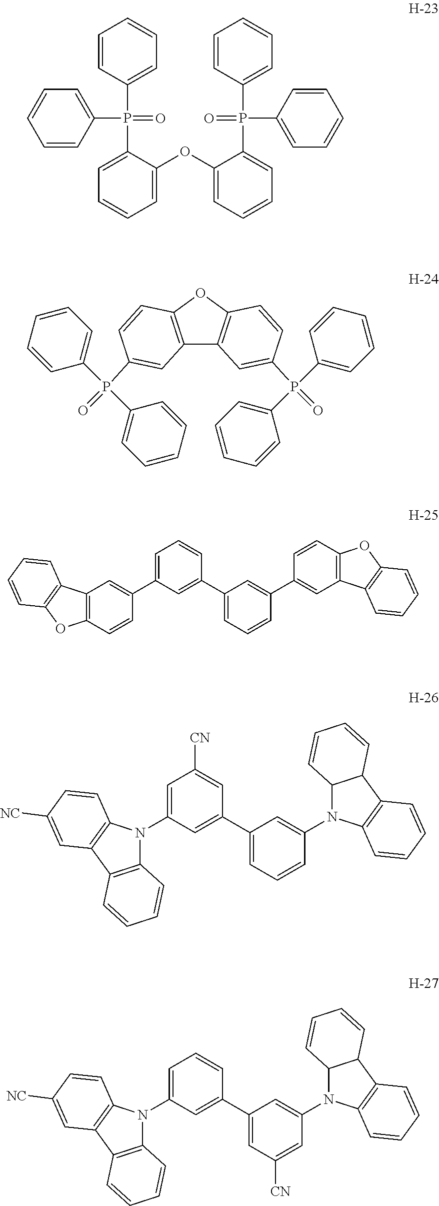 [SMILES: O=P(c1ccccc1)(c1ccccc1)c1ccccc1Oc1ccccc1P(=O)(c1ccccc1)c1ccccc1 ; O=P(c1ccccc1)(c1ccccc1)c1ccc2oc3ccc(P(=O)(c4ccccc4)c4ccccc4)cc3c2c1 ; c1cc(-c2cccc(-c3ccc4oc5ccccc5c4c3)c2)cc(-c2ccc3oc4ccccc4c3c2)c1 ; N#Cc1cc(-c2cccc(N3c4ccccc4C4C=CC=CC43)c2)cc(-n2c3ccccc3c3cc(C#N)ccc32)c1 ; N#Cc1cc(-c2cccc(-n3c4ccccc4c4cc(C#N)ccc43)c2)cc(N2c3ccccc3C3C=CC=CC32)c1]

The fluorescent dopant may be a condensed polycyclic compound or a styryl compound.

For example, the fluorescent dopant may include a naphthalene-containing core, a fluorene-containing core, a spiro-bifluorene-containing core, a benzofluorene-containing core, a dibenzofluorene-containing core, a phenanthrene-containing core, an anthracene-containing core, a fluoranthene-containing core, a triphenylene-containing core, a pyrene-containing core, a chrysene-containing core, a naphthacene-containing core, a picene-containing core, a perylene-containing core, a pentaphene-containing core, an indenoanthracene-containing core, a tetracene-containing core, a bisanthracene-containing core, or a core represented by one of Formulae 501-1 to 501-18, but embodiments of the present disclosure are not limited thereto:

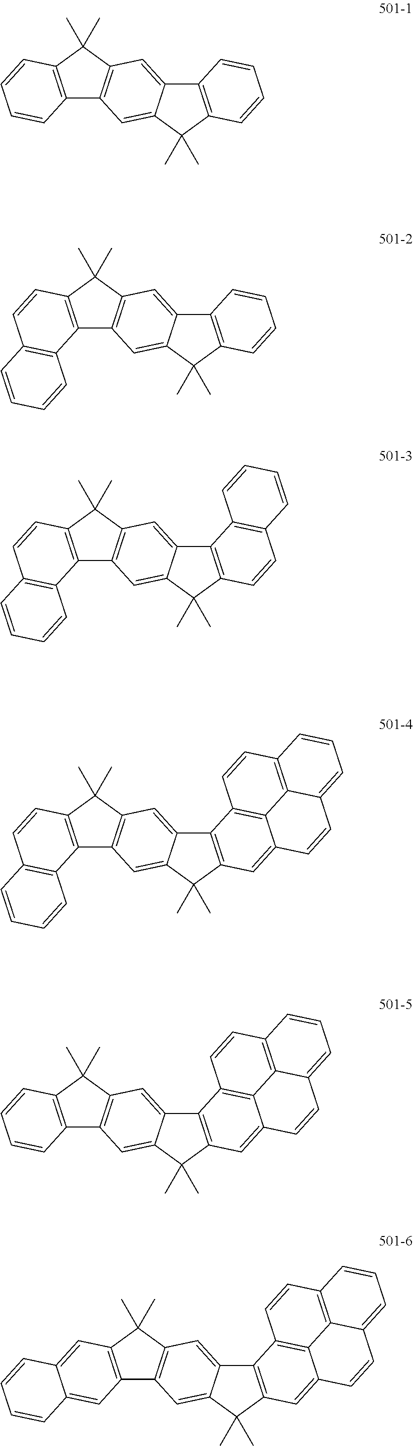

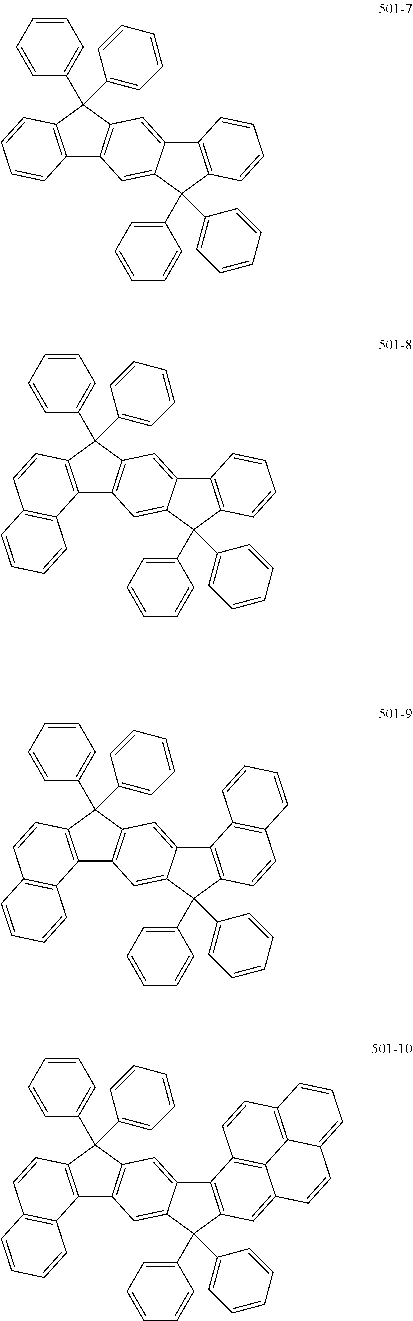

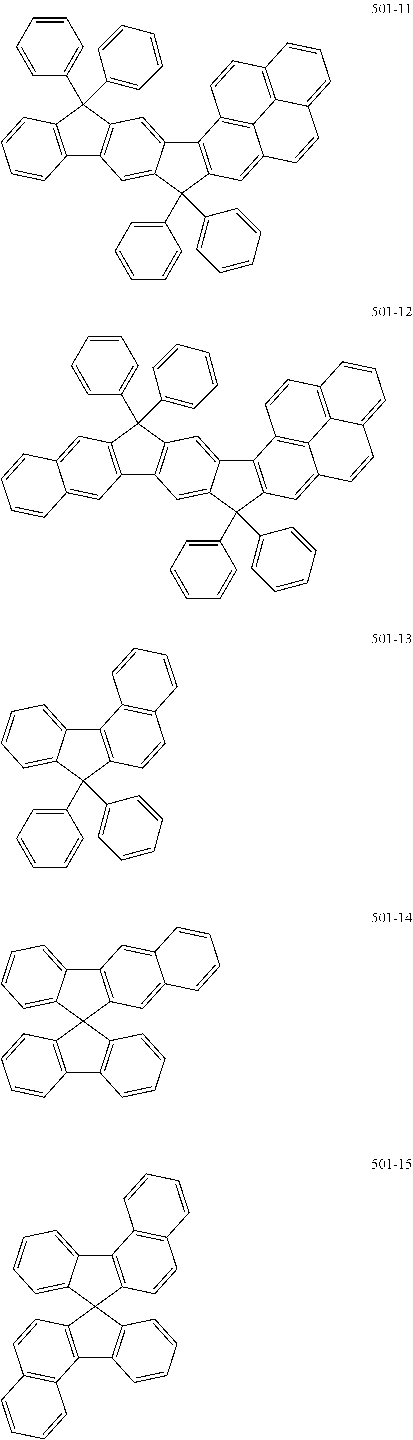

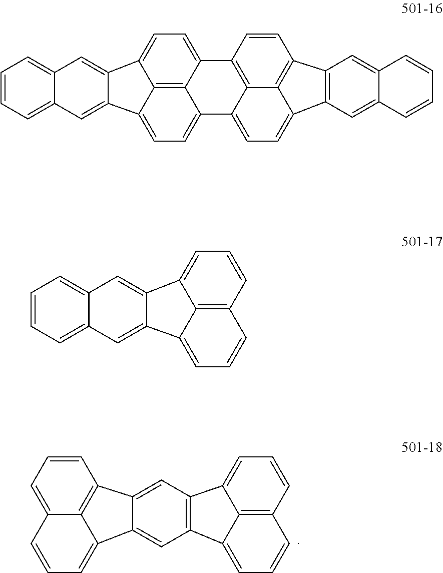

In one or more embodiments, the fluorescent dopant may be a styryl-amine compound or a styryl-carbazole compound, but embodiments of the present disclosure are not limited thereto.

In one embodiment, the fluorescent dopant may be a group represented by Formula 501:

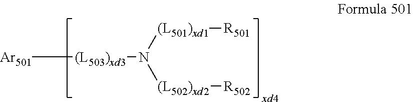

wherein, in Formula 501,
Ar₅₀₁ may be:
a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a group represented by one of Formulae 501-1 to 501-18; or
a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene an indenoanthracene group, a tetracene group, a bisanthracene, or a group represented by one of Formula 501-1 to 501-18, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₅₀₁)(Q₅₀₂)(Q₅₀₃) (wherein Q₅₀₁ to Q₅₀₃ may each independently be hydrogen, a C₁-C₆₀ alkyl group, a C₁-C₆₀ alkoxy group, a C₆-C₆₀ aryl group, a C₁-C₆₀ heteroaryl group, a monovalent non-aromatic condensed polycyclic group, or a monovalent non-aromatic condensed heteropolycyclic group), or a combination thereof;
L₅₀₁ to L₅₀₃ may each independently be a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, or a substituted or unsubstituted divalent non-aromatic condensed heteropolycyclic group,
R₅₀₁ and R₅₀₂ may each independently be:
a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, or a dibenzothiophenyl group; or
a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, or a dibenzothiophenyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, or a combination thereof;
xd1 to xd3 may each independently be 0, 1, 2, or 3, and
xd4 may be 0, 1, 2, 3, 4, 5, or 6.

For example, in Formula 501,
Ar₅₀₁ may be:
a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a group represented by one of Formulae 501-1 to 501-18; or
a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a tetracene group, a bisanthracene group, or a group represented by one of Formulae 501-1 to 501-18, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a dibenzofuranyl group, a dibenzothiophenyl group, a carbazolyl group, a pyridinyl group, a pyrimidinyl group, a triazinyl group, a quinolinyl group, an isoquinolinyl group, and —Si(Q₅₀₁)(Q₅₀₂)(Q₅₀₃) (wherein Q₅₀₁ to Q₅₀₃ may each independently be hydrogen, a C₁-C₂₀ alkyl group, a C₁-C₂₀ alkoxy group, a phenyl group, a biphenyl group, a terphenyl group, or a naphthyl group), or a combination thereof;
L₅₀₁ to L₅₀₃ are the same as described in connection with L₂₁,
xd1 to xd3 may each independently be 0, 1, or 2,
xd4 may be 0, 1, 2, or 3, but embodiments of the present disclosure are not limited thereto.

In one or more embodiments, the fluorescent dopant may include a compound represented by one of Formulae 502-1 to 502-5:

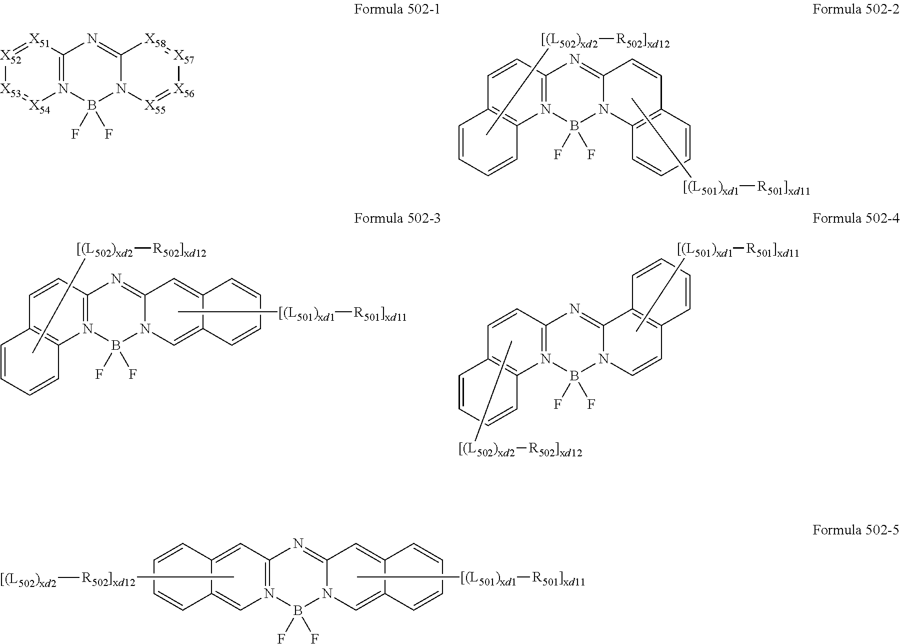

In Formulae 502-1 to 502-5,
X₅₁ may be N or C-[(L₅₀₁)_{xd1}-R₅₀₁], X₅₂ may be N or C-[(L₅₀₂)_{xd2}-R₅₀₂], X₅₃ may be N or C-[(L₅₀₀₃)_{xd3}-R₅₀₃], X₅₄ may be N or C-[(L₅₀₄)_{xd4}-R₅₀₄], X₅₅ may be N or C-[(L₅₀₅)_{xd5}-R₅₀₅], X₅₆ may be N or C-[(L₅₀₆)_{xd6}-R₅₀₆], X₅₇ may be N or C-[(L₅₀₇)_{xd7}-R₅₀₇], and X₅₈ may be N or C-[(L₅₀₈)_{xd8}-R₅₀₈],
L₅₀₁ to L₅₀₈ may each be understood by referring to the description provided in connection with L₅₀₁ in Formula 501,
xd1 to xd8 may each be understood by referring to the description provided in connection with xd1 in Formula 501,
R₅₀₁ to R₅₀₈ may each independently be
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group,
a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, or a dibenzothiophenyl group; or
a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, or a dibenzothiophenyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, a triazinyl group, a dibenzofuranyl group, a dibenzothiophenyl group, or a combination thereof;
xd11 and xd12 may each independently be an integer from 0 to 5,
two of R₅₀₁ to R₅₀₄ may optionally be combined with each other to form a saturated or unsaturated ring, and
two of R₅₀₅ to R₅₀₈ may optionally be combined with each other to form a saturated or unsaturated ring.

The fluorescent dopant may include, for example, Compounds FD(1) to FD(16) and FD1 to FD13:

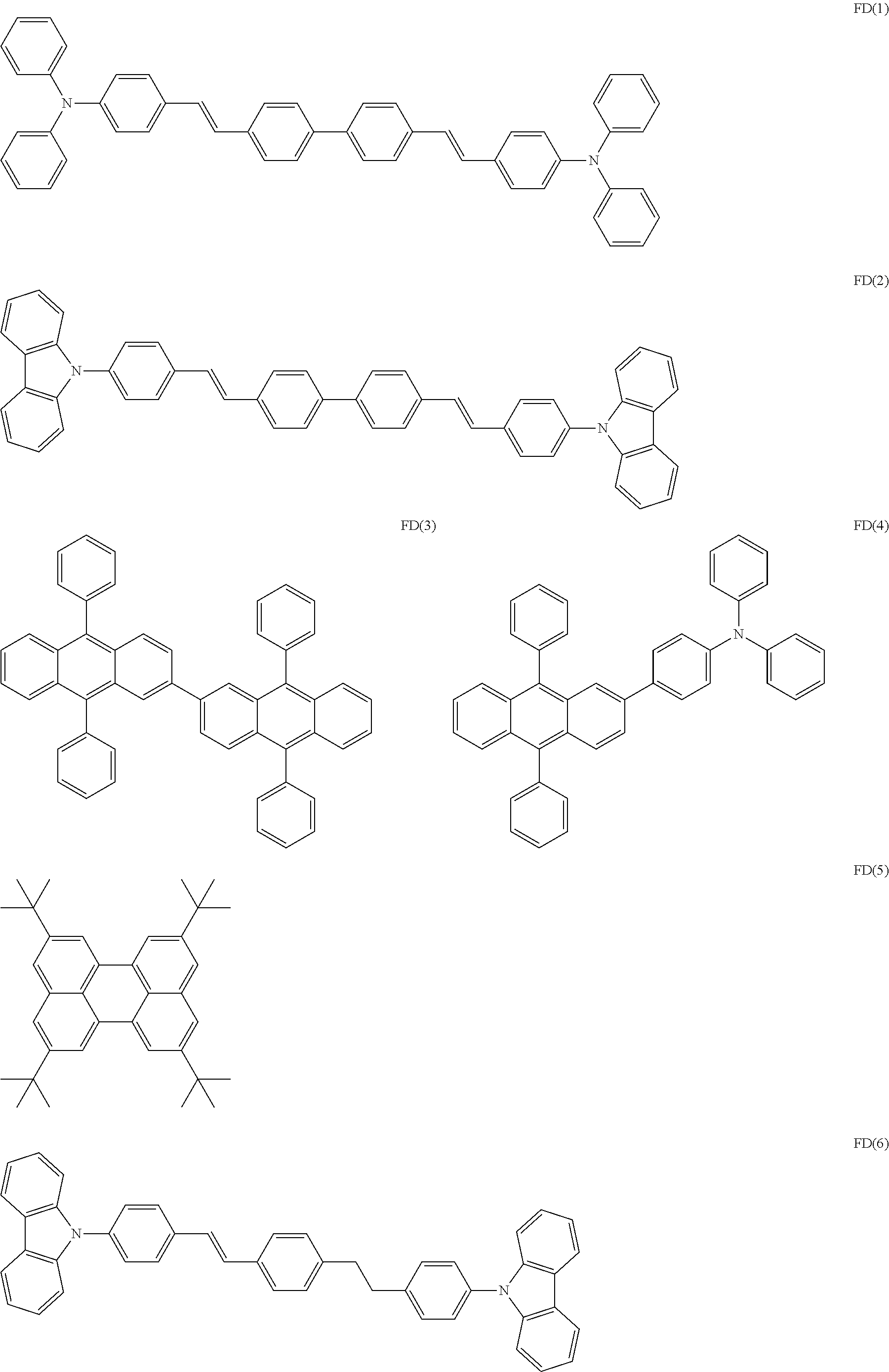

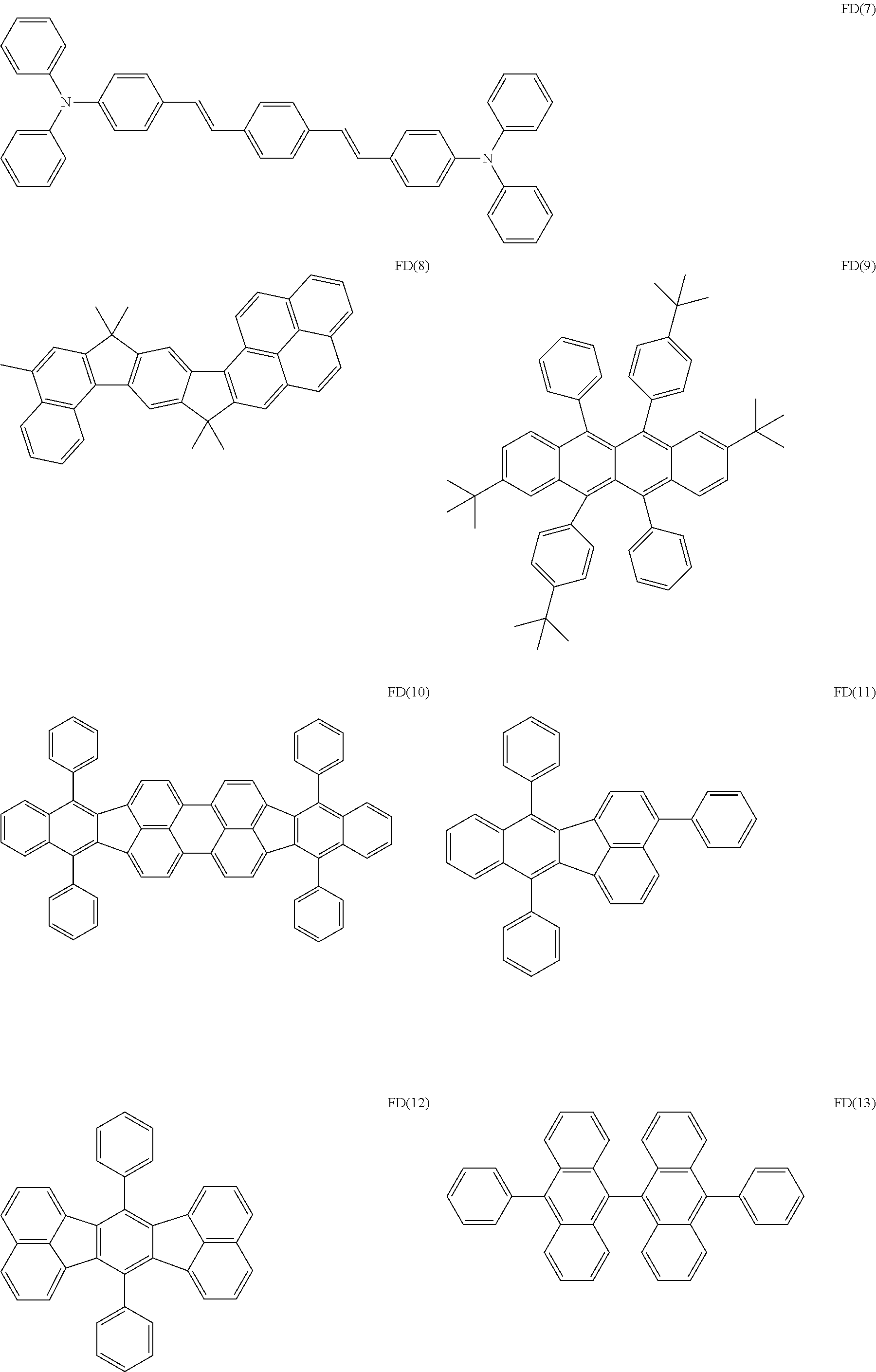

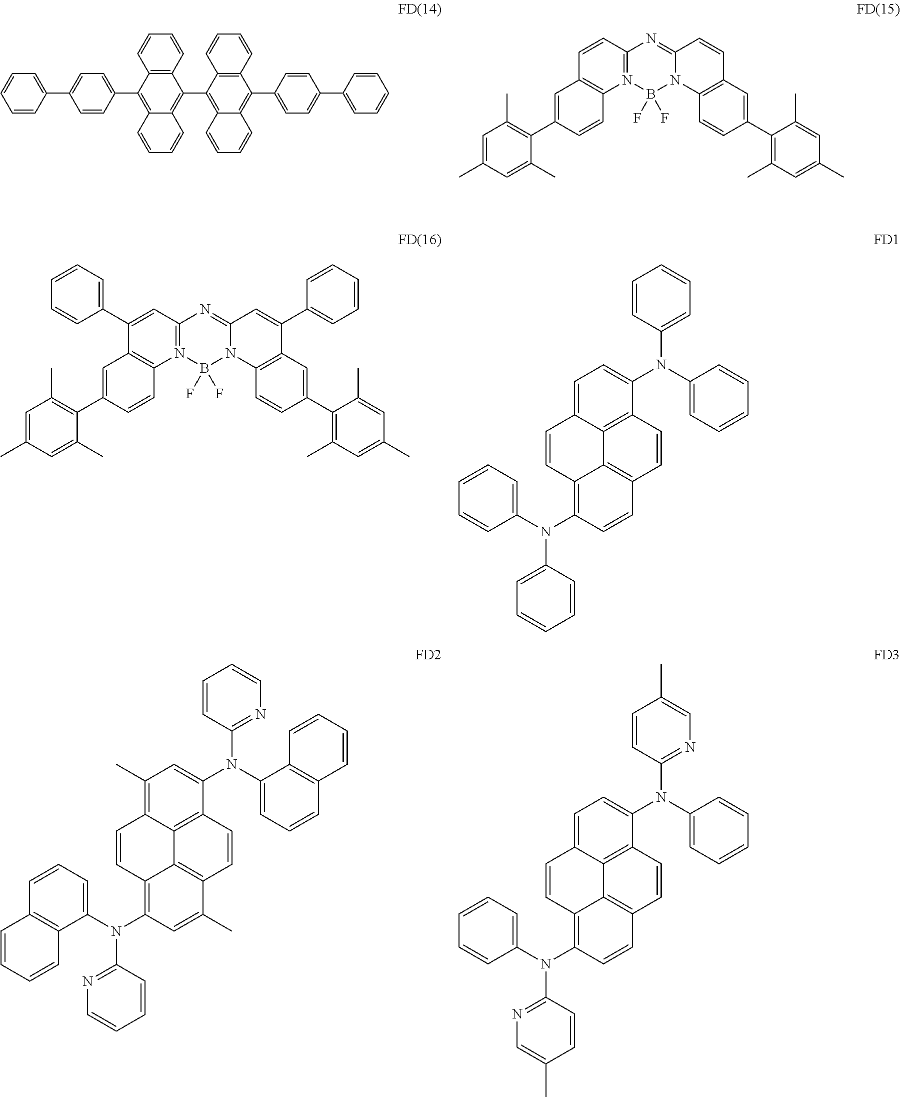

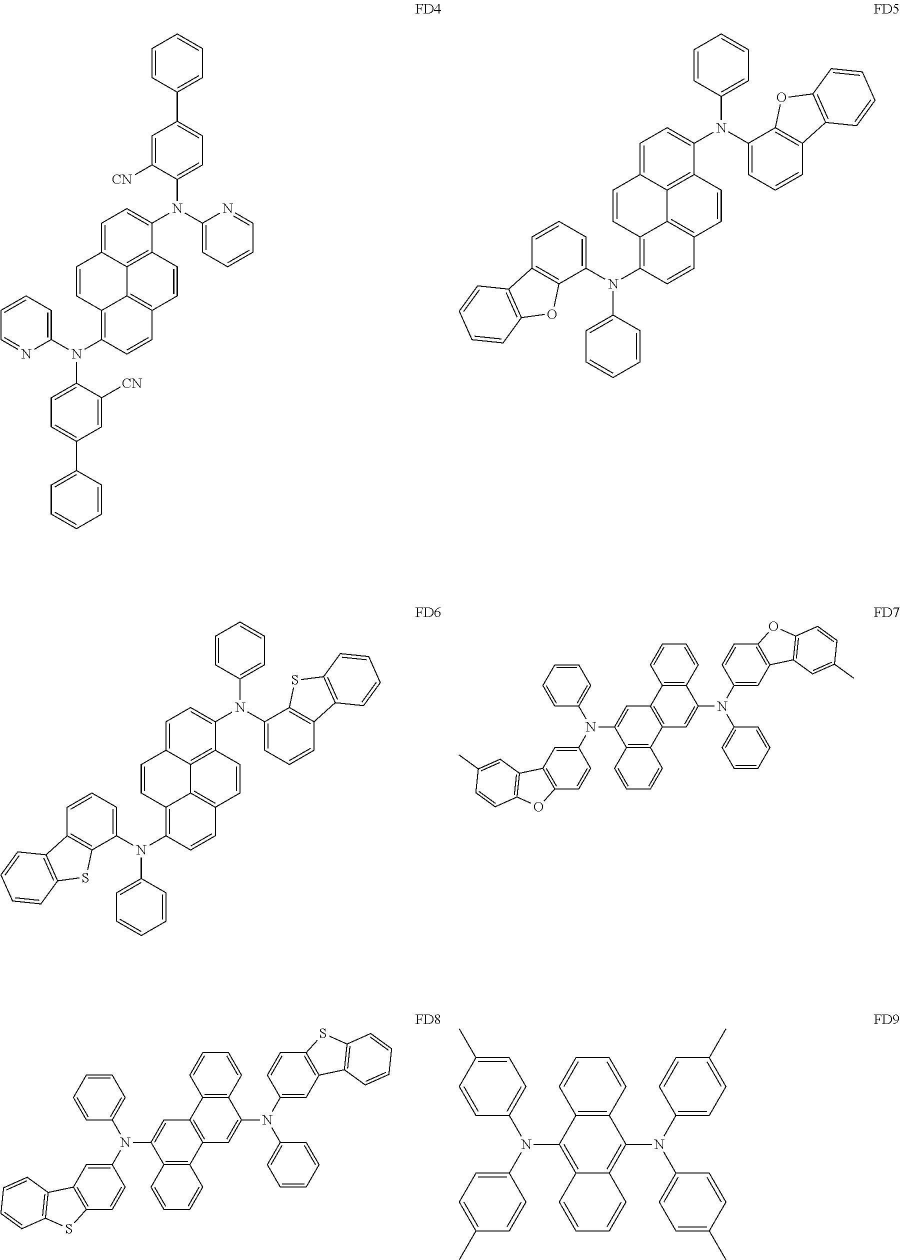

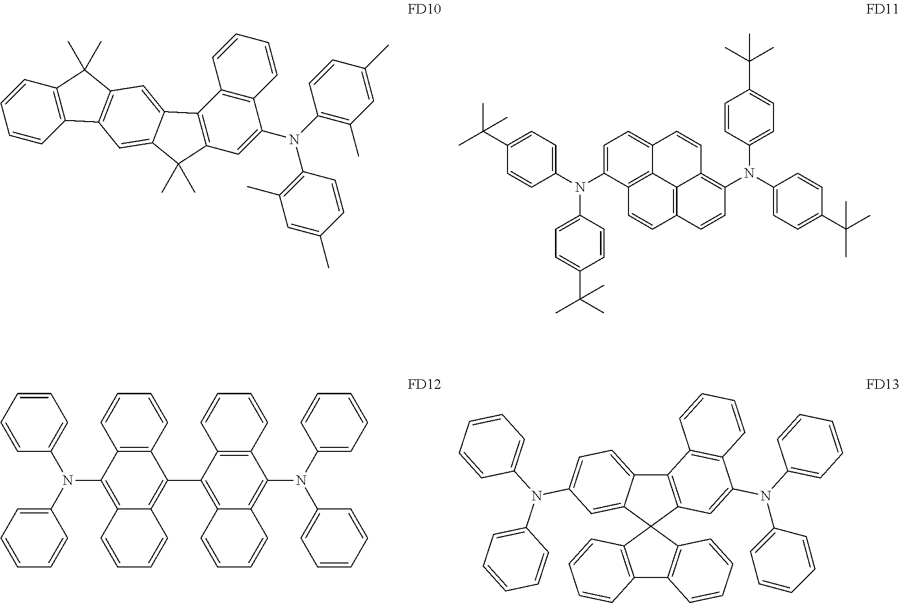 [SMILES: Cc1ccc(N(c2ccc(C)cc2C)c2cc3c(c4ccccc24)-c2cc4c(cc2C3(C)C)-c2ccccc2C4(C)C)c(C)c1 ; CC(C)(C)c1ccc(N(c2ccc(C(C)(C)C)cc2)c2ccc3ccc4c(N(c5ccc(C(C)(C)C)cc5)c5ccc(C(C)(C)C)cc5)ccc5ccc2c3c54)cc1 ; c1ccc(N(c2ccccc2)c2c3ccccc3c(-c3c4ccccc4c(N(c4ccccc4)c4ccccc4)c4ccccc34)c3ccccc23)cc1 ; c1ccc(N(c2ccccc2)c2ccc3c(c2)C2(c4ccccc4-c4ccccc42)c2cc(N(c4ccccc4)c4ccccc4)c4ccccc4c2-3)cc1]

FIGURE is a schematic view of an organic light-emitting device 10 according to one embodiment. Hereinafter, the structure of an organic light-emitting device according to an embodiment and a method of manufacturing an organic light-emitting device according to an embodiment will be described in connection with FIGURE. The organic light-emitting device 10 includes a first electrode 11, an organic layer 15, and a second electrode 19, which are sequentially stacked.

A substrate may be additionally located under the first electrode 11 or above the second electrode 19. For use as the substrate, any substrate that is used in organic light-emitting devices available in the art may be used, and the substrate may be a glass substrate or a transparent plastic substrate, each having excellent mechanical strength, thermal stability, transparency, surface smoothness, ease of handling, and water resistance.

In one or more embodiments, the first electrode 11 may be formed by depositing or sputtering a material for forming the first electrode 11 on the substrate. The first electrode 11 may be an anode. The material for forming the first electrode 11 may be a material with a high work function to facilitate hole injection. The first electrode 11 may be a reflective electrode, a semi-transmissive electrode, or a transmissive electrode. The material for forming the first electrode 11 may be indium tin oxide (ITO), indium zinc oxide (IZO), tin oxide (SnO₂), or zinc oxide (ZnO). In one or more embodiments, the material for forming the first electrode 11 may be metal, such as magnesium (Mg), aluminum (Al), aluminum-lithium (Al—Li), calcium (Ca), magnesium-indium (Mg—In), or magnesium-silver (Mg—Ag).

The first electrode 11 may have a single-layered structure or a multi-layered structure including two or more layers. For example, the first electrode 11 may have a three-layered structure of ITO/Ag/ITO, but the structure of the first electrode 11 is not limited thereto.

The organic layer 15 is located on the first electrode 11.

The organic layer 15 may include a hole transport region, an emission layer, and an electron transport region.

The hole transport region may be between the first electrode 11 and the emission layer.

The hole transport region may include a hole injection layer, a hole transport layer, an electron blocking layer, a buffer layer, or a combination thereof.

The hole transport region may include only either a hole injection layer or a hole transport layer. In one or more embodiments, the hole transport region may have a hole injection layer/hole transport layer structure or a hole injection layer/hole transport layer/electron blocking layer structure, which are sequentially stacked in this stated order from the first electrode 11.

When the hole transport region includes a hole injection layer (HIL), the hole injection layer may be formed on the first electrode 11 by using one or more suitable methods, for example, vacuum deposition, spin coating, casting, and/or Langmuir-Blodgett (LB) deposition.

When a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100° C. to about 500° C., a vacuum pressure of about 10⁻⁸ torr to about 10⁻³ torr, and a deposition rate of about 0.01 Å/sec to about 100 Å/sec. However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 rpm to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include m-MTDATA, TDATA, 2-TNATA, NPB, β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), a compound represented by Formula 201 below, a compound represented by Formula 202 below, or a combination thereof:

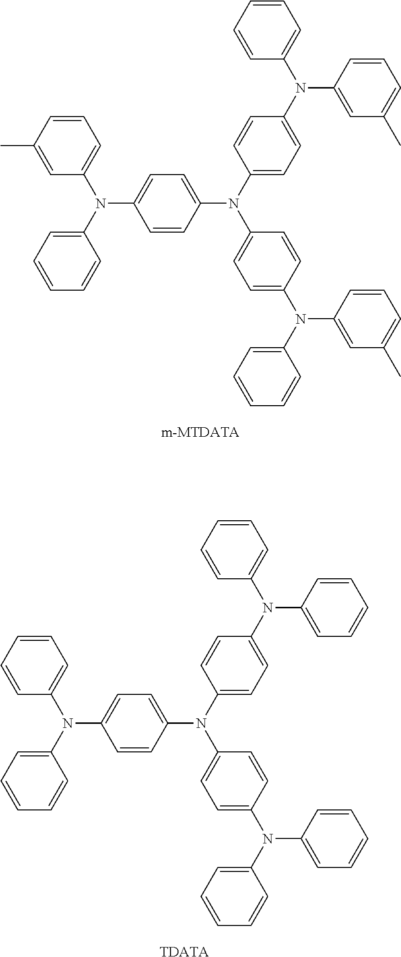

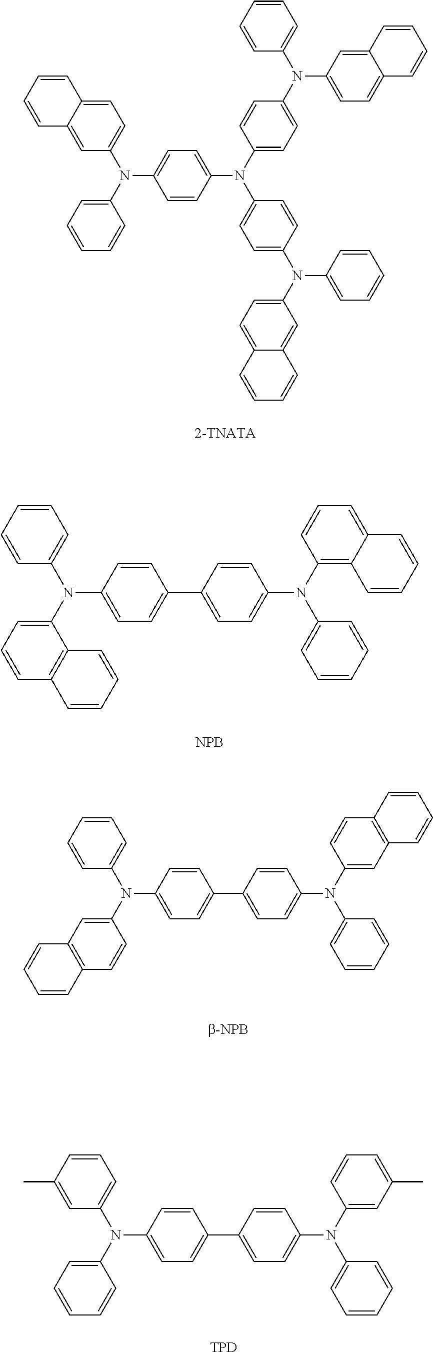

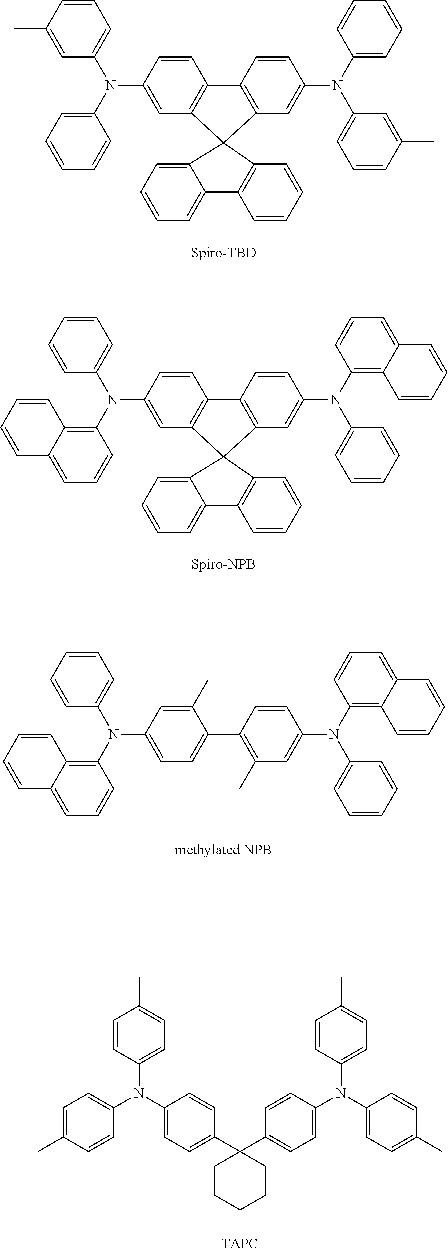

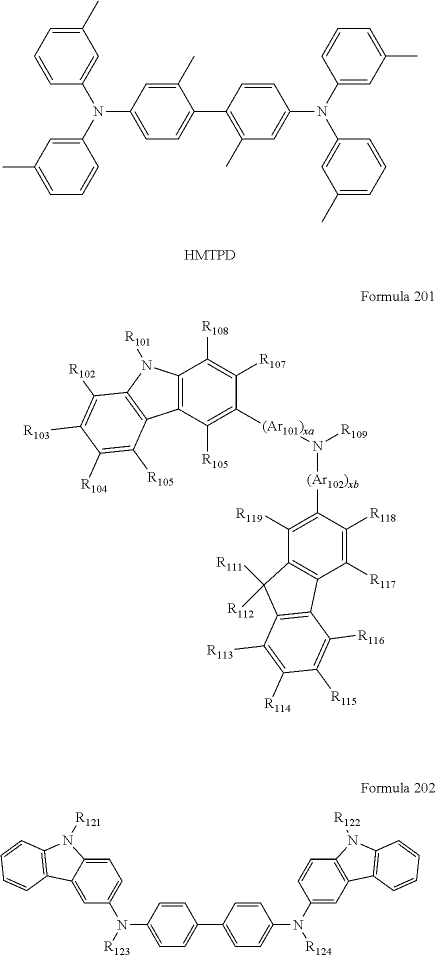

Ar₁₀₁ to Ar₁₀₂ in Formula 201 may each independently be:
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group; or
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or a combination thereof.

xa and xb in Formula 201 may each independently be an integer from 0 to 5, or 0, 1, or 2. For example, xa may be 1 and xb may be 0, but xa and xb are not limited thereto.

R₁₀₁ to R₁₀₈, R₁₁₁ to R₁₁₉ and R₁₂₁ to R₁₂₄ in Formulae 201 and 202 may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, pentyl group, a hexyl group, etc.) or a C₁-C₁₀ alkoxy group (for example, a methoxy group, an ethoxy group, a propoxy group, a butoxy group, a pentoxy group, etc.);
a C₁-C₁₀ alkyl group, or a C₁-C₁₀ alkoxy group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof and a phosphoric acid group or a salt thereof, or a combination thereof;
a phenyl group, a naphthyl group, an anthracenyl group, a fluorenyl group, or a pyrenyl group; or
a phenyl group, a naphthyl group, an anthracenyl group, a fluorenyl group, or a pyrenyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, or a combination thereof,
but embodiments of the present disclosure are not limited thereto.

R₁₀₉ in Formula 201 may be:
a phenyl group, a naphthyl group, an anthracenyl group, or a pyridinyl group; or
a phenyl group, a naphthyl group, an anthracenyl group, or a pyridinyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, a pyridinyl group, or a combination thereof.

According to an embodiment, the compound represented by Formula 201 may be represented by Formula 201A below, but embodiments of the present disclosure are not limited thereto:

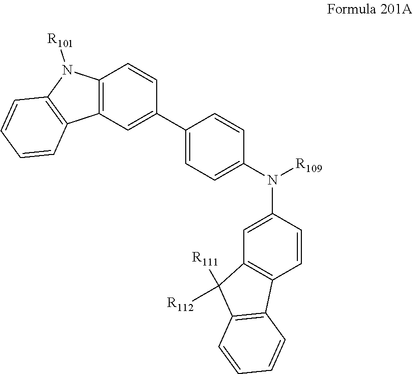

R₁₀₁, R₁₁₁, R₁₁₂, and R₁₀₉ in Formula 201A may be understood by referring to the description provided herein.

For example, the compound represented by Formula 201, and the compound represented by Formula 202 may include compounds HT1 to HT20 illustrated below, but are not limited thereto:

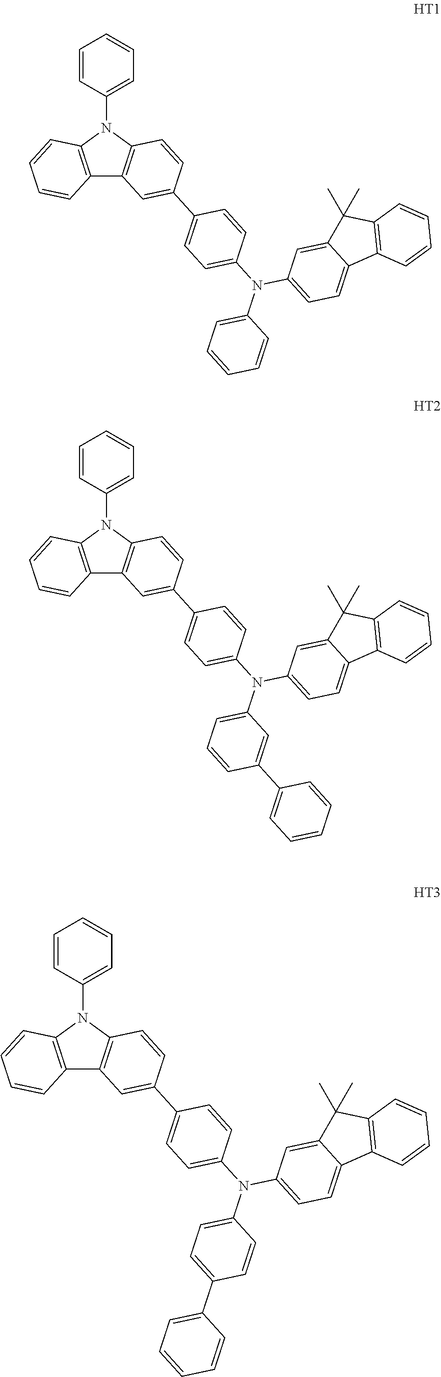 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

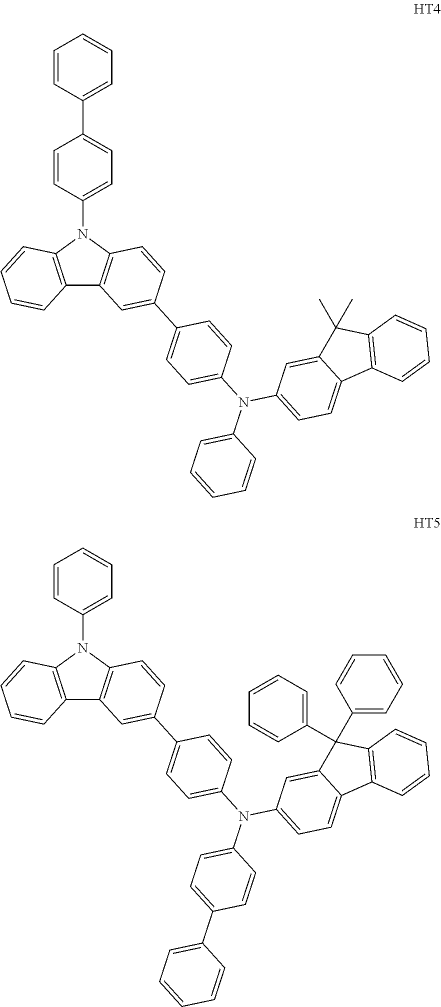 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

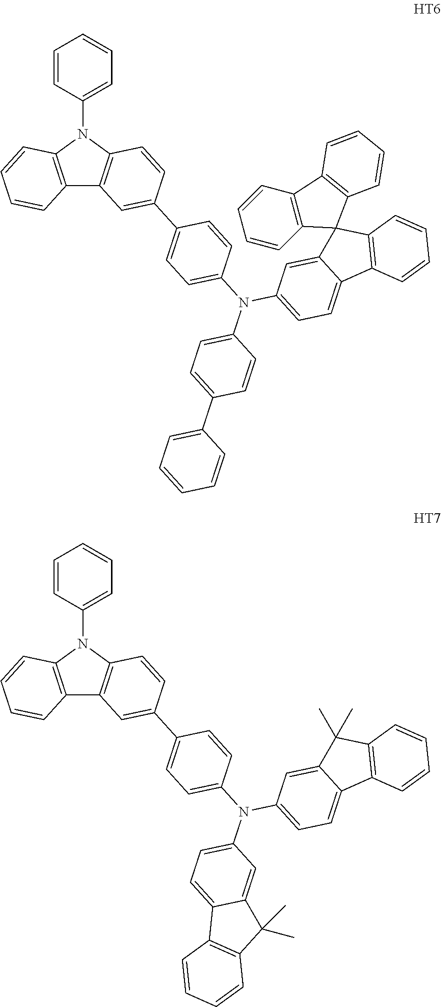 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

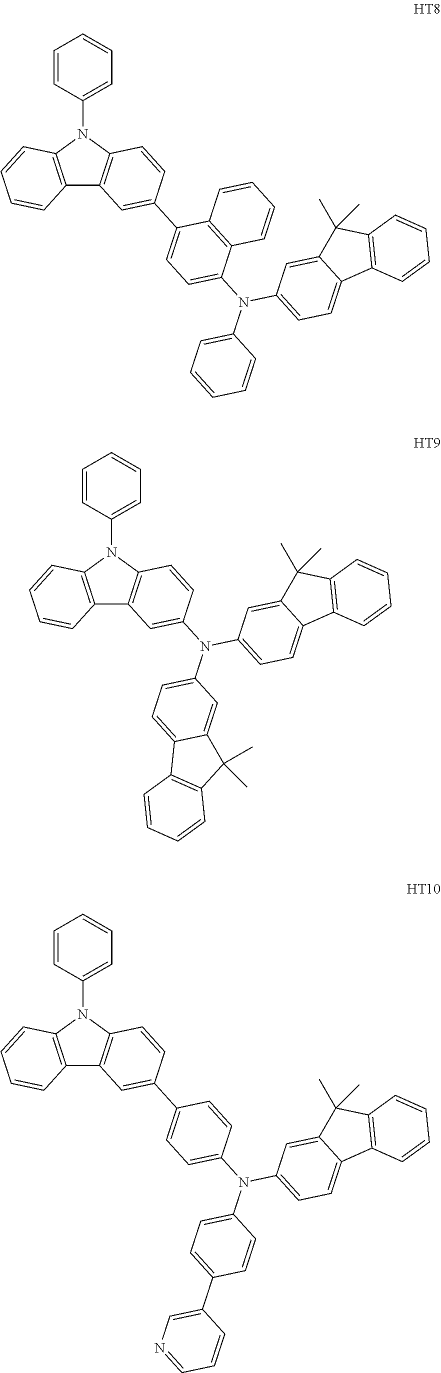 [SMILES: CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21]

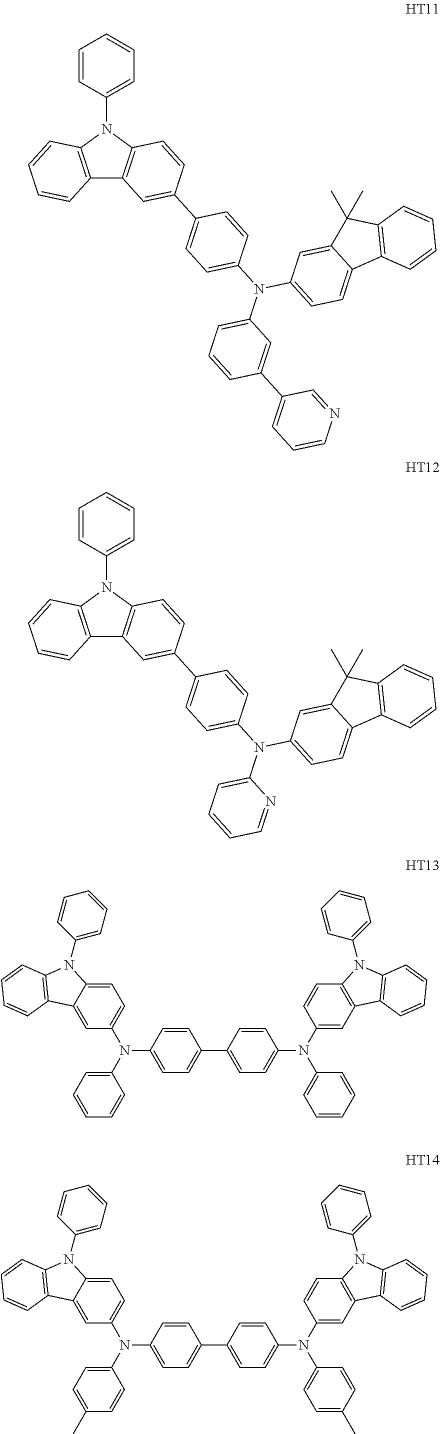 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1]

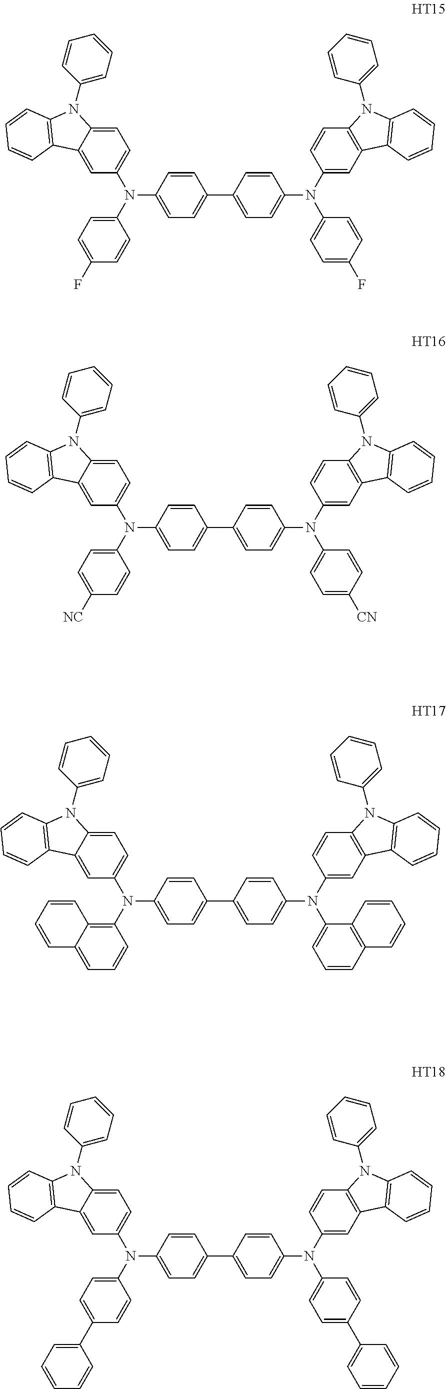 [SMILES: Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1]

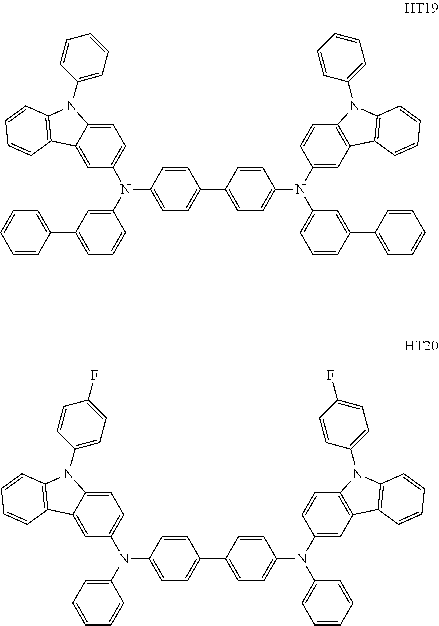 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Å to about 10,000 Å, for example, about 100 Å to about 1,000 Å. When the hole transport region includes a hole injection layer and a hole transport layer at the same time, a thickness of the hole injection layer may be in a range of about 100 Å to about 10,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one a quinone derivative, a metal oxide, or a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenum oxide; and a cyano group-containing compound, such as Compound HT-D1 or Compound HP-1 below, but are not limited thereto.

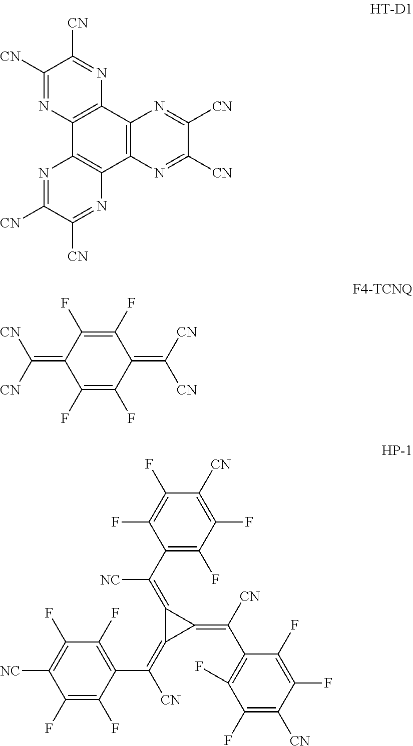

The hole transport region may include a buffer layer.

Also, the buffer layer may compensate for an optical resonance distance according to a wavelength of light emitted from the emission layer, and thus, efficiency of a formed organic light-emitting device may be improved.

The hole transport region may further include an electron blocking layer. The electron blocking layer may include a material available in the art, for example, mCP, but embodiments of the present disclosure are not limited.

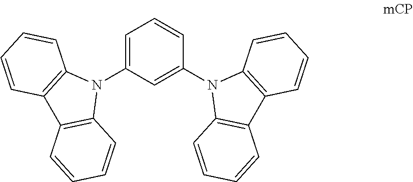 [SMILES: c1cc(-n2c3ccccc3c3ccccc32)cc(-n2c3ccccc3c3ccccc32)c1]

An emission layer (EML) may be formed on the hole transport region by vacuum deposition, spin coating, casting, LB deposition, or the like. When the emission layer is formed by vacuum deposition or spin coating, the deposition or coating conditions may be similar to those applied in forming the hole injection layer although the deposition or coating conditions may vary according to a material that is used to form the hole transport layer.

When the organic light-emitting device is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

The description of the emission layer is given in this specification.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

Then, an electron transport region may be located on the emission layer.

The electron transport region may include a hole blocking layer, an electron transport layer, an electron injection layer, or a combination thereof.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, BCP, Bphen, or a combination thereof, but embodiments of the present disclosure are not limited thereto.

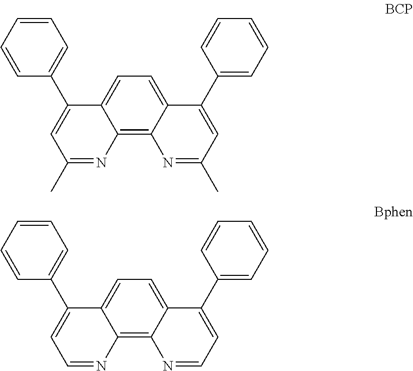

In one or more embodiments, the hole blocking layer may include a host. For example, the hole blocking layer may include Compound H19, but embodiments are not limited thereto.

A thickness of the hole blocking layer may be in a range of about 20 Å to about 1,000 Å, for example, about 30 Å to about 300 Å. When the thickness of the hole blocking layer is within these ranges, the hole blocking layer may have excellent hole blocking characteristics without a substantial increase in driving voltage.

The electron transport layer may further include BCP, Bphen, Alq₃, BAlq, TAZ, NTAZ, or a combination thereof.

In one or more embodiments, the electron transport layer may include ET1, ET2, and ET3, or a combination thereof, but is not limited thereto:

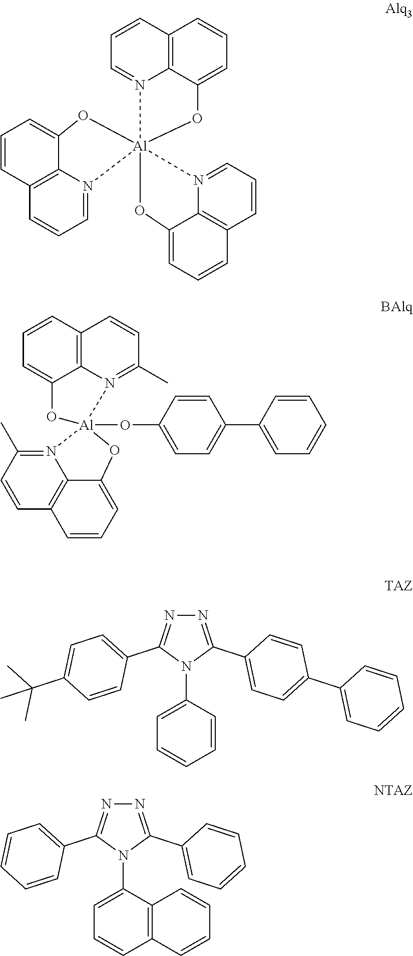

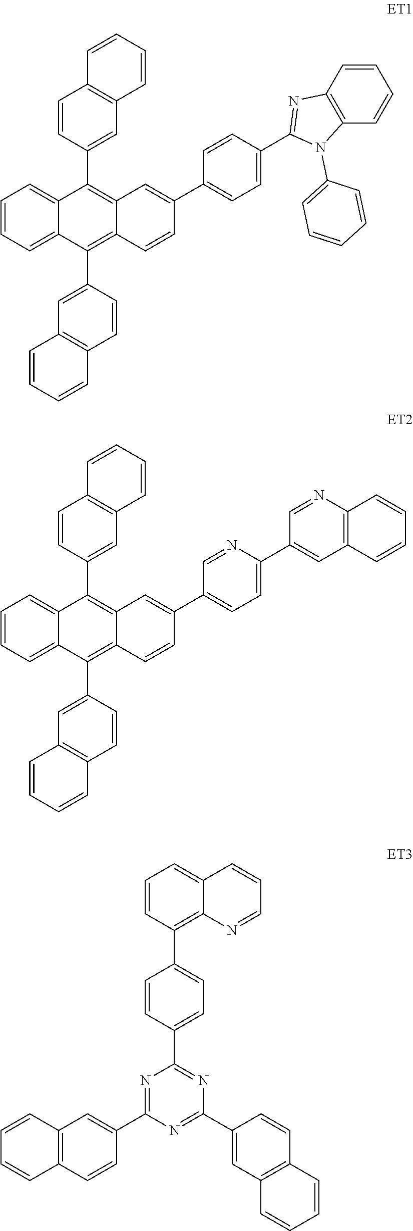 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

A thickness of the electron transport layer may be in a range of about 100 Å to about 1,000 Å, for example, about 150 Å to about 500 Å. When the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (lithium 8-hydroxyquinolate, LiQ) or ET-D2.

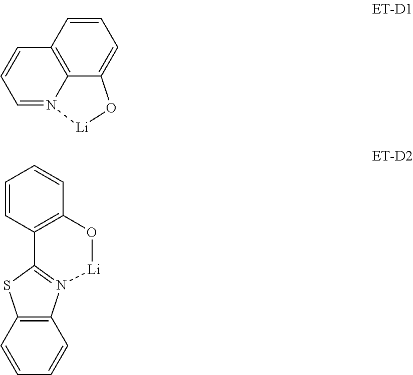

The electron transport region may include an electron injection layer (EIL) that promotes flow of electrons from the second electrode 19 thereinto.

The electron injection layer may include LiF, NaCl, CsF, Li₂O, BaO, or a combination thereof.

A thickness of the electron injection layer may be in a range of about 1 Å to about 100 Å, for example, about 3 Å to about 90 Å. When the thickness of the electron injection layer is within the range described above, the electron injection layer may have satisfactory electron injection characteristics without a substantial increase in driving voltage.

The second electrode 19 is located on the organic layer 15. The second electrode 19 may be a cathode. A material for forming the second electrode 19 may be metal, an alloy, an electrically conductive compound, or a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be formed as the material for forming the second electrode 19. To manufacture a top-emission type light-emitting device, a transmissive electrode formed using ITO or IZO may be used as the second electrode 19.

Hereinbefore, the organic light-emitting device has been described with reference to FIGURE, but embodiments of the present disclosure are not limited thereto.

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” used herein refers to a divalent group having the same structure as that of the C₁-C₆₀ alkyl group.

The term “C₁-C₆₀ alkoxy group” used herein refers to a monovalent group represented by −OA₁₀₁ (wherein A₁₀₁ is the C₁-C₆₀ alkyl group), and examples thereof include a methoxy group, an ethoxy group, and an isopropyloxy group.

The term “C₂-C₆₀ alkenyl group” as used herein refers to a hydrocarbon group formed by substituting a carbon-carbon double bond in the middle or at the terminus of the C₂-C₆₀ alkyl group, and examples thereof include an ethenyl group, a propenyl group, and a butenyl group. The term “C₂-C₆₀ alkenylene group” used herein refers to a divalent group having the same structure as that of the C₂-C₆₀ alkenyl group.

The term “C₂-C₆₀ alkynyl group” as used herein refers to a hydrocarbon group formed by substituting a carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as that of the C₂-C₆₀ alkynyl group.

The term “C₃-C₁₀ cycloalkyl group” as used herein refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as that of the C₃-C₁₀ cycloalkyl group.

The term “C₁-C₁₀ heterocycloalkyl group” as used herein refers to a monovalent saturated monocyclic group having N, O, P, Si, Se, S, or a combination thereof as ring-forming atoms and 1 to 10 carbon atoms, and non-limiting examples thereof include a tetrahydrofuranyl group, and a tetrahydrothiophenyl group. The term “C₁-C₁o heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and a carbon-carbon double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has N, O, P, Si, Se, S, or a combination thereof as ring-forming atoms, 1 to 10 carbon atoms, and a carbon-carbon double bond in its ring. Examples of the C₁-C₁₀ heterocycloalkenyl group are a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system that has N, O, P, Si, Se, S, or a combination thereof as ring-forming atoms, and 1 to 60 carbon atoms. The term “C₁-C₆₀ heteroarylene group” as used herein refers to a divalent group having a heterocyclic aromatic system that has N, O, P, Si, Se, S, or a combination thereof as ring-forming atoms, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₆-C₆₀ aryloxy group” as used herein refers to —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “C₁-C₆₀ heteroaryloxy group” as used herein refers to —OA₁₀₄ (wherein A₁₀₄ is the C₁-C₆₀ heteroaryl group), and the term “C₆-C₆₀ arylthio group” as used herein indicates —SA₁₀₅ (wherein A₁₀₅ is the C₁-C₆₀ heteroaryl group).

The term “C₇-C₆₀ arylalkyl group” as used herein refers to an aryl group linked to an alkyl group. An example of a C₇ arylalkyl group is a benzyl group.

The term “C₂-C₆₀ heteroarylalkyl group” as used herein refers to a heteroaryl group linked to an alkyl group. An example of a C₇ heteroarylalkyl group is a —CH₂-pyridine group.

The term “monovalent non-aromatic condensed polycyclic group” used herein refers to a monovalent group in which two or more rings are condensed with each other, only carbon is used as a ring-forming atom (for example, the number of carbon atoms may be 8 to 60) and the entire group is non-aromatic. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group having two or more rings condensed to each other, N, O, P, Si, Se, S, or a combination thereof as ring forming atoms in addition to carbon atoms (for example, having 2 to 60 carbon atoms), as ring-forming atoms, and the entire group is not aromatic. Non-limiting examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

The term “C₅-C₆₀ carbocyclic group” as used herein refers to a saturated or unsaturated cyclic group having, as a ring-forming atom, 5 to 60 carbon atoms only. The C₅-C₆₀ carbocyclic group may be a monocyclic group or a polycyclic group, and depending on the structure of formula, may be a monovalent, bivalent, trivalent, tetravalent, pentavalent, or hexavalent group.

The term “C₂-C₆₀ heterocyclic group” as used herein refers to a saturated or unsaturated cyclic group having, as ring-forming atoms, N, O, Si, P, Se, S, or a combination thereof other than 2 to 60 carbon atoms. The C₂-C₆₀ heterocyclic group may be a monocyclic group or a polycyclic group, and depending on the structure of formula, may be a monovalent, bivalent, trivalent, tetravalent, pentavalent, or hexavalent group.

Substituents of the substituted C₅-C₆₀ carbocyclic group, the substituted C₂-C₆₀ heterocyclic group, the substituted π electron-deficient nitrogen-containing C₂-C₆₀ heterocyclic group, the substituted C₁-C₆₀ alkyl group, the substituted C₂-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₆₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁o heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), or a combination thereof; or
—N(Q₃₁)(Q₃₂), —Si(Q₃₃)(Q₃₄)(Q₃₅), —B(Q₃₆)(Q₃₇), or —P(═O)(Q₃₈)(Q₃₉),
wherein Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, an unsubstituted C₆-C₆₀ aryl group, a C₆-C₆₀ aryl group substituted with a C₁-C₆₀ alkyl group, a C₆-C₆₀ aryl group, or a combination thereof, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group.

The “room temperature” used herein refers to the temperature of about 25° C. Hereinafter, a compound and an organic light-emitting device according to embodiments are described in detail with reference to the Synthesis Example and Examples. However, the organic light-emitting device is not limited thereto. The wording “B was used instead of A” used in describing Synthesis Examples means that an amount of A used was identical to an amount of B used, in terms of a molar equivalent.

### EXAMPLES

### Synthesis Example 1: Synthesis of Compound 5

Compound 5 was synthesized according to the following reaction scheme.

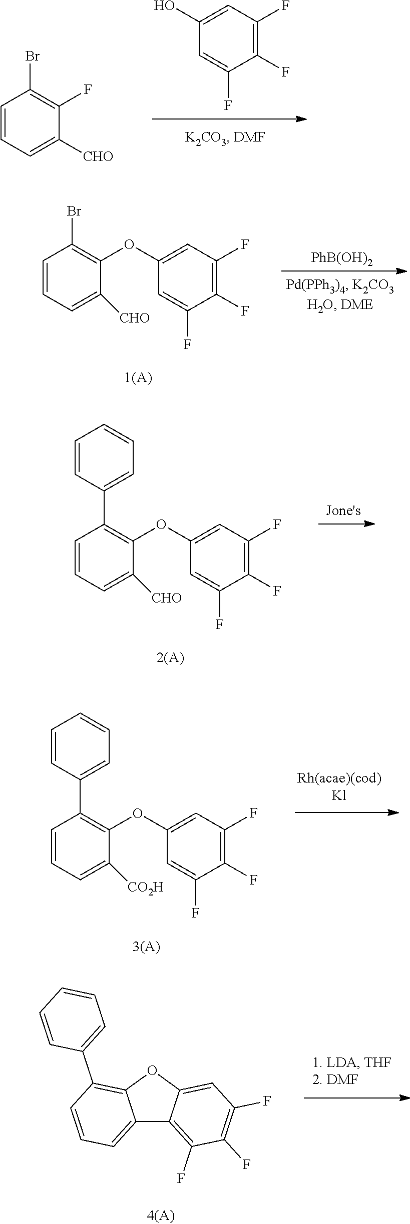

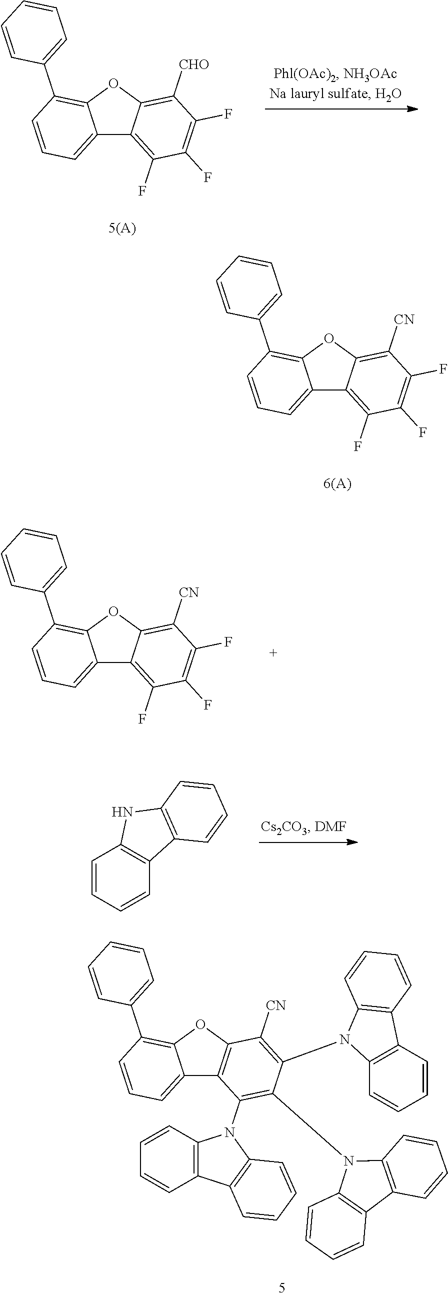

(1) Synthesis of Intermediate 1(A)

3-bromo-2-fluorobenzaldehyde (4 g, 24.59 mmol), 3,4,5-trifluorophenol (4.00 g, 27.05 mmol), potassium carbonate (K₂CO₃) (3.74 g, 27.05 mmol), and N-methyl-2-pyrrolidinone (NMP) were mixed at a temperature of 200° C. for 4 hours. After completion of the reaction, the reaction mixture was cooled to room temperature and then purified by silica gel column chromatography to obtain 8.1 g (yield: 99%) of Intermediate 1(A).

(2) Synthesis of Intermediate 2(A)

3-bromo-2-(3,4,5-trifluorophenoxy)benzaldehyde (8 g, 73.64 mmol), phenylboronic acid (3.24 g, 26.58 mmol), palladium (0) tetrakis(triphenylphosphine) [Pd(PPh₃)₄)] (1.396 g, 1.21 mmol), and potassium carbonate (K₂CO₃) (4 g, 29.00 mmol) were added to 60 ml of tetrahydrofuran and 60 ml of distilled water, and the resulting mixture was refluxed. After completion of the reaction, the reaction mixture was cooled to room temperature, and then methanol was added thereto, followed by filtration through silica gel. The resulting organic layer was concentrated, and then methanol was added thereto to perform a precipitation process, thereby obtaining 7.35 g (93%) of Intermediate 2(A) as a white solid.

(3) Synthesis of Intermediate 3(A)

2-(3,4,5-trifluorophenoxy)-[1,1′-biphenyl]-3-carbaldehyde (1 g, 3.05 mmol), Jones reagent (0.54 mL/mmol of reagents), and acetone were mixed at 0° C. for 20 minutes. Then, 10 mL of distilled water was added thereto and stirred. After completion of the reaction, the reaction mixture was heated to room temperature and then purified by silica gel column chromatography to obtain 0.87 g (yield: 83%) of Intermediate 3(A).

(4) Synthesis of Intermediate 4(A)

2-(3,4,5-trifluorophenoxy)-[1,1′-biphenyl]-3-carboxylic acid (3 g, 8.71 mmol), potassium iodide (KI) (1.59 g, 9.58 mmol), (acetylacetonato) (1,5-cyclooctadiene)rhodium (I) [RH(acac)(cod)] (0.14 g, 0.44 mmol), and 50 mL of ethyl acetate were mixed and stirred at 100° C. for 1 hour. Then, 100 mL of distilled water was added thereto and stirred. After completion of the reaction, the reaction mixture was cooled to room temperature and then purified by silica gel column chromatography to obtain 2.35 (yield: 90%) of Intermediate 4(A).

(5) Synthesis of Intermediate 5(A)

1,2,3-trifluoro-6-phenyldibenzo[b,d]furan (2 g, 6.71 mmol) was dissolved in tetrahydrofuran, and then, cooled by using dry ice bath to −78° C. Lithium diisopropylamide (LDA) (0.862 g, 8.05 mmol) was slowly added dropwise thereto, and the reaction mixture was stirred at −78° C. of 30 minutes. N,N-dimethylformamide (DMF) was added to the reaction container, and then, the temperature was slowly raised to room temperature, followed by 6 hours of stirring. After completion of the reaction, the solvent was removed therefrom, and the reaction mixture was acidified at room temperature for 2 hours by using aqueous sulfuric acid. After completion of the reaction, the reaction mixture was neutralized with aqueous sodium hydroxide and extracted with dichloromethane. The obtained organic layer was concentrated and purified by silica gel column chromatography to obtain 1.68 g (yield: 77%) of Intermediate 5 (A).

(6) Synthesis of Intermediate 6(A)

(Diacetoxyiodo)benzene (0.025 g, 0.08 mmol), ammonium acetate (0.142 g, 1.84 mmol), sodium lauryl sulfate (0.486 g, 1.69 mmol), and 10 mL of DMF were added to 1,2,3-trifluoro-6-phenyldibenzo[b,d]furan-4-carbaldehyde (0.5 g, 1.53 mmol), and then, the resultant mixture was stirred 150° C. for 4 hours. After completion of the reaction, the reaction mixture was cooled to room temperature and then purified by silica gel column chromatography to obtain 0.2 g (yield: 40%) of Intermediate 6(A).

(6) Synthesis of Compound 5

Intermediate 6(A) (5.0 g, 15.47 mmol), 9H-carbazole (3.103 g, 18.56 mmol), and cesium carbonate (Cs₂CO₃) (7.055 g, 21.65 mmol) were added to 77 ml of DMF, and then, the resultant mixture was stirred at 165° C. for 20 hours. After completion of the reaction, the reaction mixture was cooled to room temperature, and then methanol was added thereto, followed by filtration through silica gel. The resulting organic layer was concentrated, the crude residue was dissolved in toluene, filtered through silica gel, and concentrated. Recrystallization (ethyl acetate/ethanol) was performed thereon to obtain 2.7 g (yield: 23%) of Compound 5 as a yellow solid.

LC-Mass (calculated: 764.26 g/mol, found: 764.36 g/mol (M+1))

### Synthesis Example 2: Synthesis of Compound 6

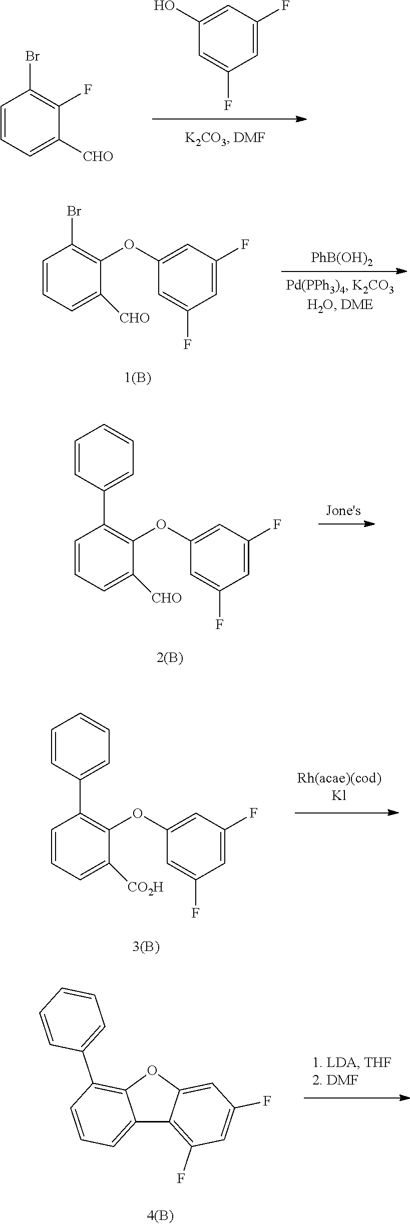

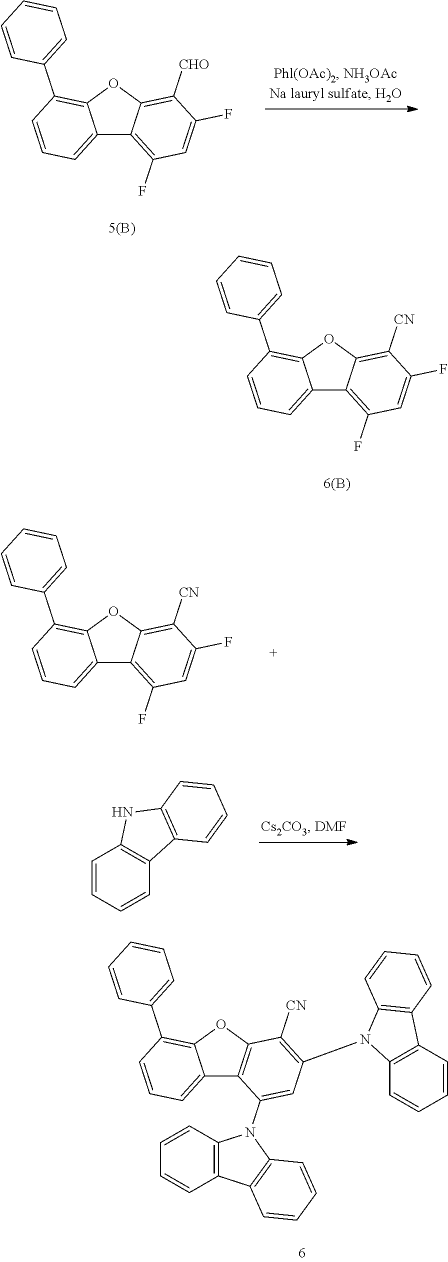

Compound 6 was obtained in an amount of 3.5 g (yield: 36%) in the same manner as used to synthesize Compound 5, except that 3.5 difluorophenol was used instead of 3,4,5-trifluorophenol.

LC-Mass (calculated: 599.20 g/mol, found: 599.3 g/mol (M+1))

### Synthesis Example 3: Synthesis of Compound 7

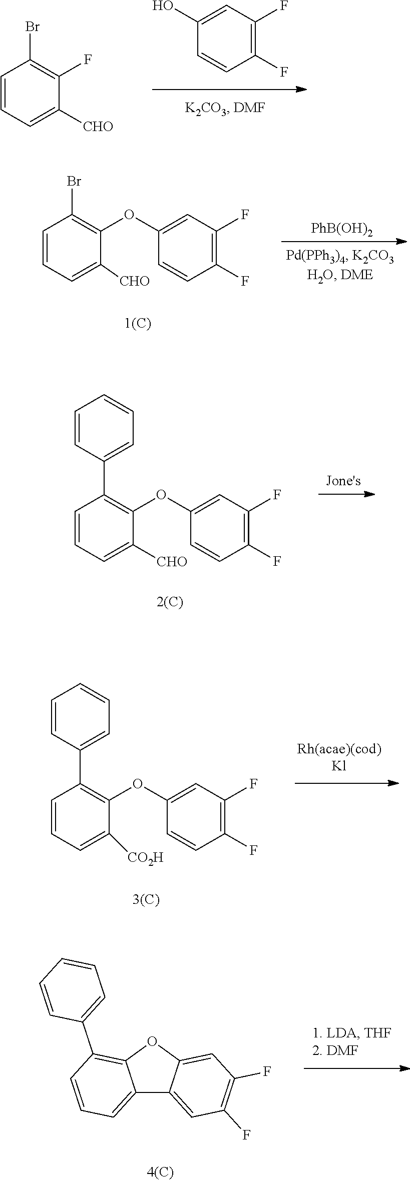

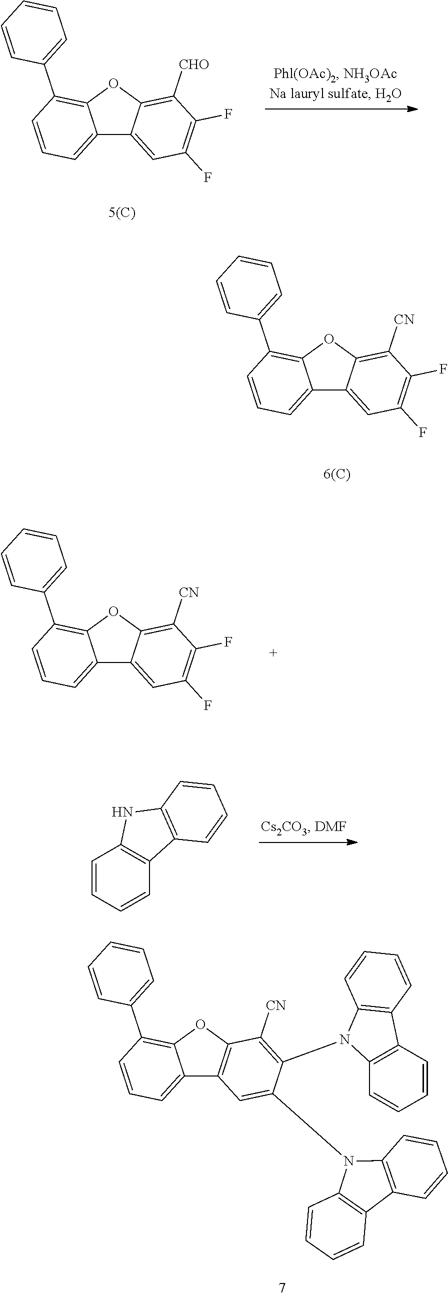

Compound 7 was obtained in an amount of 2.9 g (yield: 30%) in the same manner as used to synthesize Compound 5, except that 3,4-difluorophenol was used instead of 3,4,5-trifluorophenol.

LC-Mass (calculated: 599.20 g/mol, found: 599.3 g/mol (M+1))

### Evaluation Example 1

According to the methods shown in Table 2, the emission spectrum, HOMO, LUMO, singlet (S₁) energy level, triplet (T₁) energy level, and ΔE_{ST} of the compounds shown in Table 3 were evaluated. The results are shown in Table 3.

TABLE 2

| | |
|---|---|
| photoluminescence(PL) | Each compound was diluted in toluene at the |
| spectrum | concentration of 10⁻⁵ M, and the PL spectrum |
| | was measured (at 298K) by using the |
| | Xenon lamp-equipped Hitachi Model No. |
| | F7000 Spectrofluorometer |
| S₁ energy level | The PL spectrum of a mixture of toluene and |
| evaluation method | each compound (diluted to a concentration of |
| | 1 × 10⁻⁴ M) was measured at room temperature |
| | by using a PL spectrometer and identified peaks |
| | were analyzed to calculate on-set S₁ energy level |
| T₁ energy level | A mixture of toluene and each compound |
| evaluation method | (diluted to aconcentration of 1 × 10⁻⁴ M) was |
| | placed in a quartz cell and placed in liquid |
| | nitrogen (77 K). The PL spectrum thereof was |
| | measured by using a PL spectrometer, and the |
| | PL spectrometer was compared with a PL |
| | spectrometer measured at room temperature |
| | to identify peaks that appeared only at room |
| | temperature to calculate on-set T₁ energy level. |
| ΔE_{ST} | Calculated the difference between S₁ energy |
| | level and T₁ energy level. |

TABLE 3

| | | | | | | The maximum |
|---|---|---|---|---|---|---|
| | | | S₁ | T₁ | | emission |
| | | | energy | energy | | wavelength |
| Compound | HOMO | LUMO | level | Level | ΔE_{ST} | (nm) of the |
| No. | (eV) | (eV) | (eV) | (eV) | (eV) | PL spectrum |
| Compound 5 | −6.016 | −2.837 | 3.01 | 2.76 | 0.153 | 412 |
| Compound 6 | −5.77 | −2.092 | 2.883 | 2.653 | 0.32 | 430 |
| Compound 7 | −5.74 | −2.18 | 2.917 | 2.795 | 0.122 | 425 |
| A | −5.66 | −2.14 | 3.19 | 3.0 | 0.19 | 389 |
| B | −5.78 | −2.35 | 3.3 | 2.92 | 0.38 | 376 |

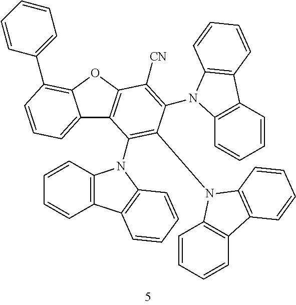 [SMILES: N#Cc1c(-n2c3ccccc3c3ccccc32)c(-n2c3ccccc3c3ccccc32)c(-n2c3ccccc3c3ccccc32)c2c1oc1c(-c3ccccc3)cccc12]

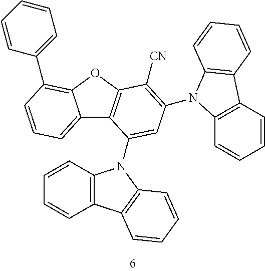 [SMILES: N#Cc1c(-n2c3ccccc3c3ccccc32)cc(-n2c3ccccc3c3ccccc32)c2c1oc1c(-c3ccccc3)cccc12]

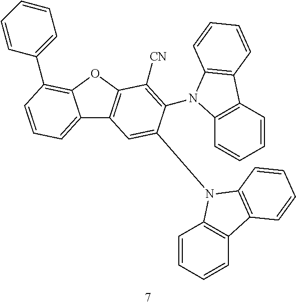 [SMILES: N#Cc1c(-n2c3ccccc3c3ccccc32)c(-n2c3ccccc3c3ccccc32)cc2c1oc1c(-c3ccccc3)cccc12]

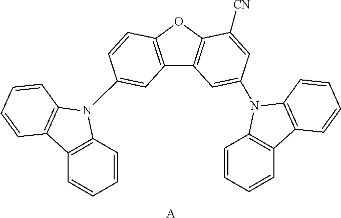

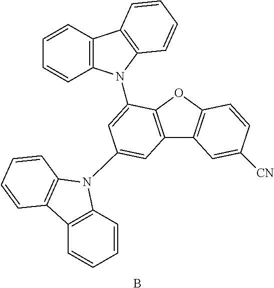

From Table 3, it can be seen that Compounds 5 to 7 can emit deep blue light, have a small ΔE_{ST} and emit thermal activation delayed fluorescence light. Light of Comparative Examples A and B had extremely short wavelengths in the wavelength range of purple, and it would be difficult to utilize them as actual light emitting materials due to their low luminous efficiency.

### Evaluation Example 2

Compound H19 and Compound 5 (15% by weight) were co-deposited on a quartz cell to prepare Film 1 having a thickness of 100 Å. Films 2, 3, A, and B were fabricated in the same manner as described above by using Compound 6, 7, A, and B, respectively, instead of Compound 5. Then, Films 1 to 3, A, and B were excited with excitation light having a wavelength of 340 nm under a nitrogen atmosphere by using C9920-02 and PMA-11 of Hamamatsu photonics to measure luminous efficiency (PL yield) of each film, and the results are shown in Table 4 below.

TABLE 4

| Film | | Luminous |
|---|---|---|
| No. | Film component | efficiency (%) |
| 1 | Compound 5 + H19 | 17.4 |
| 2 | Compound 6 + H19 | 58.1 |
| 3 | Compound 7 + H19 | 68 |
| A | Compound A + H19 | <1 |
| B | Compound B + H19 | <1 |

From Table 4, it can be seen that Films 1 to 3 have higher luminous efficiencies than Films A and B.

### Example 1

A glass substrate, on which a 1500 Å-thick indium tin oxide (ITO) electrode (first electrode, anode) was formed, was cleaned by distilled water ultrasonication. After the distilled water ultrasonication was completed, ultrasonic cleaning was performed sequentially with isopropyl alcohol, acetone, and methanol, once each, and the glass substrate was dried and transferred to a plasma cleaner. The glass substrate was cleaned by using oxygen plasma for 5 minutes, and then transferred to a vacuum laminator.

Compound HT3 was co-deposited on the ITO electrode on the glass substrate to form a first hole injection layer having a thickness of 100 Å, Compound HT-D1 was deposited on the first hole injection layer to form a second hole injection layer having a thickness of 100 Å, and mCP was deposited on the second hole injection layer to form an electron blocking layer having a thickness of 150 Å, thereby completing the formation of a hole transport region.

Compound H19 (host) and Compound 5 (dopant) were co-deposited on the hole transport region at a volumetric ratio of 85:15 to form an emission layer having a thickness of 300 Å.

Compound ET3 was vacuum deposited on the emission layer to form an electron transport layer having a thickness of 300 Å, and then, ET-D1 (Liq) was deposited on the electron transport layer to form an electron injection layer having a thickness of 10 Å, and an Al second electrode (cathode) having a thickness of 1200 Å was formed on the electron injection layer, thereby completing the manufacture of an organic light-emitting device.

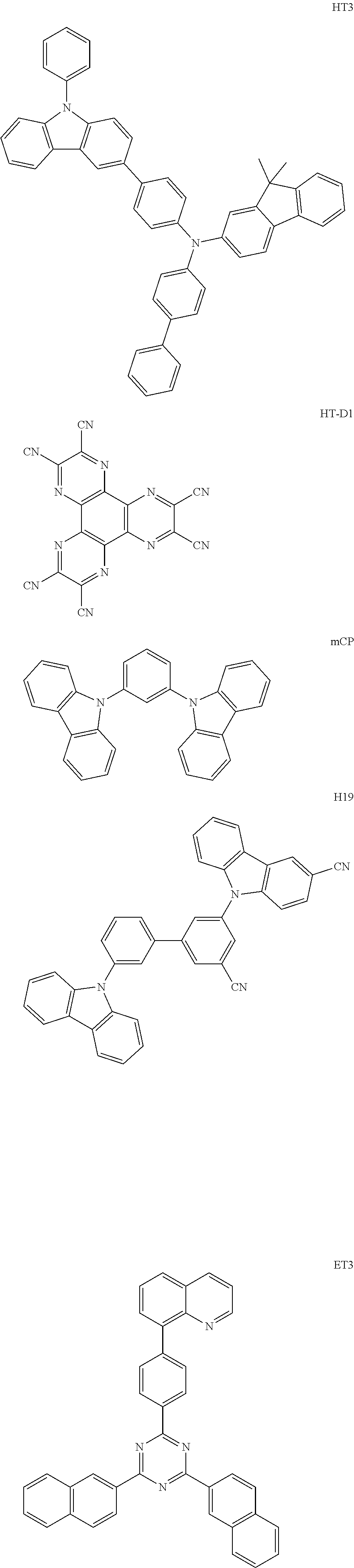

### Examples 2 and 3 and Comparative Examples A and B

Organic light-emitting devices were manufactured in the same manner as in Example 1, except that in forming an emission layer, for use as a dopant, corresponding compounds shown in Table 5 were used instead of Compound 1.

### Evaluation Example 3

The driving voltage and external quantum efficiency of the organic light-emitting devices manufactured according to Examples 1 to 3 and Comparative Examples A and B were measured by using a current-voltage meter (Keithley 2400) and a luminance meter (Minolta Cs-1000A) at 500 cd/m². Results thereof are shown in Table 5.

TABLE 5

| | | | Driving | External quantum |
|---|---|---|---|---|
| Example | | | voltage | efficiency |
| No. | Host | Dopant | (V) | (Relative value (%)) |
| Example | Compound | Compound 5 | 5.85 | 100 |
| 1 | H19 | | | |
| Example | Compound | Compound 6 | 6.99 | 143 |
| 2 | H19 | | | |
| Example | Compound | Compound 7 | 8.87 | 170 |
| 3 | H19 | | | |
| Comparative | Compound | Compound A | 10.61 | 9.8 |
| Example A | H19 | | | |
| Comparative | Compound | Compound B | 9.65 | 10.5 |
| Example B | H19 | | | |

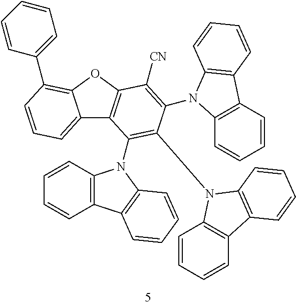 [SMILES: N#Cc1c(-n2c3ccccc3c3ccccc32)c(-n2c3ccccc3c3ccccc32)c(-n2c3ccccc3c3ccccc32)c2c1oc1c(-c3ccccc3)cccc12]

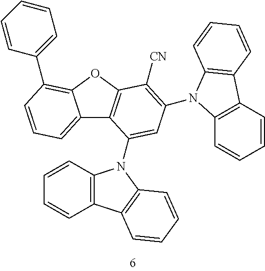 [SMILES: N#Cc1c(-n2c3ccccc3c3ccccc32)cc(-n2c3ccccc3c3ccccc32)c2c1oc1c(-c3ccccc3)cccc12]

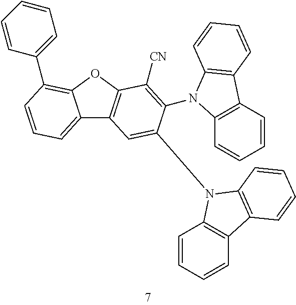 [SMILES: N#Cc1c(-n2c3ccccc3c3ccccc32)c(-n2c3ccccc3c3ccccc32)cc2c1oc1c(-c3ccccc3)cccc12]

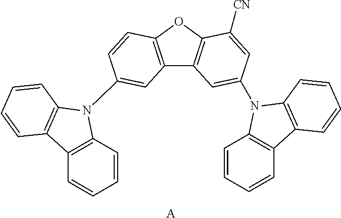

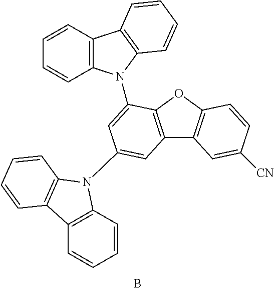

From Table 5, it can be seen that the organic light-emitting devices of Examples 1 to 3 have better driving voltage and/or external quantum efficiency characteristics than the organic light-emitting devices of Comparative Examples A and B.

The condensed-cyclic compound has excellent delayed fluorescent emission characteristics, and an organic light-emitting device employing the condensed-cyclic compound may have high efficiency and/or long lifespan.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the FIGURES, it will be understood by those of ordinary skill in the art that various changes in form and details may be made therein without departing from the spirit and scope as defined by the following claims.

What is claimed is:

1. A condensed-cyclic compound represented by Formula 1: 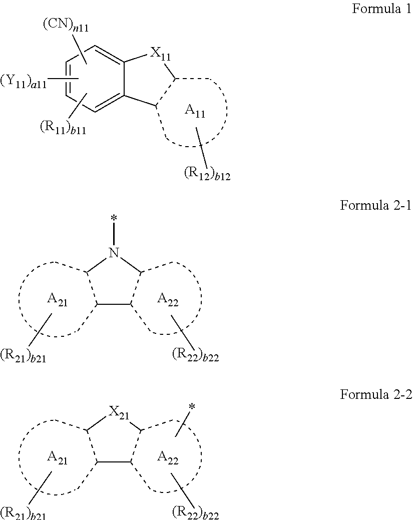
wherein, in Formulae 1, 2-1, and 2-2,
n11 is 1 or 2;
Y₁₁ is a group represented by Formulae 2-1 or 2-2;
a11 is 2 or 3;
X₁₁ is O, S, N(R₁₆), or C(R₁₆)(R₁₇);
A₁₁, A₂₁, and A₂₂ are each independently a C₅-C₆₀ carbocyclic group or a C₁-C₆₀ heterocyclic group;
X₂₁ is O, S, N(R₂₃), or C(R₂₃)(R₂₄);
R₁₁, R₁₂, and R₂₁ to R₂₄ are each independently hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b11 is 0 or 1;
b12, b21, and b22 are each independently 1, 2, 3, 4, 5, 6, 7, 8, 9, or 10;
the sum of n11, a11, and b11 is 4;
Q₁ to Q₃ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, a C₁-C₆₀ alkyl group which is substituted with deuterium, —F, a cyano group, a C₁-C₆₀ alkyl group, a C₆-C₆₀ aryl group, or a combination thereof, or a C₆-C₆₀ aryl group which is substituted with deuterium, —F, a cyano group, a C₁-C₆₀ alkyl group, a C₆-C₆₀ aryl group, or a combination thereof; and
* indicates a binding site to a neighboring atom.

2. The condensed-cyclic compound of claim 1, wherein
n11 is 1.

3. The condensed-cyclic compound of claim 1, wherein
X₁₁ is O, S, or N(R₁₆).

4. The condensed-cyclic compound of claim 1, wherein
A₁₁, A₂₁, and A₂₂ are each independently a benzene group, a naphthalene group, an anthracene group, a phenanthrene group, a phenalene group, a triphenylene group, a pyridine group, a pyrimidine group, a pyrazine group, a pyridazine group, a triazine group, a quinoline group, an isoquinoline group, 2,6-naphthyridine group, 1,8-naphthyridine group, 1,5-naphthyridine group, 1,6-naphthyridine group, 1,7-naphthyridine group, 2,7-naphthyridine group, a quinoxaline group, a phthalazine group, a quinazoline group, a phenanthroline group, a benzoquinoline group, a benzoisoquinoline group, a benzoquinoxaline group, a benzoquinazoline group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an indenopyridine group, an indolopyridine group, a benzofuropyridine group, a benzothienopyridine group, a benzosilolopyridine group, an indenopyrimidine group, an indolopyrimidine group, a benzofuropyrimidine group, a benzothienopyrimidine group, or a benzosilolopyrimidine group.

5. The condensed-cyclic compound of claim 1, wherein
A₁₁ is a benzene group or a naphthalene group;
A₂₁ and A₂₂ are each independently a benzene group, a naphthalene group, a fluorene group, a carbazole group, a dibenzofuran group, or a dibenzothiophene group.

6. The condensed-cyclic compound of claim 1, wherein
R₁₁, R₁₂, and R₂₁ to R₂₄ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cycloctyl group, a bicyclo[2.2.1]heptyl group, an adamantyl group, a norbornyl group, a norbornenyl group, a cyclopentenyl group, a cyclohexenyl group, a cycloheptenyl group, a phenyl group, a biphenyl group, a C₇-C₂₀ alkylphenyl group, a naphthyl group, a pyridinyl group, a pyrimidinyl group, or a combination thereof;
a cyclopentyl group, a cyclohexyl group, a cycloheptyl group, a cycloctyl group, a bicyclo[2.2.1]heptyl group, an adamantyl group, a norbornyl group, a norbornenyl group, a cyclopentenyl group, a cyclohexenyl group, a cycloheptenyl group, a phenyl group, a biphenyl group, a C₇-C₂₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, or an imidazopyrimidinyl group;
a cyclopentyl group, a cyclohexyl group, a cycloheptyl group, a cycloctyl group, a bicyclo[2.2.1]heptyl group, an adamantyl group, a norbornyl group, a norbornenyl group, a cyclopentenyl group, a cyclohexenyl group, a cycloheptenyl group, a phenyl group, a biphenyl group, a C₇-C₂₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, or an imidazopyrimidinyl group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cycloctyl group, a bicyclo[2.2.1]heptyl group, an adamantyl group, a norbornyl group, a norbornenyl group, a cyclopentenyl group, a cyclohexenyl group, a cycloheptenyl group, a phenyl group, a biphenyl group, a C₇-C₂₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, —Si(Q₁₁)(Q₁₂)(Q₁₃), —B(Q₁₁)(Q₁₂), —N(Q₁₁)(Q₁₂), or a combination thereof; or
—Si(Q₁)(Q₂)(Q₃), —B(Q₁)(Q₂), -or N(Q₁)(Q₂),
wherein Q₁ to Q₃ and Q₁₁ to Q₁₃ are each independently
a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, 3-pentyl group, 3-methyl-2-butyl group, a phenyl group, a biphenyl group, a C₇-C₂₀ alkylphenyl group, or a naphthyl group; or
a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, 3-pentyl group, 3-methyl-2-butyl group, a phenyl group, or a naphthyl group, each substituted with deuterium, a phenyl group, or a combination thereof.

7. The condensed-cyclic compound of claim 1, wherein
Y₁₁ is a group represented by one of Formulae 2-11 to 2-17: 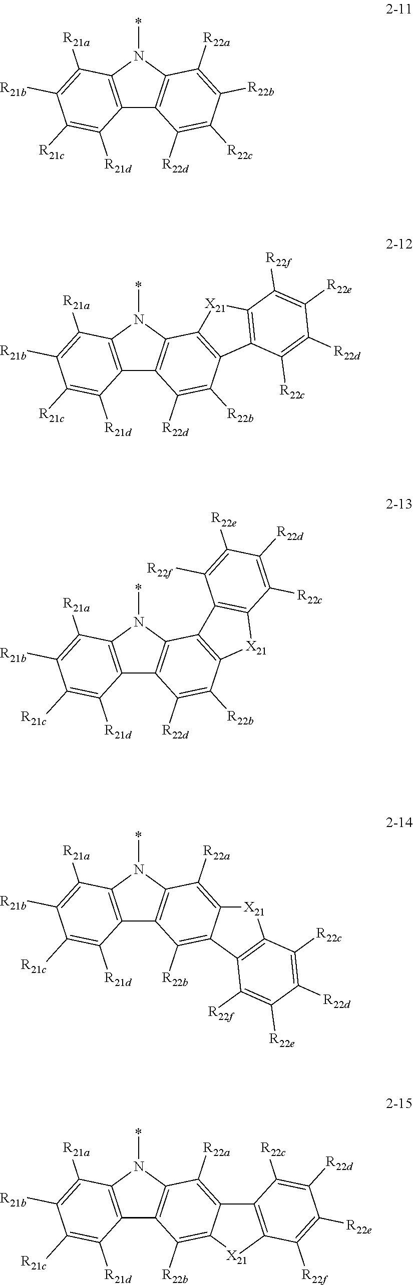 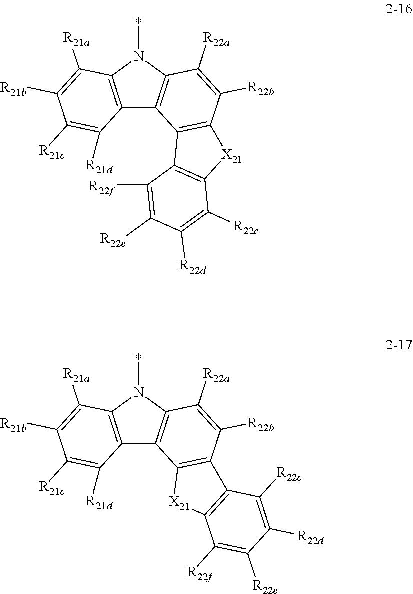
wherein, in Formulae 2-11 to 2-17,
X₂₁ is O, S, N(R_{22g}), or C(R_{22g})(R₂₂ₕ);
R₂₁ₐ to R_{21d} are understood by referring to the definition of R₂₁ in claim 1;
R₂₂ₐ to R₂₂ₕ are understood by referring to the definition of R₂₂ in claim 1; and
* indicates a binding site to a neighboring atom.

8. The condensed-cyclic compound of claim 1, wherein
the condensed-cyclic compound is represented by one of Formulae 1-1 to 1-6: 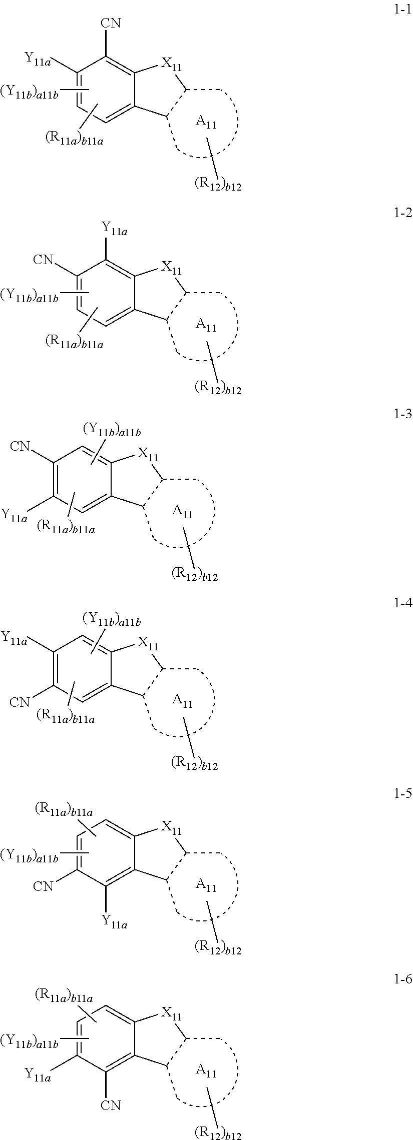
wherein, in Formulae 1-1 to 1-6,
Y₁₁ₐ and Y_{11b} are each understood by referring to the definition of Y₁₁ in claim 1,
R₁₁ₐ is understood by referring to the definition of R₁₁ in claim 1,
a11b is 1 or 2;
X₁₁, A₁₁, R₁₂, and b12 are each understood by referring to claim 1,
A₂₁, A₂₂, X₂₁, R₂₁ to R₂₄, b21, and b22 are each understood by referring to claim 1, and
the sum of a11b and b11a is 3.

9. The condensed-cyclic compound of claim 8, wherein
A₁₁ is a benzene group.

10. The condensed-cyclic compound of claim 8, wherein
Y₁₁ is represented by one of Formulae 2-11 to 2-17: 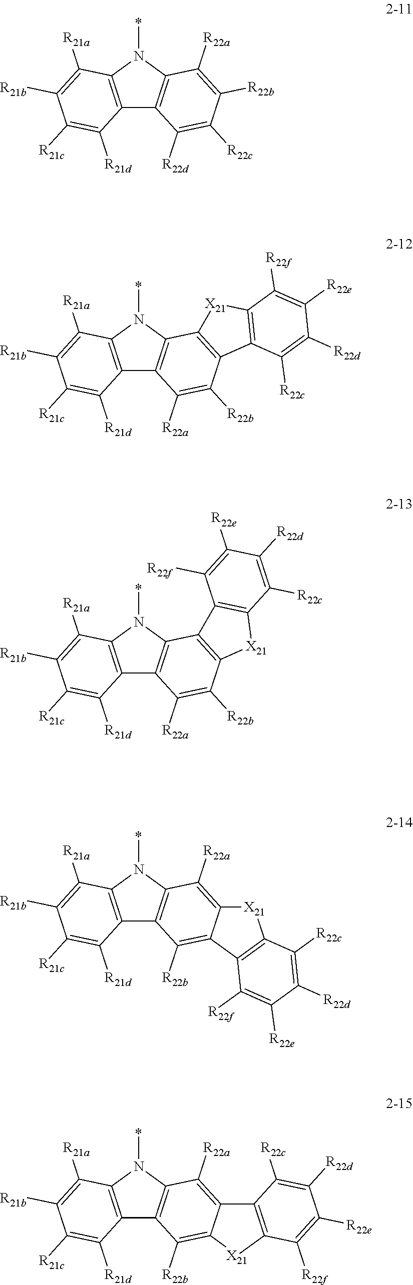 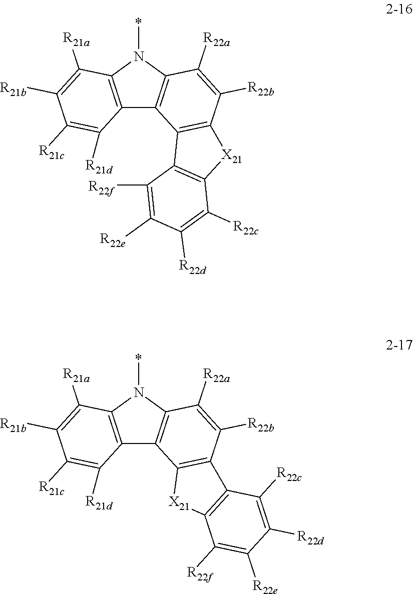
wherein, in Formulae 2-11 to 2-17,
X₂₁ is O, S, N(R_{22g}), or C(R_{22g})(R₂₂ₕ);
R₂₁ₐ to R_{21d} are understood by referring to the definition of R₂₁ in claim 1;
R₂₂ₐ to R₂₂ₕ are understood by referring to the definition of R₂₂ in claim 1; and
* indicates a binding site to a neighboring atom.

11. The condensed-cyclic compound of claim 1, wherein
the condensed-cyclic compound is represented by one of Formulae 1-11 to 1-23: 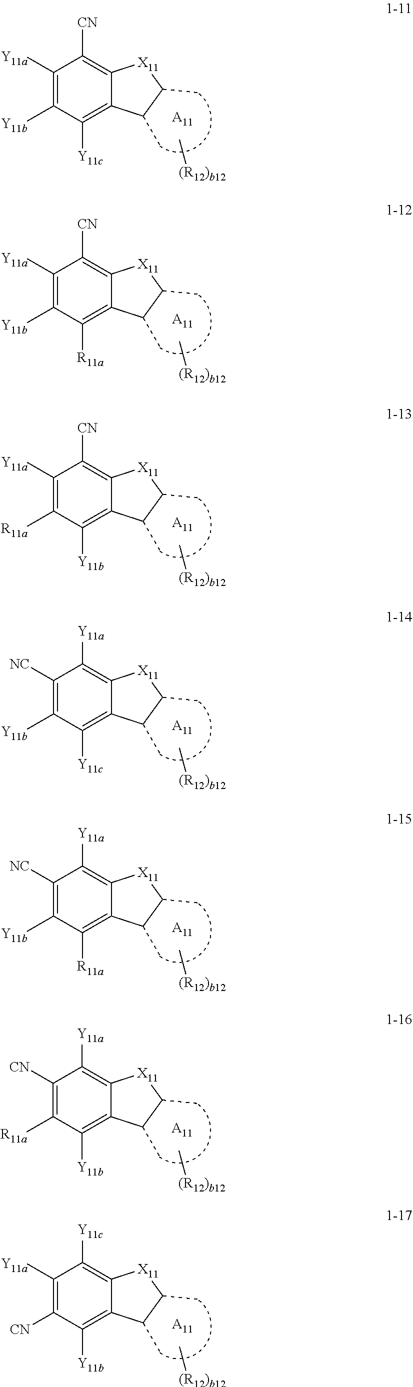 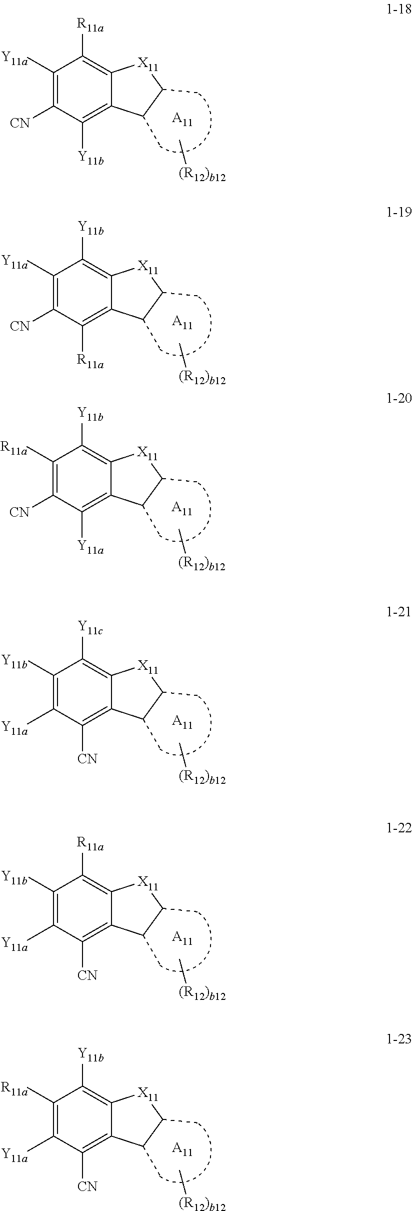
wherein, in Formulae 1-11 to 1-23,
Y₁₁ₐ, Y_{11b}, and Y₁₁ₑ are each understood by referring to the definition of Y₁₁ in claim 1,
R₁₁ₐ is understood by referring to the definition of R₁₁ in claim 1,
X₁₁, A₁₁, R₁₂, and b12 are each understood by referring to claim 1, and
A₂₁, A₂₂, X₂₁, R₂₁ to R₂₄, b21, and b22 are each understood by referring to claim 1.

12. The condensed-cyclic compound of claim 1, wherein
the condensed-cyclic compound is Compounds 1 to 336:

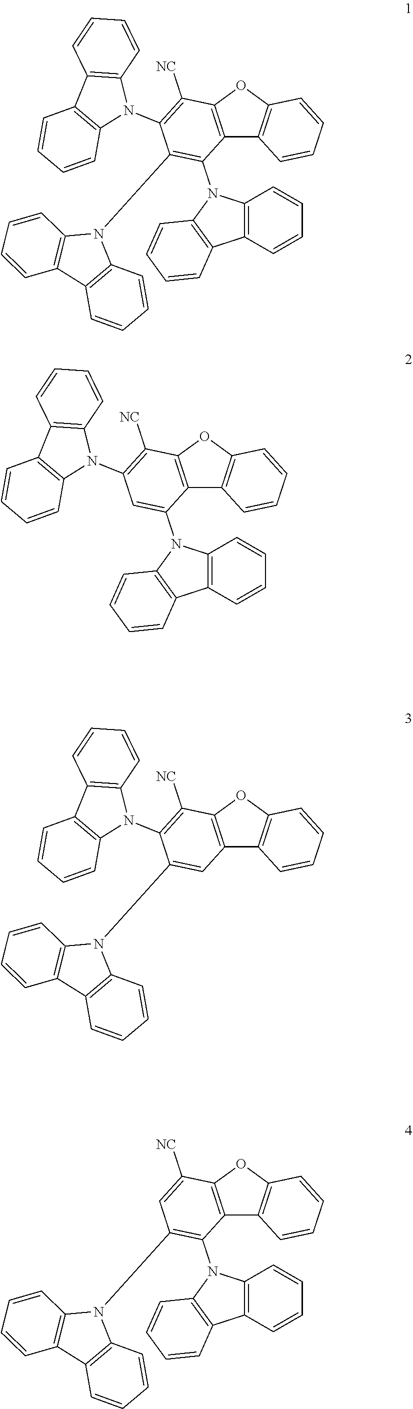 [SMILES: N#Cc1c(-n2c3ccccc3c3ccccc32)c(-n2c3ccccc3c3ccccc32)c(-n2c3ccccc3c3ccccc32)c2c1oc1ccccc12 ; N#Cc1c(-n2c3ccccc3c3ccccc32)cc(-n2c3ccccc3c3ccccc32)c2c1oc1ccccc12 ; N#Cc1c(-n2c3ccccc3c3ccccc32)c(-n2c3ccccc3c3ccccc32)cc2c1oc1ccccc12 ; N#Cc1cc(-n2c3ccccc3c3ccccc32)c(-n2c3ccccc3c3ccccc32)c2c1oc1ccccc12]

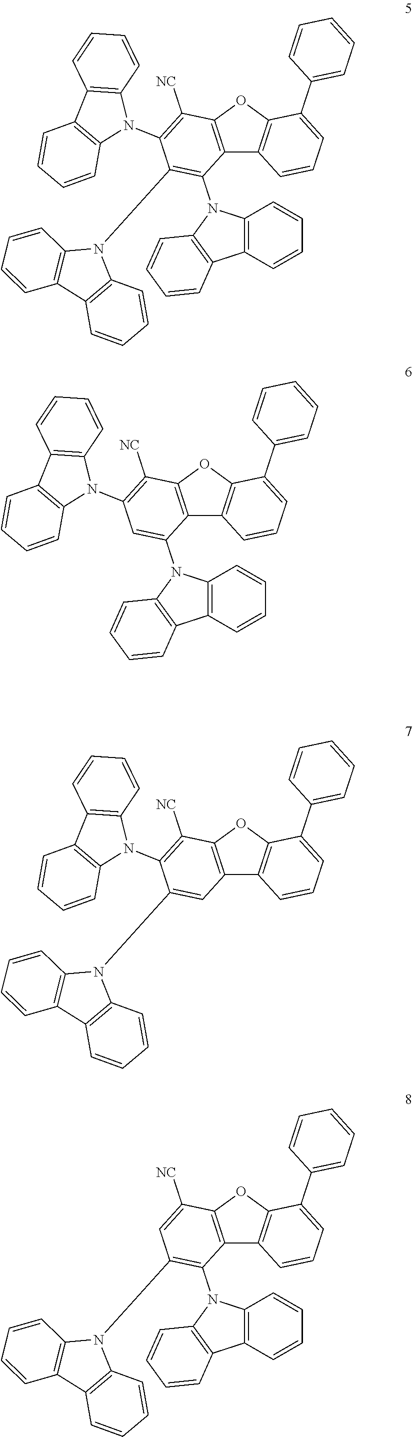 [SMILES: N#Cc1c(-n2c3ccccc3c3ccccc32)c(-n2c3ccccc3c3ccccc32)c(-n2c3ccccc3c3ccccc32)c2c1oc1c(-c3ccccc3)cccc12 ; N#Cc1c(-n2c3ccccc3c3ccccc32)cc(-n2c3ccccc3c3ccccc32)c2c1oc1c(-c3ccccc3)cccc12 ; N#Cc1c(-n2c3ccccc3c3ccccc32)c(-n2c3ccccc3c3ccccc32)cc2c1oc1c(-c3ccccc3)cccc12 ; N#Cc1cc(-n2c3ccccc3c3ccccc32)c(-n2c3ccccc3c3ccccc32)c2c1oc1c(-c3ccccc3)cccc12]

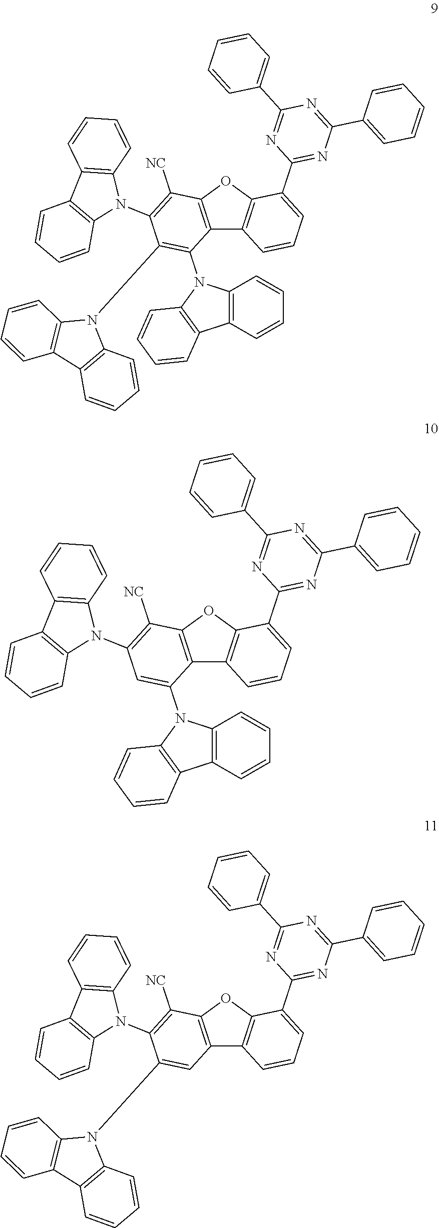 [SMILES: N#Cc1c(-n2c3ccccc3c3ccccc32)c(-n2c3ccccc3c3ccccc32)c(-n2c3ccccc3c3ccccc32)c2c1oc1c(-c3nc(-c4ccccc4)nc(-c4ccccc4)n3)cccc12 ; N#Cc1c(-n2c3ccccc3c3ccccc32)cc(-n2c3ccccc3c3ccccc32)c2c1oc1c(-c3nc(-c4ccccc4)nc(-c4ccccc4)n3)cccc12 ; N#Cc1c(-n2c3ccccc3c3ccccc32)c(-n2c3ccccc3c3ccccc32)cc2c1oc1c(-c3nc(-c4ccccc4)nc(-c4ccccc4)n3)cccc12]

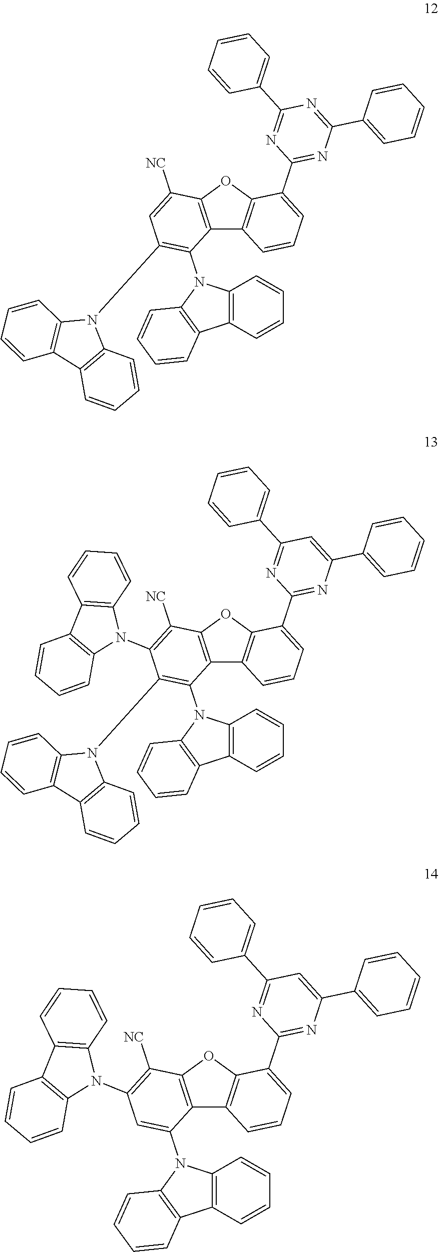 [SMILES: N#Cc1cc(-n2c3ccccc3c3ccccc32)c(-n2c3ccccc3c3ccccc32)c2c1oc1c(-c3nc(-c4ccccc4)nc(-c4ccccc4)n3)cccc12 ; N#Cc1c(-n2c3ccccc3c3ccccc32)c(-n2c3ccccc3c3ccccc32)c(-n2c3ccccc3c3ccccc32)c2c1oc1c(-c3nc(-c4ccccc4)cc(-c4ccccc4)n3)cccc12 ; N#Cc1c(-n2c3ccccc3c3ccccc32)cc(-n2c3ccccc3c3ccccc32)c2c1oc1c(-c3nc(-c4ccccc4)cc(-c4ccccc4)n3)cccc12]

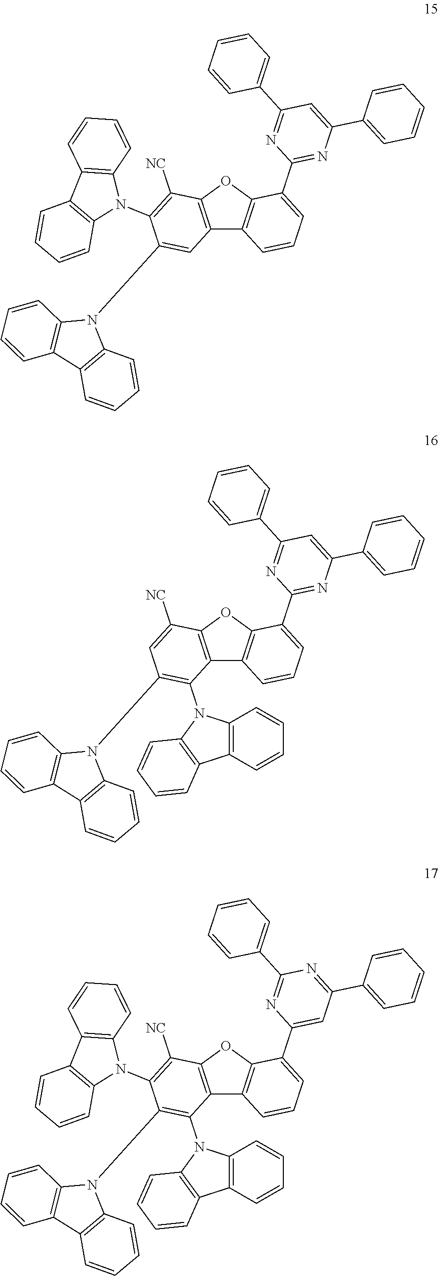 [SMILES: N#Cc1c(-n2c3ccccc3c3ccccc32)c(-n2c3ccccc3c3ccccc32)cc2c1oc1c(-c3nc(-c4ccccc4)cc(-c4ccccc4)n3)cccc12 ; N#Cc1cc(-n2c3ccccc3c3ccccc32)c(-n2c3ccccc3c3ccccc32)c2c1oc1c(-c3nc(-c4ccccc4)cc(-c4ccccc4)n3)cccc12 ; N#Cc1c(-n2c3ccccc3c3ccccc32)c(-n2c3ccccc3c3ccccc32)c(-n2c3ccccc3c3ccccc32)c2c1oc1c(-c3cc(-c4ccccc4)nc(-c4ccccc4)n3)cccc12]

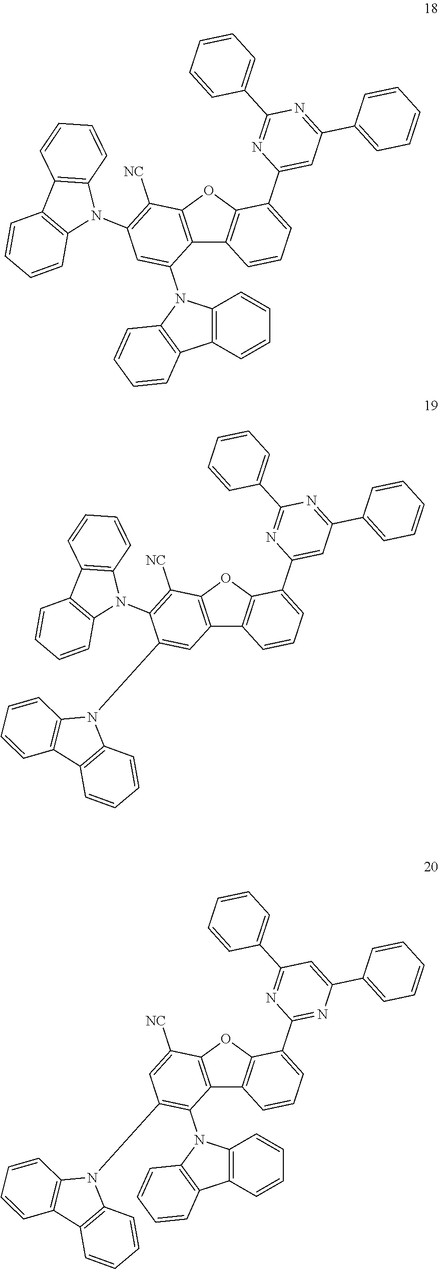 [SMILES: N#Cc1c(-n2c3ccccc3c3ccccc32)cc(-n2c3ccccc3c3ccccc32)c2c1oc1c(-c3cc(-c4ccccc4)nc(-c4ccccc4)n3)cccc12 ; N#Cc1c(-n2c3ccccc3c3ccccc32)c(-n2c3ccccc3c3ccccc32)cc2c1oc1c(-c3cc(-c4ccccc4)nc(-c4ccccc4)n3)cccc12 ; N#Cc1cc(-n2c3ccccc3c3ccccc32)c(-n2c3ccccc3c3ccccc32)c2c1oc1c(-c3nc(-c4ccccc4)cc(-c4ccccc4)n3)cccc12]

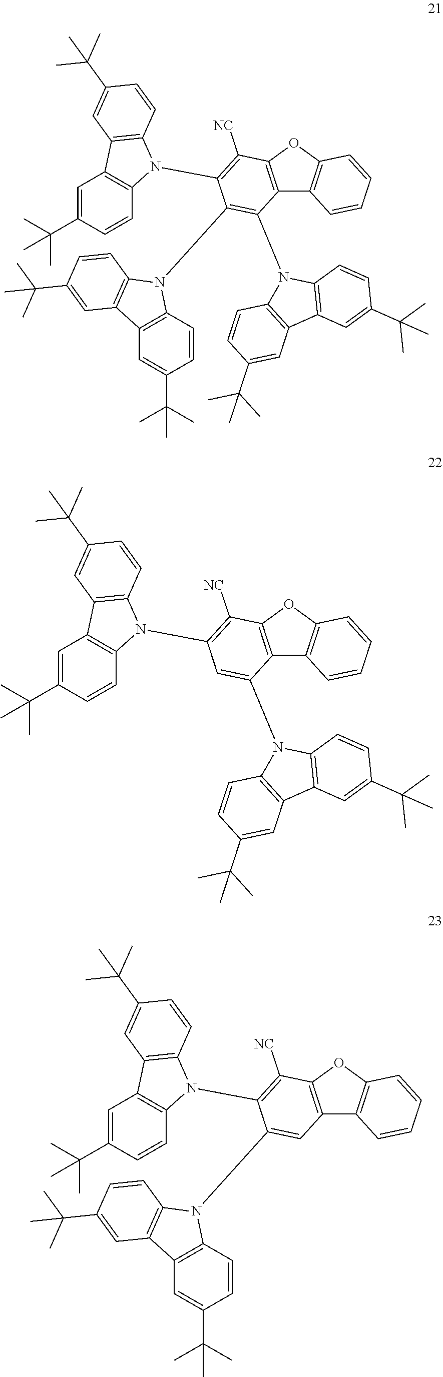

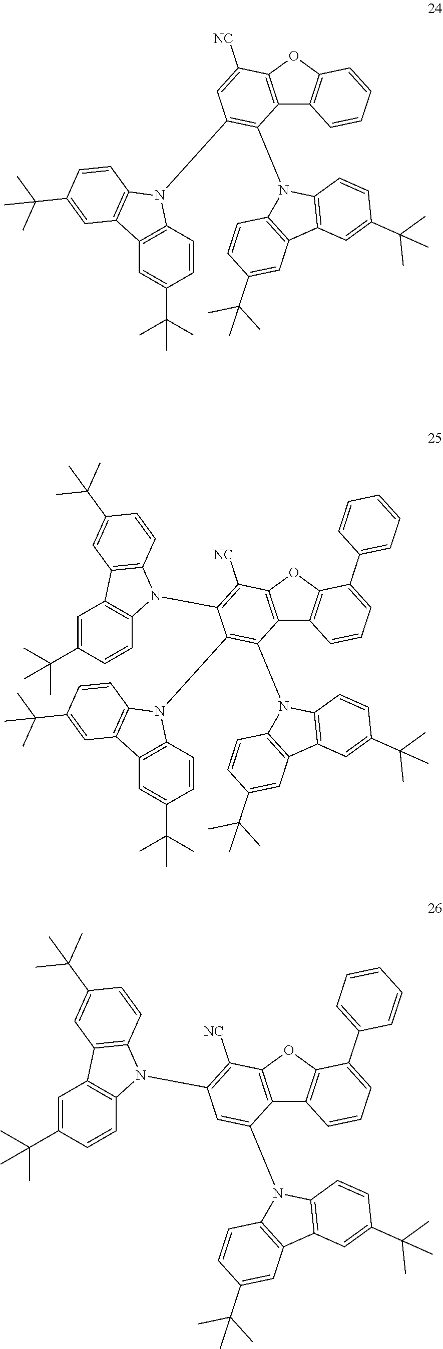

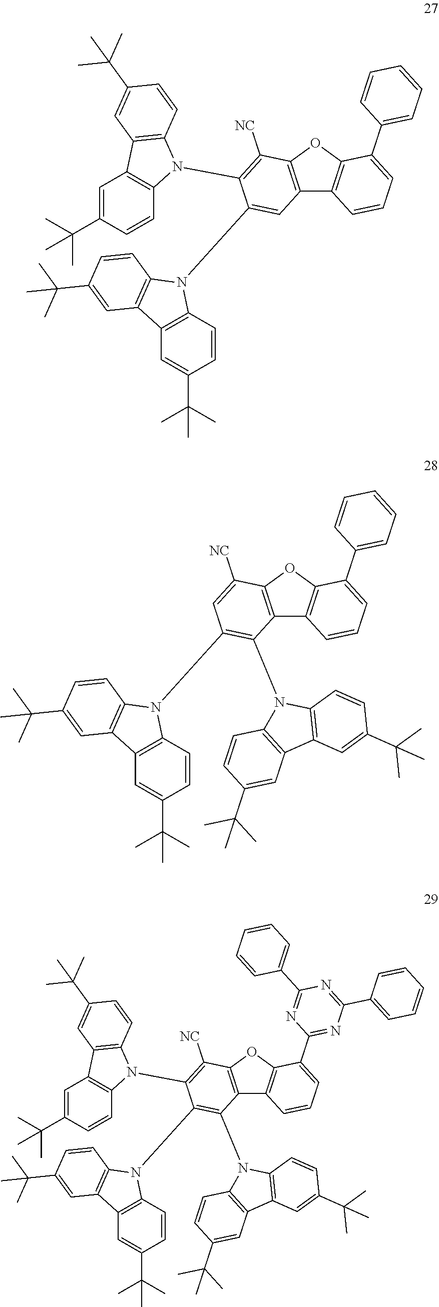

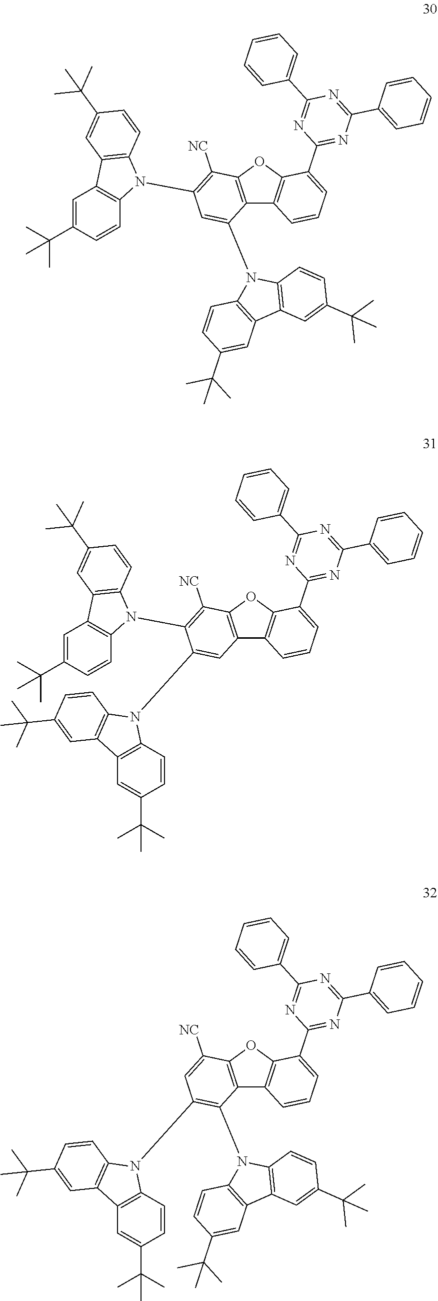 [SMILES: CC(C)(C)c1ccc2c(c1)c1cc(C(C)(C)C)ccc1n2-c1cc(-n2c3ccc(C(C)(C)C)cc3c3cc(C(C)(C)C)ccc32)c2c(oc3c(-c4nc(-c5ccccc5)nc(-c5ccccc5)n4)cccc32)c1C#N ; CC(C)(C)c1ccc2c(c1)c1cc(C(C)(C)C)ccc1n2-c1cc2c(oc3c(-c4nc(-c5ccccc5)nc(-c5ccccc5)n4)cccc32)c(C#N)c1-n1c2ccc(C(C)(C)C)cc2c2cc(C(C)(C)C)ccc21 ; CC(C)(C)c1ccc2c(c1)c1cc(C(C)(C)C)ccc1n2-c1cc(C#N)c2oc3c(-c4nc(-c5ccccc5)nc(-c5ccccc5)n4)cccc3c2c1-n1c2ccc(C(C)(C)C)cc2c2cc(C(C)(C)C)ccc21]

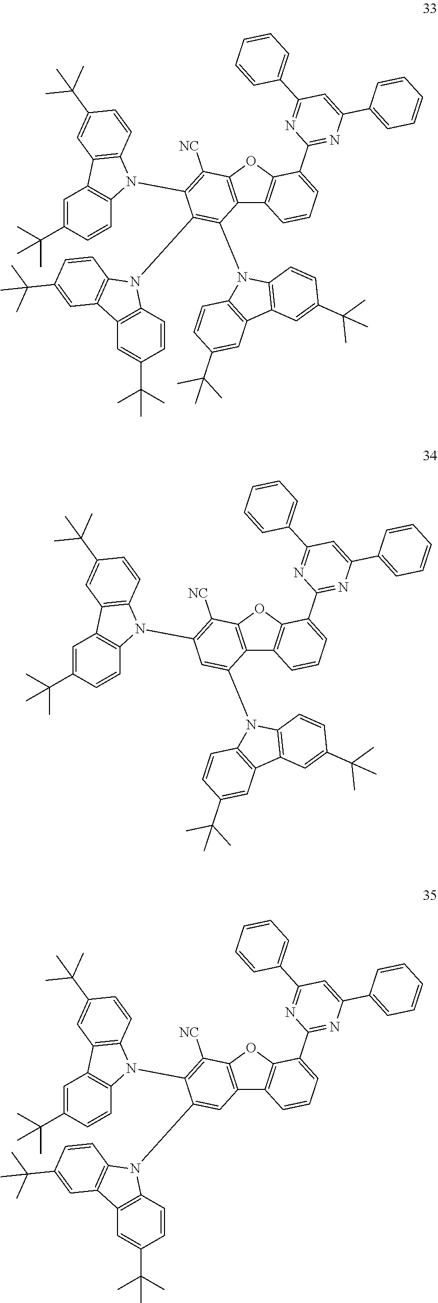 [SMILES: CC(C)(C)c1ccc2c(c1)c1cc(C(C)(C)C)ccc1n2-c1c(-n2c3ccc(C(C)(C)C)cc3c3cc(C(C)(C)C)ccc32)c(-n2c3ccc(C(C)(C)C)cc3c3cc(C(C)(C)C)ccc32)c2c(oc3c(-c4nc(-c5ccccc5)cc(-c5ccccc5)n4)cccc32)c1C#N ; CC(C)(C)c1ccc2c(c1)c1cc(C(C)(C)C)ccc1n2-c1cc(-n2c3ccc(C(C)(C)C)cc3c3cc(C(C)(C)C)ccc32)c2c(oc3c(-c4nc(-c5ccccc5)cc(-c5ccccc5)n4)cccc32)c1C#N ; CC(C)(C)c1ccc2c(c1)c1cc(C(C)(C)C)ccc1n2-c1cc2c(oc3c(-c4nc(-c5ccccc5)cc(-c5ccccc5)n4)cccc32)c(C#N)c1-n1c2ccc(C(C)(C)C)cc2c2cc(C(C)(C)C)ccc21]

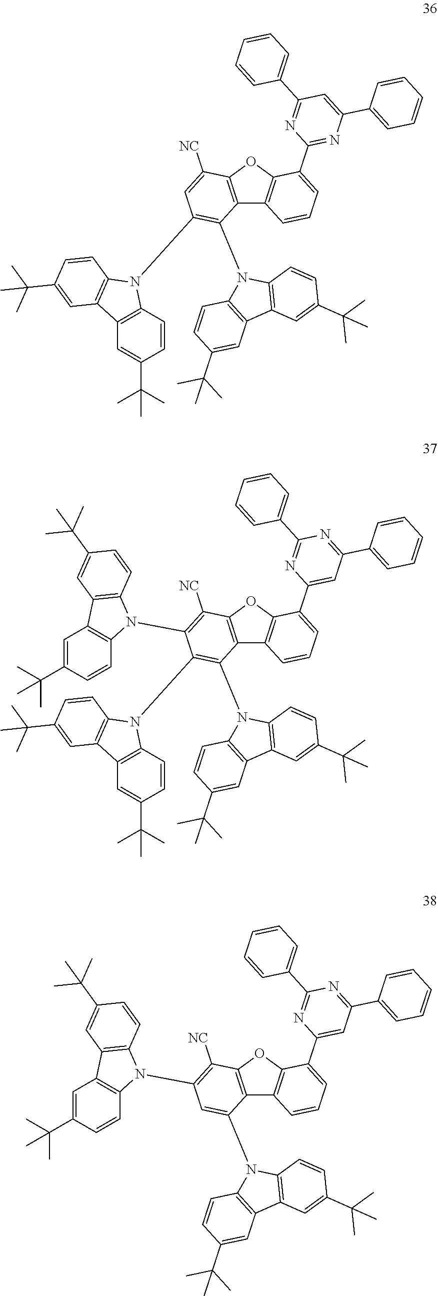 [SMILES: CC(C)(C)c1ccc2c(c1)c1cc(C(C)(C)C)ccc1n2-c1cc(C#N)c2oc3c(-c4nc(-c5ccccc5)cc(-c5ccccc5)n4)cccc3c2c1-n1c2ccc(C(C)(C)C)cc2c2cc(C(C)(C)C)ccc21 ; CC(C)(C)c1ccc2c(c1)c1cc(C(C)(C)C)ccc1n2-c1c(-n2c3ccc(C(C)(C)C)cc3c3cc(C(C)(C)C)ccc32)c(-n2c3ccc(C(C)(C)C)cc3c3cc(C(C)(C)C)ccc32)c2c(oc3c(-c4cc(-c5ccccc5)nc(-c5ccccc5)n4)cccc32)c1C#N ; CC(C)(C)c1ccc2c(c1)c1cc(C(C)(C)C)ccc1n2-c1cc(-n2c3ccc(C(C)(C)C)cc3c3cc(C(C)(C)C)ccc32)c2c(oc3c(-c4cc(-c5ccccc5)nc(-c5ccccc5)n4)cccc32)c1C#N]

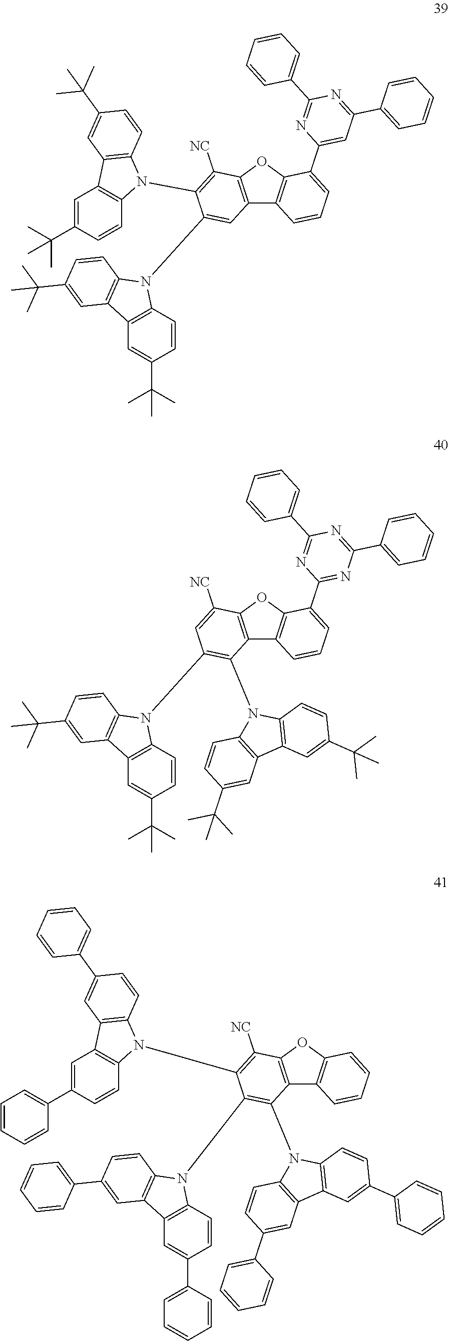 [SMILES: CC(C)(C)c1ccc2c(c1)c1cc(C(C)(C)C)ccc1n2-c1cc2c(oc3c(-c4cc(-c5ccccc5)nc(-c5ccccc5)n4)cccc32)c(C#N)c1-n1c2ccc(C(C)(C)C)cc2c2cc(C(C)(C)C)ccc21 ; CC(C)(C)c1ccc2c(c1)c1cc(C(C)(C)C)ccc1n2-c1cc(C#N)c2oc3c(-c4nc(-c5ccccc5)nc(-c5ccccc5)n4)cccc3c2c1-n1c2ccc(C(C)(C)C)cc2c2cc(C(C)(C)C)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1ccccc12]

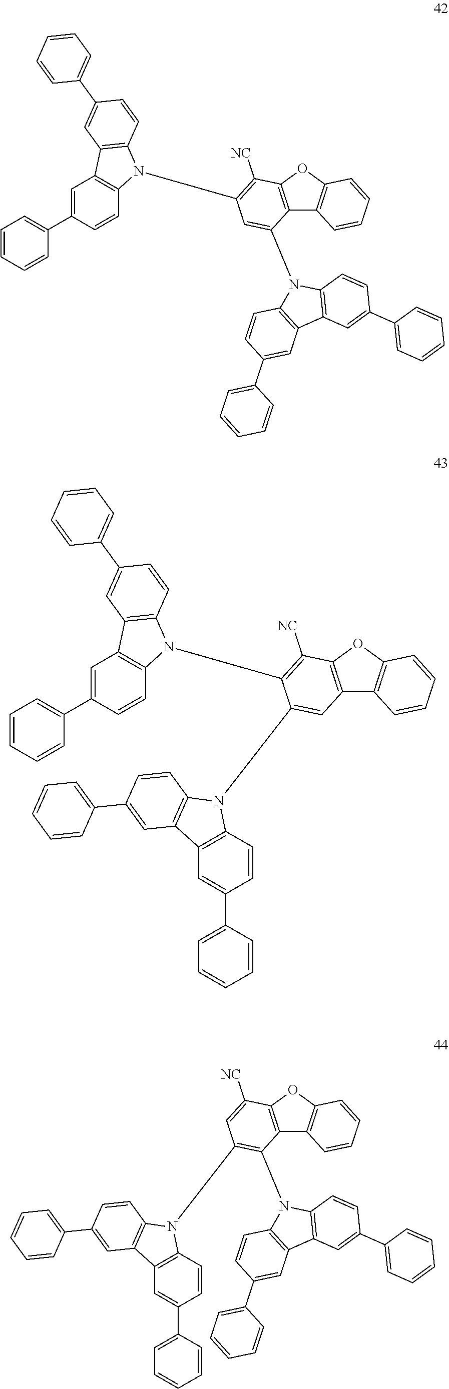 [SMILES: N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2c1oc1ccccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2c1oc1ccccc12 ; N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1ccccc12]

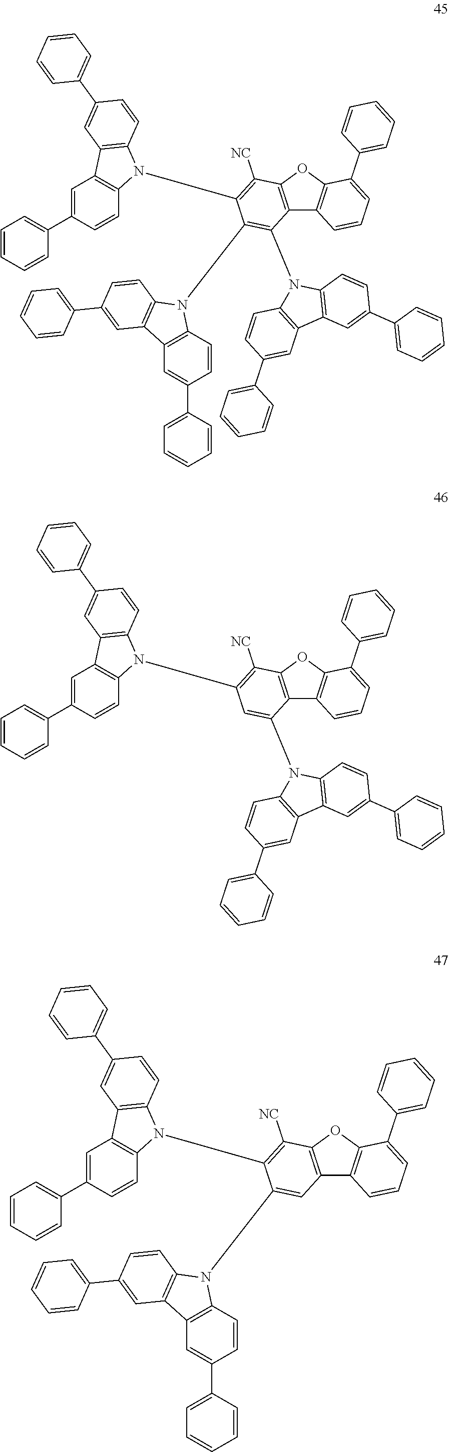 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3ccccc3)cccc12 ; N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2c1oc1c(-c3ccccc3)cccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2c1oc1c(-c3ccccc3)cccc12]

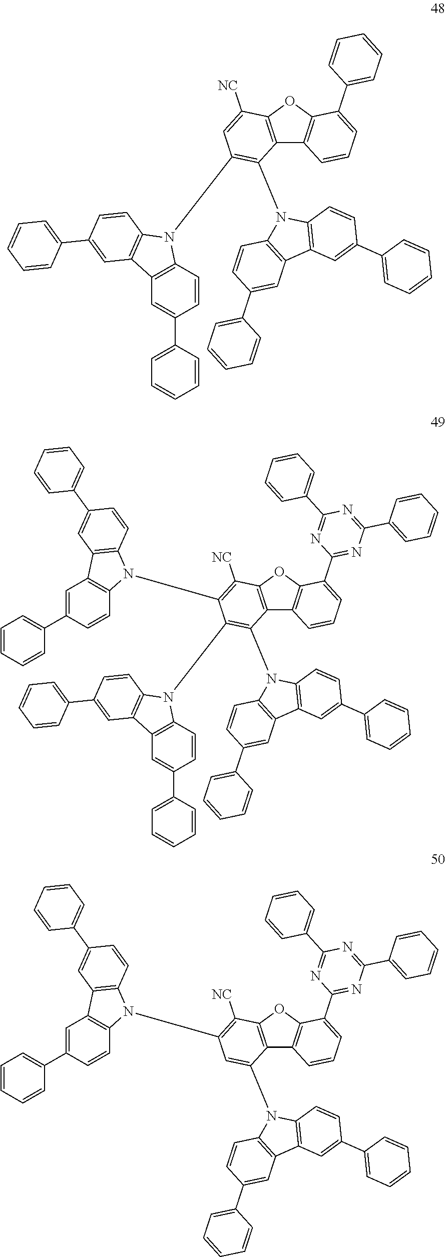 [SMILES: N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3ccccc3)cccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3nc(-c4ccccc4)nc(-c4ccccc4)n3)cccc12 ; N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2c1oc1c(-c3nc(-c4ccccc4)nc(-c4ccccc4)n3)cccc12]

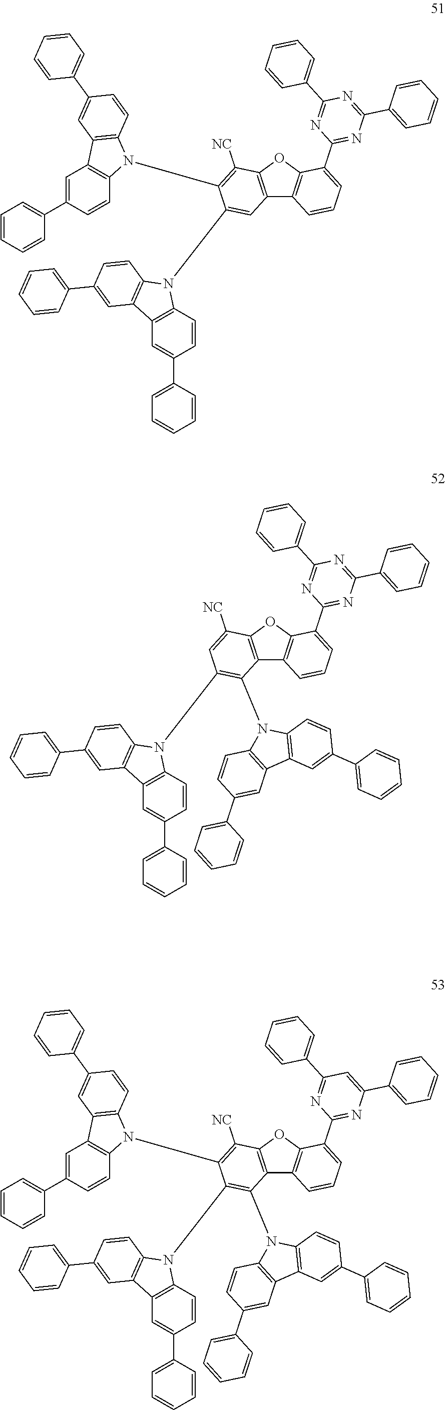 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2c1oc1c(-c3nc(-c4ccccc4)nc(-c4ccccc4)n3)cccc12 ; N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3nc(-c4ccccc4)nc(-c4ccccc4)n3)cccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3nc(-c4ccccc4)cc(-c4ccccc4)n3)cccc12]

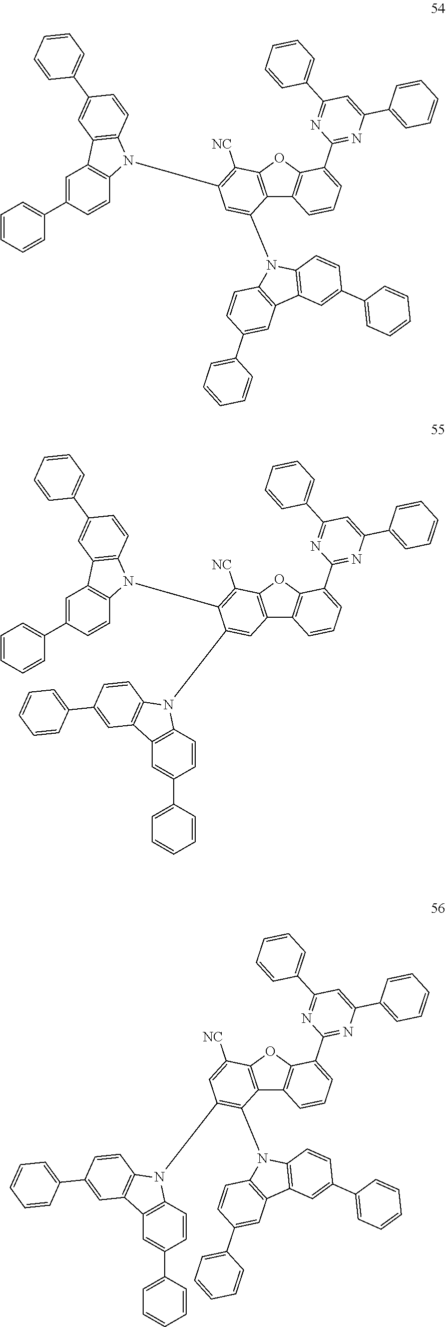 [SMILES: N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2c1oc1c(-c3nc(-c4ccccc4)cc(-c4ccccc4)n3)cccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2c1oc1c(-c3nc(-c4ccccc4)cc(-c4ccccc4)n3)cccc12 ; N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3nc(-c4ccccc4)cc(-c4ccccc4)n3)cccc12]

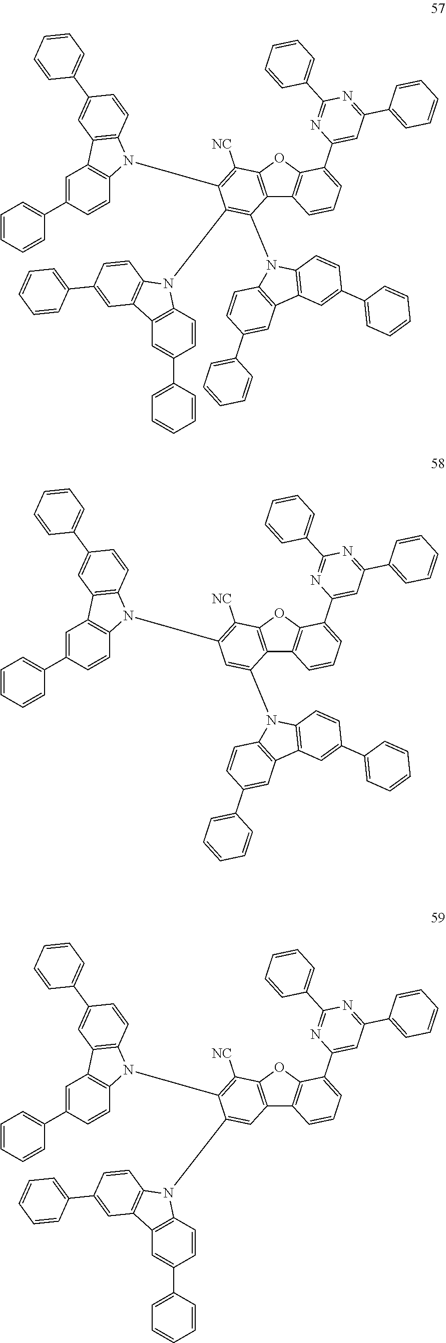 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3cc(-c4ccccc4)nc(-c4ccccc4)n3)cccc12 ; N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2c1oc1c(-c3cc(-c4ccccc4)nc(-c4ccccc4)n3)cccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2c1oc1c(-c3cc(-c4ccccc4)nc(-c4ccccc4)n3)cccc12]

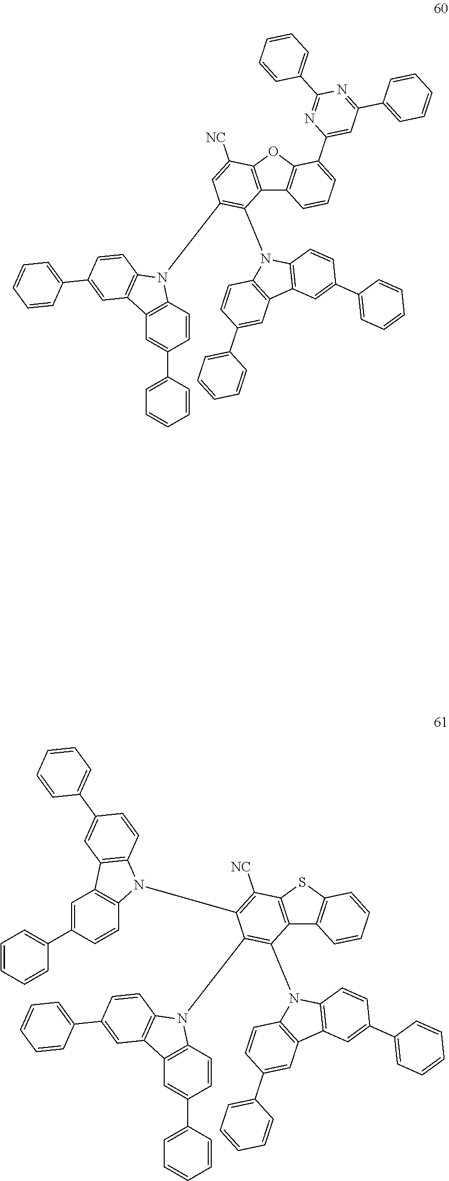 [SMILES: N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2c1oc1c(-c3cc(-c4ccccc4)nc(-c4ccccc4)n3)cccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2c1sc1ccccc12]

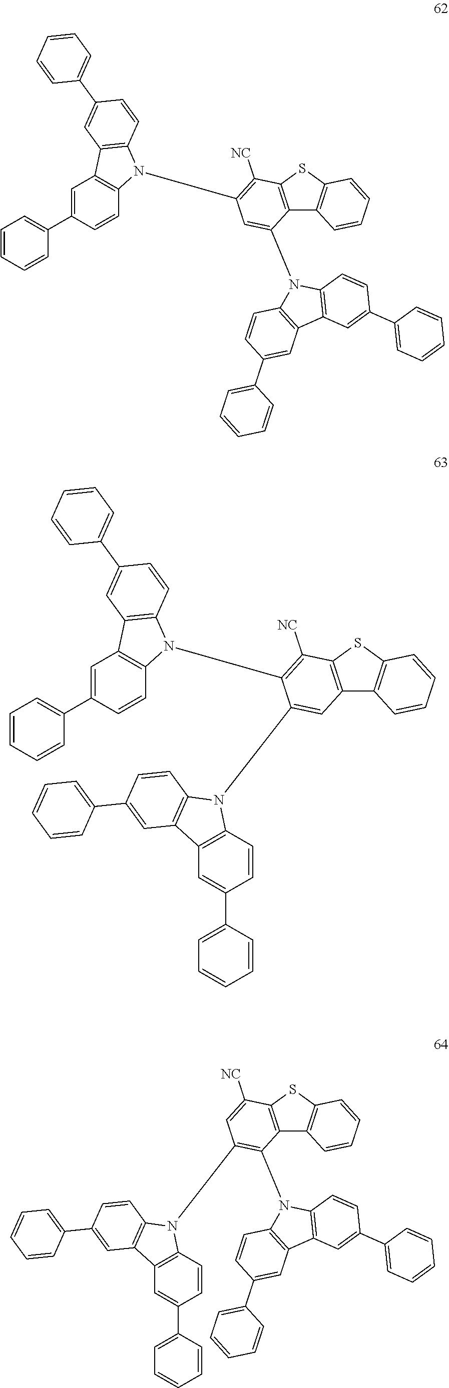 [SMILES: N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2c1sc1ccccc12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2c1sc1ccccc12 ; N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2c1sc1ccccc12]

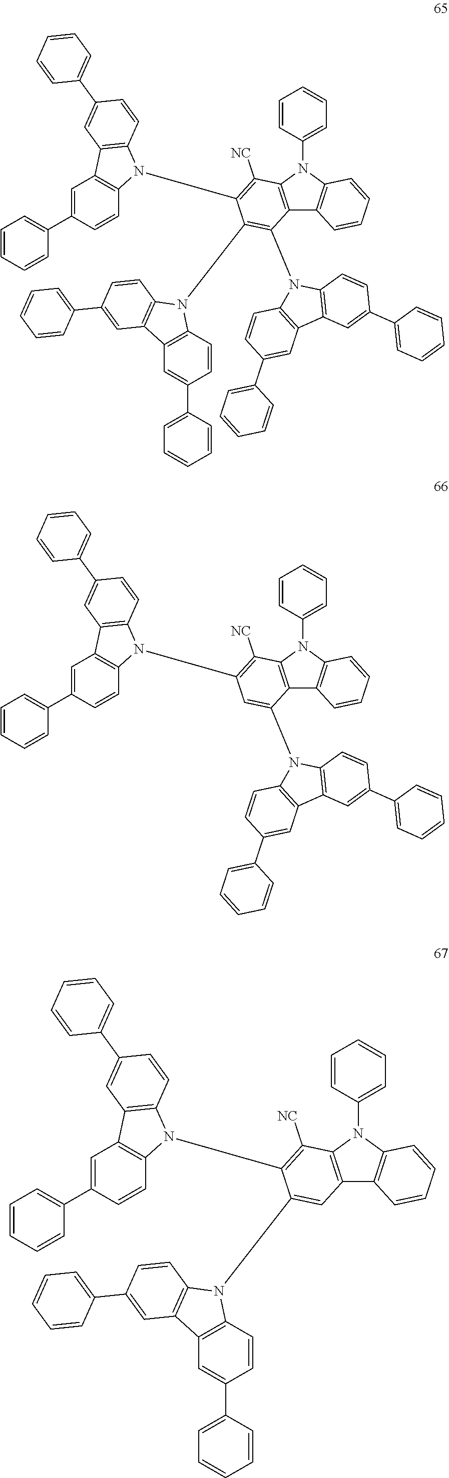 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2c3ccccc3n(-c3ccccc3)c12 ; N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2c3ccccc3n(-c3ccccc3)c12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2c3ccccc3n(-c3ccccc3)c12]

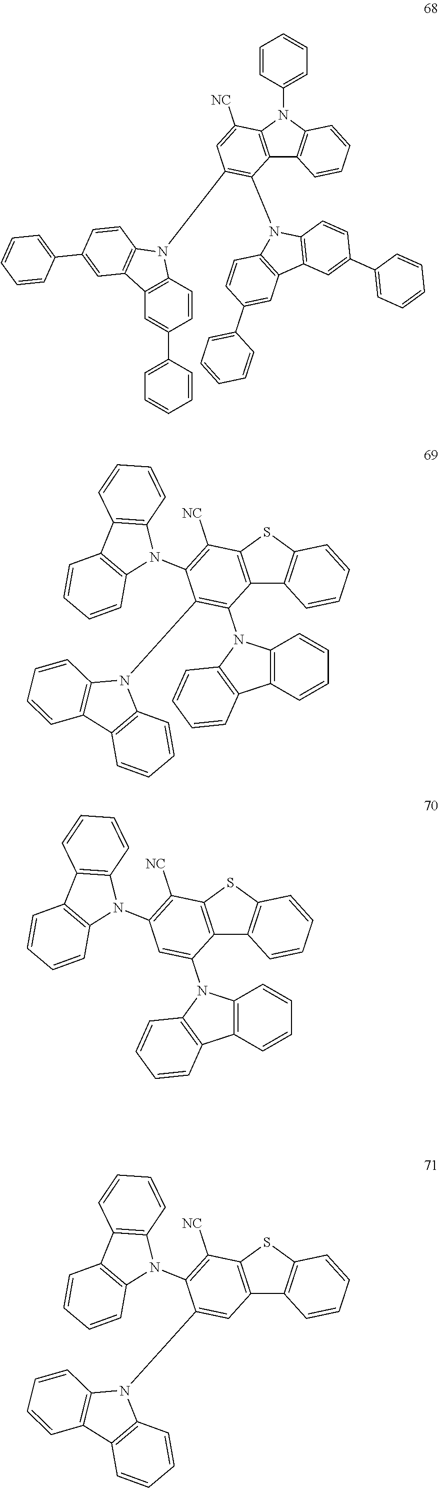 [SMILES: N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2c3ccccc3n(-c3ccccc3)c12 ; N#Cc1c(-n2c3ccccc3c3ccccc32)c(-n2c3ccccc3c3ccccc32)c(-n2c3ccccc3c3ccccc32)c2c1sc1ccccc12 ; N#Cc1c(-n2c3ccccc3c3ccccc32)cc(-n2c3ccccc3c3ccccc32)c2c1sc1ccccc12 ; N#Cc1c(-n2c3ccccc3c3ccccc32)c(-n2c3ccccc3c3ccccc32)cc2c1sc1ccccc12]

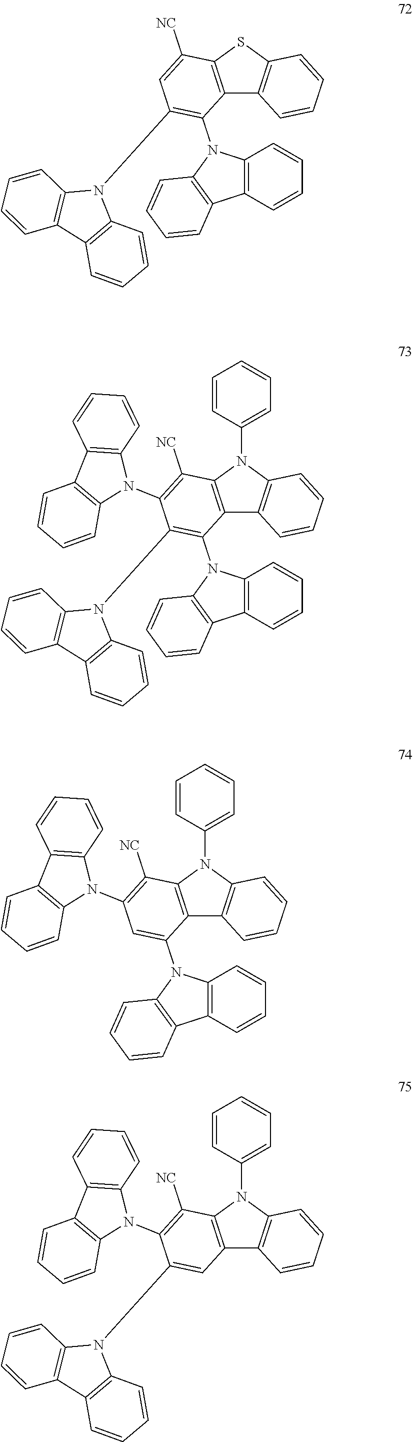 [SMILES: N#Cc1cc(-n2c3ccccc3c3ccccc32)c(-n2c3ccccc3c3ccccc32)c2c1sc1ccccc12 ; N#Cc1c(-n2c3ccccc3c3ccccc32)c(-n2c3ccccc3c3ccccc32)c(-n2c3ccccc3c3ccccc32)c2c3ccccc3n(-c3ccccc3)c12 ; N#Cc1c(-n2c3ccccc3c3ccccc32)cc(-n2c3ccccc3c3ccccc32)c2c3ccccc3n(-c3ccccc3)c12 ; N#Cc1c(-n2c3ccccc3c3ccccc32)c(-n2c3ccccc3c3ccccc32)cc2c3ccccc3n(-c3ccccc3)c12]

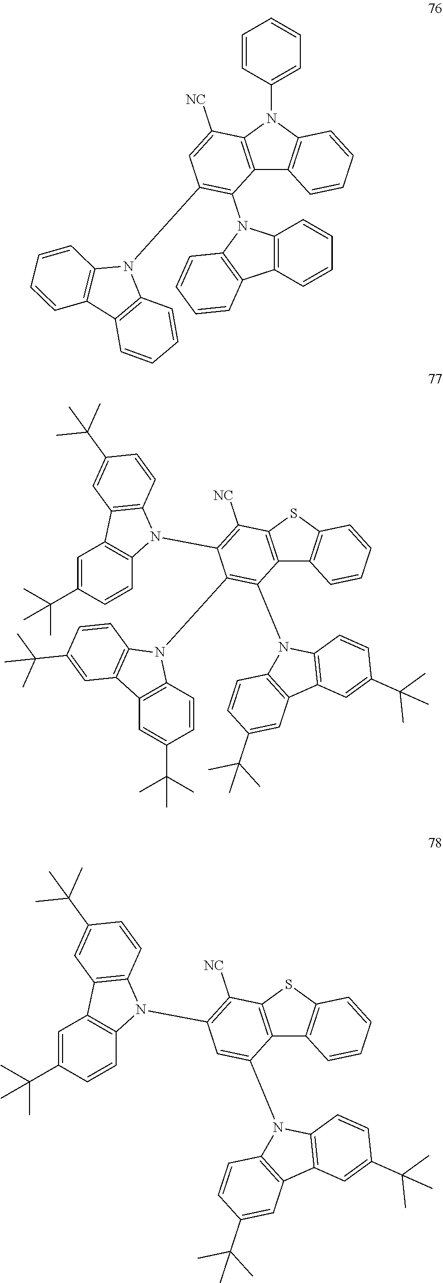

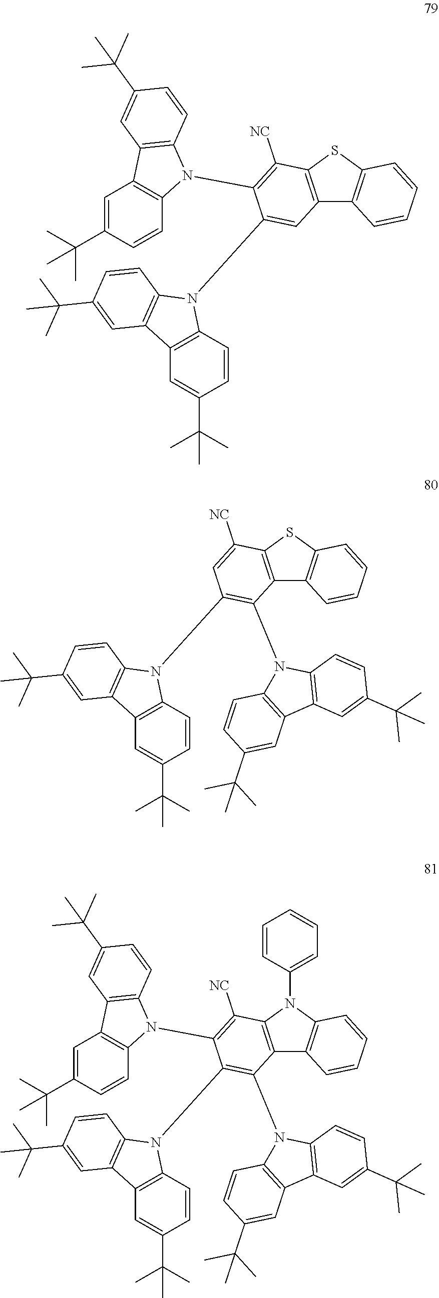

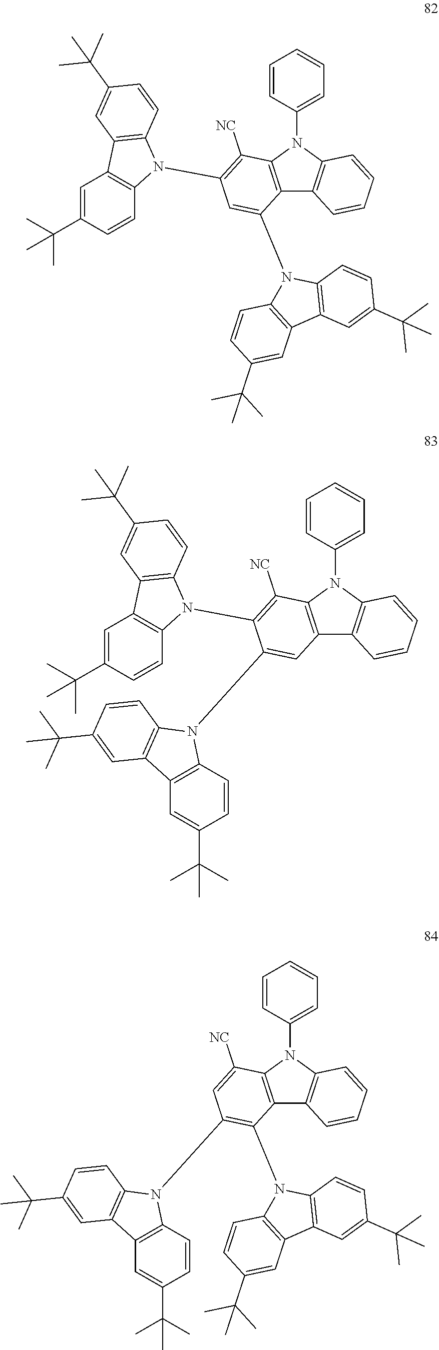

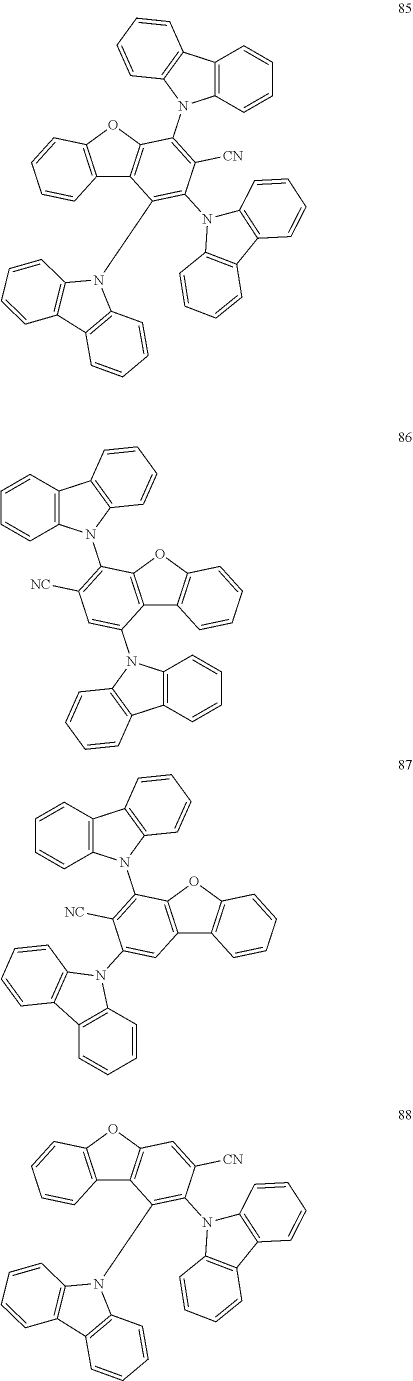

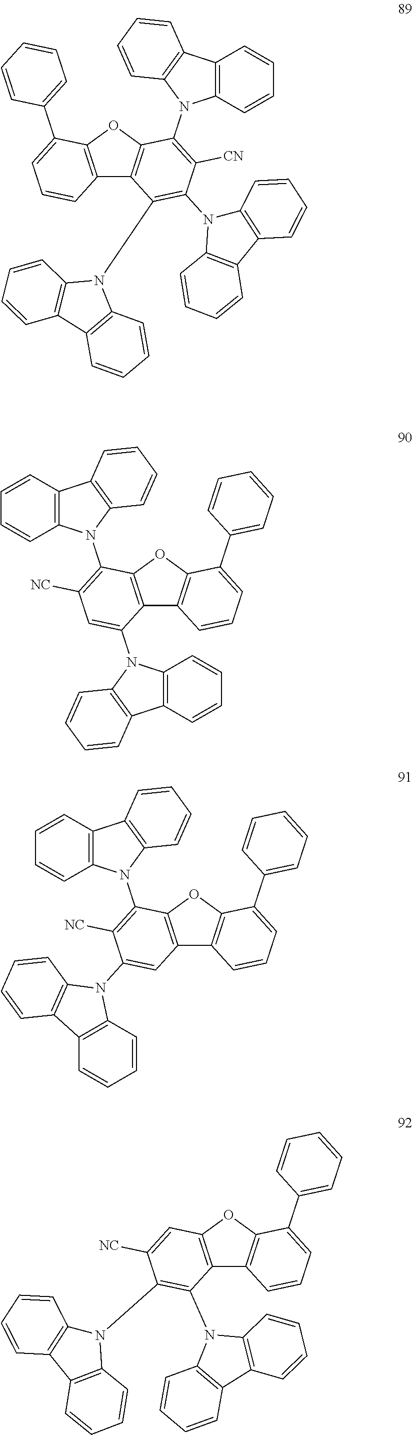 [SMILES: N#Cc1c(-n2c3ccccc3c3ccccc32)c(-n2c3ccccc3c3ccccc32)c2c(oc3c(-c4ccccc4)cccc32)c1-n1c2ccccc2c2ccccc21 ; N#Cc1cc(-n2c3ccccc3c3ccccc32)c2c(oc3c(-c4ccccc4)cccc32)c1-n1c2ccccc2c2ccccc21 ; N#Cc1c(-n2c3ccccc3c3ccccc32)cc2c(oc3c(-c4ccccc4)cccc32)c1-n1c2ccccc2c2ccccc21 ; N#Cc1cc2oc3c(-c4ccccc4)cccc3c2c(-n2c3ccccc3c3ccccc32)c1-n1c2ccccc2c2ccccc21]

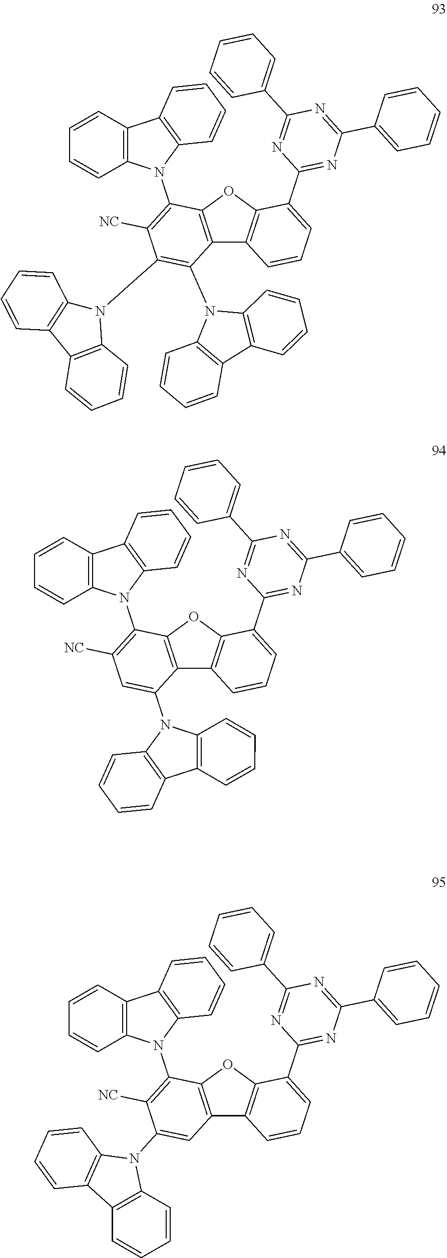 [SMILES: N#Cc1c(-n2c3ccccc3c3ccccc32)c(-n2c3ccccc3c3ccccc32)c2c(oc3c(-c4nc(-c5ccccc5)nc(-c5ccccc5)n4)cccc32)c1-n1c2ccccc2c2ccccc21 ; N#Cc1cc(-n2c3ccccc3c3ccccc32)c2c(oc3c(-c4nc(-c5ccccc5)nc(-c5ccccc5)n4)cccc32)c1-n1c2ccccc2c2ccccc21 ; N#Cc1c(-n2c3ccccc3c3ccccc32)cc2c(oc3c(-c4nc(-c5ccccc5)nc(-c5ccccc5)n4)cccc32)c1-n1c2ccccc2c2ccccc21]

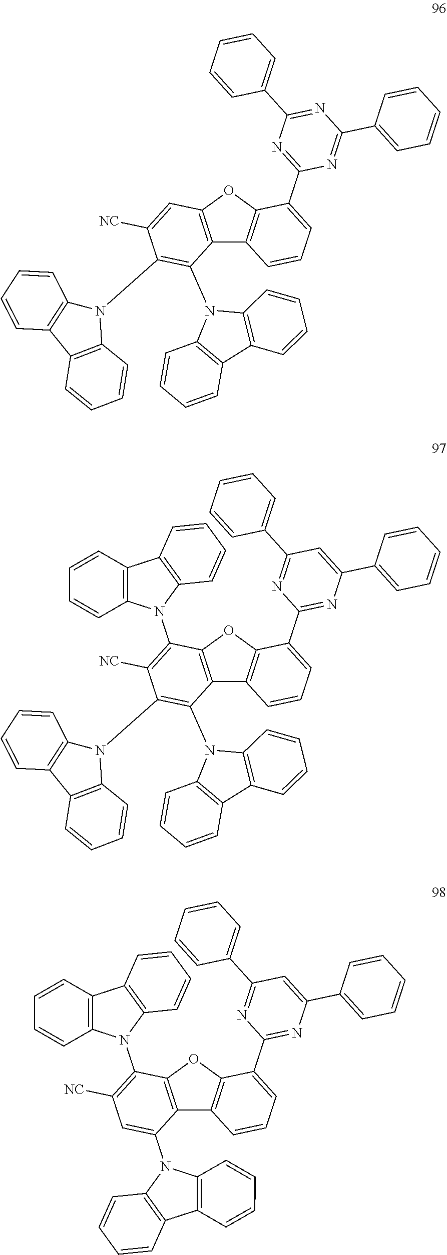 [SMILES: N#Cc1cc2oc3c(-c4nc(-c5ccccc5)nc(-c5ccccc5)n4)cccc3c2c(-n2c3ccccc3c3ccccc32)c1-n1c2ccccc2c2ccccc21 ; N#Cc1c(-n2c3ccccc3c3ccccc32)c(-n2c3ccccc3c3ccccc32)c2c(oc3c(-c4nc(-c5ccccc5)cc(-c5ccccc5)n4)cccc32)c1-n1c2ccccc2c2ccccc21 ; N#Cc1cc(-n2c3ccccc3c3ccccc32)c2c(oc3c(-c4nc(-c5ccccc5)cc(-c5ccccc5)n4)cccc32)c1-n1c2ccccc2c2ccccc21]

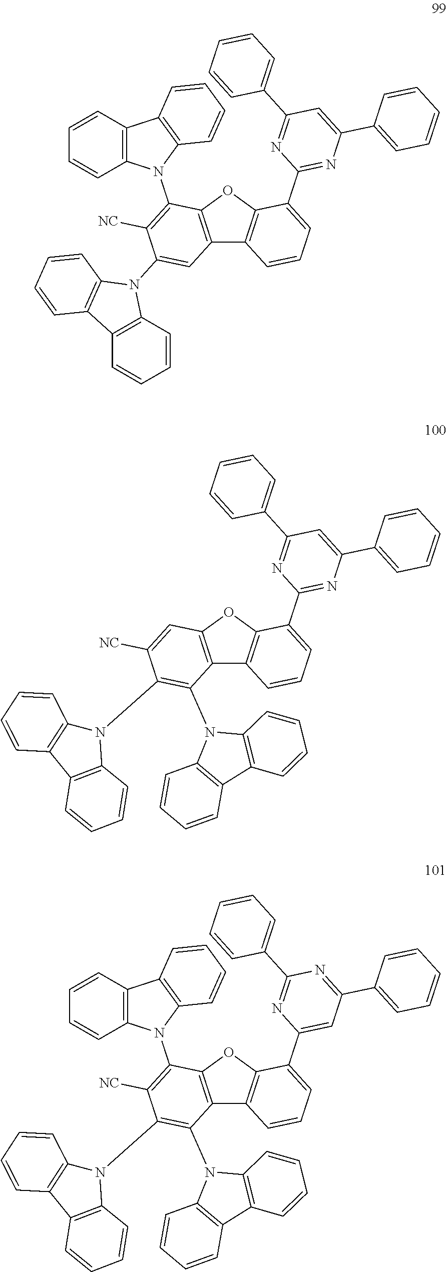 [SMILES: N#Cc1c(-n2c3ccccc3c3ccccc32)cc2c(oc3c(-c4nc(-c5ccccc5)cc(-c5ccccc5)n4)cccc32)c1-n1c2ccccc2c2ccccc21 ; N#Cc1cc2oc3c(-c4nc(-c5ccccc5)cc(-c5ccccc5)n4)cccc3c2c(-n2c3ccccc3c3ccccc32)c1-n1c2ccccc2c2ccccc21 ; N#Cc1c(-n2c3ccccc3c3ccccc32)c(-n2c3ccccc3c3ccccc32)c2c(oc3c(-c4cc(-c5ccccc5)nc(-c5ccccc5)n4)cccc32)c1-n1c2ccccc2c2ccccc21]

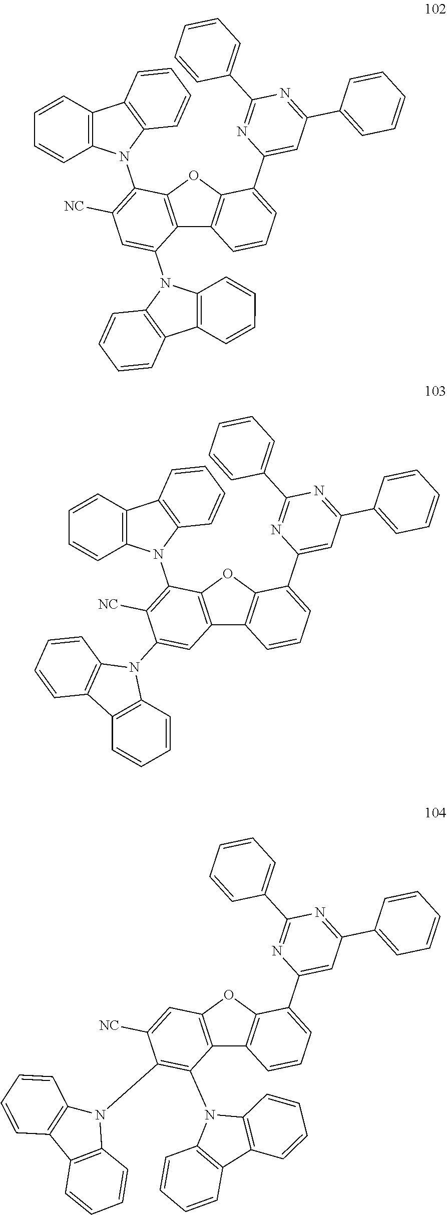 [SMILES: N#Cc1cc(-n2c3ccccc3c3ccccc32)c2c(oc3c(-c4cc(-c5ccccc5)nc(-c5ccccc5)n4)cccc32)c1-n1c2ccccc2c2ccccc21 ; N#Cc1c(-n2c3ccccc3c3ccccc32)cc2c(oc3c(-c4cc(-c5ccccc5)nc(-c5ccccc5)n4)cccc32)c1-n1c2ccccc2c2ccccc21 ; N#Cc1cc2oc3c(-c4cc(-c5ccccc5)nc(-c5ccccc5)n4)cccc3c2c(-n2c3ccccc3c3ccccc32)c1-n1c2ccccc2c2ccccc21]

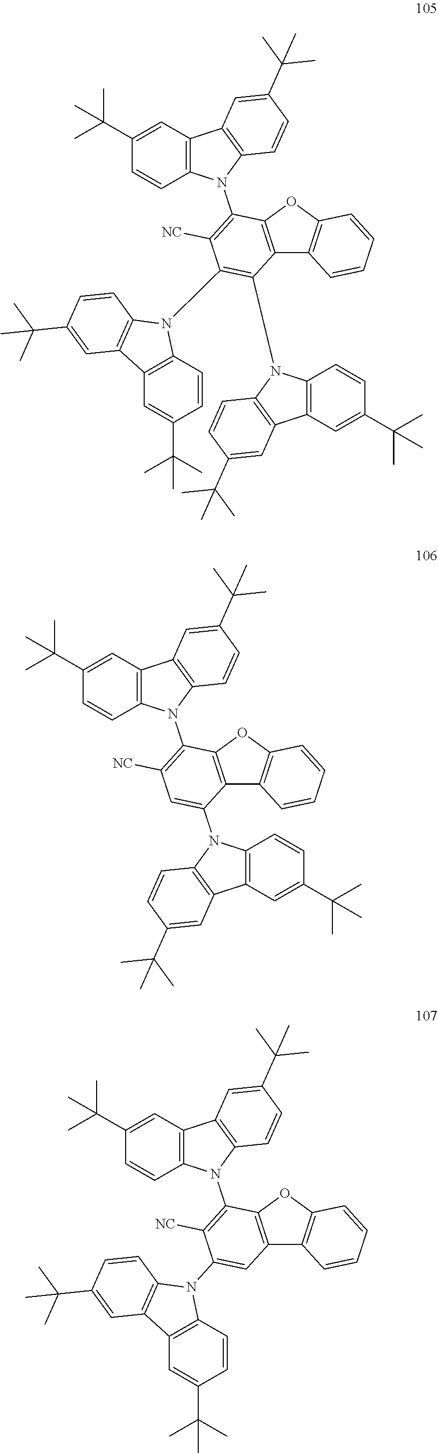

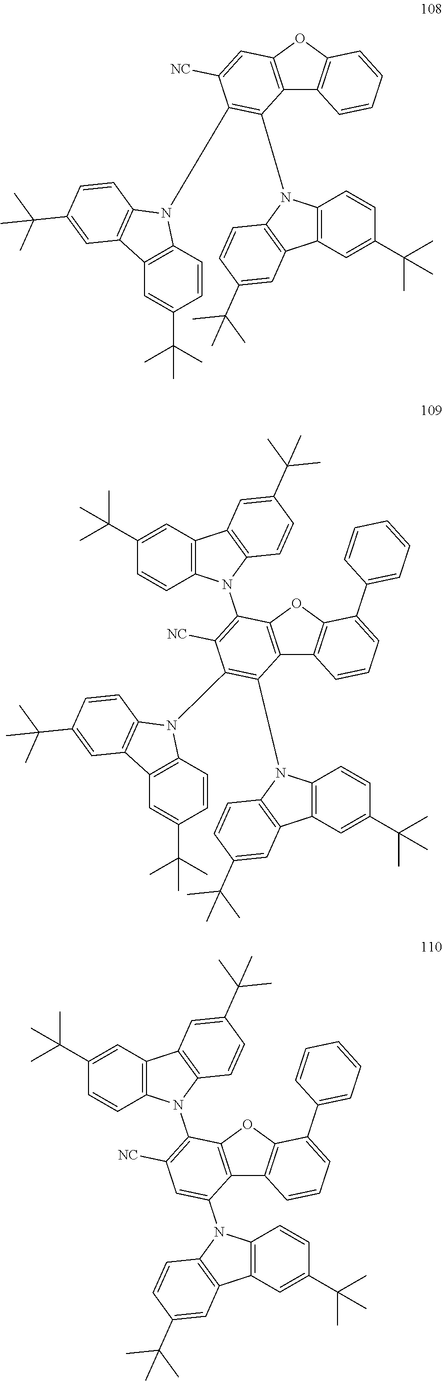

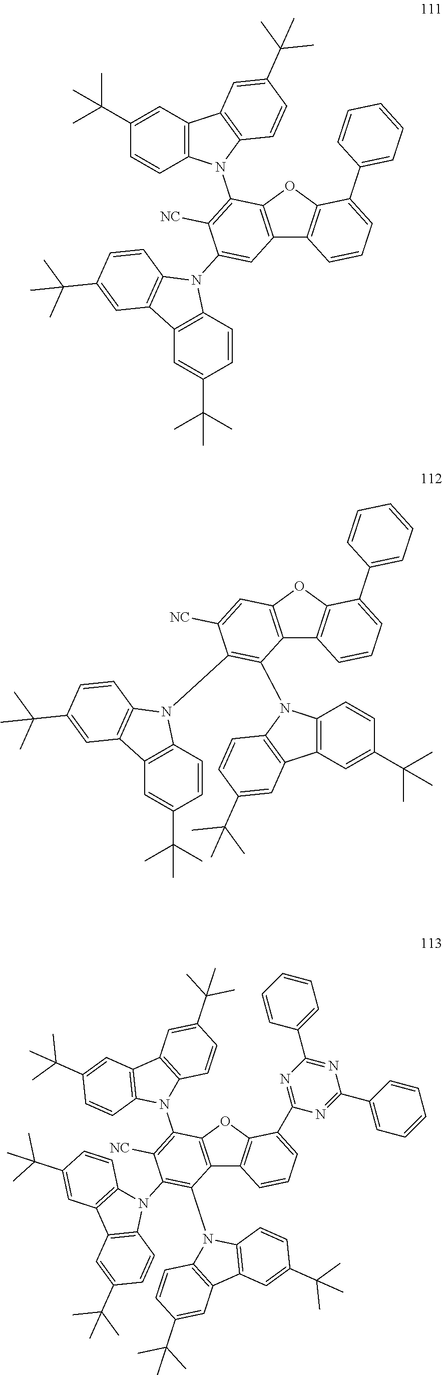

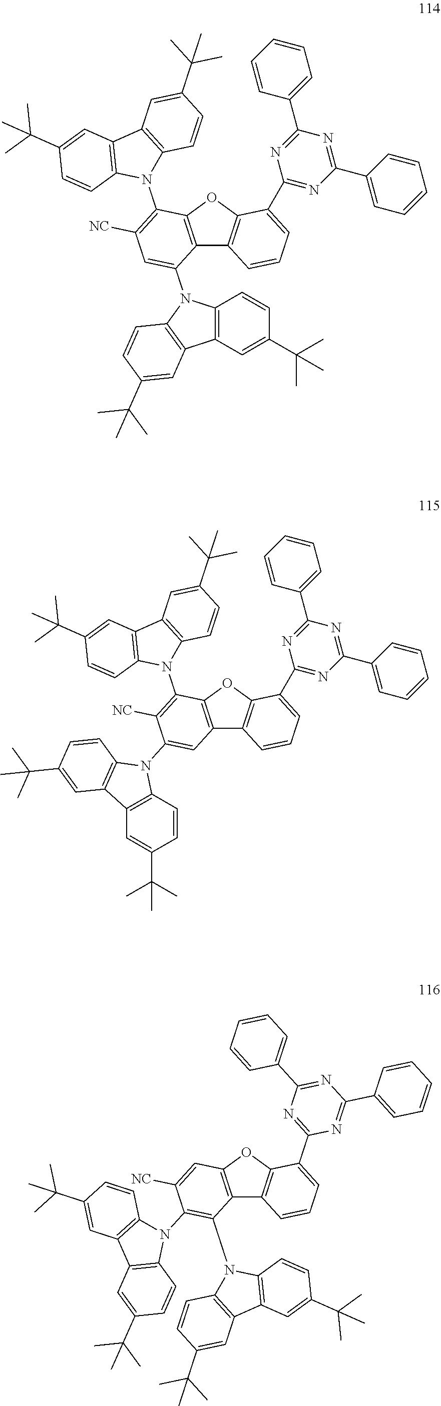 [SMILES: CC(C)(C)c1ccc2c(c1)c1cc(C(C)(C)C)ccc1n2-c1c(C#N)cc(-n2c3ccc(C(C)(C)C)cc3c3cc(C(C)(C)C)ccc32)c2c1oc1c(-c3nc(-c4ccccc4)nc(-c4ccccc4)n3)cccc12 ; CC(C)(C)c1ccc2c(c1)c1cc(C(C)(C)C)ccc1n2-c1cc2c(oc3c(-c4nc(-c5ccccc5)nc(-c5ccccc5)n4)cccc32)c(-n2c3ccc(C(C)(C)C)cc3c3cc(C(C)(C)C)ccc32)c1C#N ; CC(C)(C)c1ccc2c(c1)c1cc(C(C)(C)C)ccc1n2-c1c(C#N)cc2oc3c(-c4nc(-c5ccccc5)nc(-c5ccccc5)n4)cccc3c2c1-n1c2ccc(C(C)(C)C)cc2c2cc(C(C)(C)C)ccc21]

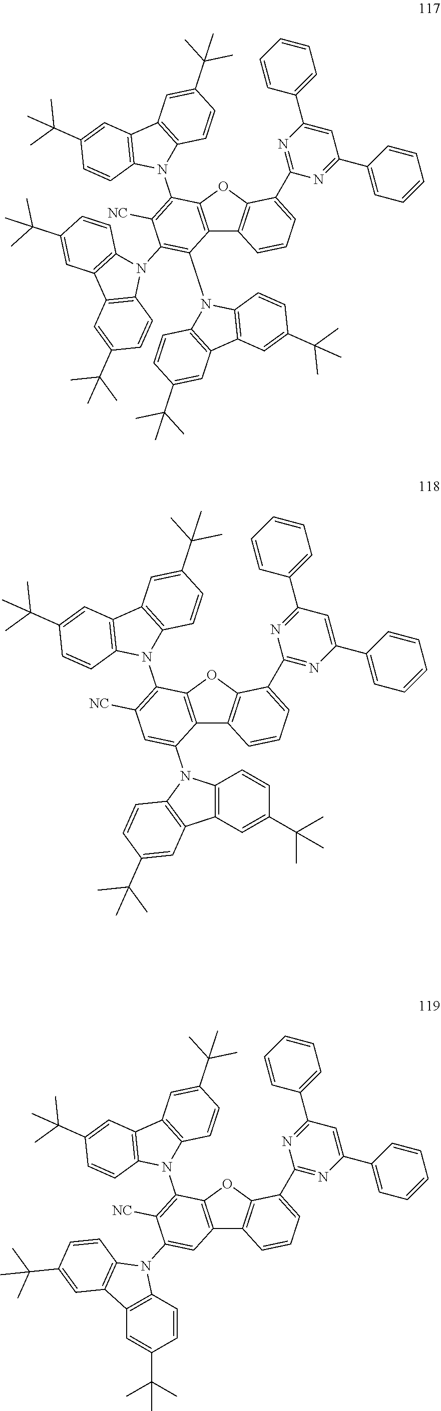 [SMILES: CC(C)(C)c1ccc2c(c1)c1cc(C(C)(C)C)ccc1n2-c1c(C#N)c(-n2c3ccc(C(C)(C)C)cc3c3cc(C(C)(C)C)ccc32)c2oc3c(-c4nc(-c5ccccc5)cc(-c5ccccc5)n4)cccc3c2c1-n1c2ccc(C(C)(C)C)cc2c2cc(C(C)(C)C)ccc21 ; CC(C)(C)c1ccc2c(c1)c1cc(C(C)(C)C)ccc1n2-c1c(C#N)cc(-n2c3ccc(C(C)(C)C)cc3c3cc(C(C)(C)C)ccc32)c2c1oc1c(-c3nc(-c4ccccc4)cc(-c4ccccc4)n3)cccc12 ; CC(C)(C)c1ccc2c(c1)c1cc(C(C)(C)C)ccc1n2-c1cc2c(oc3c(-c4nc(-c5ccccc5)cc(-c5ccccc5)n4)cccc32)c(-n2c3ccc(C(C)(C)C)cc3c3cc(C(C)(C)C)ccc32)c1C#N]

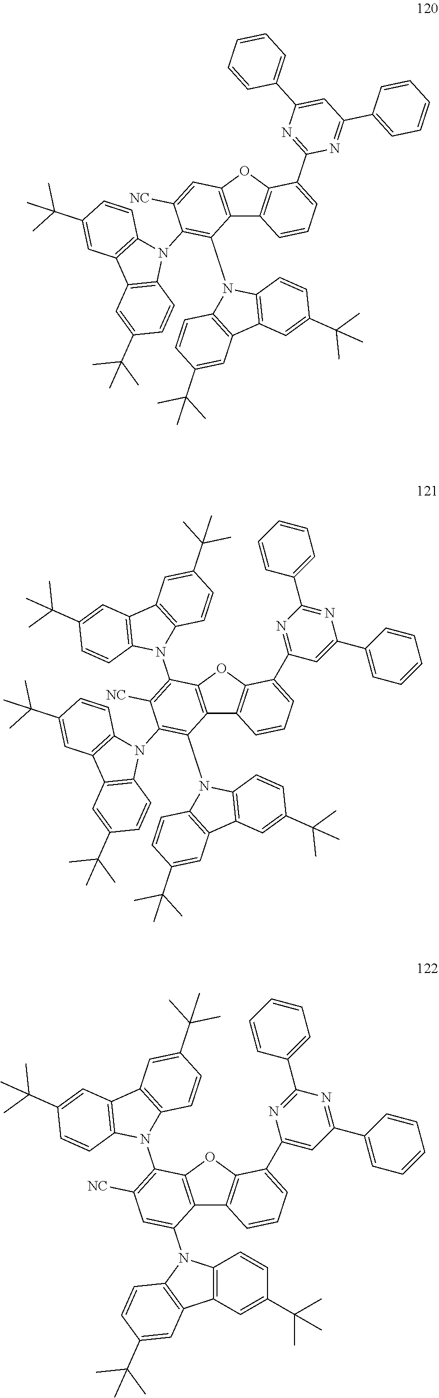 [SMILES: CC(C)(C)c1ccc2c(c1)c1cc(C(C)(C)C)ccc1n2-c1c(C#N)cc2oc3c(-c4nc(-c5ccccc5)cc(-c5ccccc5)n4)cccc3c2c1-n1c2ccc(C(C)(C)C)cc2c2cc(C(C)(C)C)ccc21 ; CC(C)(C)c1ccc2c(c1)c1cc(C(C)(C)C)ccc1n2-c1c(C#N)c(-n2c3ccc(C(C)(C)C)cc3c3cc(C(C)(C)C)ccc32)c2oc3c(-c4cc(-c5ccccc5)nc(-c5ccccc5)n4)cccc3c2c1-n1c2ccc(C(C)(C)C)cc2c2cc(C(C)(C)C)ccc21 ; CC(C)(C)c1ccc2c(c1)c1cc(C(C)(C)C)ccc1n2-c1c(C#N)cc(-n2c3ccc(C(C)(C)C)cc3c3cc(C(C)(C)C)ccc32)c2c1oc1c(-c3cc(-c4ccccc4)nc(-c4ccccc4)n3)cccc12]

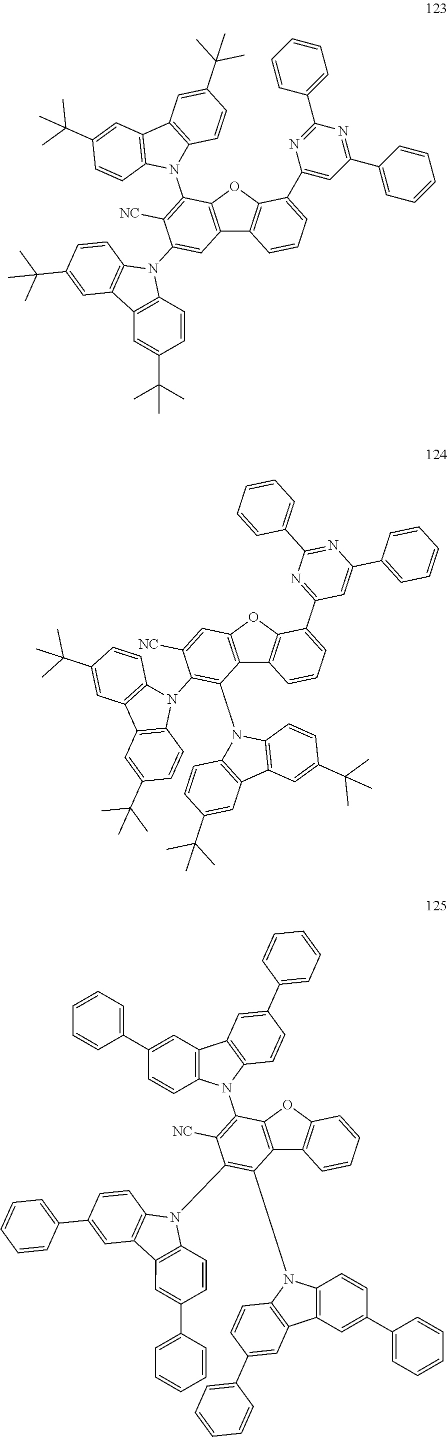 [SMILES: CC(C)(C)c1ccc2c(c1)c1cc(C(C)(C)C)ccc1n2-c1cc2c(oc3c(-c4cc(-c5ccccc5)nc(-c5ccccc5)n4)cccc32)c(-n2c3ccc(C(C)(C)C)cc3c3cc(C(C)(C)C)ccc32)c1C#N ; CC(C)(C)c1ccc2c(c1)c1cc(C(C)(C)C)ccc1n2-c1c(C#N)cc2oc3c(-c4cc(-c5ccccc5)nc(-c5ccccc5)n4)cccc3c2c1-n1c2ccc(C(C)(C)C)cc2c2cc(C(C)(C)C)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2c(oc3ccccc32)c1-n1c2ccc(-c3ccccc3)cc2c2cc(-c3ccccc3)ccc21]

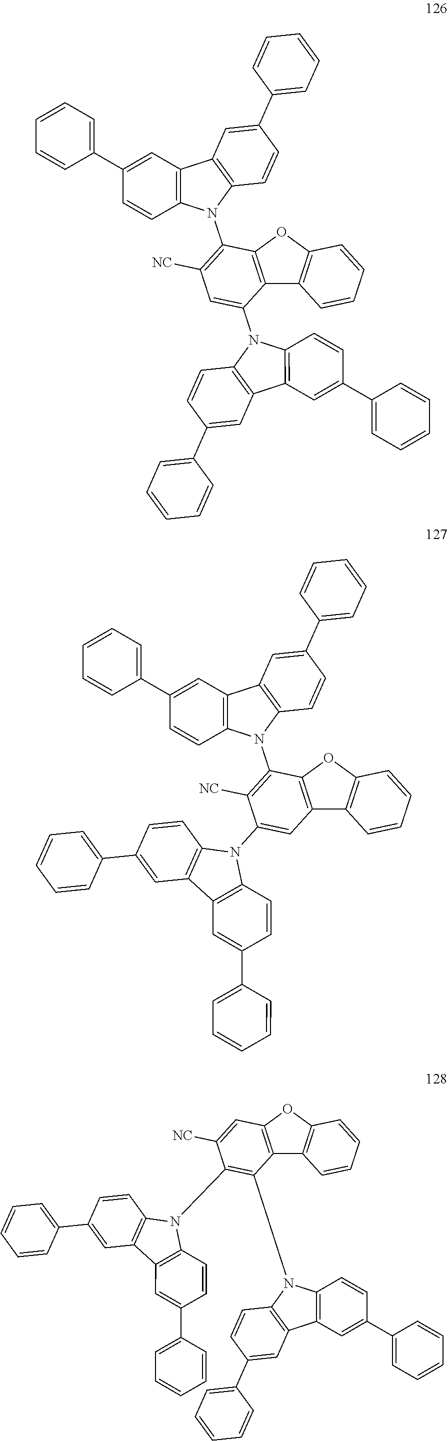 [SMILES: N#Cc1cc(-n2c3ccc(-c4ccccc4)cc3c3cc(-c4ccccc4)ccc32)c2c(oc3ccccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2c(oc3ccccc32)c1-n1c2ccc(-c3ccccc3)cc2c2cc(-c3ccccc3)ccc21 ; N#Cc1cc2oc3ccccc3c2c(-n2c3ccc(-c4ccccc4)cc3c3cc(-c4ccccc4)ccc32)c1-n1c2ccc(-c3ccccc3)cc2c2cc(-c3ccccc3)ccc21]

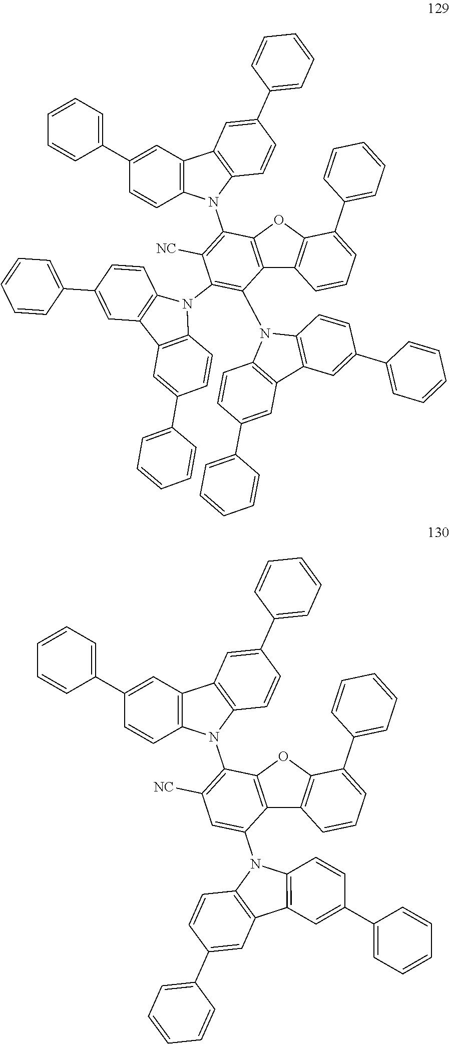 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2c(oc3c(-c4ccccc4)cccc32)c1-n1c2ccc(-c3ccccc3)cc2c2cc(-c3ccccc3)ccc21 ; N#Cc1cc(-n2c3ccc(-c4ccccc4)cc3c3cc(-c4ccccc4)ccc32)c2c(oc3c(-c4ccccc4)cccc32)c1-n1c2ccc(-c3ccccc3)cc2c2cc(-c3ccccc3)ccc21]

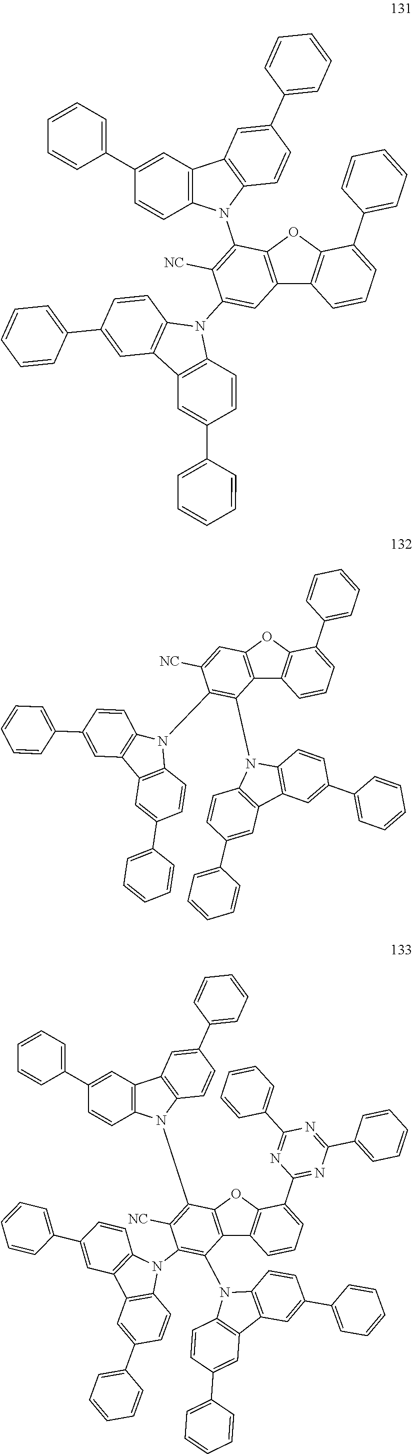 [SMILES: N#Cc1c(-n2c3ccc(-c4ccccc4)cc3c3cc(-c4ccccc4)ccc32)cc2c(oc3c(-c4ccccc4)cccc32)c1-n1c2ccc(-c3ccccc3)cc2c2cc(-c3ccccc3)ccc21 ; N#Cc1cc2oc3c(-c4ccccc4)cccc3c2c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2c(oc3c(-c4nc(-c5ccccc5)nc(-c5ccccc5)n4)cccc32)c1-n1c2ccc(-c3ccccc3)cc2c2cc(-c3ccccc3)ccc21]

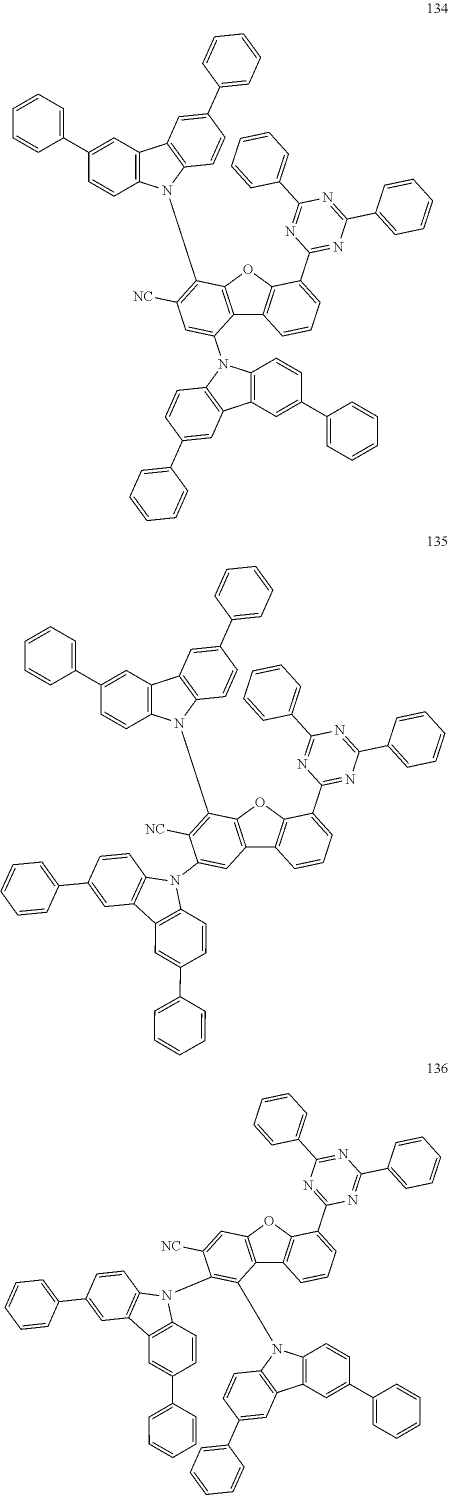 [SMILES: N#Cc1cc(-n2c3ccc(-c4ccccc4)cc3c3cc(-c4ccccc4)ccc32)c2c(oc3c(-c4nc(-c5ccccc5)nc(-c5ccccc5)n4)cccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2c(oc3c(-c4nc(-c5ccccc5)nc(-c5ccccc5)n4)cccc32)c1-n1c2ccc(-c3ccccc3)cc2c2cc(-c3ccccc3)ccc21 ; N#Cc1cc2oc3c(-c4nc(-c5ccccc5)nc(-c5ccccc5)n4)cccc3c2c(-n2c3ccc(-c4ccccc4)cc3c3cc(-c4ccccc4)ccc32)c1-n1c2ccc(-c3ccccc3)cc2c2cc(-c3ccccc3)ccc21]

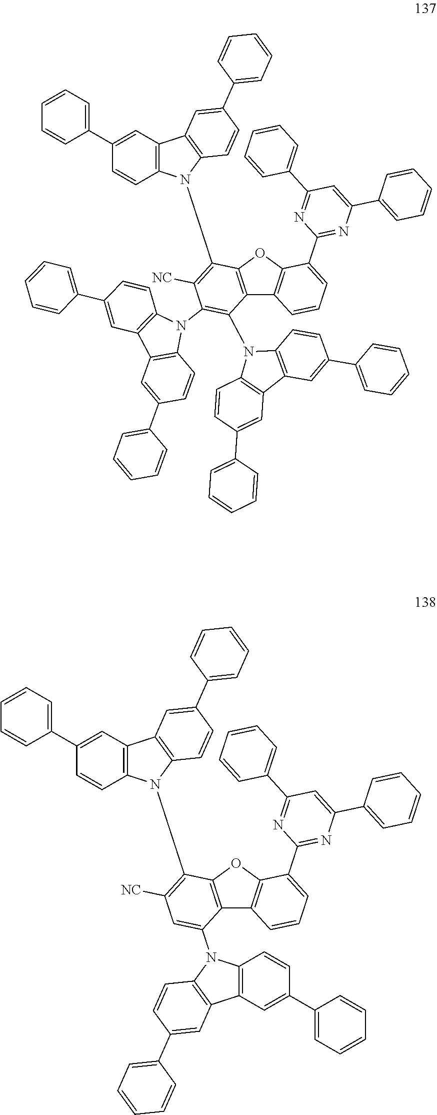 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2c(oc3c(-c4nc(-c5ccccc5)cc(-c5ccccc5)n4)cccc32)c1-n1c2ccc(-c3ccccc3)cc2c2cc(-c3ccccc3)ccc21 ; N#Cc1cc(-n2c3ccc(-c4ccccc4)cc3c3cc(-c4ccccc4)ccc32)c2c(oc3c(-c4nc(-c5ccccc5)cc(-c5ccccc5)n4)cccc32)c1-n1c2ccc(-c3ccccc3)cc2c2cc(-c3ccccc3)ccc21]

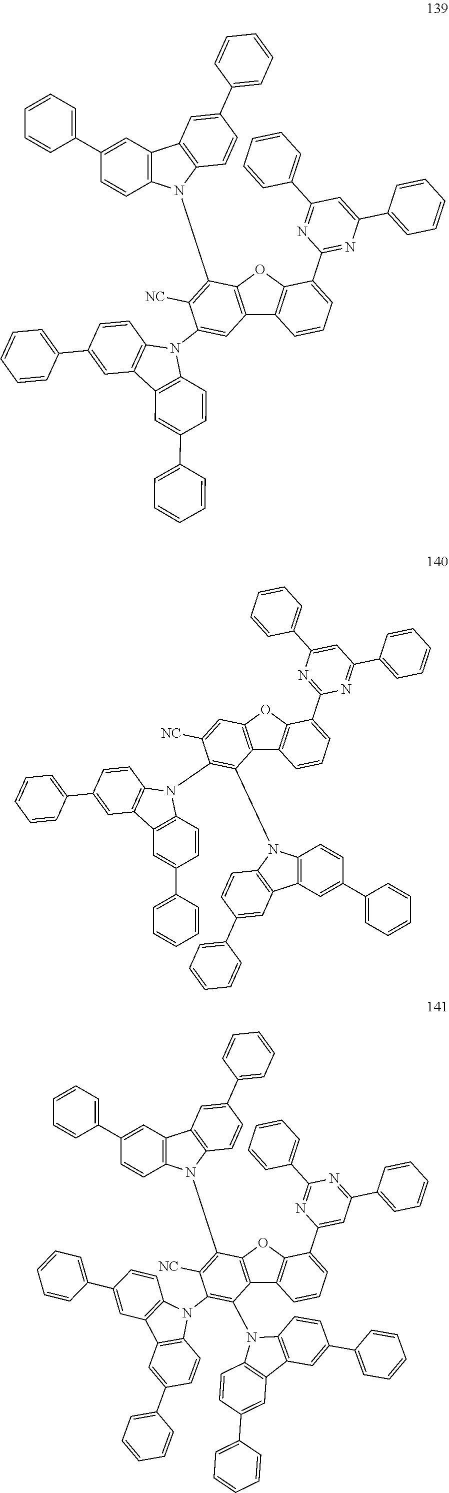 [SMILES: N#Cc1c(-n2c3ccc(-c4ccccc4)cc3c3cc(-c4ccccc4)ccc32)cc2c(oc3c(-c4nc(-c5ccccc5)cc(-c5ccccc5)n4)cccc32)c1-n1c2ccc(-c3ccccc3)cc2c2cc(-c3ccccc3)ccc21 ; N#Cc1cc2oc3c(-c4nc(-c5ccccc5)cc(-c5ccccc5)n4)cccc3c2c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2c(oc3c(-c4cc(-c5ccccc5)nc(-c5ccccc5)n4)cccc32)c1-n1c2ccc(-c3ccccc3)cc2c2cc(-c3ccccc3)ccc21]

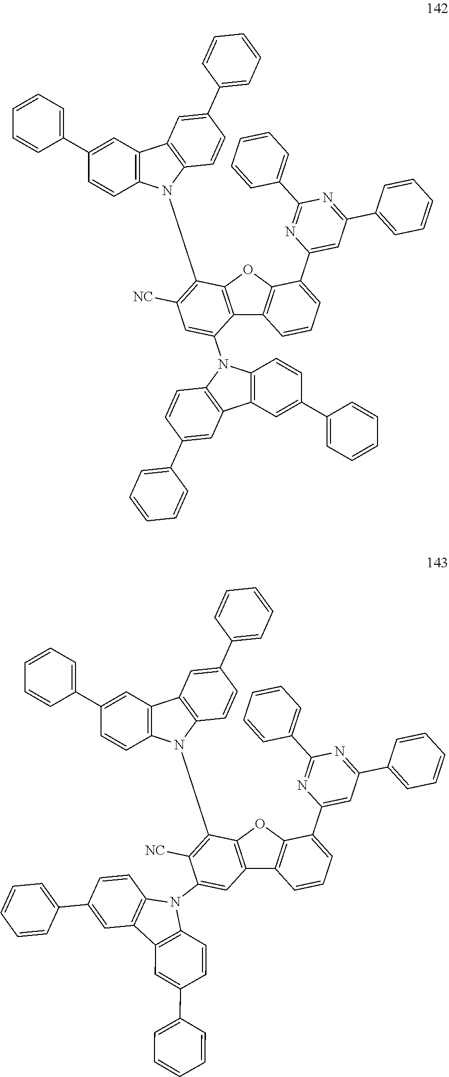 [SMILES: N#Cc1cc(-n2c3ccc(-c4ccccc4)cc3c3cc(-c4ccccc4)ccc32)c2c(oc3c(-c4cc(-c5ccccc5)nc(-c5ccccc5)n4)cccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2c(oc3c(-c4cc(-c5ccccc5)nc(-c5ccccc5)n4)cccc32)c1-n1c2ccc(-c3ccccc3)cc2c2cc(-c3ccccc3)ccc21]

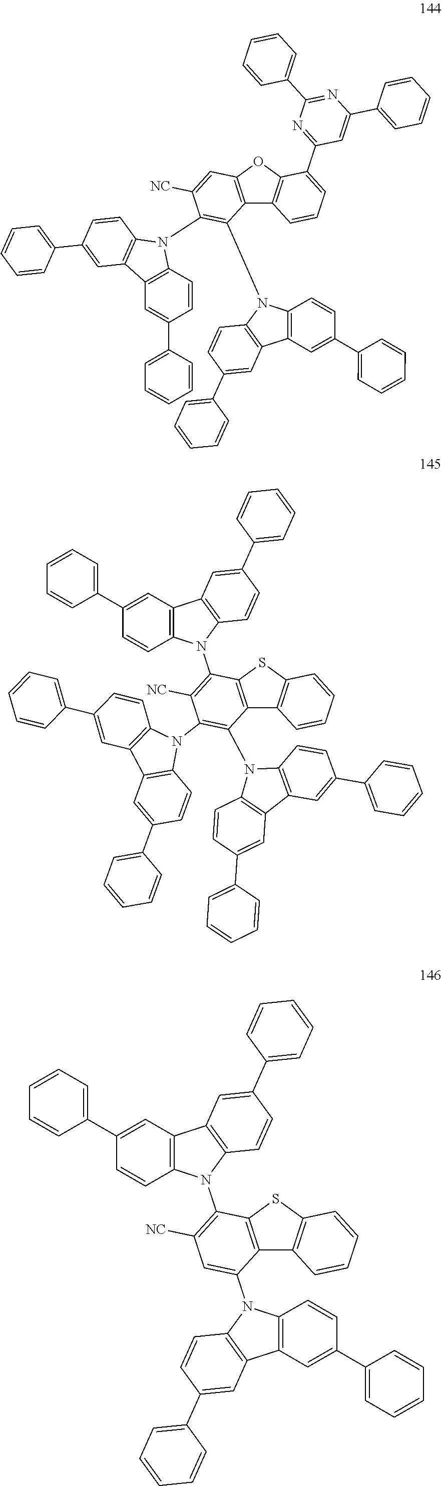 [SMILES: N#Cc1cc2oc3c(-c4cc(-c5ccccc5)nc(-c5ccccc5)n4)cccc3c2c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2c(sc3ccccc32)c1-n1c2ccc(-c3ccccc3)cc2c2cc(-c3ccccc3)ccc21 ; N#Cc1cc(-n2c3ccc(-c4ccccc4)cc3c3cc(-c4ccccc4)ccc32)c2c(sc3ccccc32)c1-n1c2ccc(-c3ccccc3)cc2c2cc(-c3ccccc3)ccc21]

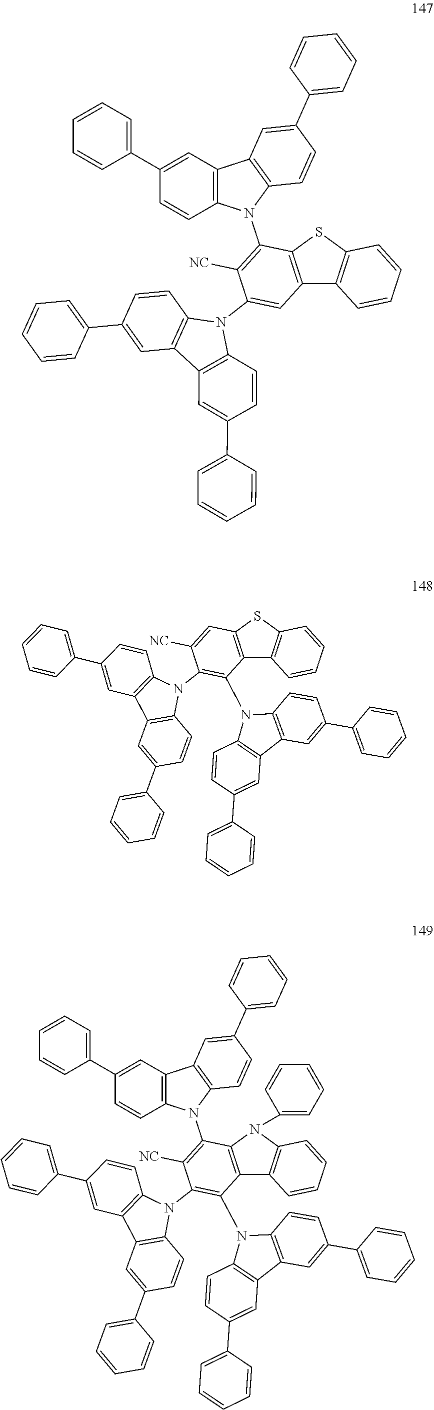 [SMILES: N#Cc1c(-n2c3ccc(-c4ccccc4)cc3c3cc(-c4ccccc4)ccc32)cc2c(sc3ccccc32)c1-n1c2ccc(-c3ccccc3)cc2c2cc(-c3ccccc3)ccc21 ; N#Cc1cc2sc3ccccc3c2c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2c3ccccc3n(-c3ccccc3)c2c1-n1c2ccc(-c3ccccc3)cc2c2cc(-c3ccccc3)ccc21]

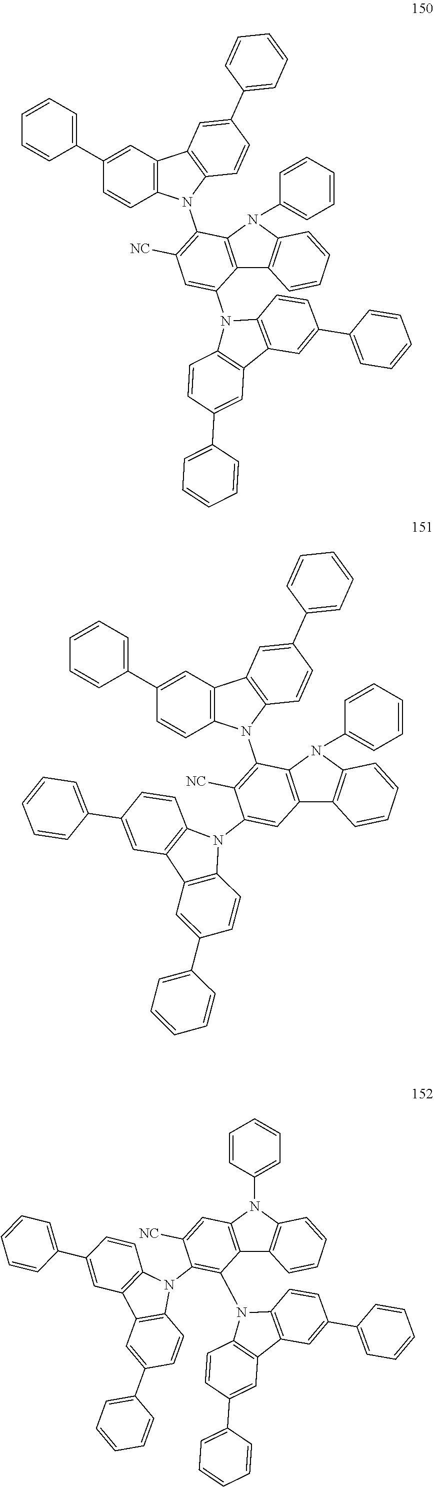 [SMILES: N#Cc1cc(-n2c3ccc(-c4ccccc4)cc3c3cc(-c4ccccc4)ccc32)c2c3ccccc3n(-c3ccccc3)c2c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2c3ccccc3n(-c3ccccc3)c2c1-n1c2ccc(-c3ccccc3)cc2c2cc(-c3ccccc3)ccc21 ; N#Cc1cc2c(c(-n3c4ccc(-c5ccccc5)cc4c4cc(-c5ccccc5)ccc43)c1-n1c3ccc(-c4ccccc4)cc3c3cc(-c4ccccc4)ccc31)c1ccccc1n2-c1ccccc1]

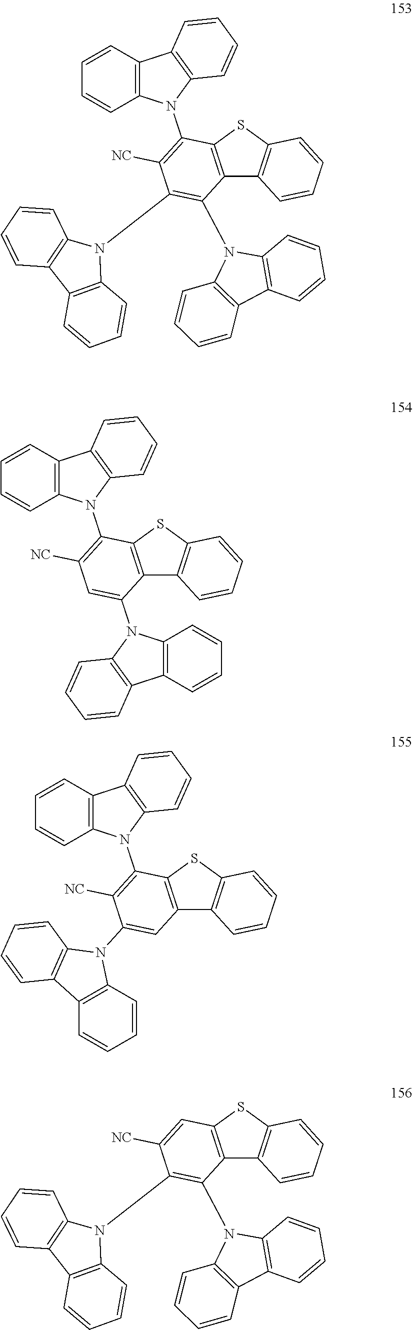

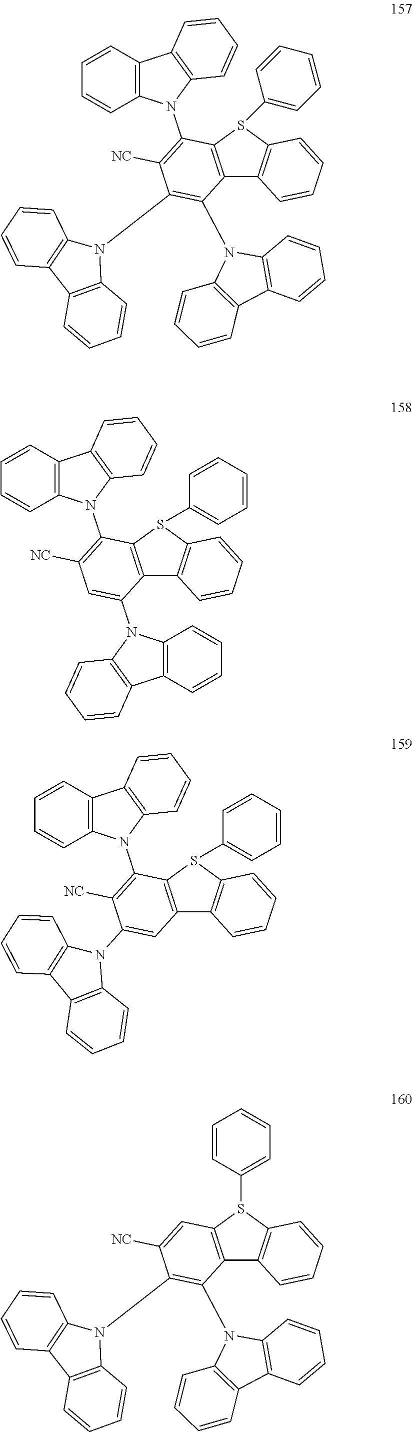

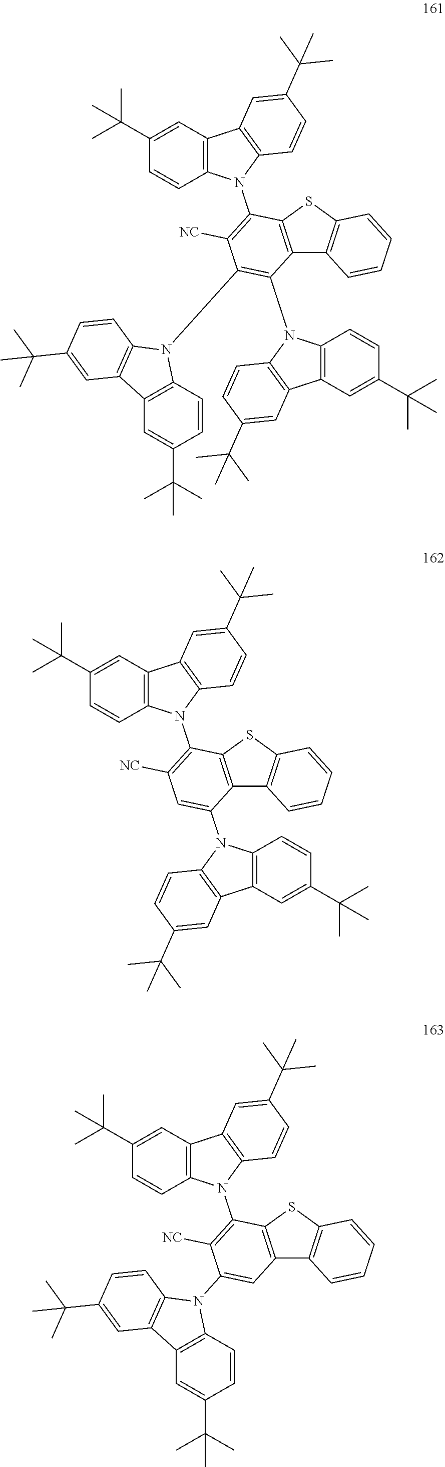

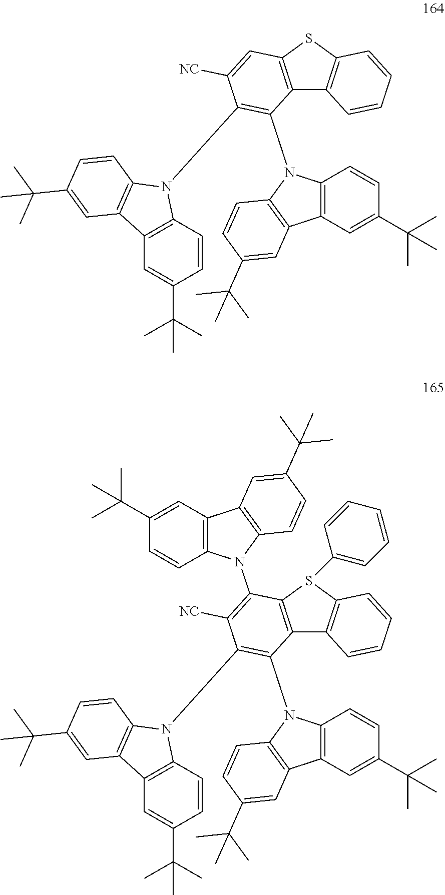

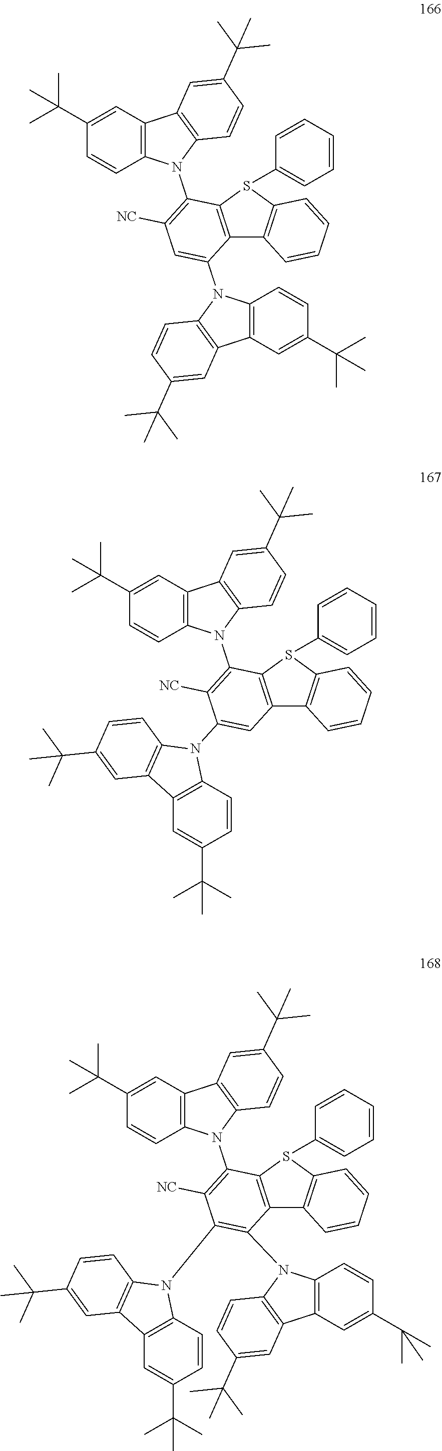

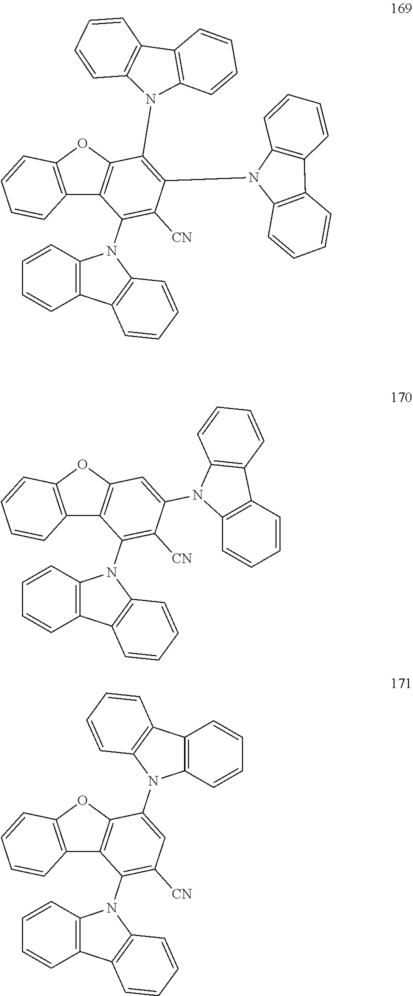

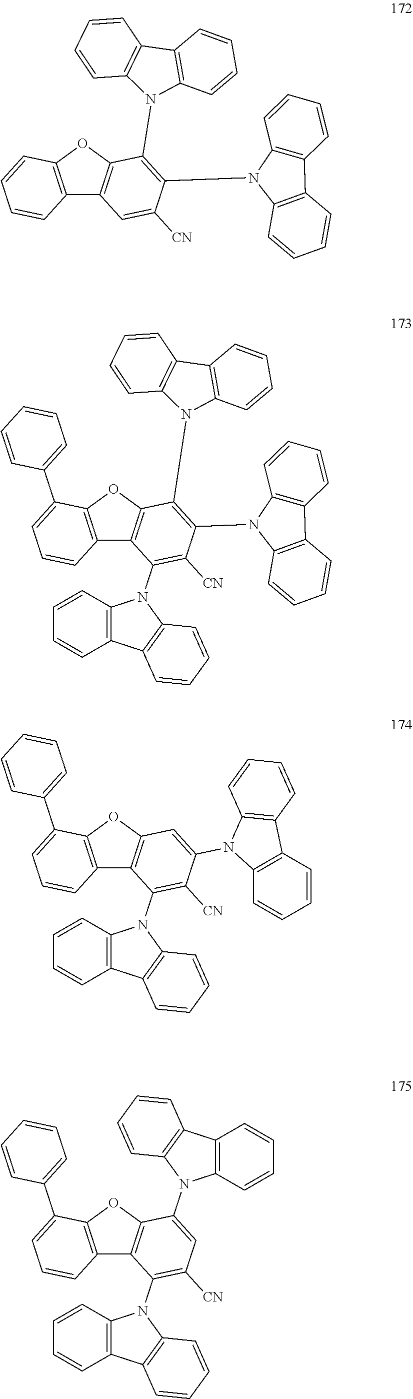 [SMILES: N#Cc1cc2c(oc3ccccc32)c(-n2c3ccccc3c3ccccc32)c1-n1c2ccccc2c2ccccc21 ; N#Cc1c(-n2c3ccccc3c3ccccc32)c(-n2c3ccccc3c3ccccc32)c2oc3c(-c4ccccc4)cccc3c2c1-n1c2ccccc2c2ccccc21 ; N#Cc1c(-n2c3ccccc3c3ccccc32)cc2oc3c(-c4ccccc4)cccc3c2c1-n1c2ccccc2c2ccccc21 ; N#Cc1cc(-n2c3ccccc3c3ccccc32)c2oc3c(-c4ccccc4)cccc3c2c1-n1c2ccccc2c2ccccc21]

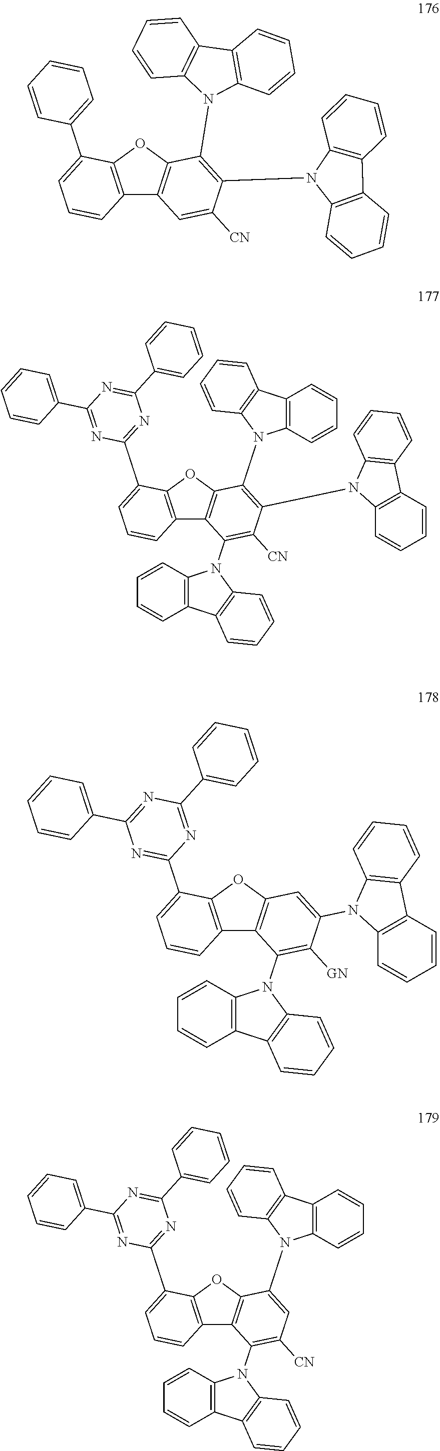

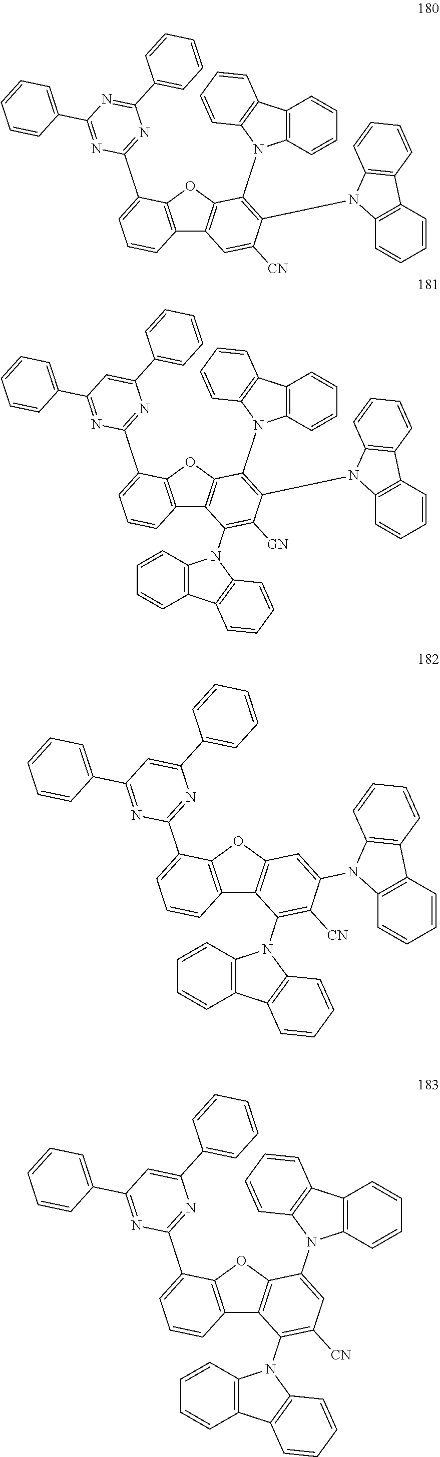

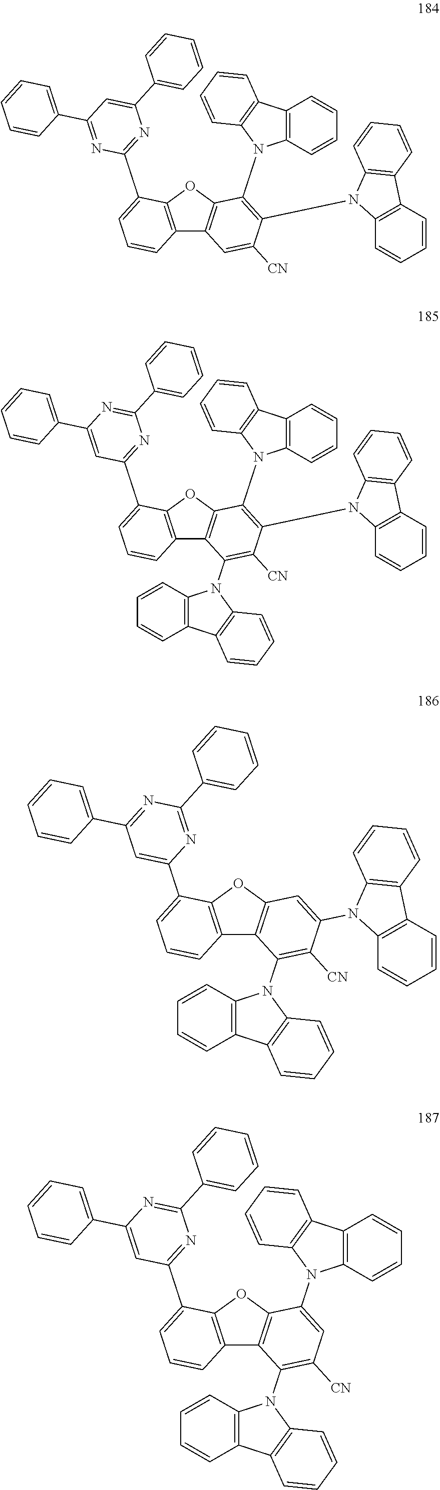 [SMILES: N#Cc1cc2c(oc3c(-c4nc(-c5ccccc5)cc(-c5ccccc5)n4)cccc32)c(-n2c3ccccc3c3ccccc32)c1-n1c2ccccc2c2ccccc21 ; N#Cc1c(-n2c3ccccc3c3ccccc32)c(-n2c3ccccc3c3ccccc32)c2oc3c(-c4cc(-c5ccccc5)nc(-c5ccccc5)n4)cccc3c2c1-n1c2ccccc2c2ccccc21 ; N#Cc1c(-n2c3ccccc3c3ccccc32)cc2oc3c(-c4cc(-c5ccccc5)nc(-c5ccccc5)n4)cccc3c2c1-n1c2ccccc2c2ccccc21 ; N#Cc1cc(-n2c3ccccc3c3ccccc32)c2oc3c(-c4cc(-c5ccccc5)nc(-c5ccccc5)n4)cccc3c2c1-n1c2ccccc2c2ccccc21]

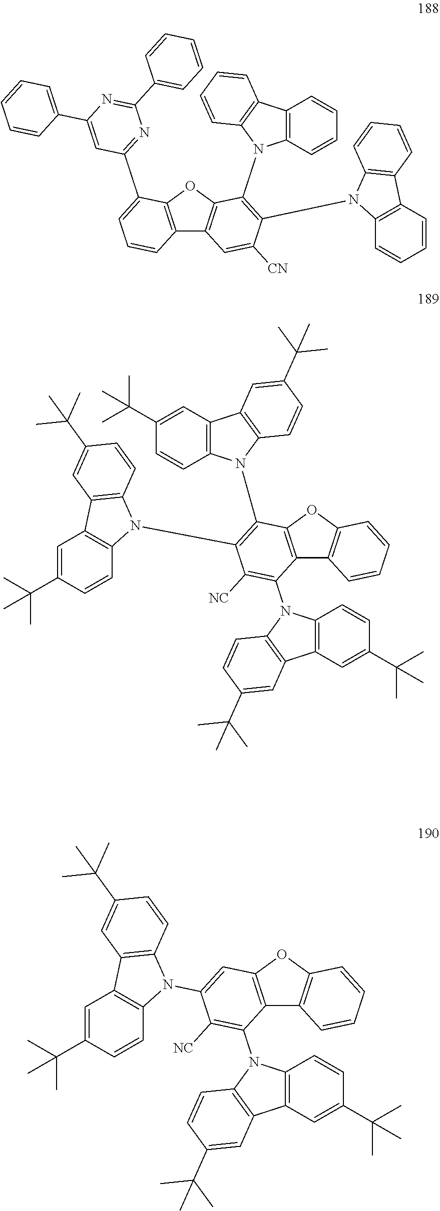

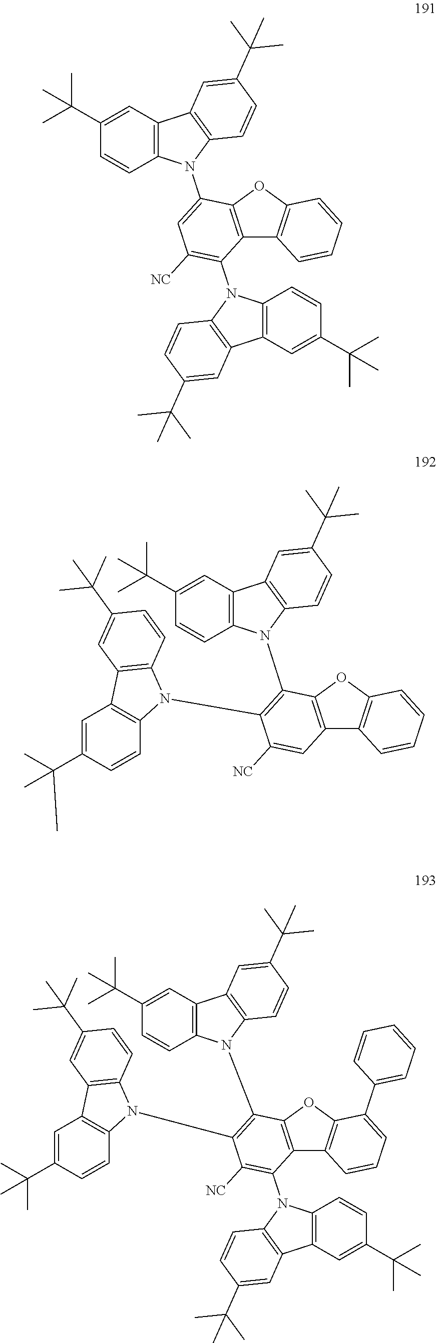

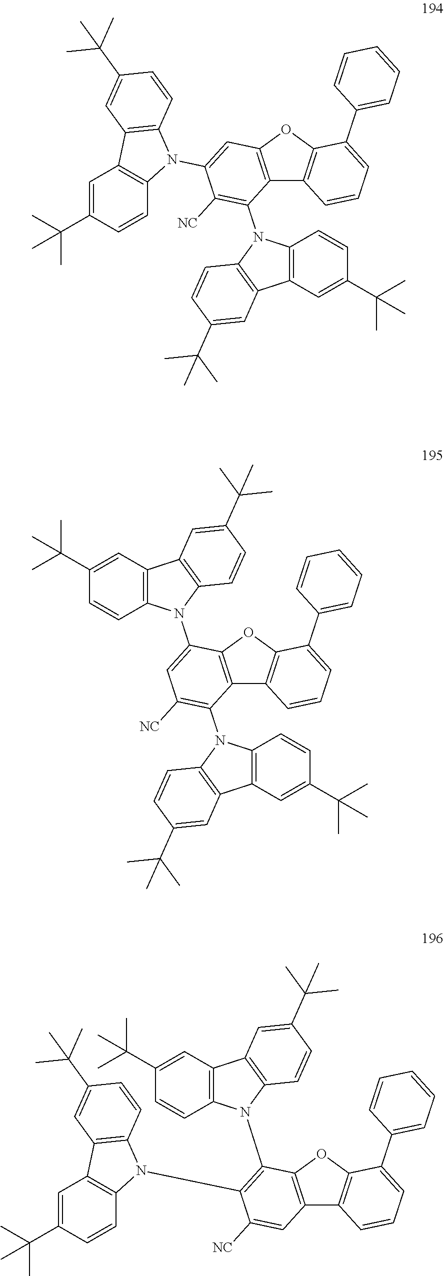

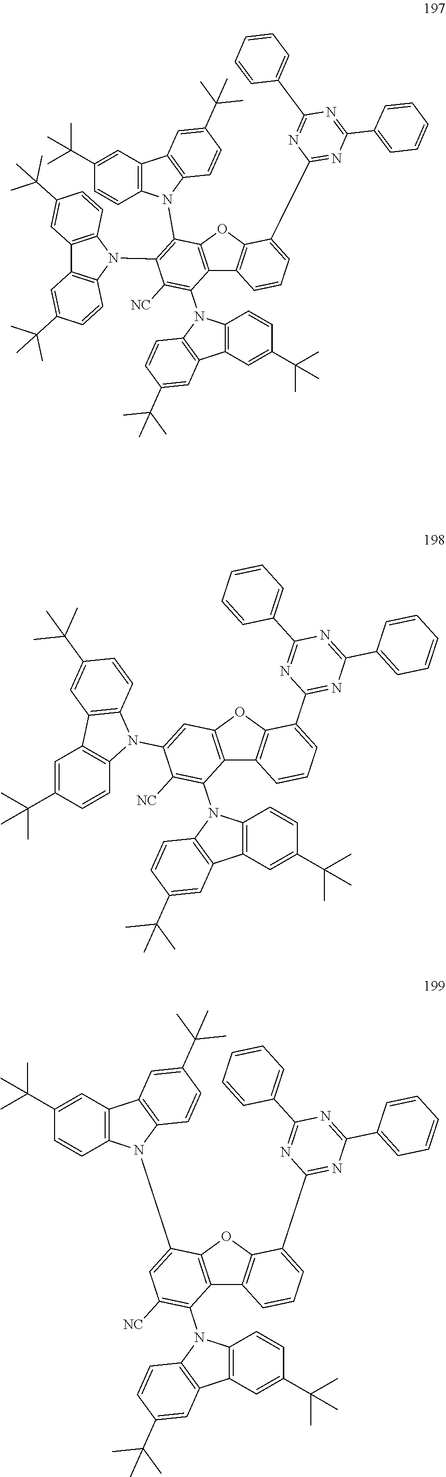 [SMILES: CC(C)(C)c1ccc2c(c1)c1cc(C(C)(C)C)ccc1n2-c1c(C#N)c(-n2c3ccc(C(C)(C)C)cc3c3cc(C(C)(C)C)ccc32)c2c(oc3c(-c4nc(-c5ccccc5)nc(-c5ccccc5)n4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1cc2oc3c(-c4nc(-c5ccccc5)nc(-c5ccccc5)n4)cccc3c2c(-n2c3ccc(C(C)(C)C)cc3c3cc(C(C)(C)C)ccc32)c1C#N ; CC(C)(C)c1ccc2c(c1)c1cc(C(C)(C)C)ccc1n2-c1cc(C#N)c(-n2c3ccc(C(C)(C)C)cc3c3cc(C(C)(C)C)ccc32)c2c1oc1c(-c3nc(-c4ccccc4)nc(-c4ccccc4)n3)cccc12]

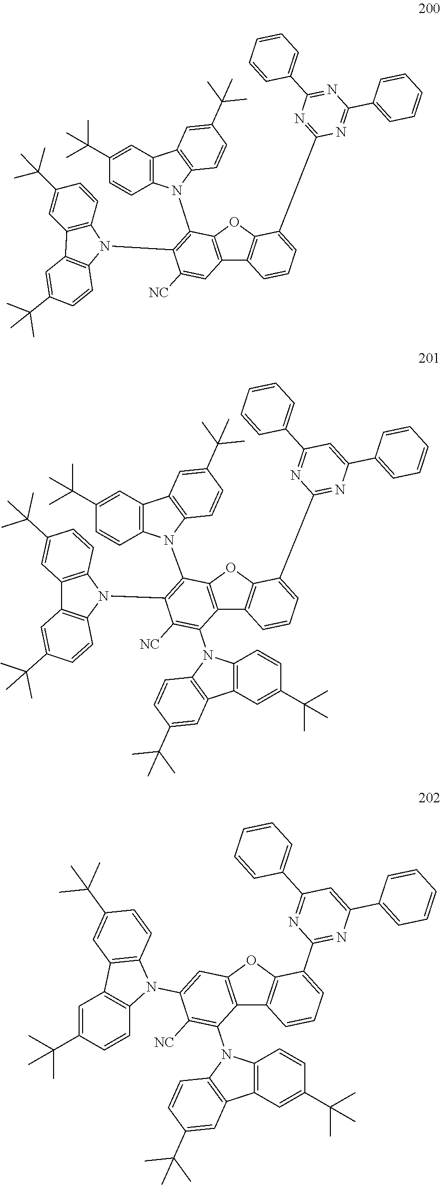 [SMILES: CC(C)(C)c1ccc2c(c1)c1cc(C(C)(C)C)ccc1n2-c1c(C#N)cc2c(oc3c(-c4nc(-c5ccccc5)nc(-c5ccccc5)n4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1c(C#N)c(-n2c3ccc(C(C)(C)C)cc3c3cc(C(C)(C)C)ccc32)c2c(oc3c(-c4nc(-c5ccccc5)cc(-c5ccccc5)n4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1cc2oc3c(-c4nc(-c5ccccc5)cc(-c5ccccc5)n4)cccc3c2c(-n2c3ccc(C(C)(C)C)cc3c3cc(C(C)(C)C)ccc32)c1C#N]

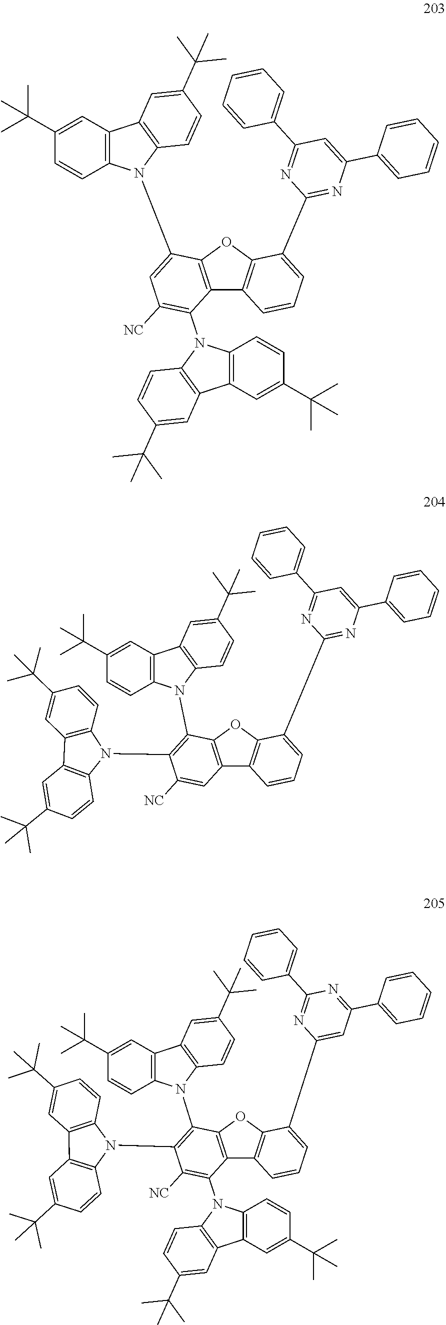 [SMILES: CC(C)(C)c1ccc2c(c1)c1cc(C(C)(C)C)ccc1n2-c1cc(C#N)c(-n2c3ccc(C(C)(C)C)cc3c3cc(C(C)(C)C)ccc32)c2c1oc1c(-c3nc(-c4ccccc4)cc(-c4ccccc4)n3)cccc12 ; CC(C)(C)c1ccc2c(c1)c1cc(C(C)(C)C)ccc1n2-c1c(C#N)cc2c(oc3c(-c4nc(-c5ccccc5)cc(-c5ccccc5)n4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1c(C#N)c(-n2c3ccc(C(C)(C)C)cc3c3cc(C(C)(C)C)ccc32)c2c(oc3c(-c4cc(-c5ccccc5)nc(-c5ccccc5)n4)cccc32)c1-n1c2ccc(C(C)(C)C)cc2c2cc(C(C)(C)C)ccc21]

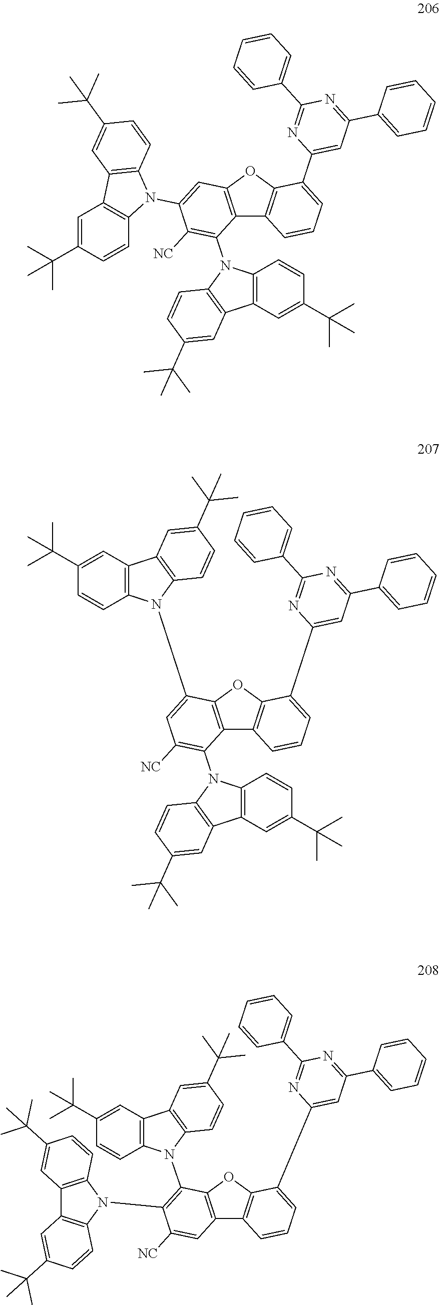 [SMILES: CC(C)(C)c1ccc2c(c1)c1cc(C(C)(C)C)ccc1n2-c1cc2oc3c(-c4cc(-c5ccccc5)nc(-c5ccccc5)n4)cccc3c2c(-n2c3ccc(C(C)(C)C)cc3c3cc(C(C)(C)C)ccc32)c1C#N ; CC(C)(C)c1ccc2c(c1)c1cc(C(C)(C)C)ccc1n2-c1cc(C#N)c(-n2c3ccc(C(C)(C)C)cc3c3cc(C(C)(C)C)ccc32)c2c1oc1c(-c3cc(-c4ccccc4)nc(-c4ccccc4)n3)cccc12 ; CC(C)(C)c1ccc2c(c1)c1cc(C(C)(C)C)ccc1n2-c1c(C#N)cc2c(oc3c(-c4cc(-c5ccccc5)nc(-c5ccccc5)n4)cccc32)c1-n1c2ccc(C(C)(C)C)cc2c2cc(C(C)(C)C)ccc21]

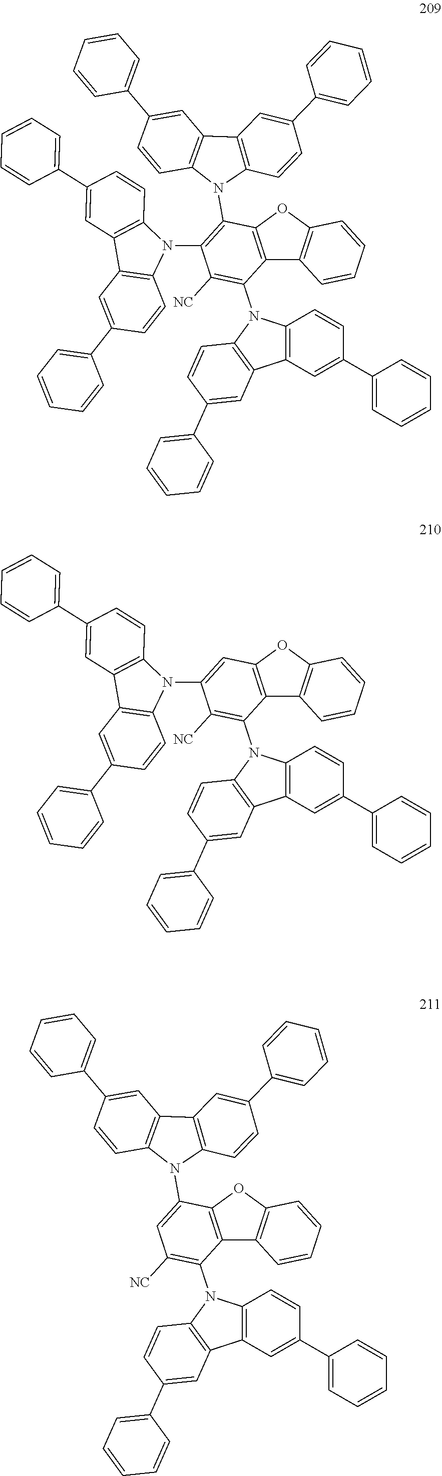 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2oc3ccccc3c2c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2oc3ccccc3c2c1-n1c2ccc(-c3ccccc3)cc2c2cc(-c3ccccc3)ccc21 ; N#Cc1cc(-n2c3ccc(-c4ccccc4)cc3c3cc(-c4ccccc4)ccc32)c2oc3ccccc3c2c1-n1c2ccc(-c3ccccc3)cc2c2cc(-c3ccccc3)ccc21]

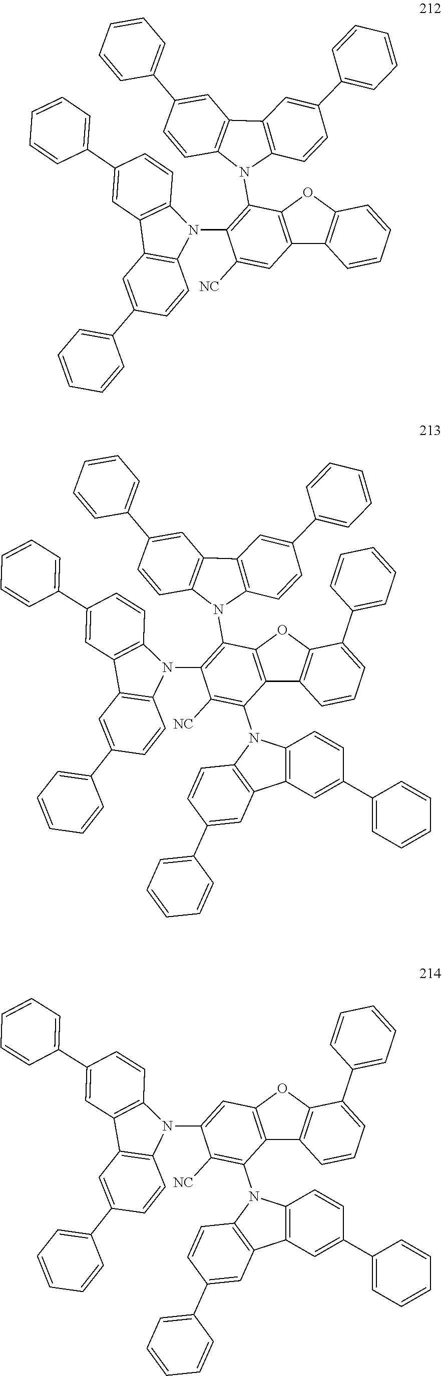 [SMILES: N#Cc1cc2c(oc3ccccc32)c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4ccccc4)cccc3c2c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2oc3c(-c4ccccc4)cccc3c2c1-n1c2ccc(-c3ccccc3)cc2c2cc(-c3ccccc3)ccc21]

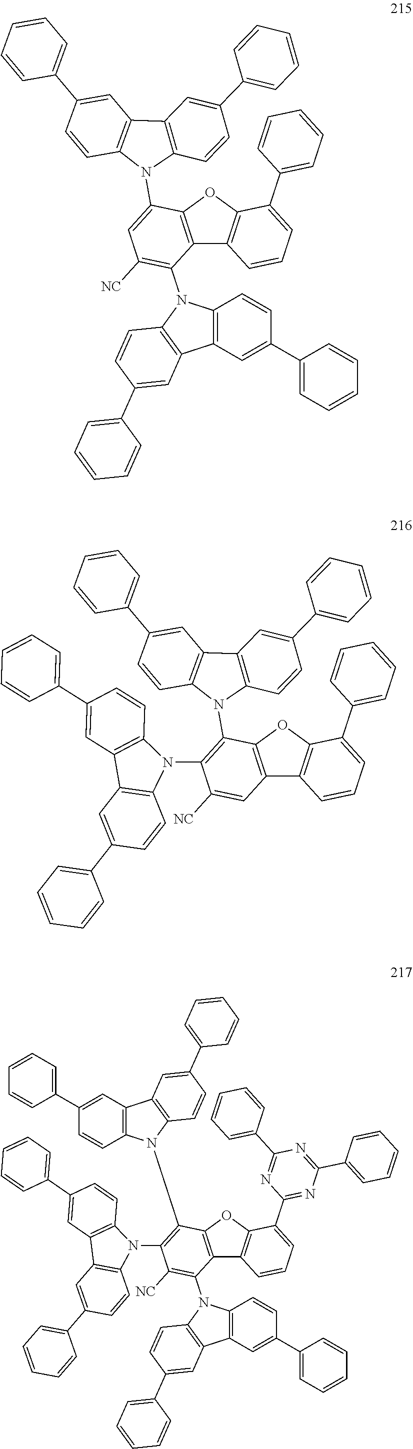 [SMILES: N#Cc1cc(-n2c3ccc(-c4ccccc4)cc3c3cc(-c4ccccc4)ccc32)c2oc3c(-c4ccccc4)cccc3c2c1-n1c2ccc(-c3ccccc3)cc2c2cc(-c3ccccc3)ccc21 ; N#Cc1cc2c(oc3c(-c4ccccc4)cccc32)c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4nc(-c5ccccc5)nc(-c5ccccc5)n4)cccc3c2c1-n1c2ccc(-c3ccccc3)cc2c2cc(-c3ccccc3)ccc21]

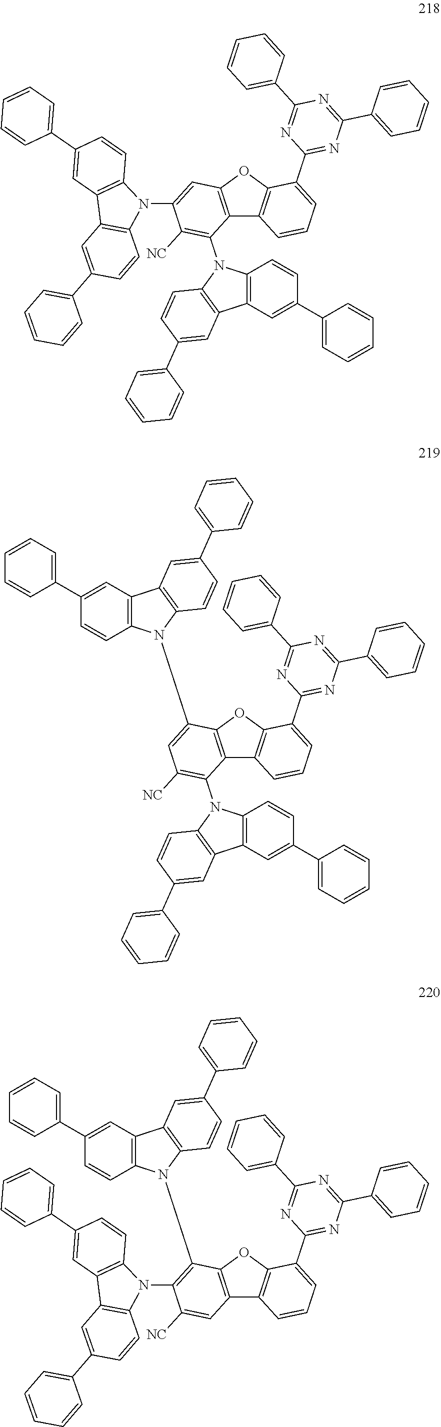 [SMILES: N#Cc1c(-n2c3ccc(-c4ccccc4)cc3c3cc(-c4ccccc4)ccc32)cc2oc3c(-c4nc(-c5ccccc5)nc(-c5ccccc5)n4)cccc3c2c1-n1c2ccc(-c3ccccc3)cc2c2cc(-c3ccccc3)ccc21 ; N#Cc1cc(-n2c3ccc(-c4ccccc4)cc3c3cc(-c4ccccc4)ccc32)c2oc3c(-c4nc(-c5ccccc5)nc(-c5ccccc5)n4)cccc3c2c1-n1c2ccc(-c3ccccc3)cc2c2cc(-c3ccccc3)ccc21 ; N#Cc1cc2c(oc3c(-c4nc(-c5ccccc5)nc(-c5ccccc5)n4)cccc32)c(-n2c3ccc(-c4ccccc4)cc3c3cc(-c4ccccc4)ccc32)c1-n1c2ccc(-c3ccccc3)cc2c2cc(-c3ccccc3)ccc21]

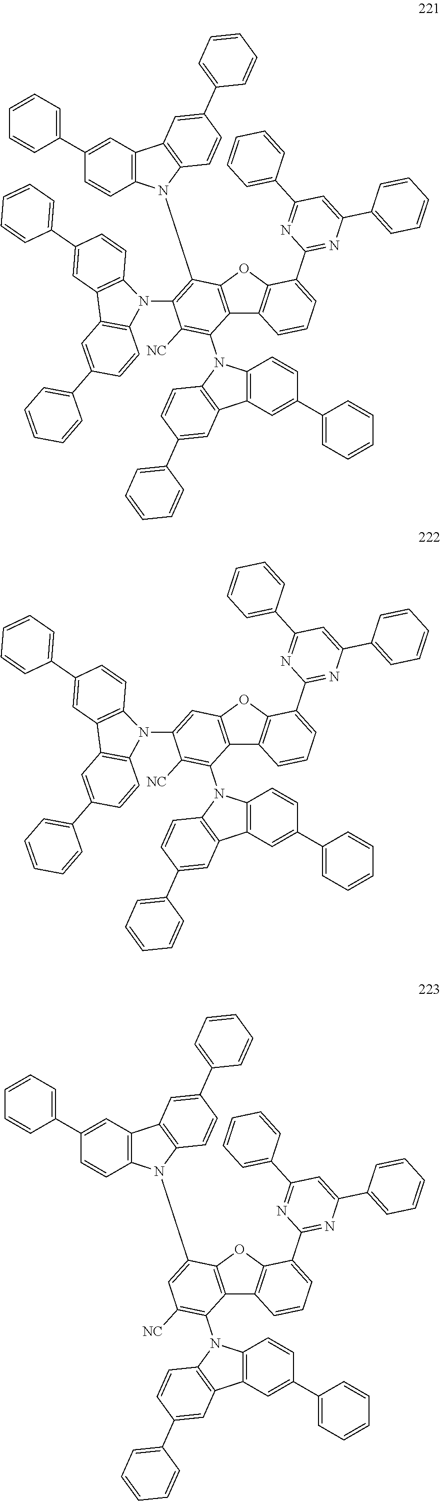 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4nc(-c5ccccc5)cc(-c5ccccc5)n4)cccc3c2c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2oc3c(-c4nc(-c5ccccc5)cc(-c5ccccc5)n4)cccc3c2c1-n1c2ccc(-c3ccccc3)cc2c2cc(-c3ccccc3)ccc21 ; N#Cc1cc(-n2c3ccc(-c4ccccc4)cc3c3cc(-c4ccccc4)ccc32)c2oc3c(-c4nc(-c5ccccc5)cc(-c5ccccc5)n4)cccc3c2c1-n1c2ccc(-c3ccccc3)cc2c2cc(-c3ccccc3)ccc21]

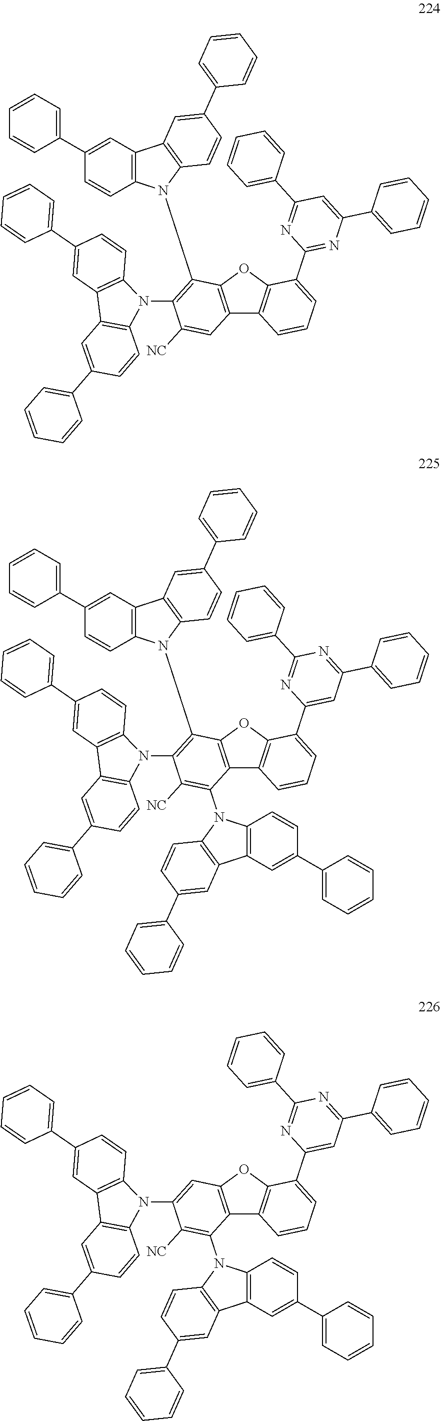 [SMILES: N#Cc1cc2c(oc3c(-c4nc(-c5ccccc5)cc(-c5ccccc5)n4)cccc32)c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4cc(-c5ccccc5)nc(-c5ccccc5)n4)cccc3c2c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2oc3c(-c4cc(-c5ccccc5)nc(-c5ccccc5)n4)cccc3c2c1-n1c2ccc(-c3ccccc3)cc2c2cc(-c3ccccc3)ccc21]

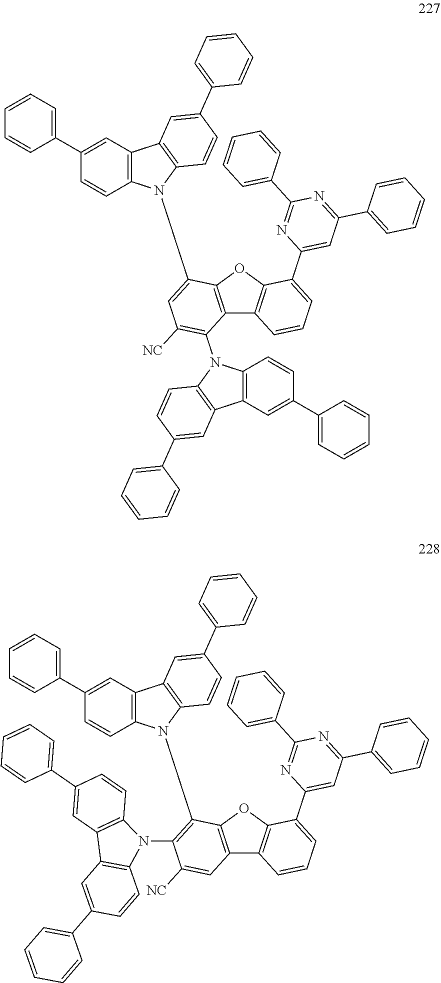 [SMILES: N#Cc1cc(-n2c3ccc(-c4ccccc4)cc3c3cc(-c4ccccc4)ccc32)c2oc3c(-c4cc(-c5ccccc5)nc(-c5ccccc5)n4)cccc3c2c1-n1c2ccc(-c3ccccc3)cc2c2cc(-c3ccccc3)ccc21 ; N#Cc1cc2c(oc3c(-c4cc(-c5ccccc5)nc(-c5ccccc5)n4)cccc32)c(-n2c3ccc(-c4ccccc4)cc3c3cc(-c4ccccc4)ccc32)c1-n1c2ccc(-c3ccccc3)cc2c2cc(-c3ccccc3)ccc21]

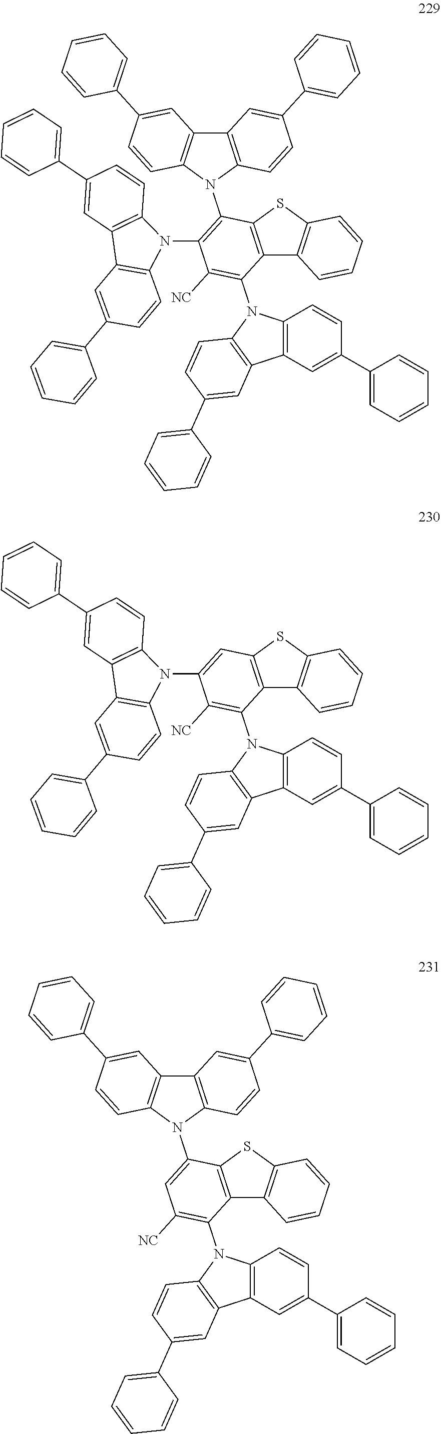 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2sc3ccccc3c2c1-n1c2ccc(-c3ccccc3)cc2c2cc(-c3ccccc3)ccc21 ; N#Cc1c(-n2c3ccc(-c4ccccc4)cc3c3cc(-c4ccccc4)ccc32)cc2sc3ccccc3c2c1-n1c2ccc(-c3ccccc3)cc2c2cc(-c3ccccc3)ccc21 ; N#Cc1cc(-n2c3ccc(-c4ccccc4)cc3c3cc(-c4ccccc4)ccc32)c2sc3ccccc3c2c1-n1c2ccc(-c3ccccc3)cc2c2cc(-c3ccccc3)ccc21]

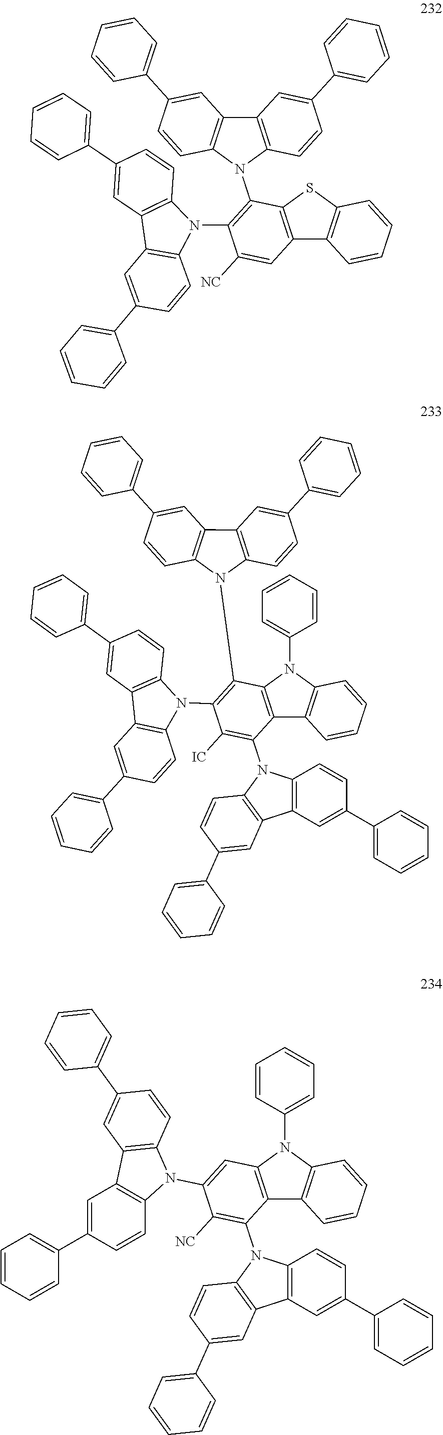

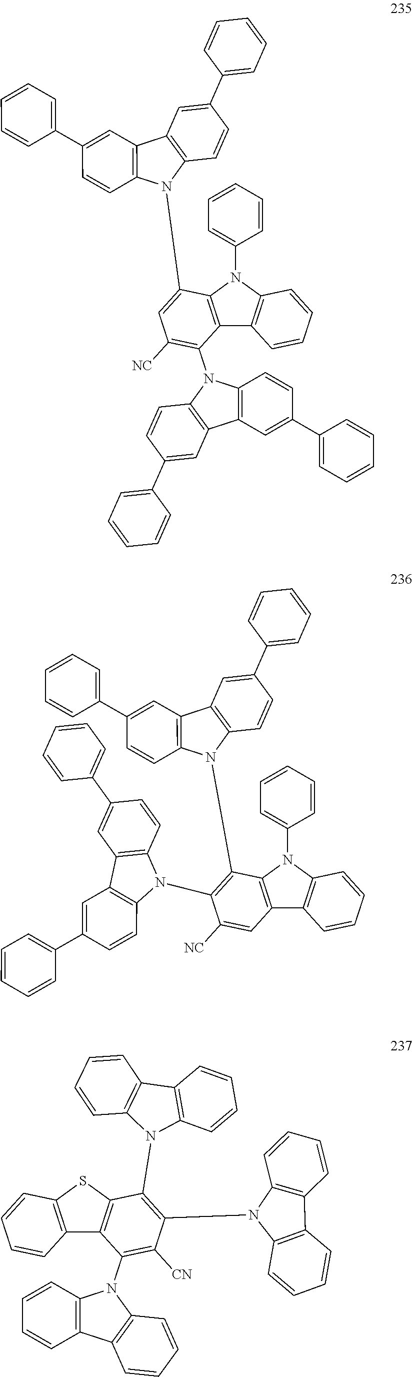 [SMILES: N#Cc1cc(-n2c3ccc(-c4ccccc4)cc3c3cc(-c4ccccc4)ccc32)c2c(c1-n1c3ccc(-c4ccccc4)cc3c3cc(-c4ccccc4)ccc31)c1ccccc1n2-c1ccccc1 ; N#Cc1cc2c3ccccc3n(-c3ccccc3)c2c(-n2c3ccc(-c4ccccc4)cc3c3cc(-c4ccccc4)ccc32)c1-n1c2ccc(-c3ccccc3)cc2c2cc(-c3ccccc3)ccc21 ; N#Cc1c(-n2c3ccccc3c3ccccc32)c(-n2c3ccccc3c3ccccc32)c2sc3ccccc3c2c1-n1c2ccccc2c2ccccc21]

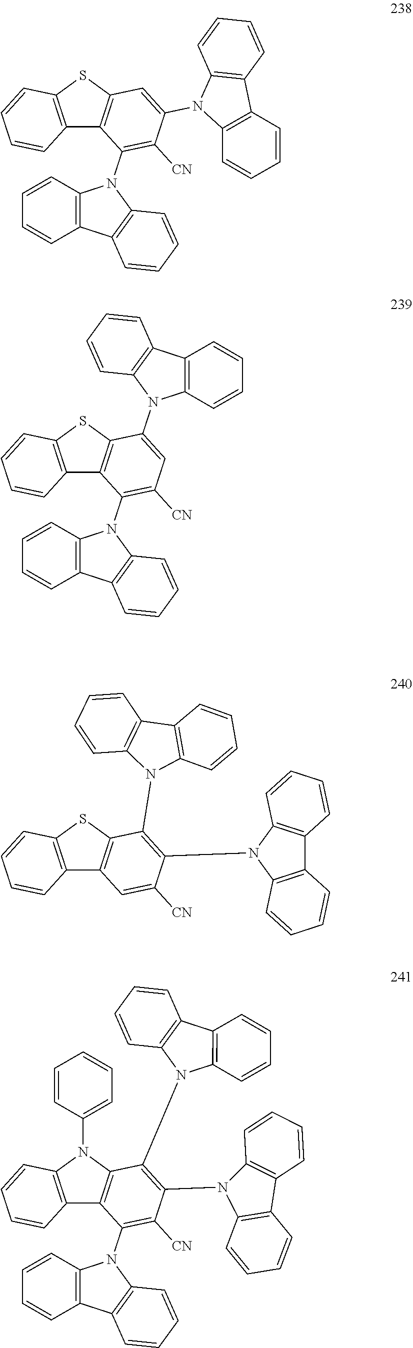 [SMILES: N#Cc1c(-n2c3ccccc3c3ccccc32)cc2sc3ccccc3c2c1-n1c2ccccc2c2ccccc21 ; N#Cc1cc(-n2c3ccccc3c3ccccc32)c2sc3ccccc3c2c1-n1c2ccccc2c2ccccc21 ; N#Cc1cc2c(sc3ccccc32)c(-n2c3ccccc3c3ccccc32)c1-n1c2ccccc2c2ccccc21 ; N#Cc1c(-n2c3ccccc3c3ccccc32)c(-n2c3ccccc3c3ccccc32)c2c(c1-n1c3ccccc3c3ccccc31)c1ccccc1n2-c1ccccc1]

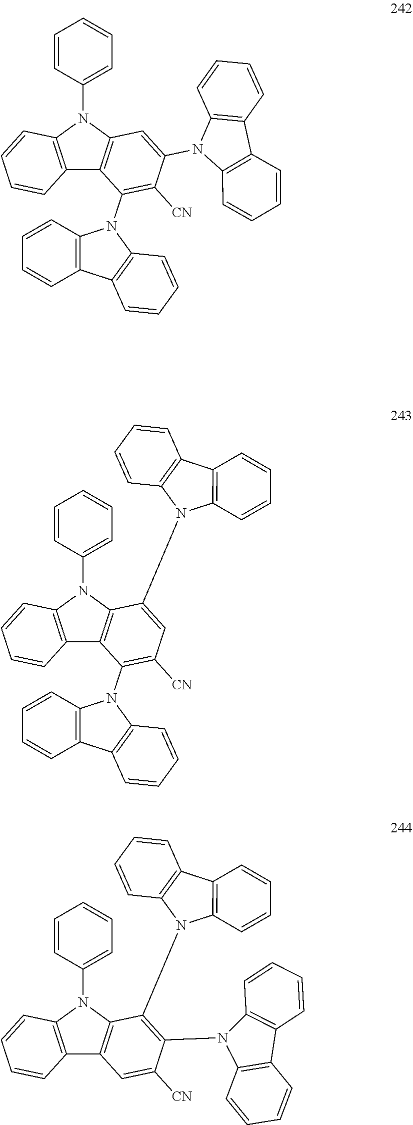 [SMILES: N#Cc1c(-n2c3ccccc3c3ccccc32)cc2c(c1-n1c3ccccc3c3ccccc31)c1ccccc1n2-c1ccccc1 ; N#Cc1cc(-n2c3ccccc3c3ccccc32)c2c(c1-n1c3ccccc3c3ccccc31)c1ccccc1n2-c1ccccc1 ; N#Cc1cc2c3ccccc3n(-c3ccccc3)c2c(-n2c3ccccc3c3ccccc32)c1-n1c2ccccc2c2ccccc21]

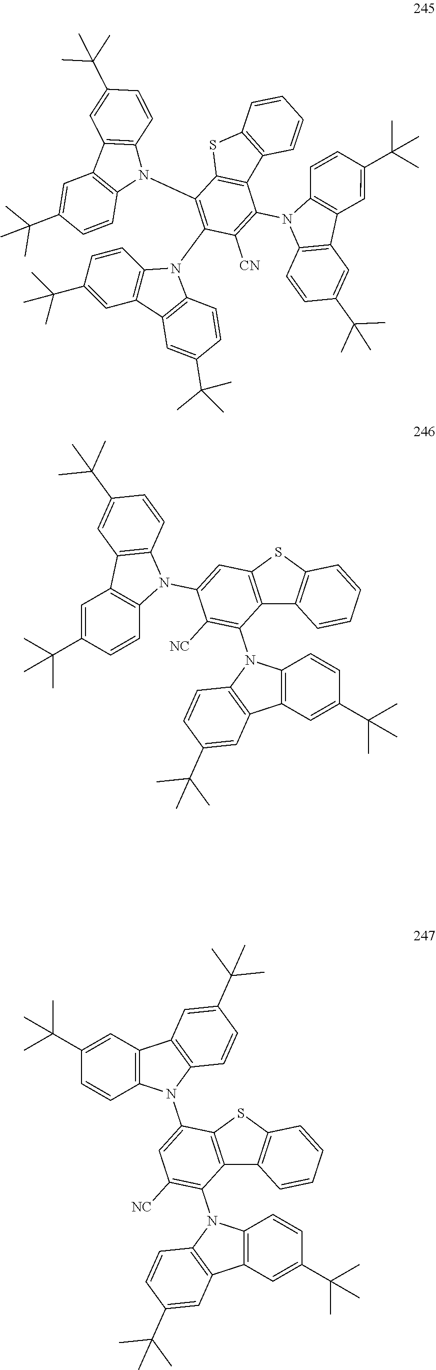

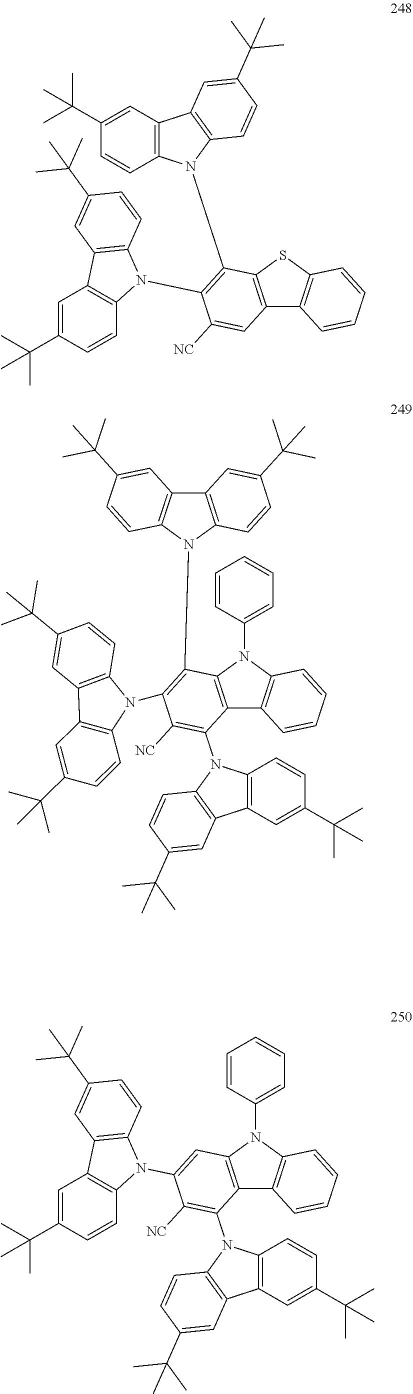

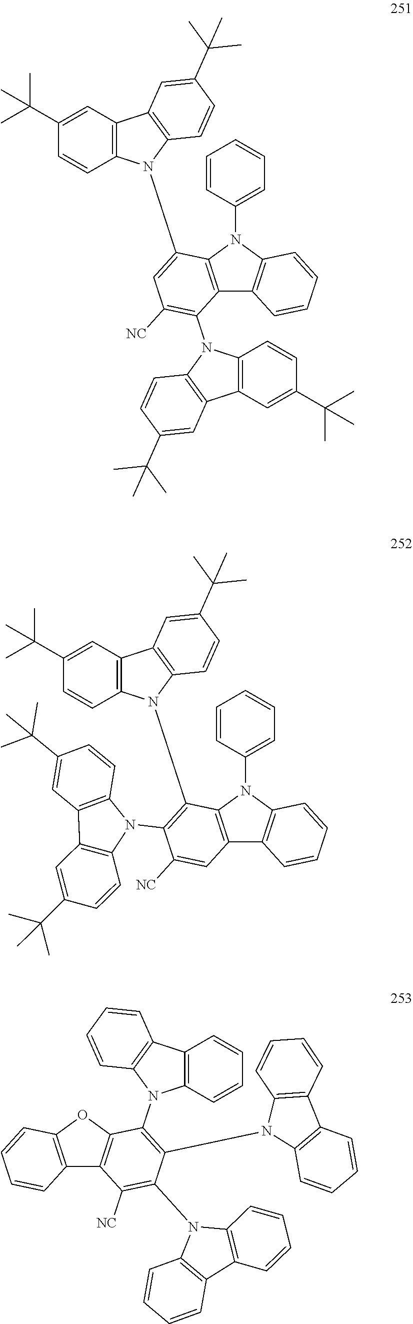

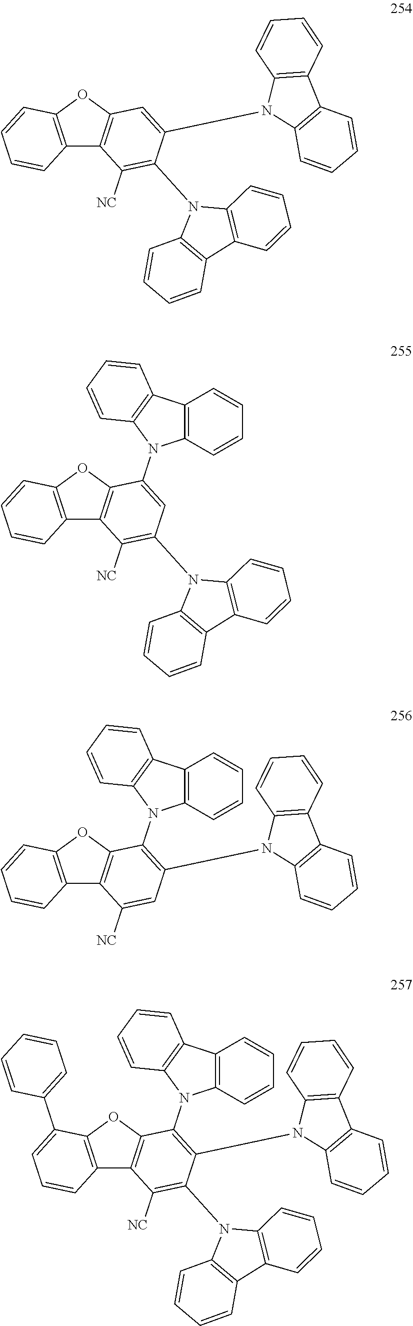 [SMILES: N#Cc1c(-n2c3ccccc3c3ccccc32)c(-n2c3ccccc3c3ccccc32)cc2oc3ccccc3c12 ; N#Cc1c(-n2c3ccccc3c3ccccc32)cc(-n2c3ccccc3c3ccccc32)c2oc3ccccc3c12 ; N#Cc1cc(-n2c3ccccc3c3ccccc32)c(-n2c3ccccc3c3ccccc32)c2oc3ccccc3c12 ; N#Cc1c(-n2c3ccccc3c3ccccc32)c(-n2c3ccccc3c3ccccc32)c(-n2c3ccccc3c3ccccc32)c2oc3c(-c4ccccc4)cccc3c12]

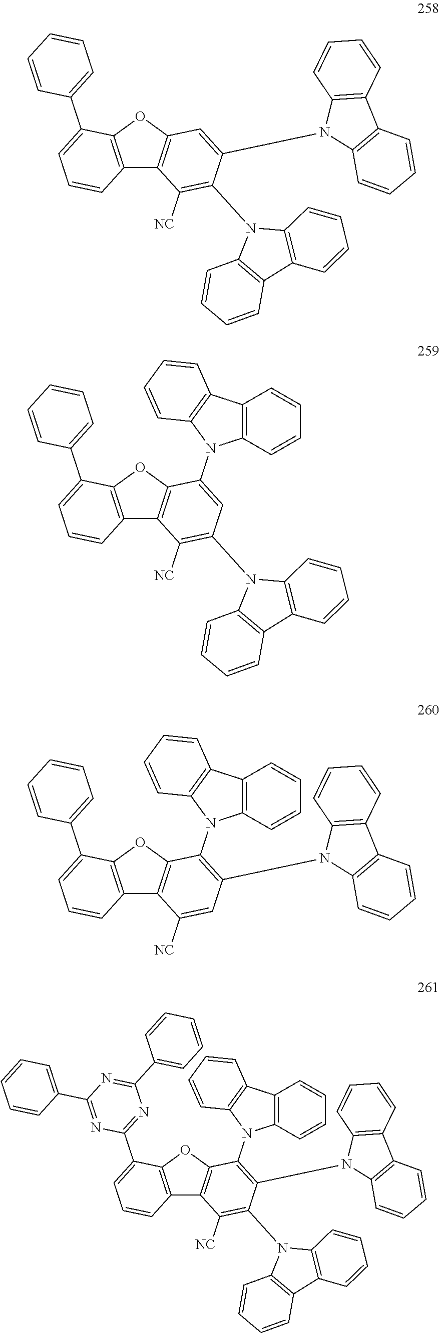 [SMILES: N#Cc1c(-n2c3ccccc3c3ccccc32)c(-n2c3ccccc3c3ccccc32)cc2oc3c(-c4ccccc4)cccc3c12 ; N#Cc1c(-n2c3ccccc3c3ccccc32)cc(-n2c3ccccc3c3ccccc32)c2oc3c(-c4ccccc4)cccc3c12 ; N#Cc1cc(-n2c3ccccc3c3ccccc32)c(-n2c3ccccc3c3ccccc32)c2oc3c(-c4ccccc4)cccc3c12 ; N#Cc1c(-n2c3ccccc3c3ccccc32)c(-n2c3ccccc3c3ccccc32)c(-n2c3ccccc3c3ccccc32)c2oc3c(-c4nc(-c5ccccc5)nc(-c5ccccc5)n4)cccc3c12]

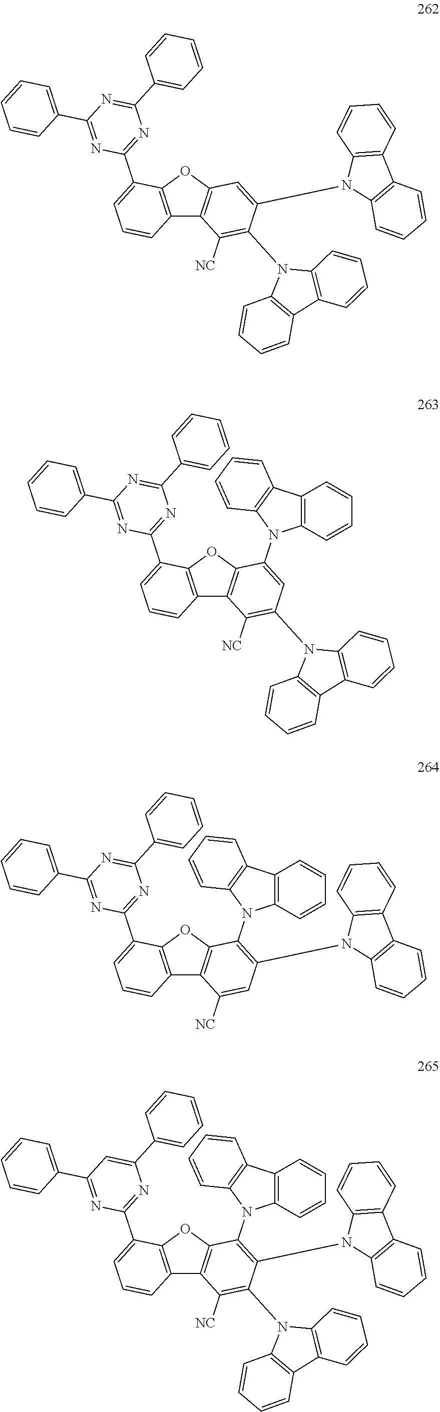 [SMILES: N#Cc1c(-n2c3ccccc3c3ccccc32)c(-n2c3ccccc3c3ccccc32)cc2oc3c(-c4nc(-c5ccccc5)nc(-c5ccccc5)n4)cccc3c12 ; N#Cc1c(-n2c3ccccc3c3ccccc32)cc(-n2c3ccccc3c3ccccc32)c2oc3c(-c4nc(-c5ccccc5)nc(-c5ccccc5)n4)cccc3c12 ; N#Cc1cc(-n2c3ccccc3c3ccccc32)c(-n2c3ccccc3c3ccccc32)c2oc3c(-c4nc(-c5ccccc5)nc(-c5ccccc5)n4)cccc3c12 ; N#Cc1c(-n2c3ccccc3c3ccccc32)c(-n2c3ccccc3c3ccccc32)c(-n2c3ccccc3c3ccccc32)c2oc3c(-c4nc(-c5ccccc5)cc(-c5ccccc5)n4)cccc3c12]

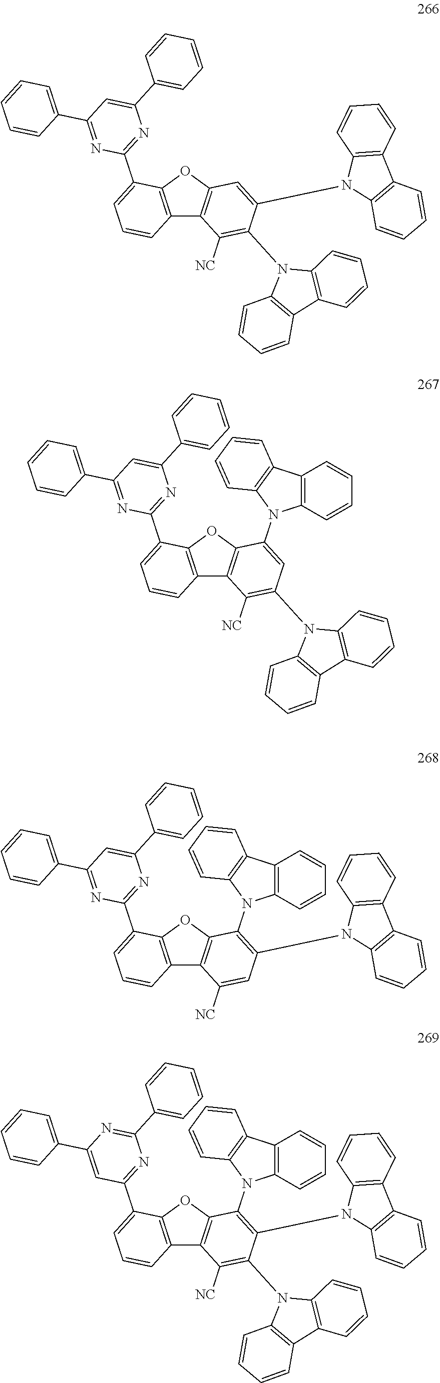 [SMILES: N#Cc1c(-n2c3ccccc3c3ccccc32)c(-n2c3ccccc3c3ccccc32)cc2oc3c(-c4nc(-c5ccccc5)cc(-c5ccccc5)n4)cccc3c12 ; N#Cc1c(-n2c3ccccc3c3ccccc32)cc(-n2c3ccccc3c3ccccc32)c2oc3c(-c4nc(-c5ccccc5)cc(-c5ccccc5)n4)cccc3c12 ; N#Cc1cc(-n2c3ccccc3c3ccccc32)c(-n2c3ccccc3c3ccccc32)c2oc3c(-c4nc(-c5ccccc5)cc(-c5ccccc5)n4)cccc3c12 ; N#Cc1c(-n2c3ccccc3c3ccccc32)c(-n2c3ccccc3c3ccccc32)c(-n2c3ccccc3c3ccccc32)c2oc3c(-c4cc(-c5ccccc5)nc(-c5ccccc5)n4)cccc3c12]

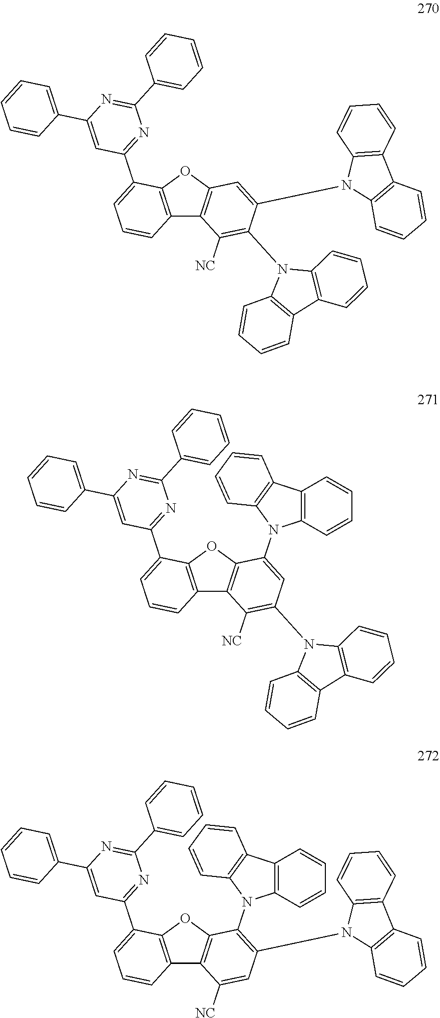 [SMILES: N#Cc1c(-n2c3ccccc3c3ccccc32)c(-n2c3ccccc3c3ccccc32)cc2oc3c(-c4cc(-c5ccccc5)nc(-c5ccccc5)n4)cccc3c12 ; N#Cc1c(-n2c3ccccc3c3ccccc32)cc(-n2c3ccccc3c3ccccc32)c2oc3c(-c4cc(-c5ccccc5)nc(-c5ccccc5)n4)cccc3c12 ; N#Cc1cc(-n2c3ccccc3c3ccccc32)c(-n2c3ccccc3c3ccccc32)c2oc3c(-c4cc(-c5ccccc5)nc(-c5ccccc5)n4)cccc3c12]

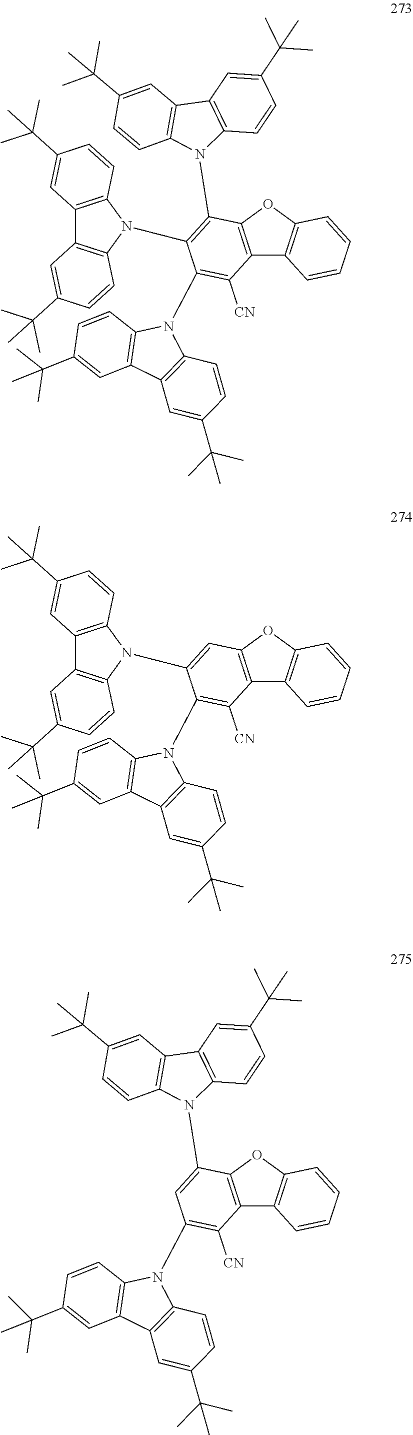 [SMILES: CC(C)(C)c1ccc2c(c1)c1cc(C(C)(C)C)ccc1n2-c1c(-n2c3ccc(C(C)(C)C)cc3c3cc(C(C)(C)C)ccc32)c(C#N)c2c(oc3ccccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1cc2oc3ccccc3c2c(C#N)c1-n1c2ccc(C(C)(C)C)cc2c2cc(C(C)(C)C)ccc21 ; CC(C)(C)c1ccc2c(c1)c1cc(C(C)(C)C)ccc1n2-c1cc(-n2c3ccc(C(C)(C)C)cc3c3cc(C(C)(C)C)ccc32)c2oc3ccccc3c2c1C#N]

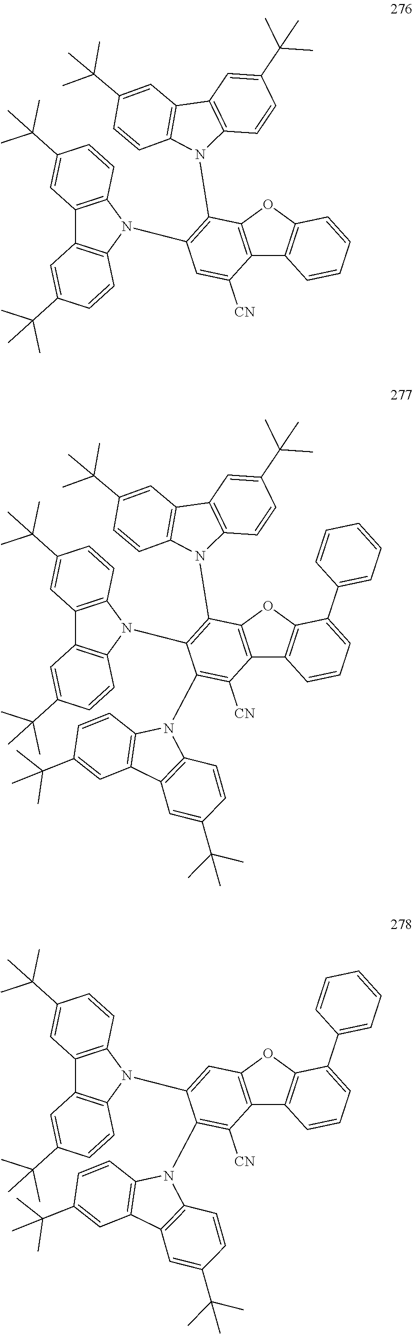 [SMILES: CC(C)(C)c1ccc2c(c1)c1cc(C(C)(C)C)ccc1n2-c1cc(C#N)c2c(oc3ccccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1c(-n2c3ccc(C(C)(C)C)cc3c3cc(C(C)(C)C)ccc32)c(C#N)c2c(oc3c(-c4ccccc4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1cc2oc3c(-c4ccccc4)cccc3c2c(C#N)c1-n1c2ccc(C(C)(C)C)cc2c2cc(C(C)(C)C)ccc21]

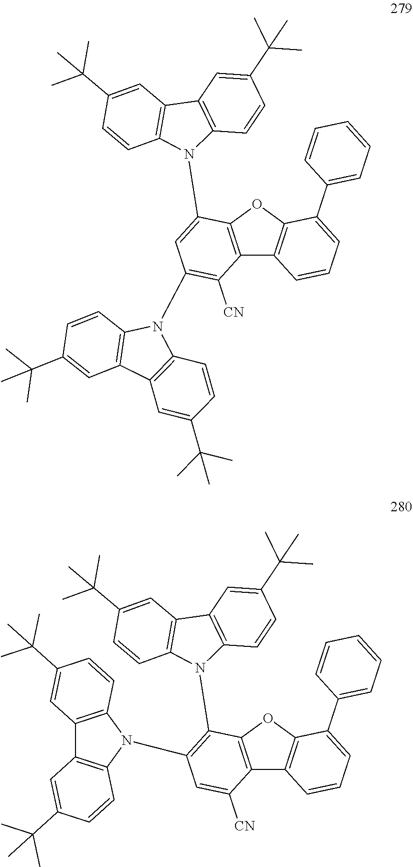 [SMILES: CC(C)(C)c1ccc2c(c1)c1cc(C(C)(C)C)ccc1n2-c1cc(-n2c3ccc(C(C)(C)C)cc3c3cc(C(C)(C)C)ccc32)c2oc3c(-c4ccccc4)cccc3c2c1C#N ; CC(C)(C)c1ccc2c(c1)c1cc(C(C)(C)C)ccc1n2-c1cc(C#N)c2c(oc3c(-c4ccccc4)cccc32)c1-n1c2ccc(C(C)(C)C)cc2c2cc(C(C)(C)C)ccc21]

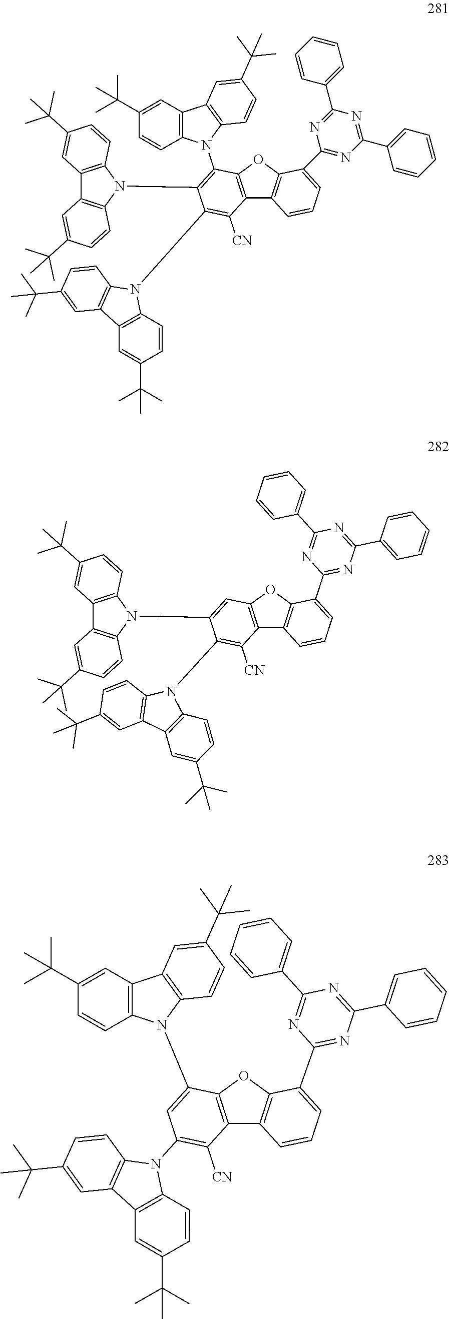 [SMILES: CC(C)(C)c1ccc2c(c1)c1cc(C(C)(C)C)ccc1n2-c1c(-n2c3ccc(C(C)(C)C)cc3c3cc(C(C)(C)C)ccc32)c(C#N)c2c(oc3c(-c4nc(-c5ccccc5)nc(-c5ccccc5)n4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1cc2oc3c(-c4nc(-c5ccccc5)nc(-c5ccccc5)n4)cccc3c2c(C#N)c1-n1c2ccc(C(C)(C)C)cc2c2cc(C(C)(C)C)ccc21 ; CC(C)(C)c1ccc2c(c1)c1cc(C(C)(C)C)ccc1n2-c1cc(-n2c3ccc(C(C)(C)C)cc3c3cc(C(C)(C)C)ccc32)c2oc3c(-c4nc(-c5ccccc5)nc(-c5ccccc5)n4)cccc3c2c1C#N]

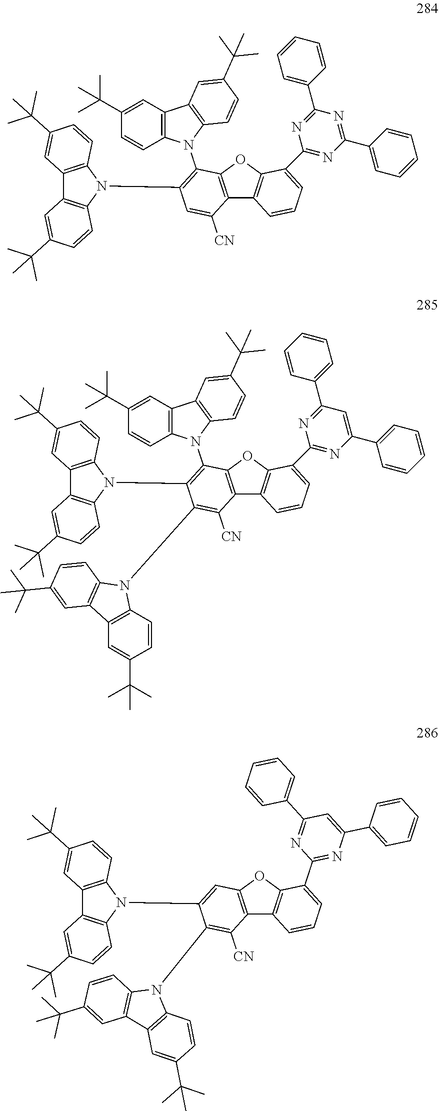 [SMILES: CC(C)(C)c1ccc2c(c1)c1cc(C(C)(C)C)ccc1n2-c1cc(C#N)c2c(oc3c(-c4nc(-c5ccccc5)nc(-c5ccccc5)n4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1c(-n2c3ccc(C(C)(C)C)cc3c3cc(C(C)(C)C)ccc32)c(C#N)c2c(oc3c(-c4nc(-c5ccccc5)cc(-c5ccccc5)n4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1cc2oc3c(-c4nc(-c5ccccc5)cc(-c5ccccc5)n4)cccc3c2c(C#N)c1-n1c2ccc(C(C)(C)C)cc2c2cc(C(C)(C)C)ccc21]

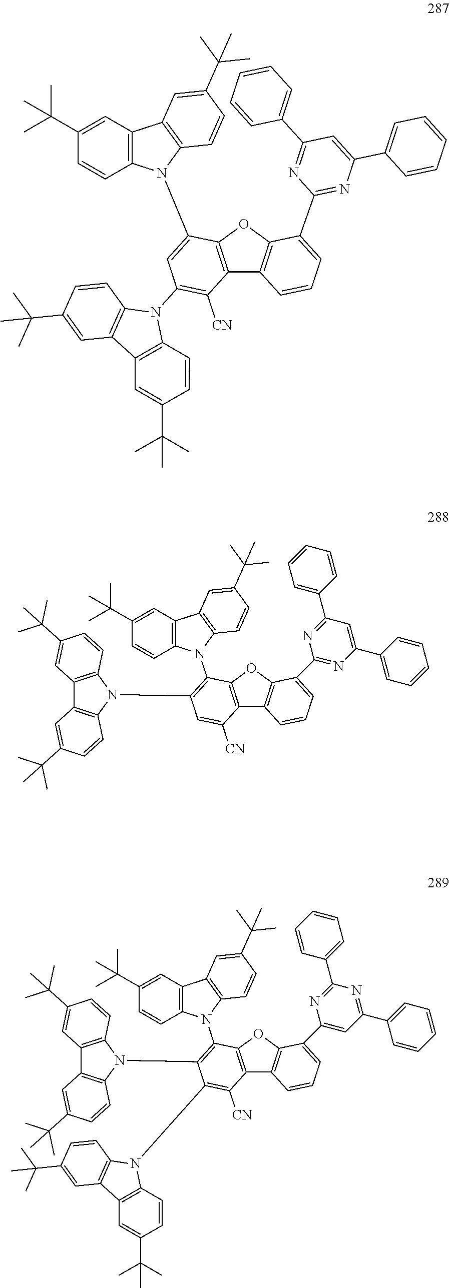 [SMILES: CC(C)(C)c1ccc2c(c1)c1cc(C(C)(C)C)ccc1n2-c1cc(-n2c3ccc(C(C)(C)C)cc3c3cc(C(C)(C)C)ccc32)c2oc3c(-c4nc(-c5ccccc5)cc(-c5ccccc5)n4)cccc3c2c1C#N ; CC(C)(C)c1ccc2c(c1)c1cc(C(C)(C)C)ccc1n2-c1cc(C#N)c2c(oc3c(-c4nc(-c5ccccc5)cc(-c5ccccc5)n4)cccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1c(-n2c3ccc(C(C)(C)C)cc3c3cc(C(C)(C)C)ccc32)c(C#N)c2c(oc3c(-c4cc(-c5ccccc5)nc(-c5ccccc5)n4)cccc32)c1-n1c2ccc(C(C)(C)C)cc2c2cc(C(C)(C)C)ccc21]

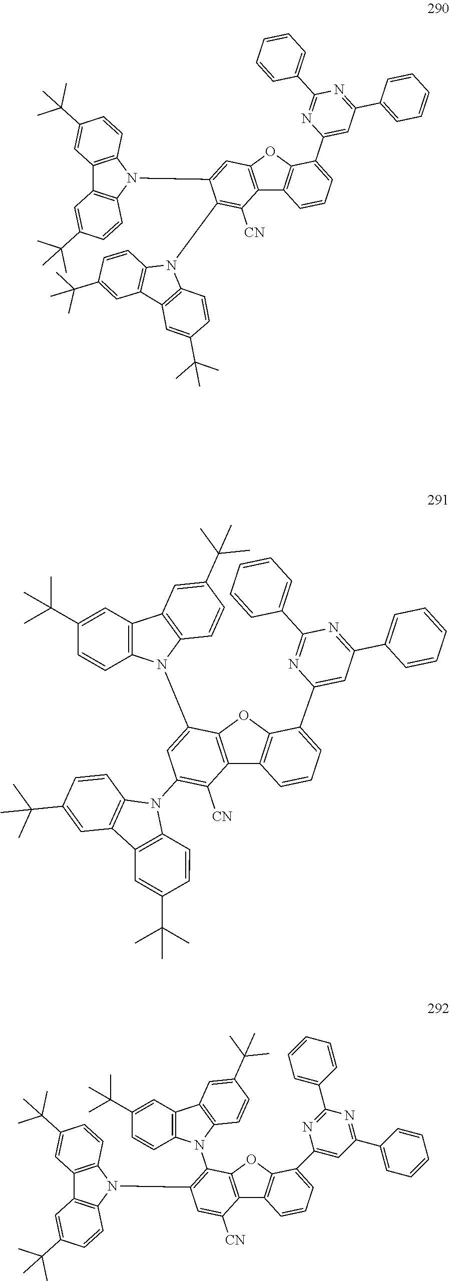 [SMILES: CC(C)(C)c1ccc2c(c1)c1cc(C(C)(C)C)ccc1n2-c1cc2oc3c(-c4cc(-c5ccccc5)nc(-c5ccccc5)n4)cccc3c2c(C#N)c1-n1c2ccc(C(C)(C)C)cc2c2cc(C(C)(C)C)ccc21 ; CC(C)(C)c1ccc2c(c1)c1cc(C(C)(C)C)ccc1n2-c1cc(-n2c3ccc(C(C)(C)C)cc3c3cc(C(C)(C)C)ccc32)c2oc3c(-c4cc(-c5ccccc5)nc(-c5ccccc5)n4)cccc3c2c1C#N ; CC(C)(C)c1ccc2c(c1)c1cc(C(C)(C)C)ccc1n2-c1cc(C#N)c2c(oc3c(-c4cc(-c5ccccc5)nc(-c5ccccc5)n4)cccc32)c1-n1c2ccc(C(C)(C)C)cc2c2cc(C(C)(C)C)ccc21]

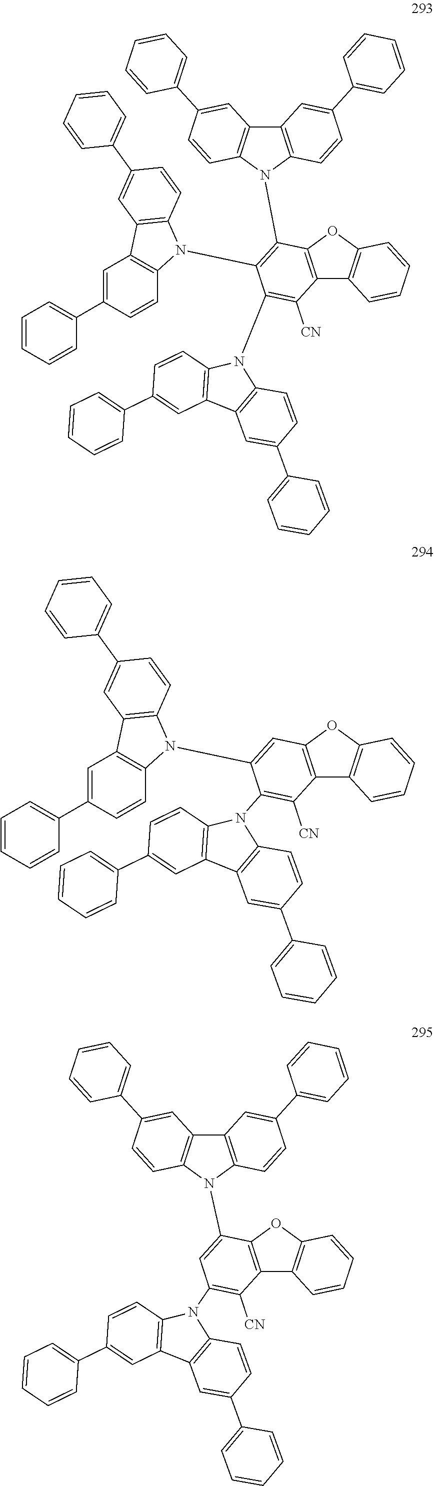

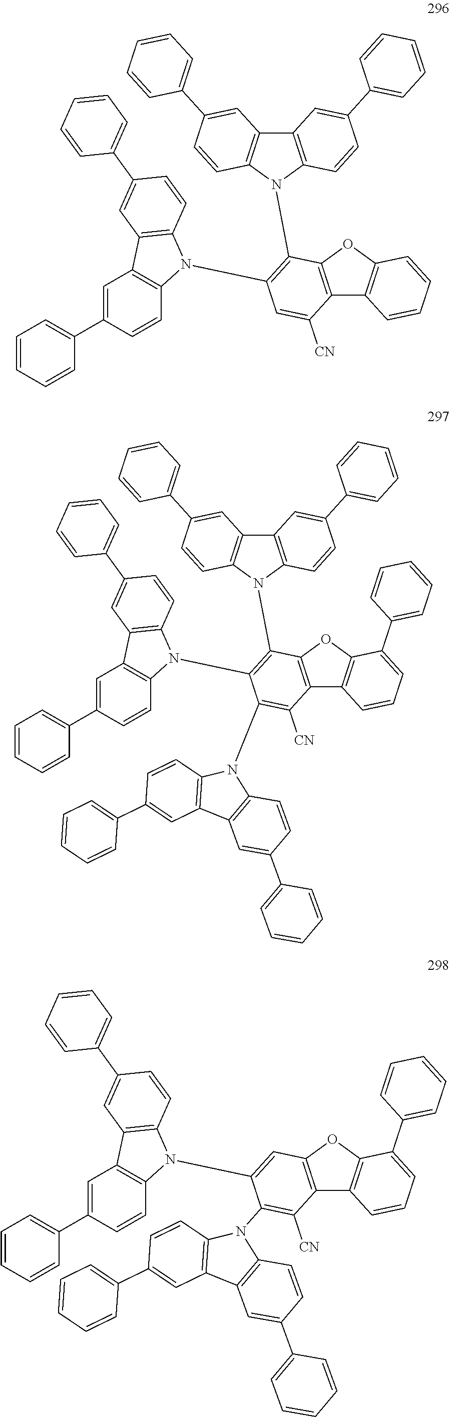 [SMILES: N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2oc3ccccc3c12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4ccccc4)cccc3c12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2oc3c(-c4ccccc4)cccc3c12]

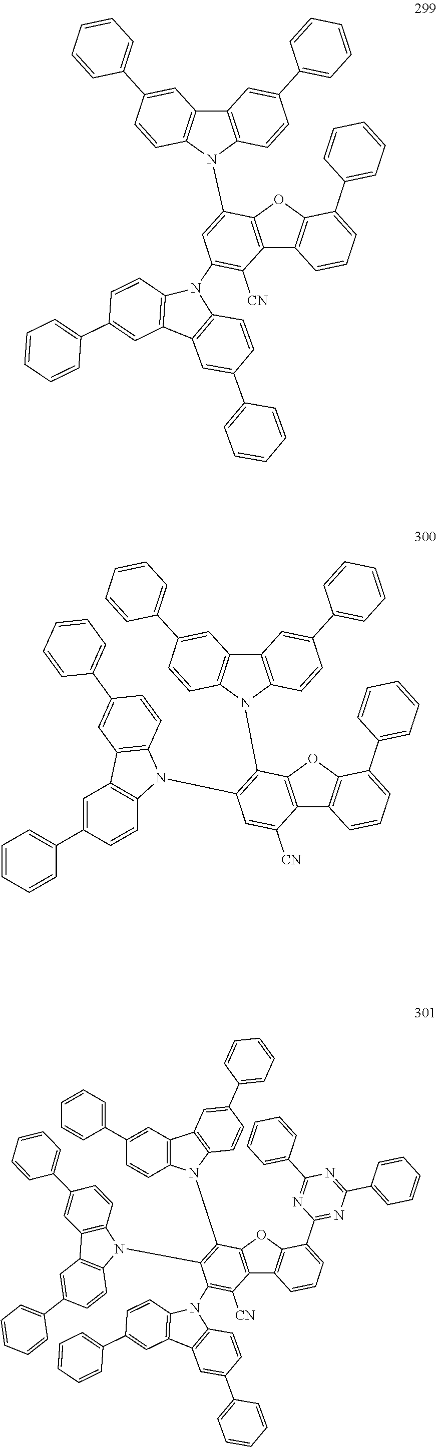 [SMILES: N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2oc3c(-c4ccccc4)cccc3c12 ; N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4ccccc4)cccc3c12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4nc(-c5ccccc5)nc(-c5ccccc5)n4)cccc3c12]

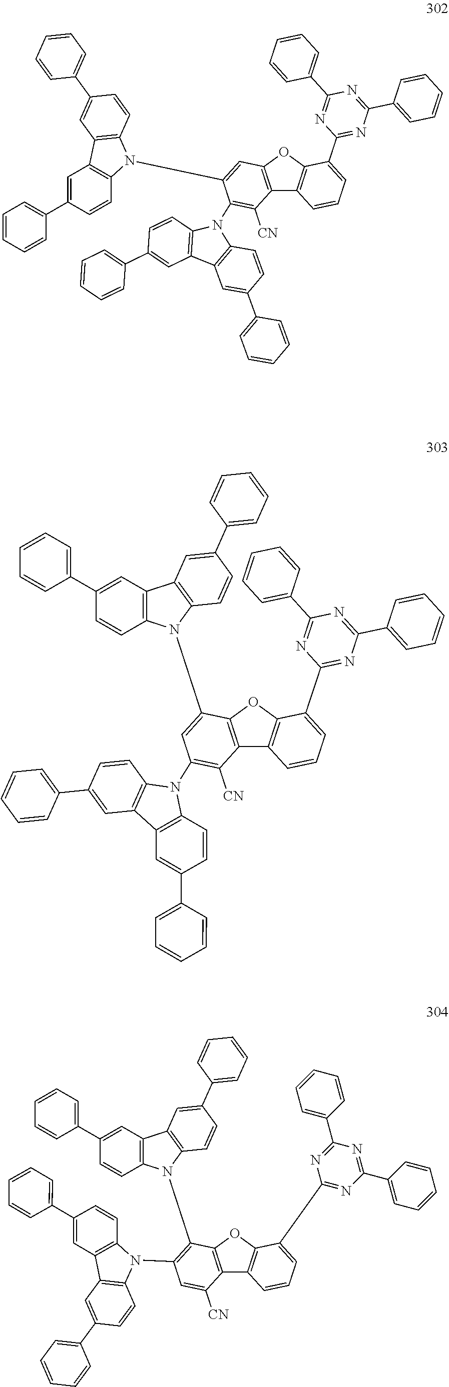 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2oc3c(-c4nc(-c5ccccc5)nc(-c5ccccc5)n4)cccc3c12 ; N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2oc3c(-c4nc(-c5ccccc5)nc(-c5ccccc5)n4)cccc3c12 ; N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4nc(-c5ccccc5)nc(-c5ccccc5)n4)cccc3c12]

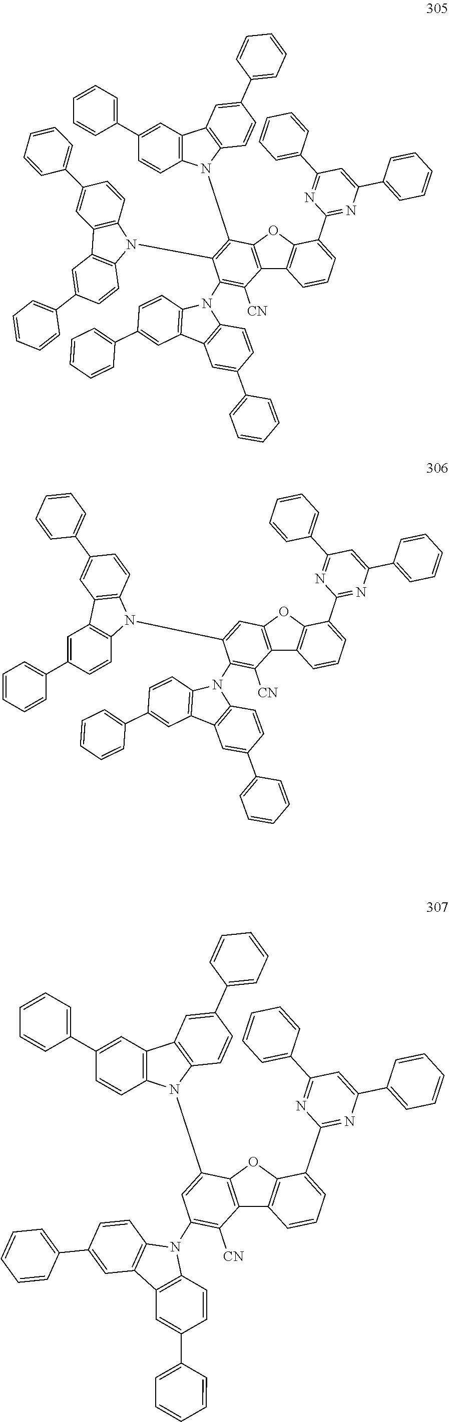 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4nc(-c5ccccc5)cc(-c5ccccc5)n4)cccc3c12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2oc3c(-c4nc(-c5ccccc5)cc(-c5ccccc5)n4)cccc3c12 ; N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2oc3c(-c4nc(-c5ccccc5)cc(-c5ccccc5)n4)cccc3c12]

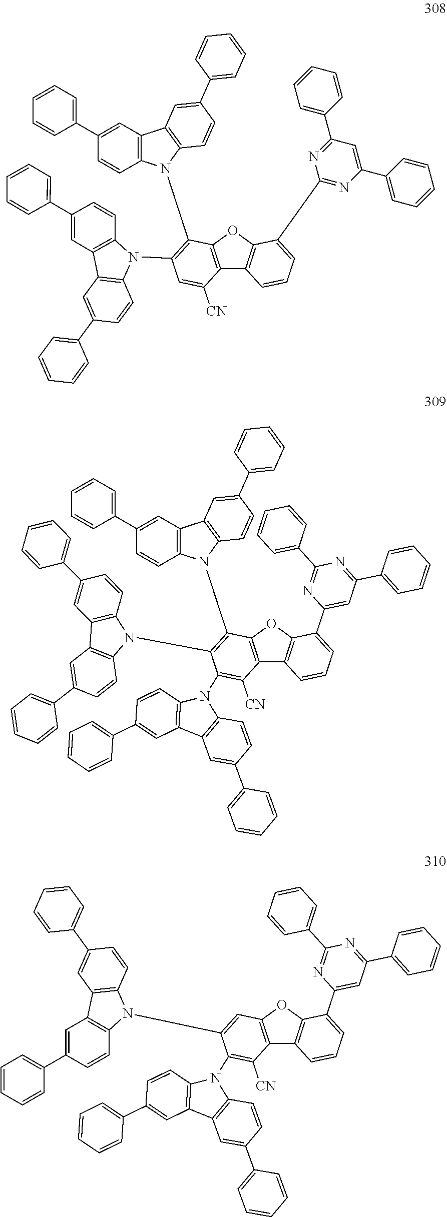 [SMILES: N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4nc(-c5ccccc5)cc(-c5ccccc5)n4)cccc3c12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4cc(-c5ccccc5)nc(-c5ccccc5)n4)cccc3c12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)cc2oc3c(-c4cc(-c5ccccc5)nc(-c5ccccc5)n4)cccc3c12]

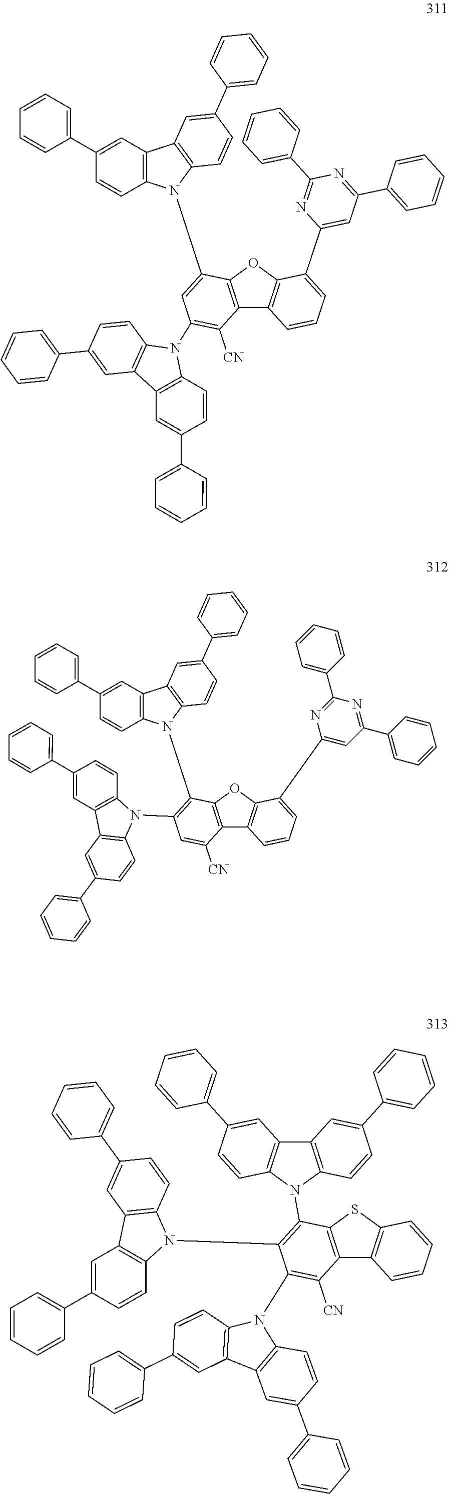 [SMILES: N#Cc1c(-n2c3ccc(-c4ccccc4)cc3c3cc(-c4ccccc4)ccc32)cc(-n2c3ccc(-c4ccccc4)cc3c3cc(-c4ccccc4)ccc32)c2oc3c(-c4cc(-c5ccccc5)nc(-c5ccccc5)n4)cccc3c12 ; N#Cc1cc(-n2c3ccc(-c4ccccc4)cc3c3cc(-c4ccccc4)ccc32)c(-n2c3ccc(-c4ccccc4)cc3c3cc(-c4ccccc4)ccc32)c2oc3c(-c4cc(-c5ccccc5)nc(-c5ccccc5)n4)cccc3c12 ; N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(-n2c3ccc(-c4ccccc4)cc3c3cc(-c4ccccc4)ccc32)c2sc3ccccc3c12]

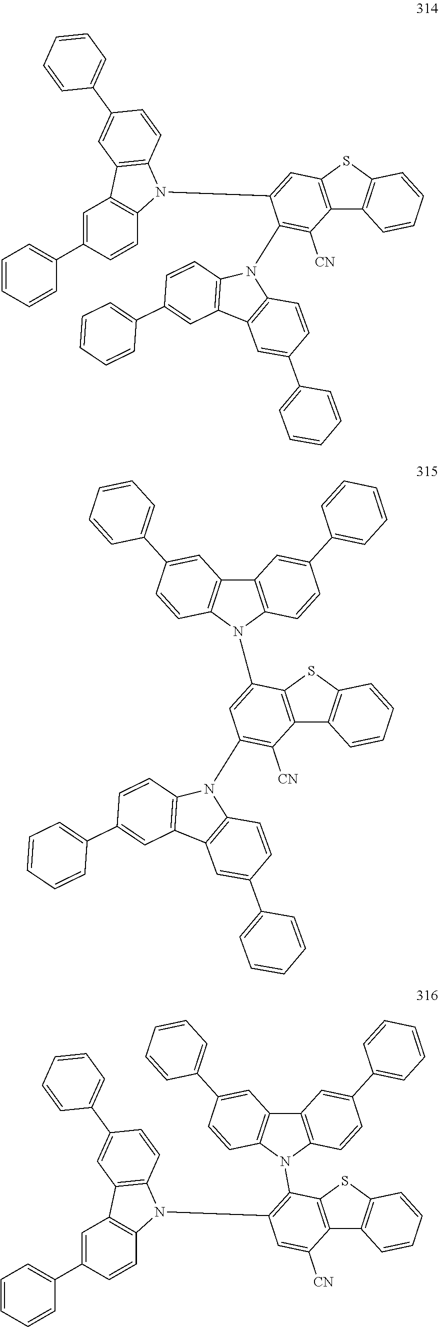

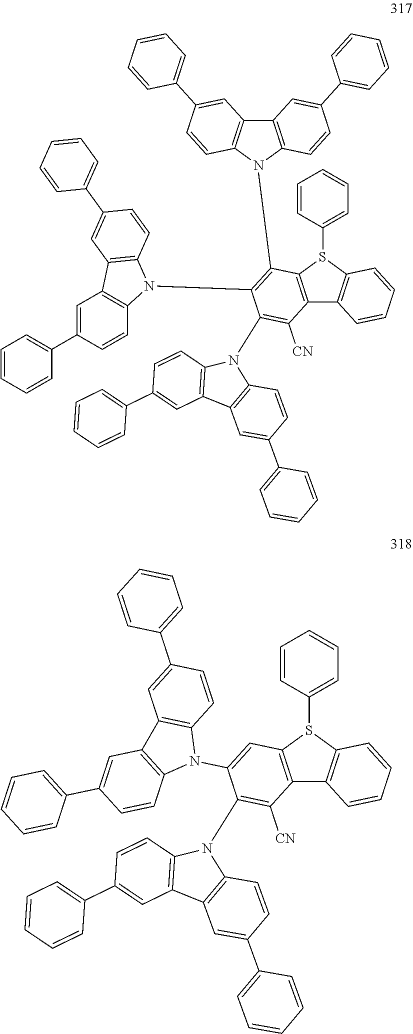

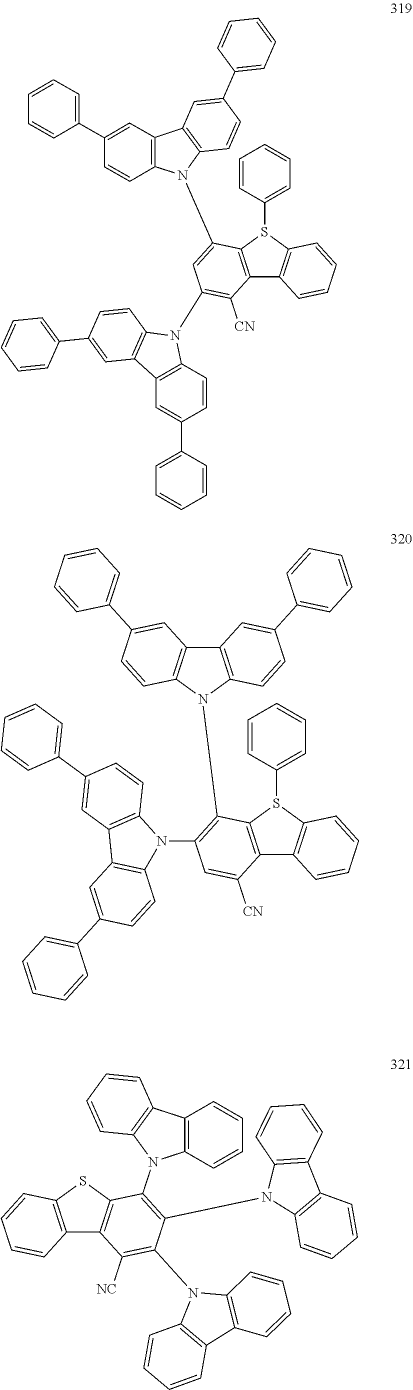

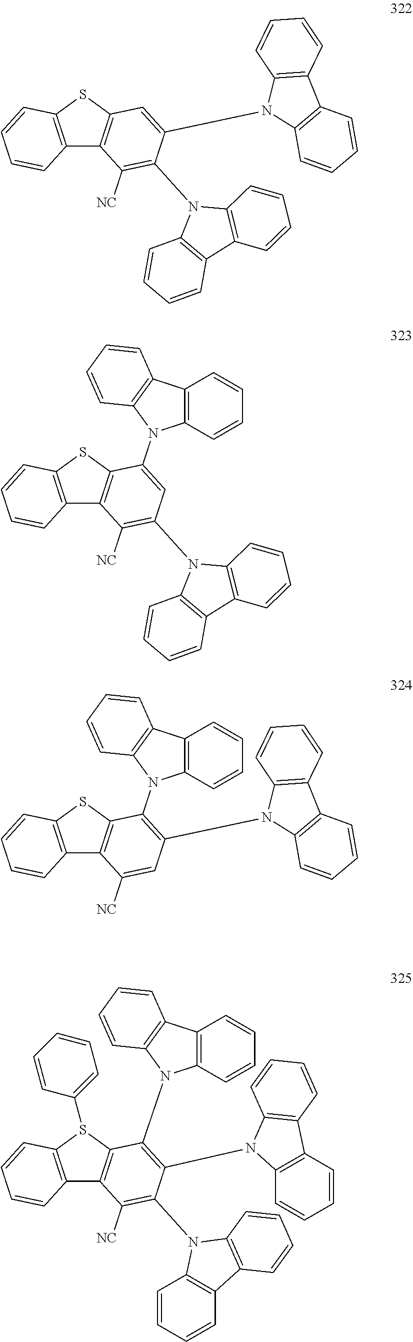

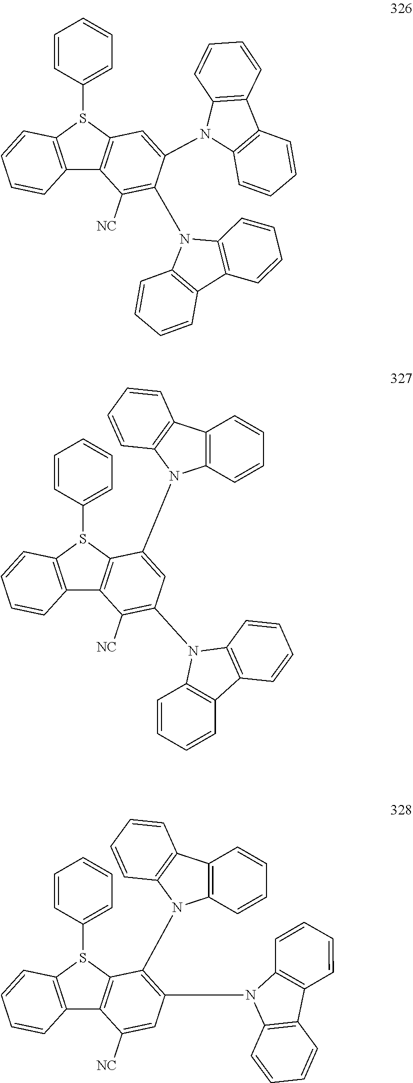

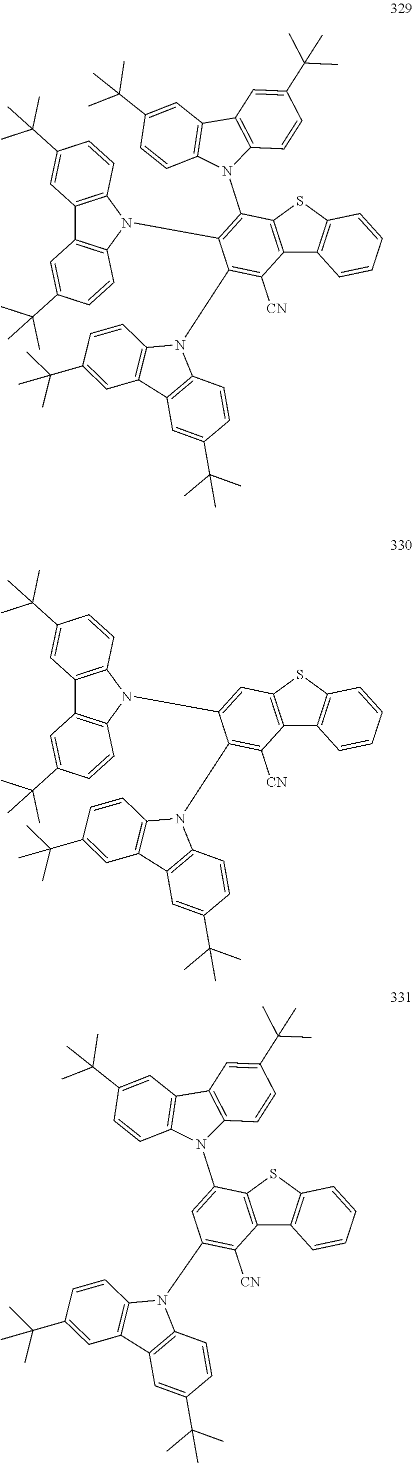 [SMILES: CC(C)(C)c1ccc2c(c1)c1cc(C(C)(C)C)ccc1n2-c1c(-n2c3ccc(C(C)(C)C)cc3c3cc(C(C)(C)C)ccc32)c(C#N)c2c(sc3ccccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1cc2sc3ccccc3c2c(C#N)c1-n1c2ccc(C(C)(C)C)cc2c2cc(C(C)(C)C)ccc21 ; CC(C)(C)c1ccc2c(c1)c1cc(C(C)(C)C)ccc1n2-c1cc(-n2c3ccc(C(C)(C)C)cc3c3cc(C(C)(C)C)ccc32)c2sc3ccccc3c2c1C#N]

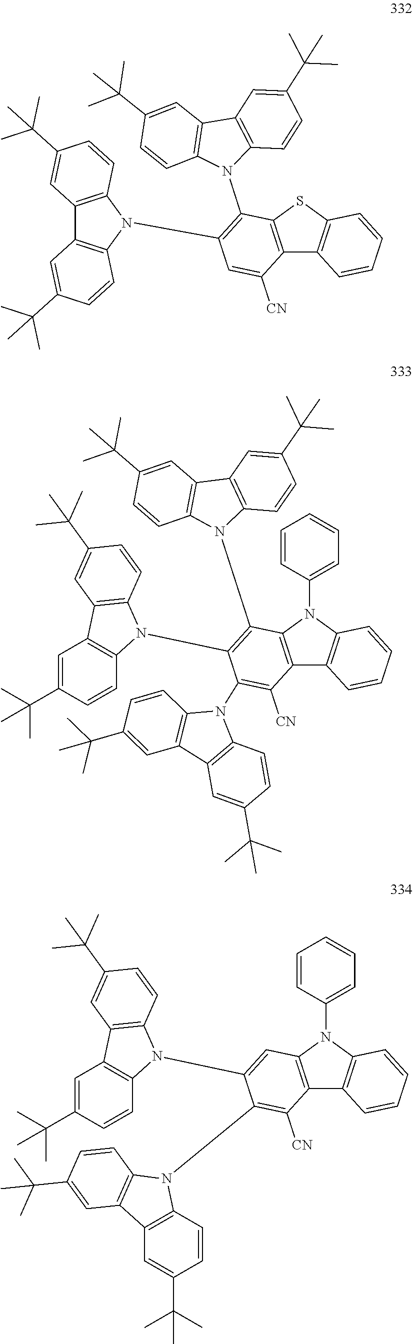 [SMILES: CC(C)(C)c1ccc2c(c1)c1cc(C(C)(C)C)ccc1n2-c1cc(C#N)c2c(sc3ccccc32)c1-n1c2ccc(C(C)(C)C)cc2c2cc(C(C)(C)C)ccc21 ; CC(C)(C)c1ccc2c(c1)c1cc(C(C)(C)C)ccc1n2-c1c(-n2c3ccc(C(C)(C)C)cc3c3cc(C(C)(C)C)ccc32)c(-n2c3ccc(C(C)(C)C)cc3c3cc(C(C)(C)C)ccc32)c2c(c1C#N)c1ccccc1n2-c1ccccc1 ; CC(C)(C)c1ccc2c(c1)c1cc(C(C)(C)C)ccc1n2-c1cc2c(c(C#N)c1-n1c3ccc(C(C)(C)C)cc3c3cc(C(C)(C)C)ccc31)c1ccccc1n2-c1ccccc1]

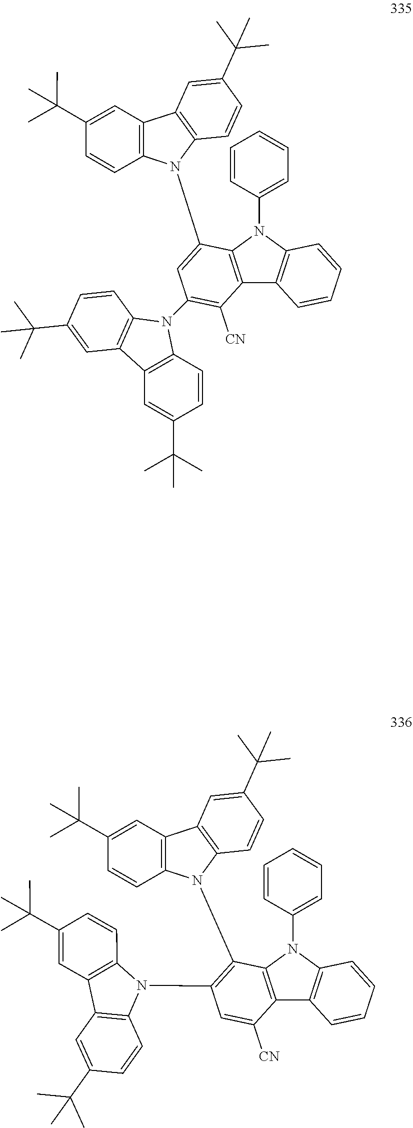 [SMILES: CC(C)(C)c1ccc2c(c1)c1cc(C(C)(C)C)ccc1n2-c1cc(-n2c3ccc(C(C)(C)C)cc3c3cc(C(C)(C)C)ccc32)c2c(c1C#N)c1ccccc1n2-c1ccccc1 ; CC(C)(C)c1ccc2c(c1)c1cc(C(C)(C)C)ccc1n2-c1cc(C#N)c2c3ccccc3n(-c3ccccc3)c2c1-n1c2ccc(C(C)(C)C)cc2c2cc(C(C)(C)C)ccc21]

13. An organic light-emitting device comprising:
a first electrode;
a second electrode; and
an organic layer between the first electrode and the second electrode and comprising an emission layer, wherein
the organic layer comprises the condensed-cyclic compound of claim 1.

14. The organic light-emitting device of claim 13, wherein
the first electrode is an anode,
the second electrode is a cathode,
the organic layer comprises a hole transport region between the first electrode and the emission layer and an electron transport region between the emission layer and the second electrode,
the hole transport region comprises a hole injection layer, a hole transport layer, an electron blocking layer, or a combination thereof, and
the electron transport region comprises a hole blocking layer, an electron transport layer, an electron injection layer, or a combination thereof.

15. The organic light-emitting device of claim 13, wherein
the emission layer comprises the condensed-cyclic compound of claim 1.

16. The organic light-emitting device of claim 15, wherein
a ratio of a fluorescent emission component to total emission components emitted from the emission layer is 90% or more.

17. The organic light-emitting device of claim 15, wherein
the condensed-cyclic compound is a fluorescent emitter, and
a ratio of an emission component emitted from the condensed-cyclic compound to total emission components emitted from the emission layer is 80% or more.

18. The organic light-emitting device of claim 17, wherein
the emission layer consists of the condensed-cyclic compound alone; or
the emission layer further comprises a host.

19. The organic light-emitting device of claim 15, wherein
the emission layer comprises a host and a dopant,
the host comprises the condensed-cyclic compound,
an amount of the host is greater than an amount of the dopant, and
a ratio of an emission component of the dopant to total emission components emitted from the emission layer is 80% or more.

20. The organic light-emitting device of claim 15, wherein
the emission layer comprises a host, an auxiliary dopant, and a dopant,
the auxiliary dopant comprises the condensed-cyclic compound, and
a ratio of the dopant to total emission components emitted from the emission layer is 80% or more.
